(19) United States
(12) Patent Application Publication
(10) Pub. No.: US 2024/0269469 A1
(43) Pub. Date: Aug. 15, 2024

(54) TREATMENT OF HYPERTENSION

(71) Applicant: Peter Forsell, Bouveret (CH)

(72) Inventor: Peter Forsell, Bouveret (CH)

(21) Appl. No.: 18/443,337

(22) Filed: Feb. 16, 2024

Related U.S. Application Data
(63) Continuation-in-part of application No. PCT/EP2022/073833, filed on Aug. 26, 2022.
(63) Continuation-in-part of application No. PCT/EP2023/053882, filed on Feb. 16, 2023.

(30) Foreign Application Priority Data
Aug. 30, 2021 (WO) PCT/EP2021/073893
Feb. 18, 2022 (SE) 2250209-0
Feb. 18, 2022 (SE) 2250220-7
Aug. 26, 2022 (WO) PCT/EP2022/073816

(51) Int. Cl. A61N 1/36; A61N 1/05; A61N 1/372

(52) U.S. Cl. CPC A61N 1/36117; A61N 1/0556; A61N 1/36139; A61N 1/36157; A61N 1/36171; A61N 1/36175; A61N 1/37247

(57) ABSTRACT

A system for affecting an effector response in a patient is disclosed. The system comprises a stimulation device configured to deliver, directly or indirectly a first stimulation signal to a sympathetic nerve innervating a first effector tissue of the patient and a second stimulation signal to a parasympathetic nerve innervating a second effector tissue, and a control unit configured to control an operation of the stimulation device such that the first stimulation signal stimulates an activity of the sympathetic nerve and the second stimulation signal inhibits an activity of the parasympathetic nerve or the first stimulation signal inhibits an activity of the sympathetic nerve and the second stimulation signal stimulates an activity of the parasympathetic nerve.

This application is a continuation in part of international Application PCT/EP2023/053882 filed on Feb. 16, 2023 which designates the US and claims priority to Swedish Application No 225020w9-0, filed Feb. 18, 2022 and to International Application No. PCT/EP2022/073816 filed Aug. 26, 2022, and a continuation in part of International Application No PCT/EP2022/073833, filed Aug. 26, 2022, which designates the US and claims priority to Swedish Application No 2250220-7, filed Feb. 18, 2022 and to International Application No. PCT/EP2021/073893 filed Aug. 30, 2021, the entire contents of each of which are hereby incorporated by reference.

### TECHNICAL FIELD

The present disclosure relates to systems and methods for stimulation of effector tissue in the human body.

### BACKGROUND

The nervous system of the body innervates various types of tissue, which may be commonly referred to as effector tissue. Generally, effector tissue may be understood as tissue that produce a response or perform work (‘effector response’) when activated by nerve signals. Examples include muscles (skeletal muscles, smooth muscle) and glands (endocrine glands and exocrine glands). They may furthermore be categorized based on their relationship with the nervous system. The somatic effectors are mostly skeletal muscles controlled by the somatic nervous system, responsible for voluntary actions, whereas the autonomic effectors typically are controlled by the autonomic nervous system and include smooth muscle tissue, cardiac muscle, and glands. These effectors are generally not under voluntary control.

The sympathetic nervous system (SNS) and the parasympathehtic nervous system (PNS) form the autonomous nervous system and may have complementary functions on the same effector tissue, such as an organ or a muscle. For example, the SNS may accelerate the heart rate, while the PNS may slow it down. Both the SNS and the PNS are typically active to some extent all the time, but their relative activities may change depending on the situation. This dynamic balance between the SNS and the PNS is commonly referred to as the “autonomic tone”. The autonomic tone hence implies there is an ongoing, background level of activity in the SNS and PNS. The body may adjust this balance as needed, ramping up sympathetic or parasympathetic activity in response to specific situations. The tone may also capture the systems' readiness to respond to stimuli. A certain “tone” or baseline activity level may ensure that the system can quickly ramp up or down its activity to adapt to different situations.

The nervous system of the body regulates and affects a great variety of bodily functions, and it is therefore of interest to control or modulate various parts of the nervous system for therapeutic purposes, such as for treating obesity, affecting sexual dysfunction, and adjusting the blood pressure.

### SUMMARY

It is an object of the present invention to provide improved technologies and methods for affecting an effector response in a human body.

According to an embodiment, a system for affecting an effector response in a patient is provided. The system comprises a stimulation device configured to deliver, directly or indirectly, a first stimulation signal to a sympathetic nerve innervating a first effector tissue of the patient, and a second stimulation signal to a parasympathetic nerve innervating a second effector tissue. The system further comprises a control unit configured to control an operation of the stimulation device such that: the first stimulation signal stimulates an activity of the sympathetic nerve and the second stimulation signal inhibits an activity of the parasympathetic nerve, or the first stimulation signal inhibits an activity of the sympathetic nerve and the second stimulation signal stimulates an activity of the parasympathetic nerve.

In an example, the control unit may be configured to control the operation of the stimulation device such that at least one of the first stimulation signal and the second stimulation signal is a periodic signal including at least one of: a variable frequency component, a variable duty cycle component, a variable amplitude component, and a variable pause component.

In an example, the first signal may be a low-frequency signal configured to stimulate the activity of the sympathetic nerve and the second signal is a high-frequency signal configured to inhibit the activity of the parasympathetic nerve. Alternatively, the first signal may be a high-frequency signal configured to inhibit the activity of the sympathetic nerve and the second signal is a low-frequency signal configured to stimulate the activity of the parasympathetic nerve.

In an example, an amplitude of the low-frequency signal may vary with a frequency in the range of 0.1-100 Hz and an amplitude of the high-frequency signal vary with a frequency in the range of 1-10 KHz.

In an example, at least one of the first and second stimulation signals may comprise a series of pulses having a negative voltage relative to ground.

In an example, the control unit may be configured to operate the stimulation device to generate a positive voltage pulse following one or more negative voltage pulses.

In an example, at least one of the first stimulation signal and second stimulation signal may be an electric signal or a vibrational signal.

In an example, the control unit may be configured to operate the stimulation device to alternatingly apply the first stimulation signal to the sympathetic nerve and the second stimulation signal to the parasympathetic nerve.

In an example, the control unit may be configured to operate the stimulation device to simultaneously apply the first stimulation signal to the sympathetic nerve and the second stimulation signal to the parasympathetic nerve.

In an example, the control unit may be configured to control the operation of the stimulation device to generate an effector response being at least one of a muscular response and a glandular response.

In an example, each of the first and second effector tissue may be a muscular tissue. Further, the control unit may be configured to control the operation of the stimulation device such that the first stimulation signal stimulates the activity of the sympathetic nerve and the second stimulation signal inhibits the activity of the parasympathetic nerve, thereby inducing contraction in the muscular tissue.

In an example, each of the first and second effector tissue may be a muscular tissue. Furthermore, the control unit may be configured to control the operation of the stimulation device such that the first stimulation signal inhibits the activity of the sympathetic nerve and the second stimulation signal stimulates the activity of the parasympathetic nerve, thereby inducing relaxation in the muscular tissue.

In an example, the first and second effector tissue may be smooth muscle tissue.

In an example, the first and second effector tissue may form part of a blood vessel, an intestine, or a urine bladder of the patient.

In an example, the system may further comprise a sensor device configured to generate a sensor signal indicating an effector response in the effector tissue and a control unit configured to receive the sensor signal and control an operation of the stimulation device based at least in part on the sensor signal.

In an example, the sensor device may comprise a sensor electrode configured to measure an electric activity in the effector tissue.

In an example, the sensor device may comprise a sensor electrode configured to measure a change in electrical impedance in the effector tissue.

In an example, the sensor device may comprise an electromyographic sensor configured to measure an electric activity in the effector tissue and an electric impedance sensor configured to measure a change in electrical impedance in the effector tissue.

In an example, the sensor electrode may be configured to be arranged at the effector tissue. The sensor may further comprise a reference electrode and be configured to generate the sensor signal based on an electrical interaction between the sensor electrode and the reference electrode.

In an example, the reference electrode may be formed by a housing of the stimulation device or the sensor device.

In an example, the sensor device may be configured to measure mechanical movement in the effector tissue.

In an example, the sensor device may comprise a strain gauge configured to measure a contraction or relaxation of the effector tissue.

In an example, the control unit may be configured to determine a response measure based on the sensor signal, the response measure being indicative of the effector response.

In an example, the control unit may be configured to compare the response measure with a predetermined reference measure and control the stimulation device to, in response to the response measure being below the reference measure, increase an intensity of the first stimulation signal to stimulate the activity in the sympathetic nerve and/or increase an intensity of the second stimulation signal to inhibit the activity of the parasympathetic nerve. Further, the control unit may be configured to control the stimulation device to, in response to the response measure exceeding the reference measure, reduce the intensity of the first stimulation signal to inhibit the activity of the sympathetic nerve and/or stimulate the activity of the parasympathetic nerve. The predetermined reference measure may be based on a previous measurement of the effector response in the patient or previous measurement of effector responses in other patients.

In an example, the control unit may be configured to monitor the response measure of the effector response over time, and to control the stimulation device based on a change rate in the effector response over time.

In an example, the control unit may be configured to determine a calibration parameter of the stimulation device based on the response measure.

In an example, the stimulation device may comprise a first electrode arrangement configured to be coupled to the sympathetic nerve to deliver the first stimulation signal and a second electrode arrangement configured to be coupled to the parasympathetic nerve to deliver the second stimulation signal.

In an example, the first electrode arrangement may comprise a first stimulation electrode and a second stimulation electrode, wherein the first stimulation electrode and the second stimulation electrode may be configured to be spaced apart along the sympathetic nerve.

In an example, the stimulation device may be configured to generate the first stimulation signal such that the first stimulation electrode serves as a cathode and the second stimulation electrode serves as an anode.

In an example, the system may further comprise a cuff configured to be at least partially arranged around the nerve and hold the first electrode arrangement in place against the sympathetic nerve.

In an example, the second electrode arrangement may comprise a third electrode and a fourth electrode, wherein the third electrode and the fourth electrode are configured to be arranged spaced apart along the parasympathetic nerve. The second electrode arrangement may further comprise a fifth electrode configured to be arranged spaced apart from the fourth electrode such that the fourth electrode is arranged between the third and fifth electrodes.

In an example, the stimulation device may be configured to generate the second stimulation signal such that the fourth electrode serves as a cathode and the third and fifth electrodes serve as anodes.

In an example, the system may further comprise a cuff configured to be at least partially arranged around the parasympathetic nerve and hold the second electrode arrangement in place against the parasympathetic nerve.

According to an embodiment, a system for affecting an effector response in a patient is provided, comprising a stimulation device configured to be coupled to at least one of an effector tissue and a nerve innervating the effector tissue of the patient, and a control unit configured to operate the stimulation device to apply at least one of a first stimulation signal and a second stimulation signal to the effector tissue. The first stimulation signal is a time-varying signal with an amplitude varying with a frequency lying in a first frequency interval and the second stimulation signal is a time-varying signal with an amplitude varying with a frequency lying in a second frequency interval. The first frequency interval is selected to inducing the effector response in the effector tissue and the second frequency interval is selected to inhibit the effector response in the effector tissue.

In an example, the first frequency interval may be 0.1-100 Hz and the second frequency interval 1-10 KHz.

In an example, at least one of the first and second stimulation signals may be an electric signal comprising a series of pulses having a negative voltage relative to ground.

In an example, the control unit may be configured to operate the stimulation device to generate a positive voltage pulse following one or more negative voltage pulses.

In an example, the control unit may be configured to operate the stimulation device to generate a first stimulation signal having a frequency of 0.5-3 Hz, and wherein the effector tissue is cardiac muscle tissue.

In an example, the control unit may be configured to operate the stimulation device to generate a first stimulation signal having a frequency of 1-10 Hz, and wherein the effector tissue is skeletal muscle tissue.

In an example, the control unit may be configured to operate the stimulation device to generate a first stimulation signal having a frequency of 0.1-100 Hz, and wherein the effector tissue is smooth muscle tissue.

In an example, at least one of the first stimulation signal and second stimulation signal may be an electric signal or a vibrational signal.

In an example, the control unit may be configured to operate the stimulation device to alternatingly apply the first stimulation signal and the second stimulation signal to the effector tissue.

In an example, the system may further comprise a sensor device configured to generate a sensor signal indicating an effector response in the effector tissue, wherein the control unit may be configured to receive the sensor signal and control an operation of the stimulation device based at least in part on the sensor signal.

In an example, the sensor device may comprise a sensor electrode configured to measure an electric activity in the effector tissue.

In an example, the sensor device may comprise a sensor electrode configured to measure a change in electrical impedance in the effector tissue.

In an example, the sensor device may comprise an electromyographic sensor electrode configured to measure an electric activity in the effector tissue and an electric impedance sensor electrode configured to measure a change in electrical impedance in the effector tissue.

In an example, the sensor electrode may be configured to be arranged at the effector tissue. The sensor may further comprise a reference electrode and be configured to generate the sensor signal based on an electrical interaction between the sensor electrode and the reference electrode.

In an example, the reference electrode may be formed by a housing of the stimulation device or the sensor device.

In an example, the sensor device may be configured to measure mechanical movement in the effector tissue.

In an example, the sensor device may comprise a strain gauge configured to measure a contraction or relaxation of the effector tissue.

In an example, the control unit may be configured to determine a response measure based on the sensor signal, the response measure being indicative of the effector response.

In an example, the control unit may be configured to compare the response measure with a predetermined reference measure and control the stimulation device to: in response to the response measure being below the reference measure, increase an intensity of the first stimulation signal to stimulate the activity in the effector tissue, and in response to the response measure exceeding the reference measure, increase the intensity of the second stimulation signal to inhibit the activity of the effector tissue. The predetermined reference measure may be based on a previous measurement of the effector response in the patient or previous measurement of effector responses in other patients.

In an example, the control unit may be configured to monitor the response measure of effector response over time and control the stimulation device based on a change rate in the effector response over time.

In an example, the control unit may be configured to determine a calibration parameter of the stimulation device based on the response measure.

In an example, the stimulation device may comprise a first electrode arrangement configured to deliver the first stimulation signal and a second electrode arrangement configured to deliver the second stimulation signal. The first electrode arrangement may comprise a first stimulation electrode and a second stimulation electrode, wherein the first stimulation electrode and the second stimulation electrode are configured to be spaced apart along the nerve innervating the effector tissue.

In an example, the stimulation device may be configured to generate the first stimulation signal such that the first stimulation electrode serves as a cathode and the second stimulation electrode serves as an anode.

In an example, the system may further comprise a cuff configured to be at least partially arranged around the nerve and hold the first electrode arrangement in place against the nerve.

In an example, the second electrode arrangement may comprise a third electrode and a fourth electrode, the third electrode and the fourth electrode being configured to be arranged spaced apart along the nerve.

In an example, the stimulation device may be configured to generate the second stimulation signal such that the third electrode serves as a cathode and the fourth electrode serves as an anode.

In an example, the system may further comprise a cuff configured to be at least partially arranged around the nerve and hold the second electrode arrangement in place against the nerve.

In an example, the system may further comprise a suppression electrode arrangement configured to be coupled to the nerve to apply a suppression signal suppressing action potentials propagating in the nerve in a direction towards the central nervous system.

In an example, the control unit may be configured to regulate the suppression signal so as to suppress the action potentials induced in response to the stimulation device applying the first stimulation signal.

In an example, the stimulation device may be configured to be coupled to the nerve at a position between the effector tissue and the suppression electrode arrangement, so as to induce action potentials travelling in the nerve in a direction towards the effector tissue.

In an example, the control unit may be configured to regulate the suppression of the action potentials so as to inhibit an undesired response of the nervous system of the patient, the undesired response being generated responsive to the stimulation device applying the first stimulation signal.

In an example, the control unit may be configured to drive the stimulation device and the suppression electrode arrangement such that each of the stimulation device and the suppression electrode arrangement is actuated in sequence, with a delay of the suppression signal timed to generally match a conduction velocity of the first stimulation signal.

In an example, the control unit may be configured to drive the stimulation device and the suppression electrode arrangement to apply the first stimulation signal and the suppression signal substantially at the same time.

In an example, the control unit may be configured to drive the stimulation device and the suppression electrode arrangement such that each of the first stimulation signal and the suppression signal is a time-varying signal, wherein the first stimulation signal is a low-frequency signal, and the suppression signal is a high-frequency signal. An amplitude of the first stimulation signal may vary with a frequency in the range of 0.1-100 Hz, while an amplitude of the suppression signal may vary with a frequency in the range of 1-10 KHz.

According to an embodiment, a system for affecting an effector response in a patient is provided. The system comprises a stimulation device comprising a first and a second electrode arrangement, wherein each is configured to be coupled to at least one of an effector tissue and a nerve innervating the effector tissue of the patient, and a control unit configured to drive the stimulation device to apply, by means of the first electrode arrangement, a stimulation signal inducing the effector response in the effector tissue and apply, by means of the second electrode arrangement, a suppression signal suppressing action potentials propagating in the nerve towards the central nervous system (CNS). The control unit is configured to regulate the suppression signal so as to suppress the action potentials induced in response to the stimulation device applying the stimulation signal.

In an example, the first electrode arrangement may be configured to be coupled to the nerve at a position between the effector tissue and the second electrode so as to induce action potentials travelling in the nerve in a direction towards the effector tissue.

In an example, the control unit may be configured to regulate the suppression of the action potentials so as to inhibit an undesired response of the nervous system of the patient, the undesired response being generated responsive to the first electrode applying the stimulation signal.

In an example, the control unit may be configured to drive the stimulation device such that each of the first and second electrode arrangements are actuated in sequence, with a delay of the suppression signal timed to generally match a conduction velocity of the stimulation signal.

In an example, the control unit may be configured to drive the stimulation device to apply the stimulation signal and the suppression signal substantially at the same time.

In an example, the control unit may be configured to drive the stimulation device such that each of the stimulation signal and the suppression signal is a time-varying signal, wherein the stimulation signal is a low-frequency signal and the suppression signal is a high-frequency signal. An amplitude of the stimulation signal may vary with a frequency in the range of 0.1-100 Hz, while an amplitude of the suppression signal may vary with a frequency in the range of 1-10 KHz.

In an example, the first and second electrode arrangements may be configured to be spaced apart along nerve.

In an example, the first electrode arrangement may comprise a first stimulation electrode and a second stimulation electrode configured to apply the stimulation signal to the effector tissue or nerve.

In an example, the first stimulation electrode and the second stimulation electrode may be configured to be spaced apart along the nerve.

In an example, the stimulation device may be configured to generate the stimulation signal such that the first stimulation electrode serves as a cathode and the second stimulation electrode serves as an anode.

In an example, the system may further comprise a cuff configured to be at least partially arranged around the nerve and hold the first electrode arrangement in place against the nerve.

In an example, the second electrode arrangement may comprise a first suppression electrode and a second suppression electrode configured to apply the suppression signal to the nerve.

In an example, the first suppression electrode and the second suppression electrode may be configured to be spaced apart along the nerve.

In an example, the second electrode arrangement may further comprise a third suppression electrode configured to be arranged spaced apart from the second suppression electrode such that the second suppression electrode is arranged between the first and third suppression electrodes.

In an example, the stimulation device may be configured to generate the suppression signal such that the second suppression electrode serves as a cathode and the first and third suppression electrodes serve as anodes.

In an example, the system may further comprise a cuff configured to be at least partially arranged around the nerve and hold the second electrode arrangement in place against the nerve.

In an example, the system may further comprise a sensor device configured to generate a sensor signal indicating an effector response in the effector tissue. The control unit may be further configured to receive the sensor signal and control an operation of the stimulation device based at least in part on the sensor signal.

In an example, the sensor device may comprise a sensor electrode configured to measure an electric activity in the effector tissue.

In an example, the sensor device may comprise a sensor electrode configured to measure a change in electrical impedance in the effector tissue.

In an example, the sensor device may comprise an electromyographic sensor electrode configured to measure an electric activity in the effector tissue and an electric impedance sensor electrode configured to measure a change in electrical impedance in the effector tissue.

In an example, the sensor electrode may be configured to be arranged at the effector tissue. The sensor may further comprise a reference electrode and be configured to generate the sensor signal based on an electrical interaction between the sensor electrode and the reference electrode.

In an example, the reference electrode may be formed by a housing of the stimulation device or the sensor device.

In an example, the sensor device may be configured to measure mechanical movement in the effector tissue. The sensor device may comprise a strain gauge configured to measure a contraction or relaxation of the effector tissue.

In an example, the control unit may be configured to determine a response measure based on the sensor signal, the response measure being indicative of the effector response. The control unit may be configured to compare the response measure with a predetermined reference measure and control the stimulation device to: in response to the response measure being below the reference measure, increase an intensity of the stimulation signal to stimulate the activity in the effector tissue, and in response to the response measure exceeding the reference measure, reduce the intensity of the stimulation signal to inhibit the activity of the effector tissue. The predetermined reference measure may be based on a previous measurement of the effector response in the patient or previous measurement of effector responses in other patients.

In an example, the control unit may be configured to monitor the response measure of the effector response over time and control the stimulation device based on a change rate in the effector response over time.

In an example, the control unit may be configured to determine a calibration parameter of the stimulation device based on the response measure.

According to an embodiment, a system for affecting an effector response in a patient is provided, comprising a stimulation device configured to deliver, directly or indirectly, a stimulation signal to at least one of an effector tissue and a nerve innervating the effector tissue of the patient, a sensor device configured to generate a sensor signal indicating an effector response in the effector tissue, and a control unit. The control unit is configured to receive the sensor signal and control an operation of the stimulation device based at least in part on the sensor signal.

In an example, the sensor device may comprise a sensor electrode configured to measure an electric activity in the effector tissue in response to the stimulation signal.

In an example, the sensor device may comprise a sensor electrode configured to measure a change in electrical impedance in the effector tissue in response to the stimulation signal.

In an example, the sensor device may comprise an electromyographic sensor configured to measure an electric activity in the effector tissue and an electric impedance sensor configured to measure a change in electrical impedance in the effector tissue.

In an example, the sensor electrode may be configured to be arranged at the effector tissue. The sensor electrode may comprise a reference electrode and the sensor device may be configured to generate the sensor signal based on an electrical interaction between the sensor electrode and the reference electrode. The reference electrode may be formed by a housing of the stimulation device or the sensor device.

In an example, the sensor device may be configured to measure mechanical movement in the effector tissue in response to the stimulation signal. The sensor device may comprise a strain gauge configured to measure a contraction or relaxation of the effector tissue in response to the stimulation signal.

In some examples, the sensor device may be configured to measure a heart rate of the patient, a blood pressure of the patient, or a rate of respiration of the patient.

In an example, the control unit may be configured to determine a response measure based on the sensor signal, the response measure being indicative of the effector response.

In an example, the control unit may be configured to compare the response measure with a predetermined reference measure and control the stimulation device to: increase an intensity of the stimulation signal in response to the response measure being below the reference measure, and reduce the intensity of the stimulation signal in response to the response measure exceeding the reference measure.

In an example, the control unit may be configured to increase the intensity of the stimulation signal by increasing at least one of a frequency, current amplitude and voltage amplitude of the stimulation signal and reduce the intensity of the stimulation signal by reducing at least one of the frequency, current amplitude and voltage amplitude of the stimulation signal. The predetermined reference measure may be based on a previous measurement of the effector response in the patient or previous measurement of effector responses in other patients.

In an example, the control unit may be configured to monitor the level of effector response over time and control the stimulation device based on a change rate in the effector response over time.

In an example, the control unit may be configured to determine a calibration parameter of the stimulation device based on the response measure.

In an example, the control unit may be configured to control the operation of the stimulation device to generate an effector response being at least one of a muscular response and a glandular response. The effector tissue may be smooth muscle tissue, such as forming part of a blood vessel, an intestine, or a urine bladder of the patient.

In an example, the control unit may be configured to control the operation of the stimulation device such that the stimulation signal is a periodic signal including at least one of: a variable frequency component, a variable duty cycle component, a variable amplitude component, and a variable pause component. The stimulation signal may be one of a low-frequency signal with an amplitude varying in the range of 0.1-100 Hz and a high-frequency signal with an amplitude varying in the range of 1-10 kHz. The stimulation signal may comprise series of pulses having a negative voltage relative to ground.

In an example, the control unit may be configured to operate the stimulation device to generate a positive voltage pulse following one or more negative voltage pulses.

The stimulation signal may be an electric signal or a vibrational signal.

In an example, the stimulation device may comprise a first stimulation electrode and a second stimulation electrode, the first stimulation electrode and the second stimulation electrode being configured to be spaced apart along the nerve innervating the effector tissue. The stimulation device may be configured to generate the stimulation signal such that the first stimulation electrode serves as a cathode and the second stimulation electrode serves as an anode. The system may further comprise a cuff configured to be at least partially arranged around the nerve and hold the first and second stimulation electrodes in place against the nerve.

In an example, the system may further comprise a suppression electrode arrangement configured to be coupled to the nerve to apply a suppression signal suppressing action potentials propagating in the nerve in a direction towards the central nervous system. The control unit may be configured to regulate the suppression signal so as to suppress the action potentials induced in response to the stimulation device applying the stimulation signal. Furthermore, the stimulation device may be configured to be coupled to the nerve at a position between the effector tissue and the suppression electrode arrangement, so as to induce action potentials travelling in the nerve in a direction towards the effector tissue.

In an example, the control unit may be configured to regulate the suppression of the action potentials so as to inhibit an undesired response of the nervous system of the patient, the undesired response being generated responsive to the stimulation device applying the stimulation signal.

In an example, the control unit may be configured to drive the stimulation device and the suppression electrode arrangement such that each of the stimulation device and suppression electrode arrangement is actuated in sequence, with a delay of the suppression signal timed to generally match a conduction velocity of the stimulation signal in the nerve.

In an example, the control unit may be configured to drive the stimulation device and the suppression electrode arrangement to apply the first stimulation signal and the suppression signal substantially at the same time.

In an example, the control unit may be configured to drive the stimulation device and the suppression electrode arrangement such that each of the stimulation signal and the suppression signal is a time-varying signal, wherein the stimulation signal is a low-frequency signal and the suppression signal is a high-frequency signal. An amplitude of the stimulation signal may vary with a frequency in the range of 0.1-100 Hz, whereas an amplitude of the suppression signal may vary with a frequency in the range of 1-10 KHz.

According to an embodiment, a method for at least partly denervating an effector tissue of a patient is provided. The method comprises temporarily inhibiting a nerve innervating the effector tissue, determining a response measure indicative of an effector response in the effector tissue, wherein the effector response is at least partly induced by the inhibiting of the nerve, comparing the response measure with a predetermined reference measure, and at least partly denervating the effector tissue based at least in part on the comparison.

According to an embodiment, a system for at least partly denervating an effector tissue of a patient is provided, comprising an inhibition device configured to temporarily inhibit a nerve innervating the effector tissue, a sensor configured to generate a sensor signal indicative of an effector response in the effector tissue, the effector response being at least partly induced by the inhibiting of the nerve, and a processing unit configured to determine a response measure based on the sensor signal, the response measure being indicative of the effector response, compare the response measure with a predetermined reference measure, and determine, based on the comparison, whether a desired effector response has been achieved. The system further comprises a denervation device configured to at least partly denervate the effector tissue.

In an example, the inhibition device comprises a cooling device configured to cool the nerve to cause a temporary inhibition of the nerve.

In an example, the inhibition device is a toxin administration device configured to deliver a neurotoxin to the nerve to cause a temporary inhibition of the nerve. The neurotoxin may in an example comprise botulinum toxin.

In an example, the inhibition device may be a vibrational device configured to deliver an inhibition signal to the nerve to cause a temporary inhibition of the nerve.

In an example, the inhibition device may be an electric stimulation device configured to deliver an inhibition signal to the nerve to cause a temporary inhibition of the nerve.

In some examples, the inhibition signal is a periodic signal including at least one of: a variable frequency component, a variable duty cycle component, a variable amplitude component, and a variable pause component. An amplitude of the signal may vary in the range of 1-10 KHz.

In an example, the inhibition signal may be an electric signal comprising a series of pulses having a negative voltage relative to ground. A positive voltage pulse may follow one or more negative voltage pulses.

In an example, the inhibition signal may be generated such that a first inhibition electrode serves as a cathode and a second inhibition electrode serves as an anode, the first and second inhibition electrodes being arranged spaced apart along the nerve.

In an example, a suppression signal may be delivered to the nerve to suppress action potentials propagating in the nerve in a direction towards the central nervous system.

In an example, the suppression signal may be regulated to suppress the action potentials induced in the nerve in response to the inhibition signal.

In an example, the inhibition signal may be delivered at a position between the effector tissue and the position in which the suppression signal is applied to the nerve.

In an example, the suppression of the action potentials is delivered to inhibit an undesired response of the nervous system of the patient, the undesired response being generated responsive to the electric inhibition signal.

In an example, the electric inhibition signal and the suppression signal may be delivered in sequence, with a delay of the suppression signal timed to generally match a conduction velocity of the electric inhibition signal in the nerve.

In an example, a sensor signal, indicative of the effector response, may be received at the control unit which may determine the response measure based on the received sensor signal.

In an example, the sensor signal may be indicative of an electric activity in the effector tissue.

In an example, the sensor signal may be indicative of a change in electrical impedance in the effector tissue.

In some examples, the effector tissue may form part of a renal artery of the patient and the sensor signal may be indicative of a vasodilation or vasoconstriction of the renal artery.

In some examples, the effector tissue may form part of a gastrointestinal tract of the patient and the sensor signal may be indicative of a level of motility of the gastrointestinal tract.

In an example, the effector tissue may be glandular tissue and the sensor signal be indicative of a level of glandular secretion of the glandular tissue. The glandular tissue may form part of at least one of: a pancreas secreting insulin, a gallbladder secreting bile, and an adrenal gland secreting adrenaline, aldosterone, or cortisol.

In an example, the effector tissue may be muscle tissue and wherein the sensor signal be indicative of mechanical movement of the muscle tissue. The sensor signal may be generated by a strain gauge measuring a contraction or relaxation of the muscle tissue.

In some examples, the sensor signal may be indicative of a heart rate of the patient, a blood pressure of the patient, or a rate of respiration of the patient.

In an example, the at least partly denervating of the effector tissue may comprises ablating the effector tissue. The ablating may comprise at least one of: surgical ablation, radiofrequency ablation, cryoablation, laser ablation, heat ablation, laser ablation, electrocautery, and chemical ablation.

According to an embodiment, a system for stimulating an effector tissue of a patient is provided. The system comprises a stimulation device configured to deliver a stimulation signal to at least one of the effector tissue and a nerve innervating the effector tissue of the patient, a source of energy configured to energize the stimulation device, a control unit operably connected to the stimulation device and configured to control an operation of the stimulation device such that the stimulation signal causes at least one of an effector response and inhibition of the effector response in the effector tissue, and a capacitor configured to reduce a current leakage of the system to 1 μA or less, such as 0.1 μA or less. It will be appreciated that the capacitor may be implemented in any of the systems of the above-discussed aspects and examples.

In an example, the capacitor may be configured to be connected in series with the body of the patient and at least one of the stimulation device, the source of energy, and the control unit.

In an example, the stimulation device may comprise an electrode arrangement configured to be coupled to the effector tissue or the nerve. The capacitor may be configured to be connected in series with the body of the patient and the electrode arrangement.

In an example, the electrode arrangement may comprise a first stimulation electrode and a second stimulation electrode for applying the stimulation signal. The capacitor may be configured to be connected in series with the first stimulation electrode and the second stimulation electrode.

In an example, the capacitor may be integrated in a circuitry for controlling the operation of the stimulation device.

In an example, the system may further comprise a printed circuit board, PCB, supporting the capacitor and at least one of the stimulation device, the source of energy, and the control unit. The PCB may be at least one of a multi-layer PCB, a flexible PCB, and a stretchable PCB.

According to an embodiment, a system for stimulating an effector tissue of a patient is provided. The system comprises a stimulation device configured to deliver a stimulation signal to at least one of the effector tissue and a nerve innervating the effector tissue of the patient, a source of energy configured to energize the stimulation device, a control unit operably connected to the stimulation device and configured to control an operation of the stimulation device such that the stimulation signal causes at least one of an effector response and inhibition of the effector response in the effector tissue, and a printed circuit board (PCB), supporting at least one of the stimulation device, the source of energy, and the control unit. The PCB is at least one of a multi-layer PCB, a flexible PCB, a stretchable PCB. It will be appreciated that the PCB may be implemented in any of the systems of the above-discussed aspects and examples.

In an example, the PCB may comprise a first multi-layer portion and a second multi-layer portion interconnected by a stretchable portion.

In an example, the PCB may comprise a first multi-layer portion and a second multi-layer portion interconnected by a flexible portion.

According to an embodiment, a method for affecting a balance between a sympathetic tone and parasympathetic tone of the autonomic nervous system of a patient is provided. The method comprises:
delivering, by a first stimulation arrangement, a first stimulation signal to a sympathetic nerve of the patient to increase a level of activity of the sympathetic nerve and move the balance towards a sympathetic dominance, or
delivering, by a second stimulation arrangement, a second stimulation signal to the parasympathetic nerve to increase a level of activity of the parasympathetic nerve and move the balance towards a parasympathetic dominance.

In an example, each of the sympathetic nerve and the parasympathetic nerve forms part of at least one of: a cardiovascular system, a respiratory system, a gastrointestinal tract, a urinary system, an immune system, a sexual function, and a stress response system.

In an example, the first stimulation signal is delivered to the vagus nerve.

In an example, the first stimulation signal is delivered to the celiac branch of the vagus nerve.

In an example, the first stimulation signal is delivered to the sacral plexus.

In an example, at least one of the first stimulation signal and the second stimulation signal is delivered to the cardiac plexus.

In an example, at least one of the first stimulation signal and the second stimulation signal is delivered to the pulmonary plexus.

In an example, the second stimulation signal is delivered to the celiac plexus.

In an example, the second stimulation signal is delivered to the hypogastric plexus.

In an example, the first stimulation signal is delivered to the pelvic plexus.

In an example, at least one of the first stimulation signal and the second stimulation signal is delivered to the hypothalamus.

In an example, at least one of the first stimulation signal and the second stimulation signal is delivered to the brainstem.

In an example, at least one of the first stimulation signal and the second stimulation signal is delivered to the midbrain, the pons, or the medulla oblongata.

In an example, each of the first and second stimulation signals is a periodic signal including at least one of: a variable frequency component, a variable duty cycle component, a variable amplitude component, and a variable pause component.

In an example, each of the first stimulation signal and the second stimulation signal comprises an amplitude varying with a frequency in the range of 0.1-100 Hz.

In an example, at least one of the first and second stimulation signals comprises series of pulses having a negative voltage relative to ground.

In an example, the method method comprises generating a positive voltage pulse following one or more negative voltage pulses.

In an example, at least one of the first stimulation signal and the second stimulation signal is an electric signal or a vibrational signal.

In an example, the method further comprises:
receiving a sensor signal indicating an activity in the sympathetic nerve in response to the first stimulation signal, or an activity in the parasympathetic nerve in response to the second stimulation signal; and
adjusting an intensity of at least one of the first stimulation signal and the second stimulation signal based at least in part on the sensor signal.

In an example, the sensor signal indicates an electric activity in the sympathetic nerve and/or the parasympathetic nerve.

In an example, the sensor signal indicates a change in electrical impedance in the sympathetic nerve and/or the parasympathetic nerve.

In an example, the method comprises delivering the first stimulation signal by a first stimulation electrode and a second stimulation electrode arranged spaced apart along the sympathetic nerve.

In an example, the method comprises delivering the first stimulation signal such that the first stimulation electrode serves as a cathode and the second stimulation electrode serves as an anode.

In an example, the method comprises delivering the second stimulation signal by a third stimulation electrode and a fourth stimulation electrode arranged spaced apart along the parasympathetic nerve.

In an example, the method comprises delivering the second stimulation signal such that the third electrode serves as a cathode and the fourth electrodes serves as an anode.

In an example, the method comprises:
applying a first suppression signal to the sympathetic nerve to suppress action potentials generated by the first stimulation signal and propagating in a direction towards the central nervous system.

In an example, the method comprises:
regulating the suppression of the action potentials to inhibit an undesired response of the nervous system of the patient, the undesired response being generated responsive to the first stimulation signal.

In an example, the method comprises:
applying the first stimulation signal and the first suppression signal in sequence, with a delay of the first suppression signal timed to generally match a conduction velocity of the first stimulation signal in the sympathetic nerve.

In an example, an amplitude of the first suppression signal varies with frequency in the range of 1-10 KHz.

In an example, the method further comprises:
applying a second suppression signal to the parasympathetic nerve to suppress action potentials generated by the second stimulation signal and propagating in a the direction towards the central nervous system.

In an example, the method comprises:
regulating the suppression of the action potentials to inhibit an undesired response of the nervous system of the patient, the undesired response being generated responsive to the second stimulation signal.

In an example, the method comprises:
applying the second stimulation signal and the second suppression signal in sequence, with a delay of the second suppression signal timed to generally match a conduction velocity of the second stimulation signal in the parasympathetic nerve.

In an example, an amplitude of the second suppression signal varies with frequency in the range of 1-10 KHz.

According to an embodiment, a system for reducing pain in a patient is provided, comprising:
a stimulation device configured to deliver, directly or indirectly, a suppression signal to a nerve of the patient;
a control unit configured to control an operation of the stimulation device to suppress or block a propagation of action potentials in an afferent direction of the nerve, thereby reducing the patient's sensation of pain originating from the action potentials.

In an example, the pain is a phantom pain.

In an example, the control unit is configured to control the operation of the stimulation device such that the suppression signal is a time-varying signal with a frequency in the range of 1-10 KHz.

In an example, the system further comprises a sensor device configured to generate a sensor signal indicating the action potentials propagating in the nerve.

In an example, the control unit is configured to receive the sensor signal and to control an operation of the stimulation device based at least in part on the sensor signal.

In an example, the stimulation device comprises a suppression electrode configured to be coupled to the nerve to deliver the suppression signal to the nerve, and wherein the sensor device comprises a sensor electrode configured to be arranged cranial to the suppression electrode.

In an example, the control unit is configured to increase an intensity of the suppression signal in response to action potentials passing by the suppression electrode in the cranial direction.

In an example, the control unit is configured to control the operation of the stimulation device such that the suppression signal is a periodic signal including at least one of: a variable frequency component, a variable duty cycle component, a variable amplitude component, and a variable pause component.

In an example, the suppression signal comprises a series of pulses having a negative voltage relative to ground.

In an example, the control unit is configured to operate the stimulation device to generate a positive voltage pulse following one or more negative voltage pulses.

In an example, the stimulation device comprises a first suppression electrode and a second suppression electrode, the first suppression electrode and the second suppression electrode being configured to be spaced apart along the nerve.

In an example, the stimulation device is configured to generate the suppression signal such that the first suppression electrode serves as a cathode and the second suppression electrode serves as an anode.

In an example, the system further comprises a cuff configured to be at least partially arranged around the nerve and to hold the first suppression electrode and/or the second suppression electrode in place against the nerve.

According to an embodiment, a method of creating engorgement of an erectile body of a male person is provided. The method involves using a pre-implanted stimulation device comprising configured to deliver a first stimulation signal to a sympathetic nerve innervating a penile vein of the male person and to deliver a second stimulation signal to a parasympathetic nerve innervating a penile artery of the male person. The method comprising:
stimulating, by means of the first stimulation signal, the sympathetic nerve to cause vasoconstriction of the penile vein;
stimulating, by means of the second stimulation signal, the parasympathetic nerve to cause vasodilation of the penile artery;
wherein the vasodilation of the penile artery increases a blood flow entering the erectile body of the penis and the vasoconstriction of the penile vein reduces a blood flow exiting the erectile body, thereby inducing engorgement of the erectile body.

In an example, the method comprises
delivering the first stimulation signal to the sympathetic nerve at a position between a level of the T11-L2 vertebrae and the pelvic plexus; and
delivering the second stimulation signal to the parasympathetic nerve at a position between a level of sacral spinal cord segments S2-S4 and the pelvic plexus.

In an example, each of the first and second stimulation signals is a periodic signal including at least one of: a variable frequency component, a variably duty cycle component, a variable amplitude component, and a variable pause component.

In an example, the method comprises:
delivering a first inhibition signal to the sympathetic nerve to cause vasodilation of the penile vein; and
delivering a second inhibition signal to the parasympathetic nerve to cause vasoconstriction of the penile artery.

In an example, the first stimulation signal is a low-frequency signal configured to stimulate the activity of the sympathetic nerve;
the second stimulation signal is a low-frequency signal configured to stimulate the activity of the parasympathetic nerve;
the first inhibition signal is a high-frequency signal configured to inhibit the activity of the sympathetic nerve; and
the second inhibition signal is a high-frequency signal configured to inhibit the activity of the parasympathetic nerve.

In an example, the method comprises:
varying an amplitude of the low-frequency signal with a frequency in the range of 0.1-100 Hz; and
varying an amplitude of the high-frequency signal with a frequency in the range of 1-10 kHz.

In an example, at least one of the first and second stimulation signals comprises series of pulses having a negative voltage relative to ground.

In an example, a positive voltage pulse follows one or more negative voltage pulses.

In an example, at least one of the first stimulation signal and the second stimulation signal is an electric signal or a vibrational signal.

In an example, the method further comprises:
receiving a sensor signal indicating at least one of vasoconstriction of the penile vein, vasodilation of the penile artery, and engorgement of the erectile body; and
adjusting an intensity of at least one of the first and second stimulation signal based at least in part on the sensor signal.

In an example, the sensor signal indicates an electric activity in smooth muscle tissue of the penile vein and/or the penile artery.

In an example, the sensor signal indicates a change in electrical impedance in smooth muscle tissue of the penile vein and/or the penile artery.

In an example, the sensor signal is generated by a strain gauge and wherein the sensor signal indicates an engorgement of the erectile body.

In an example, the method comprises determining a response measure based on the sensor signal, the response measure being indicative of said at least one of vasoconstriction of the penile vein, vasodilation of the penile artery, and engorgement of the erectile body.

In an example, the method comprises:
comparing the response measure with a predetermined reference measure; and
in response to the response measure being below the reference measure, increasing an intensity of the first stimulation signal to stimulate the activity in the in the sympathetic nerve and/or increasing an intensity of the second stimulation signal to increase the activity of the parasympathetic nerve, and
in response to the response measure exceeding the reference measure, reducing the intensity of the first stimulation signal to inhibit the activity of the sympathetic nerve and/or reducing the intensity of the second stimulation signal to inhibit the activity of the parasympathetic nerve.

In an example, the predetermined reference measure is based on a previous measurement on the male person.

In an example, the predetermined reference measure is based on previous measurement on other male persons.

In an example, the method further comprises applying a first suppression signal to the sympathetic nerve to suppress action potentials generated by the first stimulation signal and propagating in a direction towards the central nervous system.

In an example, the method comprises applying the first stimulation signal at a position between the penile vein and a position in which the first suppression signal is applied.

In an example, the method comprises regulating the suppression of the action potentials to inhibit an undesired response of the nervous system of the male person, the undesired response being generated responsive to the first stimulation signal.

In an example, the method comprises applying the first stimulation signal and the first suppression signal in sequence, with a delay of the first suppression signal timed to generally match a conduction velocity of the first stimulation signal in the sympathetic nerve.

In an example, an amplitude of the first suppression signal varies with frequency in the range of 1-10 KHz.

In an example, the method further comprises applying a second suppression signal to the parasympathetic nerve to suppress action potentials generated by the second stimulation signal and propagating in the direction towards the central nervous system.

In an example, the method comprises applying the second stimulation signal at a position between the penile artery and a position in which the second suppression signal is applied.

In an example, the method comprises regulating the suppression of the action potentials to inhibit an undesired response of the nervous system of the male person, the undesired response being generated responsive to the second stimulation signal.

In an example, the method comprises applying the second stimulation signal and the second suppression signal in sequence, with a delay of the second suppression signal timed to generally match a conduction velocity of the second stimulation signal in the parasympathetic nerve.

In an example, an amplitude of the second suppression signal varies with frequency in the range of 1-10 KHz.

In an example, the method comprises delivering the first stimulation signal by means of a first electrode and a second electrode arranged spaced apart along the sympathetic nerve.

In an example, the method comprises stimulating the sympathetic nerve by operating the first electrode as a cathode and the second electrode as an anode.

In an example, the method comprises delivering the second stimulation signal by means of a third electrode and a fourth electrode arranged space apart along the parasympathetic nerve.

In an example, the method comprises stimulating the parasympathetic nerve by operating the third electrode as a cathode and the fourth electrode as an anode.

According to an embodiment, a system for treating erectile dysfunction in a male person is provided, comprising:
a stimulation device configured to deliver, directly or indirectly:
a first stimulation signal to a sympathetic nerve innervating a penile vein of the person;
a second stimulation signal to a parasympathetic nerve innervating a penile artery of the person; and
a control unit configured to control an operation of the stimulation device such that:
the first stimulation signal stimulates an activity of the sympathetic nerve, thereby causing vasoconstriction of the penile vein; and
the second stimulation signal stimulates an activity of the parasympathetic nerve, thereby causing vasodilation of the penile artery;
wherein the vasodilation of the penile artery increases a blood flow entering an erectile body of the penis and the vasoconstriction of the penile vein reduces a blood flow exiting the erectile body, thereby inducing engorgement of the erectile body.

In an example, the system comprises:
a first stimulation arrangement configured to deliver the first stimulation signal, the first stimulation arrangement being configured to be coupled to the sympathetic nerve at a position between a level of the T11-L2 vertebrae and the pelvic plexus; and
a second stimulation arrangement configured to deliver the second stimulation signal, the second stimulation arrangement being configured to be coupled to the second stimulation signal to the parasympathetic nerve at a position between a level of sacral spinal cord segments S2-S4 and the pelvic plexus.

In an example, the control unit is configured to control the operation of the stimulation device such that at least one of the first stimulation signal and the second stimulation signal is a periodic signal including at least one of: a variable frequency component, a variable duty cycle component, a variable amplitude component, and a variable pause component.

In an example, each of the first stimulation signal and the second stimulation signal comprises an amplitude varying with a frequency in the range of 0.1-100 Hz.

In an example, at least one of the first and second stimulation signals comprises series of pulses having a negative voltage relative to ground.

In an example, the control unit is configured to operate the stimulation device to generate a positive voltage pulse following one or more negative voltage pulses.

In an example, at least one of the first stimulation signal and the second stimulation signal is an electric signal or a vibrational signal.

In an example, the system further comprises a sensor device configured to generate a sensor signal indicating at least one of vasoconstriction of the penile vein, vasodilation of the penile artery, and engorgement of the erectile body, wherein the control unit is configured to receive the sensor signal and to control an operation of the stimulation device based at least in part on the sensor signal.

In an example, the sensor device comprises a sensor electrode configured to measure an electric activity in smooth muscle tissue of the penile vein and/or the penile artery.

In an example, the device comprises a sensor electrode configured to measure a change in electrical impedance in smooth muscle tissue of the penile vein and/or the penile artery.

In an example, the sensor electrode is configured to be arranged at the penile vein and/or the penile artery, the sensor further comprises a reference electrode, and the sensor device is configured to generate the sensor signal based on an electrical interaction between the sensor electrode and the reference electrode.

In an example, the reference electrode is formed by a housing of the stimulation device or the sensor device.

In an example, the sensor signal is generated by a strain gauge and wherein the sensor signal indicates an engorgement of the erectile body.

In an example, the control unit is configured to determine a response measure based on the sensor signal, the response measure being indicative of said at least one of vasoconstriction of the penile vein, vasodilation of the penile artery, and engorgement of the erectile body.

In an example, the control unit is configured to:
compare the response measure with a predetermined reference measure; and
in response to the response measure being below the reference measure, increase an intensity of the first stimulation signal to stimulate the activity in the in the sympathetic nerve and/or increase an intensity of the second stimulation signal to increase the activity of the parasympathetic nerve, and
in response to the response measure exceeding the reference measure, reduce the intensity of the first stimulation signal to inhibit the activity of the sympathetic nerve and/or reduce the intensity of the second stimulation signal to inhibit the activity of the parasympathetic nerve.

In an example, the predetermined reference measure is based on a previous measurement on the person.

In an example, the predetermined reference measure is based on previous measurement on other persons.

In an example, the stimulation device comprises a first stimulation arrangement configured to deliver the first stimulation signal and a second stimulation arrangement configured to deliver the second stimulation signal.

In an example, the first stimulation arrangement comprises a first stimulation electrode and a second stimulation electrode, the first stimulation electrode and the second stimulation electrode are configured to be spaced apart along the sympathetic nerve innervating the penile vein of the person.

In an example, the stimulation device is configured to generate the first stimulation signal such that the first stimulation electrode serves as a cathode and the second stimulation electrode serves as an anode.

In an example, the system further comprises a cuff configured to be at least partially arranged around the sympathetic nerve and to hold the first stimulation arrangement in place against the sympathetic nerve.

In an example, the second stimulation arrangement comprises a third electrode and a fourth electrode, the third electrode and the fourth electrode being configured to be arranged spaced apart along the parasympathetic nerve.

In an example, the stimulation device is configured to generate the second stimulation signal such that the third electrode serves as a cathode and the fourth electrodes serves as an anode.

In an example, the system further comprises a cuff configured to be at least partially arranged around the parasympathetic nerve and to hold the second stimulation arrangement in place against the parasympathetic nerve.

In an example, the system further comprises a suppression device comprising a first suppression arrangement configured to apply a first suppression signal to the sympathetic nerve to suppress action potentials generated by the first stimulation signal and propagating in a direction towards the central nervous system.

In an example, the stimulation device is configured to deliver the first stimulation signal to the sympathetic nerve at a position between the penile vein and the first suppression arrangement.

In an example, the control unit is configured to regulate the suppression of the action potentials to inhibit an undesired response of the nervous system of the person, the undesired response being generated responsive to the first stimulation signal.

In an example, the control unit is configured to apply the first stimulation signal and the first suppression signal in sequence, with a delay of the first suppression signal timed to generally match a conduction velocity of the first stimulation signal in the sympathetic nerve.

In an example, the suppression device is configured to vary an amplitude of the first suppression signal with frequency in the range of 1-10 KHz.

In an example, the first suppression arrangement comprises a first suppression electrode and a second suppression electrode configured to be spaced apart along the sympathetic nerve.

In an example, the first suppression arrangement further comprises a third suppression electrode configured to be arranged spaced apart from the first and second suppression electrodes along the sympathetic nerve such that the second suppression electrode is arranged between the first and third suppression electrodes.

In an example, the suppression device is configured to generate the first suppression signal such that the second suppression electrode serves as a cathode and the first and third suppression electrodes serve as anodes.

In an example, the system further comprises a suppression device comprising a second suppression arrangement configured to apply a second suppression signal to the parasympathetic nerve to suppress action potentials generated by the second stimulation signal and propagating in a the direction towards the central nervous system.

In an example, the stimulation device is configured to deliver the second stimulation signal to the parasympathetic nerve at a position between the penile artery and the second suppression arrangement.

In an example, the control unit is configured to regulate the suppression of the action potentials to inhibit an undesired response of the nervous system of the person, the undesired response being generated responsive to the second stimulation signal.

In an example, the suppression device is configured to apply the second stimulation signal and the second suppression signal in sequence, with a delay of the second suppression signal timed to generally match of conduction velocity of the second stimulation signal in the parasympathetic nerve.

In an example, the suppression device is configured to vary an amplitude of the second suppression signal with frequency in the range of 1-10 KHz.

In an example, the second suppression arrangement comprises a first suppression electrode and a second suppression electrode configured to be spaced apart along the parasympathetic nerve.

In an example, the first suppression arrangement further comprises a third suppression electrode configured to be arranged spaced apart from the first and second suppression electrodes along the parasympathetic nerve such that the second suppression electrode is arranged between the first and third suppression electrodes.

In an example, the suppression device is configured to generate the second suppression signal such that the second suppression electrode serves as a cathode and the first and third suppression electrodes serve as anodes.

According to an embodiment, a system for controlling appetite in a patient is provided, comprising:
a stimulation device configured to deliver, directly or indirectly a stimulation signal to the vagus nerve; and
a control unit configured to control an operation of the stimulation device to affect a propagation of action potentials in the vagus nerve such that the patient's feeling of satiety or hunger is affected.

In an example, the control unit is configured to control the operation of the stimulation device such that the stimulation signal suppresses the propagation of action potentials in a cranial direction, thereby hindering a hunger signal conveyed by the vagus nerve from reaching the patient's brain.

In an example, the control unit is configured to control the operation of the stimulation device such that the stimulation signal is a time-varying signal with an amplitude varying with a frequency in the range of 1-10 KHz.

In an example, the control unit is configured to control the operation of the stimulation device such that the stimulation signal generates action potentials propagating in a cranial direction, thereby causing a satiety signal to be conveyed by the vagus nerve to the patient's brain.

In an example, control unit is configured to control the operation of the stimulation device such that the stimulation signal is a time-varying signal with an amplitude varying with a frequency in the range of 0.1-100 Hz.

In an example, the control unit is configured to control the operation of the stimulation device such that the stimulation signal affects the propagation of action potentials in the vagus nerve to suppress a stomach peristalsis.

In an example, the control unit is configured to control the operation of the stimulation device such that the stimulation signal stimulates an activity of a parasympathetic nerve fiber of the vagus nerve innervating muscle tissue of the stomach wall.

In an example, control unit is configured to control the operation of the stimulation device such that the stimulation signal is a time-varying signal with an amplitude varying with a frequency in the range of 0.1-100 Hz.

In an example, the control unit is configured to control the operation of the stimulation device such that the stimulation signal suppresses the propagation of action potentials in a sympathetic fiber of the vagus nerve innervating muscle tissue of the stomach wall.

In an example, the control unit is configured to control the operation of the stimulation device such that the stimulation signal is a time-varying signal with an amplitude varying with a frequency in the range of 1-10 KHz.

In an example, the stimulation device comprises a stimulation arrangement configured to deliver the stimulation signal to the vagus nerve, wherein the stimulation arrangement is configured to be coupled to the vagus nerve at a position caudal to the esophageal hiatus.

In an example, the stimulation arrangement is configured to be coupled to an esophageal plexus of the vagus nerve.

In an example, the stimulation arrangement is configured to be coupled to an anterior trunk of the vagus nerve.

In an example, the stimulation arrangement is configured to be coupled to a gastric branch of the vagus nerve.

In an example, the stimulation device comprises a suppression arrangement configured to be coupled to the vagus nerve, wherein the control unit is configured to drive the stimulation device to apply, by means of the suppression arrangement, a suppression signal suppressing action potentials propagating in a direction towards the central nervous system in response to the stimulation device applying the stimulation signal.

In an example, the stimulation device comprises a stimulation arrangement configured to deliver the stimulation signal to the vagus nerve, wherein the suppression arrangement is configured to be coupled to the vagus nerve caudal to the stimulation arrangement.

In an example, the control unit is configured to regulate the suppression of the action potentials so as to inhibit an undesired response of the nervous system of the patient, the undesired response being generated responsive to the stimulation arrangement applying the stimulation signal.

In an example, the control unit is configured to drive the stimulation device such that the stimulation arrangement and the suppression arrangement are actuated in sequence, with a delay of the suppression signal timed to generally match a conduction velocity of the stimulation signal in the vagus nerve.

In an example, the control unit is configured to drive the stimulation device to apply the stimulation signal and the suppression signal substantially at the same time.

In an example, the control unit is configured to drive the stimulation device such that each f other stimulation signal and the suppression signal is a time-varying signal, wherein the stimulation signal is a low-frequency signal and the suppression signal is a high-frequency signal.

In an example, an amplitude of the stimulation signal varies with a frequency in the range of 0.1-100 Hz and wherein an amplitude of the suppression signal varies with a frequency in the range of 1-10 KHz.

In an example, the system further comprises a sensor device configured to generate a sensor signal indicating an effector response in tissue innervated by the vagus nerve and/or the action potentials propagating in the vagus nerve.

In an example, the control unit is configured to receive the sensor signal and to control an operation of the stimulation device based at least in part on the sensor signal.

In an example, the control unit is configured to determine a response measure based on the sensor signal, the response measure being indicative of the effector response.

In an example, the control unit is configured to:
compare the response measure with a predetermined reference measure; and
control the stimulation device to:
increase an intensity of the stimulation signal in response to the response measure being below the reference measure, and
reduce the intensity of the stimulation signal in response to the response measure exceeding the reference measure.

In an example, the control unit is configured to:
increase the intensity of the stimulation signal by increasing at least one of a frequency, current amplitude, and voltage amplitude of the stimulation signal; and
reduce the intensity of the stimulation signal by reducing at least one of the frequency, current amplitude, and voltage amplitude of the stimulation signal.

In an example, the predetermined reference measure is based on a previous measurement of the effector response in the patient.

In an example, the predetermined reference measure is based on previous measurements of effector responses in other patients.

In an example, the control unit is configured to monitor the level of effector response over time, and to control the stimulation device based on a change rate in the effector response over time.

According to an embodiment, there is provided an implantable vibration device comprising:
a vibration generating unit (VGU) configured to cause the implantable vibration device to vibrate, wherein the vibration generating unit comprises at least one piezoelectric material, and
a wireless energy receiver (R) configured to receive wireless energy to be used, directly or indirectly, by the vibration generating unit, and
a casing enclosing at least the vibration generating unit.

In an example, the casing further encloses the wireless energy receiver (R).

In an example, the wireless energy receiver is provided outside the casing and coupled to the vibration generating unit through a lead.

In an example, the implantable vibration device further comprises a rechargeable energy storage unit provided within the casing, for storing at least part of the received wireless energy.

In an example, the implantable vibration device comprises an internal controller.

In an example, the internal controller is configured to wirelessly receive vibration control data for controlling the vibration of the implantable vibration device.

In an example, the internal controller is configured to receive the vibration control data wirelessly via the wireless energy receiver.

In an example, the casing further encloses the internal controller.

In an example, the piezoelectric material is a ceramic piezoelectric material.

In an example, the piezoelectric material is lead zirconate titanate, PZT.

In an example, the piezoelectric material is barium titanate.

In an example, the piezoelectric material is lead titanate.

In an example, the piezoelectric material is a polymeric piezoelectric material.

In an example, the polymeric piezoelectric material is polyvinylidene fluoride, PVDF.

In an example, the piezoelectric material is comprised in a piezoelectric motor.

In an example, the piezoelectric motor is a piezoelectric inchworm motor.

In an example, the piezoelectric motor is a piezoelectric inertial motor.

In an example, the piezoelectric motor is a piezoelectric walk-drive motor.

In an example, the piezoelectric motor is a linear piezoelectric motor.

In an example, the vibration generating unit is attached to the casing, so that vibrations generated by the vibration generating unit can travel to the casing.

In an example, the piezoelectric motor is a rotational piezoelectric motor.

In an example, the vibration generating unit further comprises an weight configured to be eccentrically rotated by the rotational piezoelectric motor.

In an example, the vibration generating unit is configured to cause the implantable vibration device to vibrate at a frequency in the range of 1-150 Hz, such as in the range of 35-150 Hz.

In an example, the vibration generating unit is configured to cause the implantable vibration device to vibrate at an amplitude of at least 1 mm.

In an example, the implantable vibration device comprises an outer surface and a coating arranged on the outer surface.

In an example, the coating comprises at least one layer of a biomaterial.

In an example, the biomaterial comprises at least one drug or substance with one or more of the following characteristics: an antithrombotic, an antibacterial and an antiplatelet characteristic.

In an example, the biomaterial is fibrin-based.

In an example, further comprising a second coating arranged on the first coating.

In an example, the second coating is of a different biomaterial than said first coating.

In an example, the first coating comprises a layer of perfluorocarbon chemically attached to the surface, and wherein the second coating comprises a liquid perfluorocarbon layer.

In an example, the coating comprises a drug encapsulated in a porous material.

In an example, the surface comprises a metal.

In an example, the metal comprises at least one of the following, titanium, cobalt, nickel, copper, zinc, zirconium, molybdenum, tin or lead.

In an example, the surface comprises a micro pattern.

In an example, the surface further comprises a layer of a biomaterial coated on the micro pattern.

In an example, the vibration generating unit is substantially non-magnetic.

In an example, the vibration generating unit is substantially non-metallic.

In an example, the piezoelectric motor is a reversable piezoelectric motor.

According to an embodiment, a medical system is provided, comprising an external device configured for communication with an implantable medical device when implanted in a patient, wherein the medical device comprises a stimulation device according to any of the above embodiments.

Any embodiment, part of embodiment, example, method or part of method may be combined in any applicable way within the terms of the appended claims.

### BRIEF DESCRIPTION OF DRAWINGS

The inventive concept is now described, by way of example, with reference to the accompanying drawings, in which:

FIG. 1A shows an example of a system for affecting an effector response in a patient.

FIGS. 1B to 1E show various examples of electrodes and electrode arrangements.

FIGS. 2A and B show diagrams illustrating electric stimulation signals

FIG. 3A shows an example of a system for affecting an effector response in effector tissue of a patient.

FIGS. 3B to 3F show various examples of electrodes and electrode arrangements.

FIGS. 4A-C are diagrams illustrating signals as applied in the context of the present inventive concept.

FIG. 5A shows an example of a system comprising a sensor device for generating feedback indicative of an effector response.

FIGS. 5B to 5D show various examples of sensor devices.

FIG. 6 is a schematic outline of a system according to the inventive concept.

FIG. 7A shows an example of a multi-layer PCB.

FIG. 7B shows an example of a stretchable PCB.

FIG. 8A shows an example of a system comprising an inhibition device and a denervation device.

FIGS. 8B-D show various examples of inhibition and denervation devices.

FIG. 9 is a schematic illustration of the spinal autonomous nerves.

FIG. 10A shows an example of a system for delivering vibrations to nerves innervating erectile tissue of a male person.

FIGS. 10B-D are cross sections of erectile tissue.

FIG. 11 is an illustration of the vagal nerve and its innervation of the stomach.

FIG. 12 illustrate the autonomous innervation of the gastrointestinal tract.

FIGS. 13A-B show an example of the kidneys of a human patient, and the blood vessels supplying the kidneys with blood.

FIG. 14 shows an example of the innervation of the renal arteries leading to the kidneys.

FIGS. 15A-B show the mechanisms of vasoconstriction and vasodilation in a blood vessel.

FIGS. 16-20 show various examples of medical devices implanted to electrically or otherwise induce vasodilation in the renal artery.

FIGS. 21A-D are various examples of sensors.

FIGS. 22A-B illustrate an electrical stimulation device and a sensor implanted at the rental artery.

FIG. 23 show a system implanted at the renal artery.

FIGS. 24A-B schematically show implantable vibration devices according to the present disclosure.

FIG. 25 shows, schematically, an embodiment of an inchworm motor.

FIG. 26 illustrates, schematically, an operation cycle of a piezoelectric inchworm motor.

FIG. 27 shows, schematically, an embodiment of a piezoelectric inertial motor.

FIG. 28 shows, schematically, an embodiment of a piezoelectric walk-drive motor.

FIG. 29 illustrates, schematically, an operation cycle of a piezoelectric walk-drive motor.

FIGS. 30A-B illustrate, schematically, implantable vibration devices comprising an eccentric mechanism operated by a motor.

FIG. 31 shows, schematically, a Traveling Wave Ultrasonic Motors (TWUSM).

FIG. 32 shows, schematically, an embodiment of a Standing Wave Ultrasonic Motor (SWUSM).

FIG. 33 shows, schematically, an embodiment of a linear ultrasonic motor.

FIG. 34 shows, schematically, an embodiment of an implantable vibration device comprising a piezoelectric vibration generating unit.

FIGS. 35A-35FH show an embodiment and describe various functions of an implantable controller for controlling the implantable medical device.

FIG. 35G shows an elevated perspective view from the left of a housing unit.

FIG. 35H shows a plain view from the left of a housing unit.

FIG. 35I shows an elevated perspective view from the left of a housing unit.

FIG. 35J shows a plain view from the left of a housing unit.

FIG. 35K shows a system overview of an external device comprising a housing unit and a display device in wireless communication with an implanted medical device.

FIG. 35L shows a system having a first and a second remote control.

FIG. 35LL shows the second remote control comprised in a housing unit.

FIG. 35M schematically shows a medical implant when implanted in a patient.

FIG. 35N shows a flow chart for a method for training a medical implant to recognize a voice command, according to some embodiments.

FIG. 35O shows a flow chart for a method for using voice commands to control a medical implant, according to some embodiments.

FIGS. 35P-T illustrates implantable medical devices and external devices for transferring wireless energy to the implantable medical devices.

FIG. 35U illustrates an implantable medical device and an external device configured to transmit data using near field magnetic induction.

### DETAILED DESCRIPTION

In the following, a detailed description of embodiments of the invention will be given with reference to the accompanying drawings. It will be appreciated that the drawings are for illustration only and are not in any way restricting the scope of the invention as defined by the appended claims. Thus, any references to directions, such as “up” or “down”, are only referring to the directions shown in the figures. It should be noted that the features having the same reference numerals generally may have the same function. A feature in one embodiment could thus be exchanged for a feature from another embodiment having the same reference numeral, unless clearly contradictory. The description of the features having the same reference numerals should thus be seen as complementing each other in describing the fundamental idea of the feature and thereby showing the versatility of the feature.

Muscle tissue is generally formed of muscle cells that are joined together in tissue that can be either striated or smooth, depending on the presence or absence, respectively, of organized, regularly repeated arrangements of myofibrillar contractile proteins called myofilaments. Striated muscle tissue is further classified as either skeletal or cardiac muscle tissue. Skeletal muscle tissue is typically subject to conscious control and anchored by tendons to bone. Cardiac muscle tissue is typically found in the heart and not subject to voluntary control. A third type of muscle tissue is the so-called smooth muscle tissue, which is typically neither striated in structure nor under voluntary control. Smooth muscle tissue can be found in the walls of blood vessels and the gastrointestinal tract, for example.

The contraction of the muscle tissue may be activated both through the interaction of the nervous system as well as by hormones. The different muscle tissue types may vary in their response to neurotransmitters and endocrine substances depending on muscle type and the exact location of the muscle.

A nerve is an enclosed bundle of nerve fibers called axons, which are extensions of individual nerve cells or neurons. The axons are electrically excitable, due to maintenance of voltage gradients across their membranes, and provide a common pathway for the electrochemical nerve impulses called action potentials. An action potential may be understood as an all-or-nothing electrochemical pulse generated by the axon if the voltage across the membrane changes by a large enough amount over a short interval. The action potentials travel from one neuron to another by crossing a synapse, where the message is converted from electrical to chemical and then back to electrical.

The distal terminations of an axon are called axon terminals and comprise synaptic vesicles storing neurotransmitters. The axonal terminals are specialized to release the neurotransmitters into an interface or junction between the axon and the muscle cell. The released neurotransmitter binds to a receptor on the cell membrane of the muscle cell for a short period of time before it is dissociated and hydrolyzed by an enzyme located in the synapse. This enzyme quickly reduces the stimulus to the muscle, which allows the degree and timing of muscular contraction to be regulated delicately.

The action potential in a normal skeletal muscle cell is similar to the action potential in neurons and is typically about −90 mV. Upon activation, the intrinsic sodium/potassium channel of the cell membrane is opened, causing sodium to rush in and potassium to trickle out. As a result, the cell membrane reverses polarity and its voltage quickly jumps from the resting membrane potential of −90 mV to as high as +75 mV as sodium enters. The muscle action potential lasts roughly 2-4 ms, the absolute refractory period is roughly 1-3 ms, and the conduction velocity along the muscle is roughly 5 m/s. This change in polarity causes in turn the muscle cell to contract.

The contraction and relaxation of smooth muscle cells is typically influenced by multiple inputs such as spontaneous electrical activity, neural and hormonal inputs, local changes in chemical composition, and stretch. This in contrast to the contractile activity of skeletal and cardiac muscle cells, which may rely on a single neural input. Some types of smooth muscle cells are able to generate their own action potentials spontaneously, which usually occur following a pacemaker potential or a slow wave potential. However, the rate and strength of the contractions can be modulated by external input from the autonomic nervous system. Autonomic neurons may comprise a series of axon-like swellings, called varicosities, forming motor units through the smooth muscle tissue. The varicosities comprise vesicles with neurotransmitters for transmitting the signal to the muscle cell. The autonomic neurons may for example trigger a muscular response in the wall of the renal artery, leading to a contraction or relaxation affecting a flow resistance in the renal artery. Sympathetic stimulation (norepinephrine) has been observed to constrict some blood vessels and dilate others, depending on whether the target cells (i.e., the smooth muscle cells) has alpha- or beta-adrenergic receptors. The sympathetic nervous system can also constrict or dilate vessels just by changing firing frequency of the action potentials. An increased firing frequency may cause the smooth muscle to contract and constrict the vessel, whereas a reduced firing frequency may cause the smooth muscle cells to relax, allowing blood pressure to dilate the vessel.

The muscle cells described above, i.e., the cardiac, skeletal and smooth muscle cells are known to react to external stimuli, such as electrical stimuli applied by electrodes. A distinction can be made between stimulation transmitted by a nerve and direct electrical stimulation of the muscle tissue. In case of stimulation via a nerve, an electrical signal may be provided to the nerve at a location distant from the actual muscle tissue, or at the muscle tissue, depending on the accessibility and extension of the nerve in the body. The stimulation devices as well as the signal damping devices described in the present disclosure may employ both a direct stimulation of the muscle tissue and stimulation transmitted via a nerve to affect the vasomotor tone.

In case of direct stimulation of the muscle tissue, the electrical signal may be provided to the muscle cells by an electrode arranged in direct or close contact with the cells of the renal artery 20. However, other tissue such as fibrous tissue and nerves may of course be present at the interface between the electrode and the muscle tissue, which may result in the other tissue being subject to the electrical stimulation as well.

In the context of the present application, the electrical stimulation discussed in connection with the various aspects and embodiments may be provided to the tissue in direct or indirect contact with the implantable medical device. Preferably, the electrical stimulation is provided by one or several electrode elements arranged at the interface or contact surface between the implantable constriction device and the tissue. Thus, the electrical stimulation for exercising the tissue may, in terms of the present disclosure, be considered as a direct stimulation of the tissue. Particularly when contrasted to stimulation transmitted over a distance by a nerve, which may be referred to as an indirect stimulation or nerve stimulation.

A control unit or controller is to be understood as any implantable unit capable of controlling the operation of an electrically operated device, such as a stimulation device or a signal damping device. A controller could include an electrical power source or another operation device for operating the stimulation device and the signal damping device. A control unit may also be understood as an element comprising circuitry configured to carry out various functions, such as data storage and processing, and signal generation. The control unit may be configured to transmit the control instructions to the stimulation device over a wired channel or a wireless channel. Further, the control unit may comprise an external part configured to be arranged outside the body of the patient and an internal part configured to be implanted in the patient. The internal and external parts may be configured to communicate wirelessly with each other, for example by means of radiofrequency signals or inductive signals.

A control signal is to be understood as any signal capable of carrying information and/or electric power such that for instance the stimulation device can be directly or indirectly controlled.

An implantable operation device, sometimes also referred to as a controller, may further be understood as any device or system capable of operating an active implant. An operation device or controller could for example be an actuator such as a hydraulic actuator including for instance a hydraulic pump or a hydraulic cylinder, or a mechanical actuator, such as a mechanical element actuating an implant by pressing or pulling directly or indirectly on the implant, or an electromechanical actuator such as an electrical motor or solenoid directly or indirectly pressing or pulling on the implant. The operation device may comprise a control unit as described above, and/or circuitry configured to carry out such functions.

The sympathetic nervous system (SNS) and the parasympathetic nervous system (PNS) form part of the autonomous nervous system (ANS) of the body. The SNS and the PNS control involuntary bodily functions such as, for example, heart rate, blood pressure, digestion, breathing rate, pupil size, blood flow to the muscles, and sexual responses. The SNS is commonly described as the “fight or flight” system, preparing the body for stress or danger, whereas the PNS is commonly referred to as the “rest and digest” system, promoting relaxation, energy storage, and other non-emergency functions. Activation of the SNS may result in increased heart rate, dilated airways, inhibited digestion, dilated pupils, and redirection of blood to muscles. Activation of the PNS may result in decreased heart rate, stimulated digestion, contracted pupils, and relaxation of muscles. For the SNS, the preganglionic neurons generally originate in the thoracic and lumbar regions of the spinal cord, whereas for the PNS the preganglionic neurons generally originate in the brainstem and the sacral region of the spinal cord.

The SNS and the PNS may have complementary functions on the same effector tissue, such as an organ or a muscle. For example, the SNS may accelerate the heart rate, while the PNS may slow it down. Both the SNS and the PNS are typically active to some extent all the time, but their relative activities may change depending on the situation. This dynamic balance between the SNS and the PNS is commonly referred to as the “autonomic tone”. The autonomic tone hence implies there is an ongoing, background level of activity in the SNS and PNS. The body may adjust this balance as needed, ramping up sympathetic or parasympathetic activity in response to specific situations. The tone may also capture the systems' readiness to respond to stimuli. A certain “tone” or baseline activity level may ensure that the system can quickly ramp up or down its activity to adapt to different situations.

Therefore, it may be of interest to adjust or affect this tone for therapeutic purposes, for example to affect the general level of stress in the body. Adjusting the level of activity in the SNS and/or the PNS may, for example, be employed to treat a variety of conditions. For example, increasing the PNS activity and/or decreasing the SNS activity can help treating conditions such as anxiety, hypertension, and irritable bowel syndrome (IBS). On the other hand, increasing SNS activity and/or decreasing PNS activity can help treating conditions such as depression, chronic fatigue syndrome, and postural orthostatic tachycardia syndrome (PoTS).

In some instances, both systems work together to perform a function. For example, during sexual arousal and ejaculation, both systems are involved in different phases. During sexual arousal, the PNS may cause the arteries in the erectile tissue to dilate to increase the blood flow, whereas the SNS may cause the veins to contract to reduce the blood flow leaving the erectile tissue. The SNS and the PNS are also coopering during urination, wherein the PNS may cause the sphincters to relax and the SNS the bladder to contract. Adjusting or controlling the cooperation between the SNS and PNS may thus be employed to treat impotence and incontinence.

The SNS and the PSNS may generally be considered to work together in a dynamic balance between arousal/activation/contraction and relaxation/inhibition/relaxation in the effector tissue innervated by the SNS and the PNS. Hence, the sympathetic activity and the parasympathetic activity may affect the response in the effector tissue, which typically may be a somatic effector tissue or an autonomic effector tissue. Examples of somatic effector tissue include muscular tissue, such as skeletal muscles, whereas examples of autonomic effector tissue include smooth muscle tissue, cardiac muscle tissue, and glandular or epithelial tissue (commonly involved in the production and secretion of various substances such as hormones, enzymes, and sweat). By stimulating a sympathetic nerve and/or a parasympathetic nerve innervating the effector tissue, the effector response may be controlled or affected accordingly. In case of muscular tissue, the effector response may be a contraction or relaxation of the tissue. In case the effector tissue being glandular tissue, the effector response may be an increased or reduced production or secretion of, for example, a hormone or an enzyme. In some examples, the stimulation of the sympathetic nerve and/or parasympathetic nerve may be employed to adjust the autonomic tone discussed above.

Generally, ‘effector tissue’ refers to tissues in the body that produce a response or perform work (‘effector response’) when activated by nerve signals. Effectors may essentially be understood as the ‘end targets’ in the signalling pathways of the nervous system. As mentioned above, they may be categorized as muscles (skeletal muscles, smooth muscle) and glands (endocrine glands and exocrine glands). They may furthermore be categorized based on their relationship with the nervous system. The somatic effectors are mostly skeletal muscles controlled by the somatic nervous system, responsible for voluntary actions, whereas the autonomic effectors typically are controlled by the autonomic nervous system and include smooth muscle tissue, cardiac muscle, and glands. These effectors are generally not under voluntary control. It is to be noted that the various aspects of stimulation discussed in the present disclosure may be applied to any type of effector tissue, including somatic effectors as well as autonomic effectors.

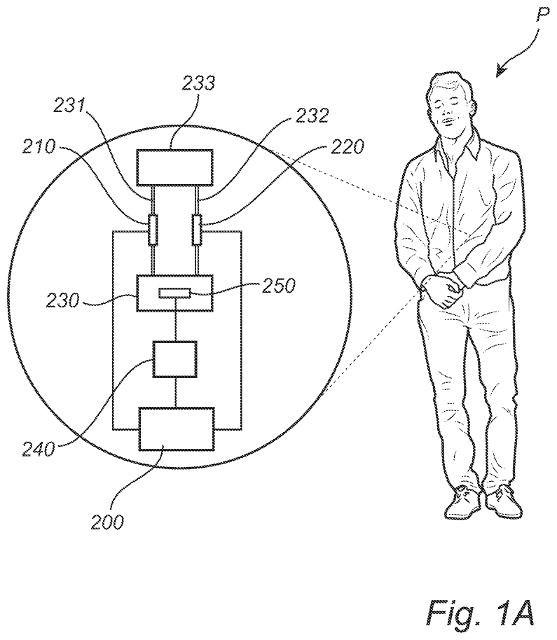

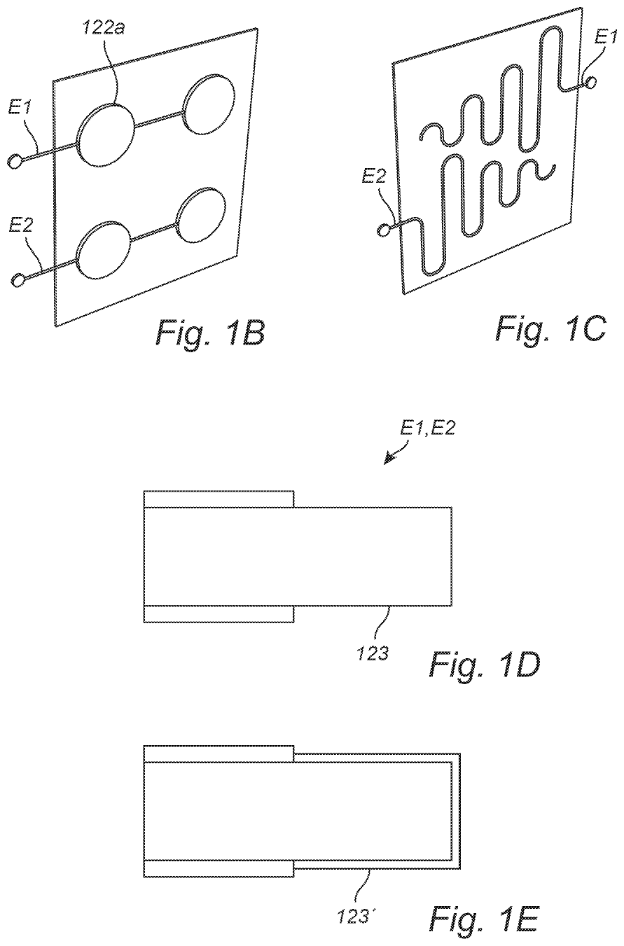

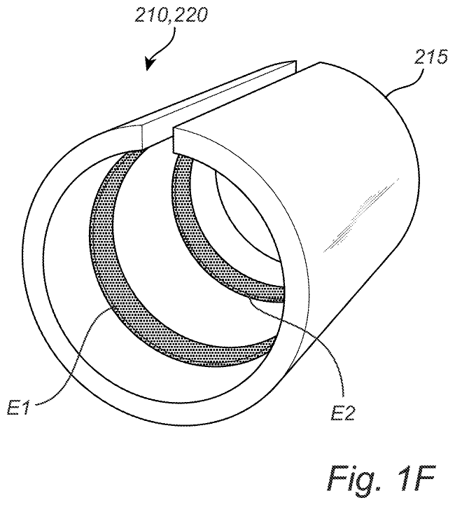

An exemplary system for affecting an effector response in a patient will now be discussed with reference to FIG. 1A to FIG. 1E. FIG. 1A is a schematic illustration of a particular example of the system and its interaction with the body of the patient P. and more particularly with an effector tissue 230 innervated by a sympathetic nerve 231 and a parasympathetic nerve 232. As indicated, the sympathetic nerve 231 and the parasympathetic nerve 232 may extend between the effector tissue 230 and the central nervous system (CNS) 233. The sympathetic nerve 231 may, for example, originate from the thoracic or lumbar regions of the spinal cord, whereas the parasympathetic nerve may originate in the brainstem or the sacral region of the spinal cord. In the present example, the origin of the illustrated nerves 231, 232 is represented by item 233. It will be appreciated that the effector tissue 230 and the CNS 233 are merely schematically indicated and that the technology described in the following may be applied to various types of nerves and effector tissues, located in various positions and parts of the body P.

In some examples, the sympathetic nerve 231 and the parasympathetic nerve 232 may innervate the same effector tissue 230, which thus may be considered to have a dual autonomous nervous system (ANS) innervation. This means that the effector tissue 230 may receive competing inputs from the sympathetic and the parasympathetic divisions of the ANS. In other examples, the sympathetic nerve 231 may innervate a first effector tissue and the parasympathetic nerve 232 may innervate a second effector tissue, the second effector tissue being different from the first effector tissue 230. In other words, the sympathetic nerve 231 and the parasympathetic nerve 232 may innervate different organs, muscles, or part of a muscle. Both these examples, i.e., in which the sympathetic and parasympathetic nerves 231, 232 innervate the same or different tissue, are represented by item 230.

As illustrated, the system comprises a stimulation device configured to deliver, directly or indirectly, a first simulation signal to the sympathetic nerve 231 innervating the first effector tissue 230 and a second stimulation signal to the parasympathetic nerve 232 innervating the second effector tissue 230. The effector tissue 230 may hence be the same effector tissue, forming part of the same muscle or organ, or different effector tissues 230, forming part of different muscles or organs. The system further comprises a control unit or controller 240, configured to control an operation of the stimulation device such that the first stimulation signal stimulates an activity of the sympathetic nerve 231 and the second stimulation signal inhibits an activity of the parasympathetic nerve 232 or such that the first stimulation signal inhibits an activity of the sympathetic nerve 231 and the second stimulation signal stimulates an activity of the parasympathetic nerve 232. Hence, each of the first and second stimulation signals may result either in an activation or an inhibition, depending on the characteristics of the stimulation signal. A signal that results in an activation of the nerve (and/or the effector tissue) may be referred to as an activation signal, whereas a signal that results in an inhibition of the nerve (and/or the effector tissue) may be referred to as an inhibition signal. “Activation of a nerve” is generally to be understood as the generation of a nerve signal, i.e., action potentials travelling in the nerve, whereas “inhibition of a nerve” is generally to be understood as blocking or hindering any nerve signals from propagating through the nerve. Inhibition may also be referred to as a suppression or blocking of the nerve and/or its signals. It should be noted that the blocking may not always be complete; on the contrary, there may still be some activity in the nerve. However, it is preferable to suppress the nerve signal to a degree that results in no or a negligible response in the effector tissue 230.

The nervous response, or effector response, may typically be determined by, inter alia, a frequency content of the signal. The signal may be a periodic signal, including at least one of: a variable frequency component, a variable duty cycle component, a variable amplitude component, and a variable pause component. Generally, a low-frequency stimulation may be more likely to result in inhibition, whereas high-frequency stimulation tends to excite neural pathways and effector tissue. Further, higher voltages and currents may more often lead to activation as compared to lower voltages and currents. The response to a stimulation signal may however vary based on other factors, such as location and target tissue. It may therefore be beneficial to measure the effect of the stimulation to determine whether the treatment has an intended effect or not and to provide feedback that can be used to adjust the characteristics of the stimulation signal. For example, the measured effect may be used as feedback in a closed loop control of the stimulation device. This will be discussed in further detail later in the present disclosure.

The activation signal and the inhibition signal may cooperate to achieve a certain response. For example, the activation and inhibition signals may be applied to an antagonistic muscle pair, where the activation signal may be applied to the agonist to cause it to contract and the inhibition signal may be applied to the antagonist muscle to cause it to relax. Hence, this is an example of the effector tissue 230 forming part of different organs, such as the agonist and the antagonist of an antagonistic muscle pair. In other examples, the activation and inhibition signals may be applied in sequence to the same effector tissue 230, such that an activation (such as a contraction) triggered by the activation signal is followed by an inhibition (such as a relaxation) triggered by the inhibition signal, or vice versa.

It is also possible to apply several stimulation signals and/or inhibition signals to several different effector tissues. A first stimulation signal may, for example, be applied to a first effector tissue (such as a first muscle of an antagonistic pair) and a second stimulation signal may be applied to a second effector tissue (such as the other muscle of the antagonistic pair). By applying the first and second stimulation signals in sequence, an improved control and stabilization of body movement may be achieved.

The present system can be employed to treat or at least reduce or alleviate muscle spasms, which may be understood as sudden, involuntary contractions of one or more muscles. By applying an inhibition signal to the nerve innervating the muscle that is contracting during the spasm, or by applying the inhibition signal directly to that muscle, the muscle may be caused to relax. Alternatively, or additionally, the activation signal may be applied to a nerve innervating a muscle counteracting the spasming muscle. As the counteracting muscle responds to the activation signal by contracting, it may help balancing or counteracting the spasming muscle. This approach may be particularly useful for addressing symptoms of, for example, a herniated disc, sciatica, spinal stenosis, as well as neurological disorders such as multiple sclerosis, Parkinson's disease, dystonia, and cerebral palsy.

A stimulation signal may have different characteristics, depending on the desired effector response to be achieved. As already mentioned, these characteristics may relate to amplitude, frequency, waveform, polarity, and duty cycle. The duty cycle may be understood as the ratio of the time that the signal is ‘on’, i.e., active, to the total time of one cycle (period). For a pulsed signal, this would correspond to the ratio of the pulse length to the length of a cycle. The duty cycle is usually expressed as a percentage or a fraction. For example, a 50% duty cycle may be understood as the signal being ‘on’ for half of the cycle and “off” for the other half. The duty cycle of an electric stimulation signal can influence the energy delivered to the tissue (or at least the energy to which the tissue is exposed). A higher duty cycle means that the tissue may be exposed to more electric charges and energy, which can increase the stimulation effect, whereas a lower duty cycle means that the tissue may be exposed to less electric charge and energy. A lower duty cycle may mean that the tissue has more time to recover and adapt to the stimulation, which can reduce the risk of tissue damage or fatigue. Different types of tissues may require different duty cycles for optimal stimulation. Electric stimulation signals with low duty cycles (less than 10%) have been shown to promote cell regeneration, proliferation, and growth, whereas electric signals with high duty cycles (more than 50%) have been shown to inhibit cell growth. Generally, duty cycles above 10% may result in a stronger and faster contraction of muscle cells, while duty cycles below 10% may result in a weaker and slower contraction of muscle cells.

Accordingly, the pause component of a stimulation signal, describing the time interval between two consecutive pulses or two consecutive pulse trains, can affect the stimulation of tissue in several ways. Increasing the pause component may facilitate recovery from the previous stimulation and reduce the risk of overstimulation or fatigue. A longer pause component can reduce the risk of tissue damage or adaptation, while a shorter pause component can increase the stimulation effect. Furthermore, the pause component may influence the net charge delivered to the tissue and the electrochemical reactions at the electrode-tissue interface. A longer pause component can allow the charge to dissipate and the pH to normalize, while a shorter pause component can cause charge accumulation and pH changes. The pause component may in some examples range from 0.1-10 seconds, such as 0.5-2 seconds, depending on the tissue type.

In an example, a nerve, such as the sympathetic nerve 231 or the parasympathetic nerve 232, may be activated or stimulated by an activation signal comprising a frequency in the range of 0.1-100 Hz, such as 1-50 Hz. Such a signal may be referred to as a low-frequency signal. The activation signal may comprise a voltage in the range of 1-15 V, such as about 10 V and a current in the range of 1-50 mA, such as 2-4 mA, depending on the target tissue. Applying such a signal to a sympathetic nerve 231 may typically result in an activation of the effector tissue 230, such as a contraction of muscle tissue or an increased secretion of a gland, whereas applying the signal to a parasympathetic nerve 232 may typically result in an inactivation of the effector tissue 230. An inactivation may typically include relaxation of a muscle or a reduced secretion of a gland. Applying such a signal directly to the effector tissue 230 may result in a similar response as applying it to the nerve.

To inhibit or inactivate the nerve, an inhibition signal comprising a frequency in the range of 1-10 kHz, such a 2-5 kHz, can be used. Similar to the activation signal, the voltage can be in the range of 1-15 V and the current in the range of 1-50 mA. Applying such as signal to a nerve 231, 232 may result in the nerve signals being blocked or at least heavily reduced. As an effect, the effector tissue 230 can be considered more or less cut off from the signals delivered by that nerve from the CNS 233.

The control unit 240 may be configured to control the operation of the stimulation device to provide a low-frequency signal for stimulating the activity of the sympathetic nerve 231 and a high-frequency signal for inhibiting the activity of the parasympathetic nerve 232. This may shift the balance between the SNS and PNS activity towards the SNS activity, which may result in a muscle contraction (in case of the effector tissue being a muscle tissue) or increased secretion of a gland (in case of the effector tissue being a glandular tissue). The control unit 240 may as well be configured to control the operation of the stimulation device to provide a high-frequency signal for inhibiting the activity of the sympathetic nerve 231 and a low-frequency signal for stimulating the activity of the parasympathetic nerve 232. This may shift the balance between the SNS and PNS activity towards the PNS activity, which may result in a muscle relaxation (in case of the effector tissue being a muscle tissue) or reduced secretion of a gland (in case of the effector tissue being a glandular tissue). The activation signal and the inhibition signal may be applied to the respective nerves 231, 232 concurrently, simultaneously, or separately, i.e., one at a time.

The stimulation device may comprise circuitry and a power source for generating the stimulation signals, which, for example, may be electrical signals or mechanical vibration signals. The circuitry and, optionally, the power source may be arranged within a housing which may be implantable in the body of the patient. Electrical leads may be provided to connect the circuitry to a signal generating means 210, 220 arranged at the respective nerves 231, 232. In case of an electric stimulation signal, the signal generating means 210, 220 may comprise a respective electrode arrangement. In case of a mechanical vibration signal the signal generating means 210, 220 may comprise a respective vibrator, such as a piezoelectric vibrator comprising one or more piezoelectric elements. The vibrations may be generated by the direct movement of the piezoelectric element, or by other mechanical elements actuated by the piezoelectric element. In an example, the vibrations may be generated by an eccentric weight that are brought to rotate by a piezoelectric actuator, such as a rotational motor.

At least parts of the stimulation device, such as a housing and/or energy source, may be implanted in fat tissue of the patient, be anchored to bone tissue, or implanted subcutaneously.

The control unit 240 may be integrated with the stimulation device, such as arranged within the same housing as the electric circuitry, or the energy source mentioned above. In other examples, the control unit 240 may be arranged separately or remotely, i.e., at a different physical location than the stimulation device. In the latter case, the control unit 240 may be communicatively coupled to the stimulation device by means of a wired or wireless connection. The control unit 240 may hence be arranged within the patient's body or externally, i.e., outside the body of the patient P.

The signal generating means may comprise a first electrode arrangement 210 configured to be coupled to the sympathetic nerve 231 to deliver the first stimulation signal (such as an activation signal or an inhibition signal) and a second electrode arrangement 220 configured to be coupled to the parasympathetic nerve 220 to deliver the second stimulation signal (such as an inhibition signal or an activation signal). FIG. 1B to FIG. 1E show examples of electrode arrangements which may be implemented in any of the stimulation devices 40 discussed in the present disclosure.

FIG. 1B is an example of a bipolar electrode arrangement comprising a first and a second electrode element E1, E2, having a plurality of contact portions 122a which can be arranged to abut the sympathetic nerve 231 or the parasympathetic nerve 232 or touch the effector tissue 230. The electrode arrangement may be operated as a bipolar electrode arrangement by connecting the first and second electrode elements E1, E2 to different electrical potentials. Thus, the first electrode element E1 can be operated as an anode and the second electrode element E2 can be operated as a cathode. The electrode elements E1, E2 may be attached directly to a support structure 126, such as a patch, or a cuff 215 as shown in FIG. 1E. The electrode arrangement may comprise one or several contact pads or contacting portions 122a, for increasing the contact surface between the electrode and the tissue when implanted. During operation, the stimulation signal may be delivered to the tissue, i.e., the nerve or the effector tissue, by means of the first and second electrode elements E1, E2 to activate or inhibit action potentials in the nerve or activate the effector tissue 230 directly.

FIG. 1C is another example of an electrode arrangement of an electrical stimulation device as discussed above. In the present example, the electrode arrangement may be operated as a unipolar electrode element or as a bipolar electrode arrangement. The electrode arrangement comprises a first electrode element E1 and a second electrode element E2 which may be formed of a wire or electrical lead arranged in a flat, coiled structure for increasing the contact surface between the electrode elements E1, E2 and the nerve tissue. The electrode elements E1, E2 may be arranged on a flexible and/or stretchable support or patch 216, allowing it to conform to the shape of the tissue to which it is attached and move with any movement of the same. The coiled configuration allows for a certain mechanical flexibility of the electrode elements E1, E2 such that they can move with any movement or deformation of the support 215.

FIG. 1D illustrates the end portion of a needle-shaped or pin-shaped electrode element E1. E2, wherein the active portion of the electrode element E1, E2 is provided as a bare electrode surface 123 at the end of the electrode element E1, E2. Thus, when implanted at or in the tissue, the active bare electrode surface 123 of the electrode element E1, E2 may form a metal-tissue interface with the tissue. The tip of the bare electrode surface 123 of the electrode element E1, E2 may be inserted into the nerve tissue 231, 232 or arranged to abut an outer surface of the nerve 231, 232. The present example may form a unipolar electrode and may require another electrode, such as a ground electrode, to be arranged elsewhere on the patient's body to form a closed electric circuit. The completing electrode may, for instance, be formed by a housing of the stimulation device.

FIG. 1E shows a similar electrode element E1, E2 as the one in FIG. 1D, with the difference that the present electrode element E1, E2 comprises an active tip portion that is covered by a dielectric material 123′ to protect the electrode material from deterioration during long-term implantation and to facilitate capacitive current transfer to the tissue. The dielectric material 123′ may for instance be electrochemically deposited tantalum oxide, which allow the electrical charge to pass through the interface but reduces the risk for electrode corrosion, gas formation and metabolite reactions.

It will be appreciated that both faradaic and capacitive mechanisms may be present at the same time, irrespectively of the type of electrode used. Thus, capacitive charge transfer may be present also for a bare electrode forming a metal-tissue interface, and faradaic charge transfer may be present also for a coated electrode forming a dielectric-tissue interface. It has been found that the faradaic portion of the current delivered to the muscle tissue can be reduced or even eliminated by reducing the duration of the pulses of the electric signal. Reducing the pulse duration has turned out to be an efficient way of increasing the portion of the signal which can be passed through the interface as a capacitive current, rather than by a faradaic current. As a result, shorter pulses may produce less electrode and tissue damage.

The capacitive portion of the current may further be increased, relative to the faradaic portion, by reducing the amplitude of the current pulses of the electrical signal. Reducing the amplitude may reduce or suppress the chemical reactions at the interface between the electrode and the tissue, thereby reducing potential damage that may be caused by compounds and ions generated by such reactions.

In one example, the electrical stimulation may be controlled in such a manner that a positive pulse of the electrical signal is followed by a negative pulse (or, put differently, a pulse of a first polarity being followed by a pulse of a second, reversed polarity), preferably of the same amplitude and/or duration. Advantageously, the subsequent negative (or reversed) pulse may be used to reverse or at least moderate chemical reactions or changes taking place in the interface in response to the first, positive pulse. By generating a reversed pulse, the risk of deterioration of the electrode and/or the tissue at the interface between the electrode and the muscle tissue may be reduced.

In the following, the interaction between an implanted electrode element and tissue of the body will be discussed. This discussion may be applied both to stimulation for inducing an effector response, such as vasodilation, inhibition or blocking of signals, as well as sensing and electrical stimulation for exercising the tissue to reduce effects of deterioration caused by presence of a long term implanted medical device.

It has been observed that the interaction between an implanted electrode element and tissue of the body is to a large extent determined by the properties at the junction between the tissue and the electrode element. The active electrically conducting surface of the electrode element (in the following referred to as “metal”, even though other materials is equally conceivable) can either be uncoated resulting in a metal-tissue interface (such as disclosed in FIG. 1D), or insulated with some type of dielectric material (such as disclosed in FIG. 1E). The uncoated metal surface of the electrode element may also be referred to as a bare electrode. The interface between the electrode element and the tissue may influence the behavior of the electrode element, since the electrical interaction with the tissue is transmitted via this interface. In the biological medium surrounding the electrode element, such as the actual tissue and any electrolyte that may be present in the junction, the current is carried by charged ions, while in the material of the electrode element the current is carried by electrons. Thus, in order for a continuous current to flow, there needs to be some type of mechanism to transfer charge between these two carriers.

In some examples, the electrode element may be a bare electrode wherein the metal may be exposed to the surrounding biological medium when implanted in, or at the muscle tissue that is to be stimulated. In this case there may be a charge transfer at a metal-electrolyte interface between the electrode element and the tissue. Due to the natural strive for thermodynamic equilibrium between the metal and the electrolyte, a voltage may be established across the interface which in turn may cause an attraction and ordering of ions from the electrolyte. This layer of charged ions at the metal surface may be referred to as a “double layer” and may physically account for some of the electrode capacitance.

Hence, both capacitive faradaic processes may take place at the electrode element. In a faradaic process, a transfer of charged particles across the metal-electrolyte interface may be considered as the predominant current transfer mechanism. Thus, in a faradaic process, after applying a constant current, the electrode charge, voltage and composition tend to go to constant values. Instead, in a capacitive (non-faradaic) process charge is progressively stored at the metal surface and the current transfer is generally limited to the amount which can be passed by charging the interface.

In some examples, the electrode element may comprise a bare electrode portion, i.e., an electrode having an uncoated surface portion facing the tissue such that a conductor-tissue interface is provided between the electrode element and the tissue when the electrode element is implanted. This allows for the electric signal to be transmitted to the tissue by means of a predominantly faradaic charge transfer process. A bare electrode may be advantageous from a power consumption perspective, since a faradaic process tend to be more efficient than a capacitive charge transfer process. Hence, a bare electrode may be used to increase the current transferred to the tissue for a given power consumption.

In some examples, the electrode element may comprise a portion that is at least partly covered by a dielectric material so as to form a dielectric-tissue interface with the muscle tissue when the electrode is implanted. This type of electrode element allows for a predominantly capacitive, or non-faradaic, transfer of the electric signal to the muscle tissue. This may be advantageous over the predominantly faradaic process associated with bare electrodes, since faradaic charge transfer may be associated with several problems. Example of problems associated with faradaic charge transfer include undesirable chemical reactions such as metal oxidation, electrolysis of water, oxidation of saline, and oxidation of organics. Electrolysis of water may be damaging since it produces gases. Oxidation of saline can produce many different compounds, some of which are toxic. Oxidation of the metal may release metal ions and salts into the tissue which may be dangerous. Finally, oxidation of organics in a situation with an electrode element directly stimulating tissue may generate chemical products that are toxic.

These problems may be alleviated if the charge transfer by faradaic mechanisms is reduced, which may be achieved by using an electrode at least partly covered by a dielectric material. Preferably, the dielectric material is chosen to have as high capacitance as possible, restricting the currents flowing through the interface to a predominantly capacitive nature.

Several types of electrode elements can be combined with the present disclosure. The electrode element can for example be a plate electrode as indicated in FIG. 1B, comprising a plate-shaped active part forming the interface with the tissue. In other examples, the electrode may be a wire electrode as indicated in FIG. 1C, formed of a conducting wire that can be brought in electrical contact with the tissue. Further examples may include needle- or pin-shaped electrodes as indicated in FIGS. 1D and E, having a point at the end which can be attached to or inserted in the muscle tissue. The electrodes may for example be encased in epoxy for electrical isolation and protection and comprise gold wires or contact pads for contacting the muscle tissue.

Preferably, the electrode may be arranged to transmit the electrical signal to the portions of the tissue that is affected, or risks to be affected, by mechanical forces exerted by the medical implant. Thus, the electrode element may be considered to be arranged between the implanted device and the tissue against which the device is arranged to rest when implanted.

During operation of the medical device, or the electrode arrangement, the electric signal may cause the muscle cells to contract and relax repeatedly. This action of the cells may be referred to as exercise and may have a positive impact in terms of preventing deterioration and damage of the tissue. Further, the exercise may help increasing tolerance of the tissue for pressure and mechanical forces generated by the medical implant. The contraction and relaxation induced for exercising purposes may thus be less than the effector response induced for the purpose of affecting bodily functions or treating symptoms. Alternatively, or additionally the exercise may involve contraction and relaxation at a relatively high frequency, hindering the blood vessel to contract to a degree that affects the vascular resistance in the vessel before it is relaxed again.

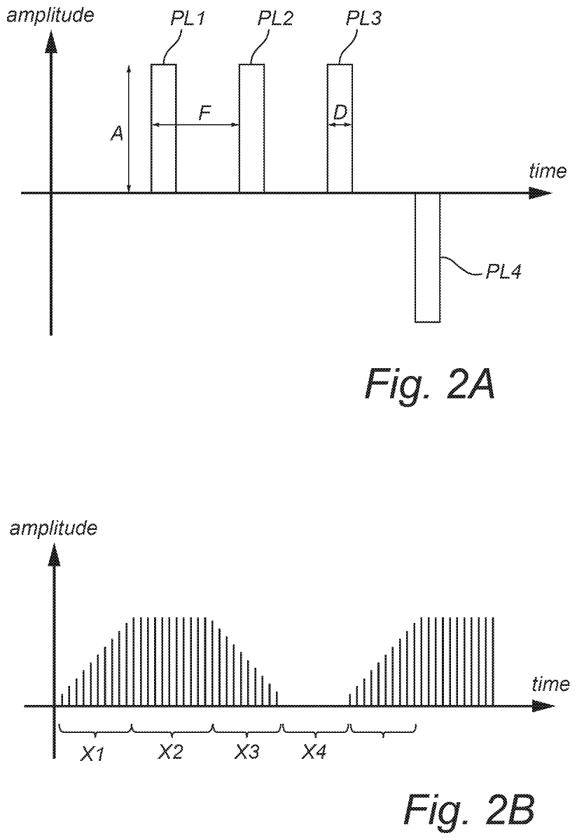

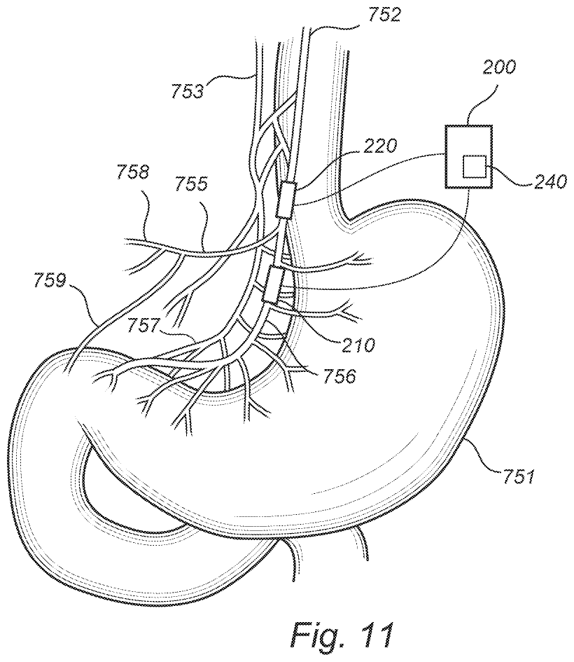

The electrical signal for exercising the tissue may be generated by a controller, such as the control unit 150 discussed above in connection with FIG. 11. The controller 150 may be configured to control the electrical stimulation such that the tissue is stimulated by a series of electrical pulses. The pulses may comprise a pulse of a first polarity followed by a pulse of a second, reversed polarity, and the pulsed electrical stimulation signal generated comprises a pulse frequency of 0.01-150 Hz. In an example, the electrical stimulation signal comprises a pulse duration of 0.01-100 ms and a pulse amplitude of 1-15 mA. Example characteristics of electric signals for exercising the tissue is discussed below with reference to FIGS. 2A and B.

The controller may be configured to receive input from a wireless remote control, directly or via a receiver of the implantable controller, for controlling the stimulation or for programming a stimulation routine for exercising the muscle tissue to improve the conditions for long term implantation of the implantable medical device. The programming of a stimulation routine could for example be the programming of the frequency of the stimulation, or the current and/or voltage of the stimulation.

It will be appreciated that both faradaic and capacitive mechanisms may be present at the same time, irrespectively of the type of electrode used and the type of stimulation provided (i.e., for the purpose of vasoconstriction/vasodilation, signal damping, or for the purpose of exercising the tissue). Thus, capacitive charge transfer may be present also for a bare electrode forming a metal-tissue interface, and faradaic charge transfer may be present also for a coated electrode forming a dielectric-tissue interface. It has been found that the faradaic portion of the current delivered to the muscle tissue can be reduced or even eliminated by reducing the duration of the pulses of the electric signal. Reducing the pulse duration has turned out to be an efficient way of increasing the portion of the signal which can be passed through the interface as a capacitive current, rather than by a faradaic current. As a result, shorter pulses may produce less electrode and tissue damage.

The capacitive portion of the current may further be increased, relative to the faradaic portion, by reducing the amplitude of the current pulses of the electrical signal. Reducing the current may reduce or suppress the chemical reactions at the interface between the electrode and the tissue, thereby reducing potential damage that may be caused by compounds and ions generated by such reactions.

In one example, the electrical stimulation may be controlled in such a manner that a positive pulse of the electrical signal is followed by a negative pulse (or, put differently, a pulse of a first polarity being followed by a pulse of a second, reversed polarity), preferably of the same amplitude and/or duration. Advantageously, the subsequent negative (or reversed) pulse may be used to reverse or at least moderate chemical reactions or changes taking place in the interface in response to the first, positive pulse. By generating a reversed pulse, the risk of deterioration of the electrode and/or the tissue at the interface between the electrode and the muscle tissue may be reduced.

FIG. 1F shows an electrode arrangement 210, 220 comprising a cuff 215 for attachment around the nerve 231, 232. The cuff 215 forms a support structure for the electrode element(s) E1. E2 and allows them to touch the outer surface of the nerve 231, 232. The cuff 215 may be configured to surround or enclose a circumference of the nerve 231, 232 at least partly and may comprise an opening or slit allowing the cuff 215 to be fitted around the nerve 231, 232.

One or more of the first and second electrode arrangements 210, 220 may comprise one or more of the electrode elements E1, E2 described above. The electrode elements E1, E2, which may also be referred to as stimulation electrodes, may be spaced apart along the sympathetic nerve 231 and/or the parasympathetic nerve 232. This allows the stimulation device to generate the stimulation signal(s) such that a first one of the stimulation electrodes E1, E2 serves as a cathode and a second one of the stimulation electrodes E1, E2 serves as an anode. It will be appreciated that further electrodes (not shown) may be provided, such as a third electrode, a fourth electrode, and a fifth electrode. Each of the third, fourth and fifth electrode may serve as an anode or a cathode during operation of the stimulation device.

In further examples, the cuff 215 may comprise a vibration device as disclosed in connection with FIGS. 24-34. The vibration device may, for example, comprise a piezoelectric element arranged to impart vibrations to the nerve 231, 232. The vibration device may be used instead of the electrodes E1, E2 or in combination with the electrodes E1, E2 and may, for example, be arranged to cause a portion of an inner surface of the cuff 215, which faces the tissue of the nerve, to reciprocate in a radial direction, i.e., back and forth towards and away from the nerve. In this way, vibrations may be imparted in the nerve to trigger action potentials to be generated (in case of the vibrational signal being an activation signal) or preventing action potentials to travel past the cuff 215 (in case of the vibrational signal being an inhibition signal, also referred to as a suppression signal or blocking signal).

It will be appreciated that the above-described concept of activating/inhibiting the sympathetic/parasympathetic nerves 231, 232 may be employed to treat a variety of symptoms, depending on where in the body the stimulation device and the electrode arrangements 210, 220 are implanted and which effector tissue is innervated by the respective nerves 231, 232.

In some examples, the nerves 231, 232 innervate smooth muscle tissue of the renal artery. This may allow the stimulation device to deliver stimulation signals inducing at least one of vasodilation and vasoconstriction in the renal artery, thereby affecting a blood pressure of the patient.

In some examples, the first effector tissue forms part of an artery supplying erectile genital tissue with blood and the second effector tissue forms part of a vein draining the blood from the genital erectile tissue. This may allow the stimulation device to deliver stimulation signals for triggering or causing vasodilation in the artery and vasoconstriction in the vein, thereby inducing erection in the erectile genitalia.

In some examples, the effector tissue 230 is smooth muscle tissue of a gastrointestinal tract of the patient. This allows the stimulation device to deliver stimulation signals affecting a level of motility of the gastrointestinal tract, thereby affecting at least one of nutrition uptake and fecal texture.

In some examples, the effector tissue 230 is a glandular tissue, which allows the stimulation device to deliver stimulation signals affecting a level of glandular secretion of the glandular tissue 230.

The stimulation device may hence be designed to stimulate various types of effector tissues in various parts of the body, depending on what type of response is desired and what type of symptom is treated. As various types of tissue (as well as individuals) may require various stimulation parameters, it may be beneficial to employ a calibration routine, in which the response to the applied stimulation signal is measured and used as feedback when controlling the stimulation parameters. However, some general observations may be made, which may serve as a starting point when choosing the stimulation parameters. For example, each type of tissue may be associated with a specific frequency range which may be used to trigger a response in the tissue.

Muscle tissue is generally formed of muscle cells that are joined together in tissue that can be either striated or smooth. Striated muscle tissue is further classified as either skeletal or cardiac muscle tissue. Skeletal muscle tissue is typically subject to conscious control, whereas cardiac muscle tissue is typically found in the heart and not subject to voluntary control. The so-called smooth muscle tissue is a third type of tissue, which is typically neither striated in structure nor under voluntary control. The contraction of the muscle tissue may be activated through electrochemical nerve impulses, i.e., action potentials. The action potentials may result in the release of neurotransmitters, causing the muscle cell to contract.

Smooth muscle cells may typically be activated, i.e., caused to contract, using a frequency in the range of 0.01-150 Hz. More specifically, the frequency may be in the ranges of 0.1-1 Hz, 1-10 Hz. 10-50 Hz and 50-150 Hz. It has been observed that a relatively low frequency component, such as pulse frequency, of about 1 Hz or less may be employed to imitate or enhance the slow wave potential associated with, e.g., pacemaker cells of the smooth muscle tissue. Furthermore, the pulse duration may be in the range of 0.01-100 ms, such as 0.1-4 mm, and preferably such as 1-5 ms. In case of an electric stimulation signal, the amplitude may be in the range of 0.1-15 mA, such as 0.5-5 mA. Furthermore, the separation between pulses, i.e., the pauses between adjacent pulses (or, in some cases, pulse trains) may be selected so as to match the wavelength of the natural pulses of the smooth muscle tissue. In different words, the distance between pulses of the stimulation signal may be selected to synchronize with the slow eave potential associated with, e.g., the pacemaker cells of the smooth muscle tissue. In other examples, the distance between the pulses may be selected to be slightly off the natural wavelength of the pulses of the smooth muscle tissue so as to drive a frequency of the natural pulses towards a higher frequency or a lower frequency.

Skeletal muscle cells may typically be activated by means of a stimulation signal having a frequency of in a range of about 0.1-100 Hz, such as 1-10 Hz or 10-100 Hz. In an example, a frequency in a range of about 50 Hz may be used. Furthermore, a pulse duration in a range of 0.01-100 ms, such as 0.1-4 mm, and preferably such as 1-5 ms, may be employed. In case of an electric stimulation signal, the current amplitude may be in a range of 0.1-15 mA. In some examples, a desired muscle contraction response has been experimentally observed within a range of 0.5-5.0 mA.

Cardiac muscle cells may typically be activated by slightly lower frequencies compared to skeletal muscle cells, such as in a range of 0.5-3 Hz. In an example, a beneficial response has been observed when applying a stimulation signal having a frequency of about 1 Hz. Similar to the skeletal muscles, the pulse duration may be in a range of 0.01-100 ms, such as 0.1-4 mm, and preferably such as 1-5 ms. In case of an electric stimulation signal, the current amplitude may be in a range of 0.1-15, such as 0.5-5.0 mA.

Consequently, it will be appreciated that the stimulation parameters, such as frequency and amplitude, may be adapted to the muscle type and the type of response desired. This applies both to electric stimulation and vibrational stimulation signal.

In the following, a detailed description will be given of a technology for electrically stimulating tissue, such as the above renal artery 20, for exercising the tissue and thereby improving the conditions for long term implantation. The body tends to react to a medical implant, partly because the implant is a foreign object, and partly because the implant interacts mechanically with tissue of the body. Exposing tissue to long-term engagement with, or pressure from, an implant may deprive the cells of oxygen and nutrients, which may lead to deterioration of the tissue, atrophy and eventually necrosis. The interaction between the implant and the tissue may also result in fibrosis, in which the implant becomes at least partially encapsulated in fibrous tissue. It is therefore desirable to stimulate or exercise the cells to stimulate blood flow and increase tolerance of the tissue for pressure from the implant.

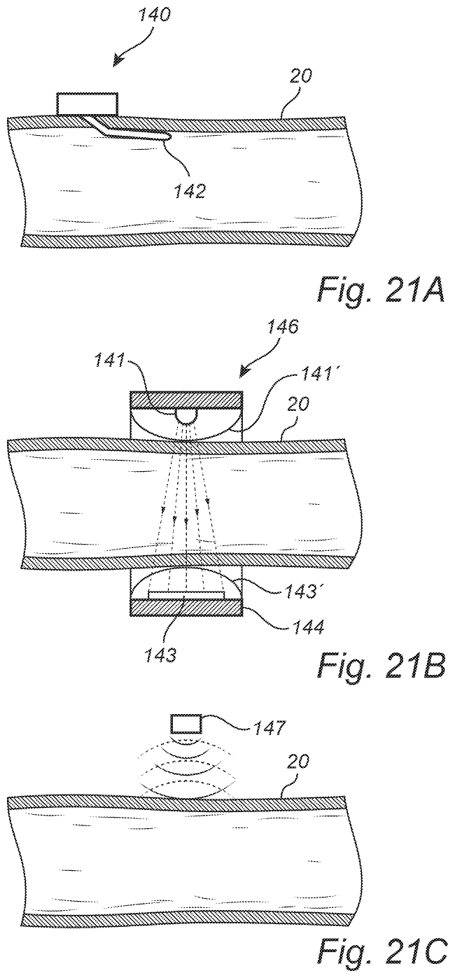

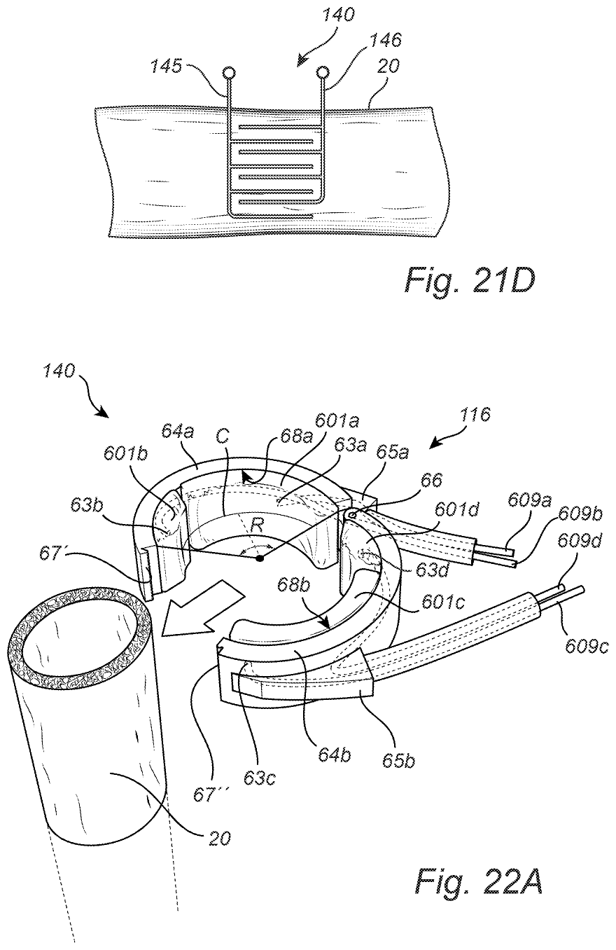

In the following, the use of electric signals for exercising tissue to improve the conditions for long term implantation will be described. It should be noted that there may be a difference between the electric stimulation signal (as well as the signal damping signal) discussed above in connection with for instance FIG. 1A, and the electric signal delivered for improving long term implantation conditions. While the former signal may be specifically adapted to trigger a muscular response for inducing vasodilation, the latter may be provided with the primary aim of preventing deterioration of the tissue and eventually necrosis of tissue of the renal artery. Preventing or reducing tissue deterioration does not necessarily require a stimulation causing the same degree of response as needed for inducing vasodilation. On the contrary, it may be advantageous to deliver an electric signal inducing a stimulation of the tissue stimulation (i.e., motoric response) without substantially affecting the flow resistance in the blood vessel. The “exercising” of the tissue to prevent deterioration may hence be combined with the stimulating causing vasodilation, and preferably cycled such that exercising cycles are performed between vasodilation cycles. The exercising, which thus may differ in effect or muscular response from the vasodilation, may be performed by delivering an exercising signal to the tissue via the electrode arrangements of the stimulation devices and/or damping devices discussed with reference to e.g. FIGS. 1A, 3A, and 5A. It may be particularly advantageous to combine the exercising of the muscle tissue with medical devices comprising support structures, such as the cuff 115, 116 shown in FIGS. 1F, 3C, 3F, 17A-D and the sensors in FIGS. 21A-D. As these may form a relatively large contact surface with the tissue against which they are arranged, there is an increased risk for a negative impact on the health of the tissue.

The electrical electrode arrangement and exercising methods described in the following may thus be implemented in any of the embodiments of the stimulation devices, signal damping devices, and sensors described above for the purpose of exercising the tissue which is in contact with such medical devices or implants.

FIG. 2A shows an example of a pulsed electrical signal to be applied to an electrode for electrically stimulating muscle tissue via an electrode-tissue interface, thereby exercising the muscle tissue, as discussed above. The electrical signal may be generated by a controller arranged outside the body or implanted in the. The characteristics of the electrical signal may be selected and varied determined on the electrical and properties at the electrode-tissue interface and on the actual response of the tissue. The electrical stimulation delivered to the muscle cells may depend on several factors, such as the configuration and placement of the electrode element at the tissue, the presence of fibrous material at the interface, the composition of the electrolyte in the interface, accumulation of non-conducting material on the electrode surfaces, etcetera. It is therefore suggested that the characteristics of the electric signal, as shown in the present figure, be selected and varied based on an observed or estimated response from the stimulated tissue.

In the present example, the electrical signal is a pulsed signal comprising square waves PL1, PL2, PL3, PL4. However, other shapes of the pulses may be employed as well. The pulse signal may be periodic, as shown, or may be intermittent (i.e., multiple series of pulses separated by periods of no pulses). The pulses may have an amplitude A, which may be measured in volts, ampere or the like. Each of the pulses of the signal may have a pulse width D. Likewise, if the signal is periodic, the pulse signal may have a period F that corresponds to a frequency of the signal. Further, the pulses may be either positive or negative in relation to a reference.

The pulse frequency may for example lie within the range of 0.01-150 hertz. More specifically, the pulse frequency may lie within at least one of the ranges of 0.1-1 Hz, 1-10 Hz, 10-50 Hz and 50-150 Hz. It has been observed that relatively low pulse frequencies may be employed to imitate or enhance the slow wave potential associated with pacemaker cells of the smooth muscle tissue. Thus, it may be advantageous to use relatively low pulse frequencies, such as 0.01-0.1 Hz or frequencies below 1 Hz or a few Hz for such applications.

The pulse duration may for example lie within the range of 0.01-100 milliseconds, such as 0.1-20 milliseconds (ms), and preferably such as 1-5 ms. The natural muscle action potential has in some studies been observed to last about 2-4 ms, so it may be advantageous to use a pulse duration imitating that range.

The amplitude may for example lie within the range of 1-15 milliamperes (mA), such as 0.5-5 mA in which range a particularly good muscle contraction response has been observed in some studies.

In a preferred, specific example the electrical stimulation may hence be performed using a pulsed signal having a pulse frequency of 10 Hz, a pulse duration of 3 ms and an amplitude of 3 mA.

In case a contraction of the smooth muscle tissue is desired, for example to cause vasoconstriction of a blood vessel or affect peristalsis in the gastrointestinal tract, it may be advantageous to select a separation between adjacent pulses based on the natural muscle action potentials or frequency of the natural muscle movements sometimes observed in smooth muscle tissue. A resonance approach, or standing wave approach, may thus be employed to amplify the movements or waves in the smooth muscle tissue. This may be achieved by matching pulse duration and/or separation with the corresponding parameters of the natural movements of the tissue.

FIG. 2B shows an example of a pulsed signal, comprising build-up period X1, in which the amplitude is gradually increasing, a stimulation period X2 during which the muscle tissue is exposed to a contracting stimulation signal, a ramp down period X3 in which the amplitude is gradually decreasing, and a stimulation pause X4 before a new build-up period is initiated. The build-up period may for example be 0.01-2 seconds, the stimulation period 1-60 seconds, the ramp-down period 0.01-2 seconds, and the stimulation pause 0.01-60 seconds. The pulse frequency may for example be 1-50 Hz, the pulse duration 0.1-10 milliseconds and the amplitude during the stimulation period be 1-15 milliampere. The stimulation of skeletal muscle tissue may for example be performed using a frequency of 50 Hz and pulses having a duration of 100 μs. The current amplitude may be 1, 2.5, 7.5 or 10 mA. In particular, a desired muscle contraction response has been experimentally observed within a range of 0.5 to 5.0 mA. In the present example, a coiled electrode may be used as a cathode. Another example design is a multi-stranded wire arranged in a helical design. They can be imbricated in the muscular wall of the luminary organ, such as the renal artery 20, and can be stimulated in any desired pattern. The stimulus parameters may for example be biphasic pulses, 10 to 40 Hz, lasting 0.1 to 5 ms, with a current density of 3 to 5 mA/cm2.

It may be beneficial to apply the stimulation with a preferred activation direction such that a majority of the action potentials generated in response to the stimulation propagate in the preferred activation direction. This may be achieved by inhibiting or blocking the nerve at a specific location such that no or at least only a minor part of the action potentials can travel beyond that location. For some applications, the preferred activation direction would be in the efferent direction, i.e., towards the effector tissue 230. Put differently, in some examples it may be beneficial if the application of the stimulation signal gives rise to action potentials propagating in the direction of the tissue in which the effector response is desired, rather than in the opposite, afferent direction (typically towards the CNS 233) to reduce adverse side effects of the application of the signal. Examples of adverse side effects include initiation of undesired or counter-productive feedback to the brain and can result in undesired sensations or activity of the patient. In some examples, it may be of interest to prevent action potentials from travelling in the efferent direction, i.e., away from the CNS 233 and towards the effector tissue 230.

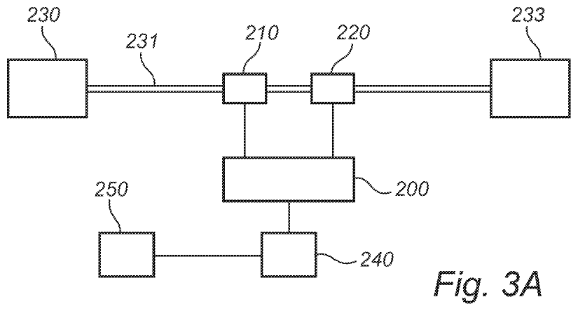

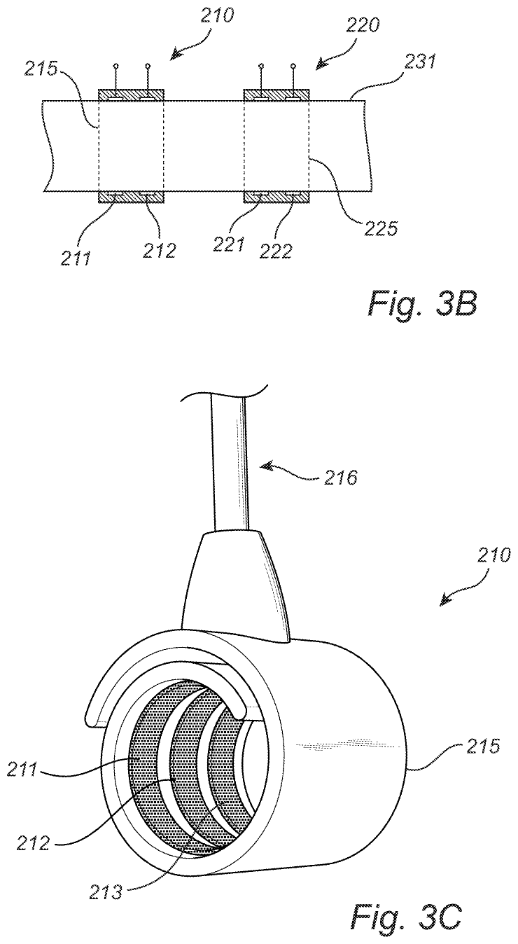

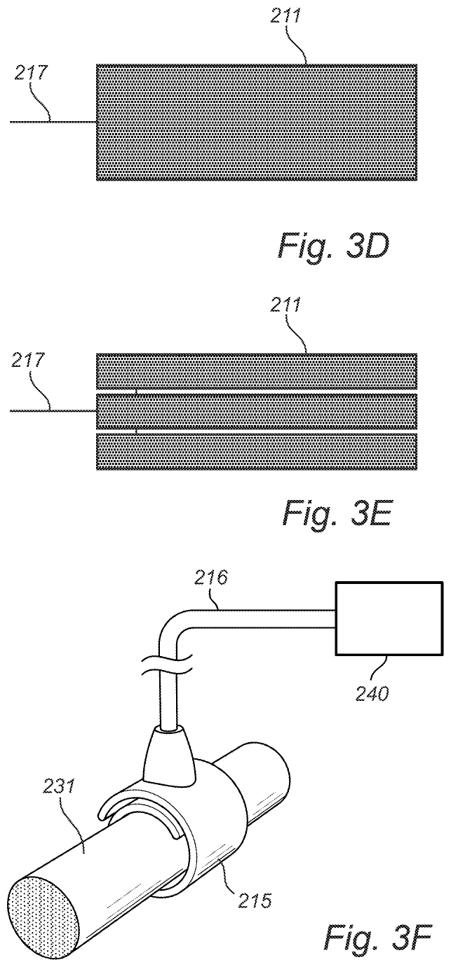

The generation of action potentials propagating in a preferred direction may be achieved by means of so-called unidirectional stimulation techniques, which will be described in the following with reference to the examples shown in FIG. 3A to FIG. 3F.

The underlying rationale is based on the application of a suppression signal for suppressing action potentials propagating in the nerve in an undesired direction, typically the afferent direction (also referred to as antidromic direction). The suppression signal may comprise a frequency component for blocking, inhibiting, or suppressing the nerve's conduction capacity in a similar manner as discussed above with reference to the inhibition signal. As mentioned above, such a frequency component may be relatively high, typically in the range of 1-10 KHz.

The suppression signal, which may also be referred to as an inhibition signal, may be an electric signal or a mechanical (vibrational) signal. Combinations of the two are also possible, in which a combination of an electric signal and a vibrational signal is provided. The combined signal may, for example, be generated by a vibrational element, such as a piezoelectric element, comprising one or more electrodes 210, 220 for applying an electric signal. Vice versa, the combined signal may as well be generated by an electrode arrangement 210, 220 comprising a piezoelectric element for imparting vibrations into the nerve. A frequency of the inhibition signal may thus be selected to block or at least reduce the nerve's ability to convey nerve signals. Therefore, the suppression signal may comprise one or more frequency components in the kilohertz range.

FIG. 3A is a schematic illustration of a system for affecting an effector response in effector tissue 230 of a patient according to some examples. The system comprises a stimulation device comprising a first electrode arrangement 210 for applying a stimulation signal to a nerve 231 innervating the effector tissue 230. The nerve may be a branch of the autonomous nervous system (ANS), such as sympathetic nerve or a parasympathetic nerve as discussed above in connection with the combined SNS and PNS stimulation. The stimulation device further comprises a second electrode arrangement 220 for delivering a suppression signal to the nerve 231 (the suppression signal may also be referred to as an inhibition signal). The second electrode arrangement 220 may be coupled to the nerve 231 at a position allowing afferent nerve signals, which are travelling towards the CNS 233, to be blocked or at least hindered. As indicated, the first electrode arrangement 210 and the second electrode arrangement 220 may be arranged spaced apart along a conduction direction of the nerve 231. The second electrode arrangement 220 may be coupled to the nerve 231 at a position between the first electrode arrangement 210 and the CNS 233. It will, however, be appreciated that the second electrode arrangement 220 in other examples may be coupled to the nerve 231 at a position between the first electrode arrangement 210 and the effector tissue 230 to suppress efferent nerve signals travelling towards the effector tissue 230.

Afferent signals may refer to signals travelling towards the CNS 233. They typically originate from sensory receptors located throughout the body and carry sensory information from the body to the brain. The term “afferent” may hence be used to denote a propagation direction generally towards the CNS 233. Accordingly, efferent signals may refer to signals travelling in the opposite direction, away from the CNS 233 and towards various effector organs, such as muscles and glands. The efferent signals typically carry instructions from the CNS 233 and the brain to the body. The term “efferent” may hence be used to denote a propagation direction generally away from the CNS 233.

The direction in which an electrical impulse travels along a neuron's axon may also be described by the terms “antidromic” and “orthodromic” and may therefore be used to refer to the direction in which the action potentials, generated by the stimulation device, travel. Orthodromic conduction may be understood as referring to the propagation of nerve impulses in the natural, physiological direction. In a motor neuron, for example, this may be from the cell body (located in the spinal cord or brain) down the axon to the axon terminals that synapse with muscle fibers or other neurons (i.e., in the efferent direction). In a sensory neuron, it may be from the sensory endings towards the cell body and then onto the spinal cord or brain (i.e., in the afferent direction). Correspondingly, antidromic conduction may be understood as the direction of propagation of nerve impulses in the opposite direction to the normal or natural flow. For a motor neuron, this may mean an impulse travelling from the axon terminals back towards the cell body (i.e., in the afferent direction). In a sensory neuron, it may be from the CNS 233 out towards the sensory endings (i.e., in the efferent direction). It will be understood that the second electrode arrangement 220 may be employed to suppress nerve signals propagating in any of the above-mentioned directions, i.e., efferent, afferent, orthodromic, and antidromic direction, depending on the type of stimulation, the type of effector tissue, and what type of response is desired.

The operation of the first electrode arrangement 210 and the second electrode arrangement 220, i.e., the generation and application of the stimulation signal and the suppression signal, respectively, may be controlled by a control unit 240 that is operably connected to the stimulation device. Further, the control unit may be configured to receive sensor input, such as from one or more sensors 250 arranged to generate a signal indicative of a response in the effector tissue 230 when stimulated by the stimulation signal. The stimulation device may hence be similarly configured as the stimulation device discussed above with reference to FIG. 1A to FIG. 1E.

The control unit 240 may be configured to drive the stimulation device such that each of the first and second electrode arrangements 210, 220 are actuated in sequence. In an example, a delay of the suppression signal may be timed to generally match a conduction velocity of the stimulation signal in the nerve 231. The blocking or suppressed conduction of the nerve 231 can therefore be provided substantially at the same time when the action potentials, generated by the stimulation signal, reach the location where the suppression signal is applied to the nerve 231.

In some examples, the control unit 240 may be configured to drive the stimulation device such that each of the first and second electrode arrangements 210, 220 apply the stimulation signal and the suppression signal substantially at the same time, such as concurrently (i.e., at least partly overlapping in time) or simultaneously.

As mentioned above in connection with the stimulation of the SNS and PNS, the stimulation signal may be a low-frequency signal with a frequency in the range of, for example, 0.1-100 Hz and the suppression signal a high-frequency signal with a frequency in the range of, for example, 1-10 kHz.

The first electrode arrangement 210 and/or the second electrode arrangement 220 may comprise a monopolar electrode delivering the stimulation signal and/or the suppression signal to the nerve 231. The monopolar electrode may be operated as an anode or a cathode, with a separate electrode forming a complementing cathode or anode for closing the electric circuit. This complementing electrode, closing the electric circuit, may be provided elsewhere, such as by a housing of the stimulation device, or may be arranged at another location in or on the patient's body.

In some examples, the first electrode arrangement 210 and/or the second electrode arrangement 220 may comprise a bipolar electrode, comprising a first electrode serving as a cathode and a second electrode serving as an anode for closing the electric circuit. A few examples will be discussed in the following with reference to the accompanying figures.

FIG. 3A illustrates a first electrode arrangement 210 and a second electrode arrangement 220 of a stimulation device according to some examples. The first electrode arrangement 210 comprises a first stimulation electrode 211 and a second stimulation electrode 212 for applying the stimulation signal to the nerve 231 (or, in some examples, directly to the effector tissue 230). The first stimulation electrode 211 and the second stimulation electrode 212 may be arranged spaced apart along the nerve such that the applied stimulation signal may propagate between the first and second stimulation electrodes 211, 212 in a conduction direction of the nerve 231. The first stimulation electrode 211 may hence serve as a cathode whereas the second stimulation electrode 212 may serve as an anode. Electrical leads, or conduction lines, may be coupled to each of the electrodes 211, 212 for supplying the respective electrode 211, 212 with electric power. The first electrode arrangement 210 may further comprise a cuff 215 configured to be at least partly arranged around the nerve 231 and hold the first electrode 211 and second electrode 212 in place against the nerve 231.

Similar to the first electrode arrangement 210, the second electrode arrangement 220 may, in some examples, comprise a first suppression electrode 221 and a second suppression electrode 222 for applying the suppression signal to the nerve 231 (or, in some examples, directly to the effector tissue 230). The first suppression electrode 221 and the second suppression electrode 222 may be arranged spaced apart along the nerve 231. The first suppression electrode 221 may hence serve as a cathode, while the second suppression electrode 222 may serve as an anode during operation. Electrical leads, or conduction lines, may be coupled to each of the electrodes 221, 222 for supplying the respective electrode 221, 222 with electric power. The second electrode arrangement 220 may further comprise a cuff 225 configured to be at least partly arranged around the nerve 231 and hold the first suppression electrode 221 and second suppression electrode 222 in place against the nerve 231.

In other examples, the electrodes 211, 212, 221, 222 may be replaced with vibration elements, such as piezoelectric elements, for providing a mechanical (vibrational) signal to the nerve 231.

It will be appreciated that further electrodes may be provided to deliver the stimulation signal and the suppression signal, respectively. An example of such a configuration is shown in FIGS. 3B and C, illustrating a cuff electrode arrangement 210, 220 which can be used to deliver the stimulation signal and/or the suppression signal. Each cuff electrode 210 shown in the present example includes a first electrode 211, a second electrode 212, and a third electrode 213. Each of the electrodes 211, 212, 213 may comprise an electrically conductive surface configured to be arranged to abut or rest against the tissue to which the electrical signal is to be delivered. The electrodes 211, 212, 213 may be individually controlled electrically. At least a first one of the electrodes 211, 212, 213 may be operated as a cathode, wherein at least a second one of the electrodes 211, 212, 213 may be operated as an anode for delivering the stimulation signal or the suppression signal to the nerve 231.

In this arrangement, the electrode arrangements 210 may comprise a lead 216 for providing the electric power required for the stimulation signal/suppression signal. The lead may be oriented across the nerve 231 or along the nerve 231, depending on the implantation site and the available space at the nerve 231.

The cuff electrode 210 in FIG. 3C may comprise a body 215 which may be molded from an elastomeric material (e.g., silicone). The electrodes 211, 212, 213 may be integrated with the body 215 during the molding process. The body 215 may be shaped or formed to normally assume a curled or tubular spiral or rolled configuration. As shown, the body 215 may in its normal, coiled condition have overlapping end portions forming a spiral which extends more than 360° end to end. The body 215 may be elastically uncoiled to increase its inner diameter and allow the cuff electrode 210 to be initially fitted about a periphery of a target nerve 231. Further, the coiled shape of the body 215 allows for the inner diameter of the cuff electrode 215 to be further adjusted to post-operative changes that might occur for example due to swelling. The elasticity of the body 215 may beneficially wrap the electrodes 211, 212, 213 snugly against the periphery of the nerve 231.

FIG. 3D and FIG. 3E show examples of an electrically conductive surface, or electrode 211, of an electrode arrangement 210, 220 as mentioned above. The electrode 211 may be formed by a single, continuous conductive surface as in FIG. 3D or a surface that is segmented into separate conductive segments that may be electrically coupled by a wire or conductive lead as in FIG. 3E. A lead 217 may be provided to power the electrode 211.

FIG. 3F shows an example wherein the cuff electrode 210 is wrapped around the target nerve 231 such that the electrically conductive surfaces, forming the stimulation electrode(s) or the suppression electrode(s), make and sustain circumferential contact substantially about the entire periphery of the target nerve 231. It will, however, be appreciated that in some examples the electrode(s) may be positioned to make contact with the target nerve 231 along the length axis of the nerve 231, i.e., the propagation direction of the nerve 231.

The conductive surfaces, which form the stimulation/suppression electrodes, may be made from strips of metal, such as platinum. In some examples, they may be formed from a thin film of metal, which may be deposited on a surface of the body 215 forming the cuff of the electrode arrangement 210, 220. In an example, each of the conductive surfaces (or strips) may measure about 10 mm in length and 2 mm in width.

It will be appreciated that in further examples, not illustrated, one or more of the electrode arrangements 210, 220 may have a configuration different from the cuff electrode design. The electrode arrangements 210, 220 may, for example, be configured to be placed against the nerve 231 without encircling or enclosing it. The electrode arrangements 210, 220 may be configured as needle electrodes arranged to protrude into the nerve 231 or lie against an outer surface of the nerve 231. In further examples, the electrode arrangements 210, 220 may be patch electrodes similar to the ones illustrated in FIG. 1B and FIG. 1C.

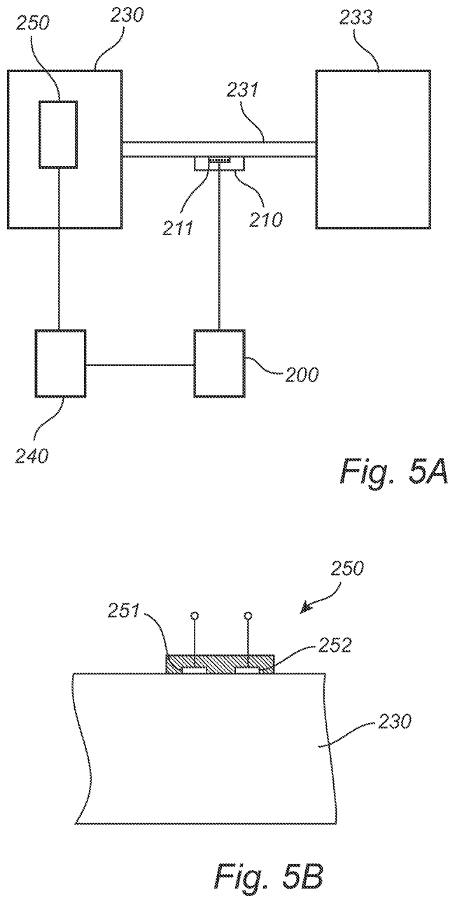

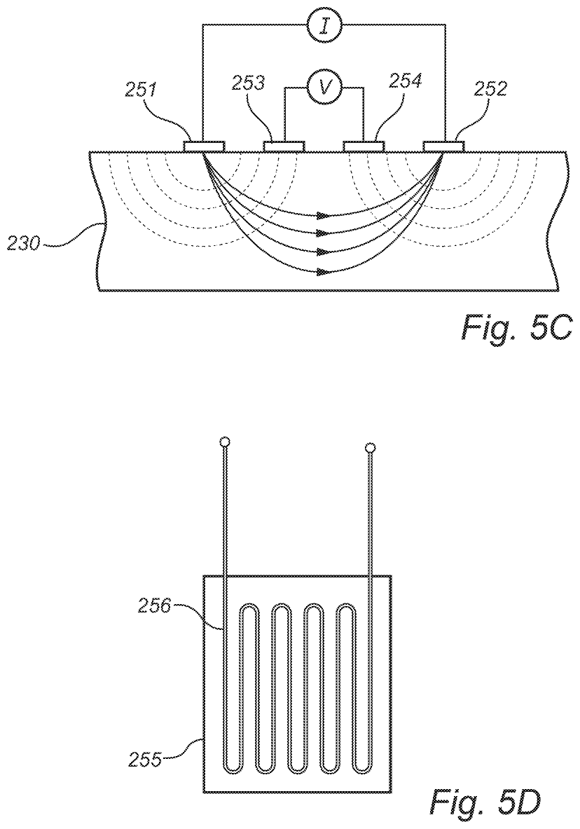

The effector response may be measured by a sensor device, such as the sensor device 250 shown in the example of FIG. 1A and FIG. 3A. The use and operation of such a sensor device 250 will be described in the following with reference to FIG. 5A to FIG. 5D.

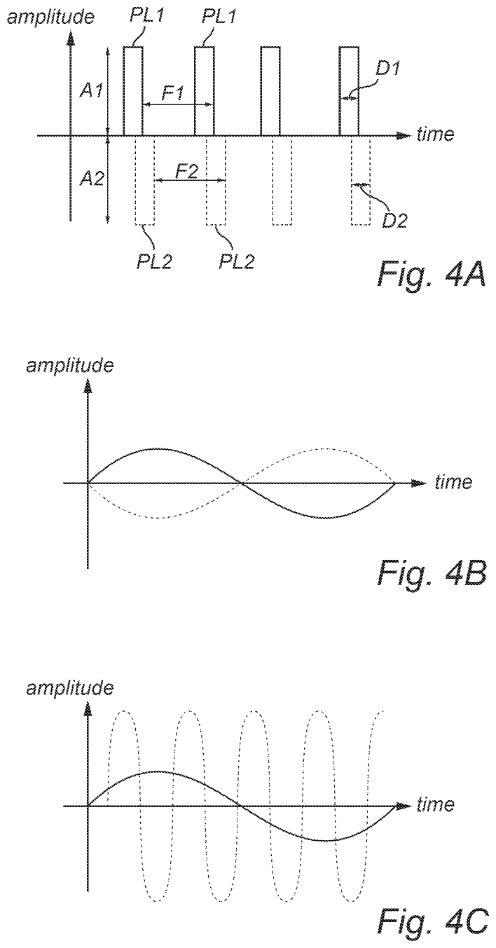

FIG. 4A is a diagram illustrating a signal damping mechanism, or phase cancellation mechanism, according to some embodiments. In some example, the mechanism may also be referred to as a nerve inhibition or blocking mechanism, in which action potentials are hindered from propagating past an application point of the damping signal. This may be a way of achieving unidirectional stimulation, in which the stimulation signal is delivered in a preferred direction in of the nerve.

FIG. 4A schematically shows an electric stimulation signal comprising a series of positive pulses PL1, and an electric damping signal comprising a series of negative pulses PL2. The stimulation signal may originate from a stimulation device 110 arranged to cause vasodilation in the renal artery 20, whereas the electric damping signal may be generated by a signal damping device 120, comprising a control unit which may be arranged outside the body or be implanted in the body. Other applications are however possible, such as electric stimulation of effector tissue in general. Examples of such tissue include smooth muscle tissue, skeletal tissue, and glands. For illustrative purposes, however, the signal damping or blocking concept will be explained by means of the particular example of vasodilation in the renal artery.

The control unit may be operatively connected to an electrode arrangement by means of one or several leads. The electrode arrangement may comprise a plurality of electrode elements attached to the muscle tissue of the renal artery wall 20, to tissue in close vicinity of the muscle tissue of the renal artery, or a nerve innervating the renal artery, such that the electrode elements are allowed to deliver the damping signal to said tissue. The electrical signals shown in the present figure may either reflect the signal as generated at the stimulation device and signal damping device, respectively, or the signal as delivered to the tissue. In the present example, the electrical signals are pulsed signals comprising square waves PL1, PL2. However, this may be considered to represent an ideal signal, and it is appreciated that other shapes of the pulses may be provided as well. The pulse signals may be periodic, as shown, or intermittent (i.e., multiple series of pulses separated by periods of no pulses). The pulses may have an amplitude A1, A2, which may be measured in volts, amperes, or the like. Each of the pulses of the signals may have a pulse width D1, D2. Likewise, if the signal is periodic, the pulsed signals may have a period F1, F2 that corresponds to a frequency of the signal. Further, the pulses may be either positive or negative in relation to a reference. In the present example, the signal originating from the propagating stimulation signal may comprise positive pulses PL1 whereas the damping signal may comprise negative pulses PL2.

In the present example, the electric stimulation signal may be a pulsed signal comprising square waves having a frequency in the range of 0.01-150 Hertz. The pulse duration may lie within the range of 0.01-100 milliseconds (ms), such as 0.1-20 ms, and preferably such as 1-6 ms. The natural muscle action potential has in some studies been observed to last about 2-4 ms, so it may be advantageous to use a pulse duration imitating that range when stimulating the tissue to cause it to relax.

The amplitude of the stimulation signal may for example lie within the range of 1-15 milliamperes (mA), such as 0.5-5 mA, in which range a particularly good muscle response has been observed in some studies.

In a preferred, specific example, the electrical stimulation delivered by the stimulation device 120 may hence be performed using a pulsed signal having a pulse frequency of 10 Hz, a pulse duration of 3 ms and an amplitude of 3 mA. The pulsed signal shown in FIG. 4A (in solid lines) may be considered to represent the characteristics of such a stimulation signal as it is propagating through the tissue of the renal artery 20.

The damping signal (indicated by dashed lines) may be designed to counteract, or mitigate, the tissue's response to the stimulation signal. In the example shown in the present figure, this may for instance be achieved by providing a series of pulses PL2 having a polarity that is reversed in relation to the pulses PL1 of the signal originating from the stimulation signal. Further, the damping signal may be phase shifted in relation to the positive pulses. The timing of the signals may hence be selected such that the negative pulses PL2 are positioned at the time of the positive peaks PL1, or slightly delayed relative the positive peaks PL1, as indicated in FIG. 4A. A negative pulse PL2 may be delivered to the tissue shortly after the positive pulse, before the cells have had time to react to the stimuli provided by the positive pulse. Put differently, the damping signal may be delivered to the cells at the onset of the change in cell polarization, thereby reducing or cancelling cell polarization. The negative pulse PL2 may thus act to counteract, or cancel, the stimuli provided by the positive pulse PL1, thereby preventing the cells from contract, or at least reducing the contraction triggered by the positive pulse PL1.

It should be understood that the signals illustrated in the above example are schematic and ideal, and not necessarily a true representation of the actual signals delivered to the tissue. The actual signals may be more complex, having a more complex frequency composition and comprising various degrees of noise. The illustration in FIG. 4A is purposely simplified to help elucidate the inventive concept of applying a damping signal to counteract or reduce the effects of the stimulation signal as it propagates to other parts of the body which are not the primary target of the stimulation. It may therefore be advantageous to provide a sensor measuring the signal, which is to be damped or counteracted, and design the damping signal based on input from the sensor. This allows for the damping signal to be generated also in cases where the stimulation signal varies over time or is difficult to estimate or model. By such a feedback loop, a more flexible damping may be provided.

As mentioned above, the signals do not necessarily have to be formed of pulses or square waves. FIG. 4B illustrates another (still simplified) example, wherein the signal originating from the stimulation signal is shaped as a sine wave, and wherein the damping signal has a corresponding shape and is phase shifted to counteract or cancel the stimulation signal. Other signal shapes are however equally possible, including square, triangle and sawtooth waves and combination thereof.

A further example is shown in FIG. 4C, in which the damping signal is configured to disturb or “scramble” the signal originating from the stimulation device 110 such that it has a reduced effect on tissue arranged remote from the electrode elements 112a of the stimulation device 110. The damping signal may for instance comprise a frequency which is higher than the frequency of the signal from the stimulation device 110, such that the resulting, superposed signal that reaches the individual tissue cells are less suitable for triggering a contraction of the smooth muscle tissue cells or a conveying of the stimulation signal by the nervous tissue cells. This is based on the observation that a stimulating signal may have a reduced impact on cells when the frequency is outside a certain interval. Put differently, the stimulation of tissue may be less efficient for higher frequencies, and the damping signal may therefore be applied to increase the frequency accordingly.

FIG. 5A is a schematic illustration of a stimulation device configured to deliver a stimulation signal to a nerve 231 innervating an effector tissue 230 of a patient. The stimulation device may form part of a system comprising a sensor device 250, configured to generate a sensor signal indicating the effector response in the effector tissue 230, as well as a control unit or controller 240, operable to receive the sensor signal and control an operation of the stimulation device based at least in part on the sensor signal. The nerve 231 may be a sympathetic nerve or a parasympathetic nerve extending between the central nervous system 233 and the effector tissue 230, as previously discussed in connection with FIG. 1A and FIG. 2A. Consequently, the stimulation signal may be an activation signal, such as the previously discussed low-frequency signal, or an inhibition signal, such as the previously discussed high-frequency signal. For illustrative purposes, the stimulation signal is applied by means of an signal generating means, such as an electrode arrangement 210 arranged to touch the nerve 231. The exemplary electrode arrangement 210 may be a unipolar electrode, comprising a first stimulation electrode 211 that may act as a cathode or anode. Another electrode (not shown) may be provided elsewhere to close the electric circuit. This circuit-closing electrode may, for example, be formed by a portion of a housing of the stimulation device. It will however be appreciated that other configurations of the stimulation device and the electrode arrangement(s) 210 are possible.

The sensor device 250 may be configured to measure the effector response in various ways. The sensor device 250 may be configured to employ one or more electrodes for measuring an electrical characteristic of the effector tissue 230. In further examples, the sensor device 250 may be configured to employ one or more mechanical sensor elements for measuring a mechanical characteristic or response in the effector tissue 230. The information provided by the sensor device 250 may thus be used to determine or monitor an activity or response in the effector tissue 230 and provide feedback that can be used for controlling the operation of the stimulation device.

FIG. 5B shows an example in which the sensor device 250 comprises one or more sensor electrodes 251, 252 configured to measure an electric activity in the effector tissue 230 in response to the mechanical or electrical stimulation signal. This approach may be referred to as electromyography (EMG). The electrode(s) may be arranged to measure the electric activity in the effector tissue 230 (typically muscle tissue). An increased activity in the effector tissue 230 may typically cause an increased electrical activity, whereas the effector tissue 230 normally does not produce any electrical activity during rest. Therefore, EMG may be used to detect and quantify changes in the electrical activity of the effector tissue 230 caused by an applied stimulation signal. In the present example, a first sensor electrode 251 and a second sensor electrode 252 are provided to generate the sensor signal. The voltage signal, which indicates the response in the effector tissue 230, may typically be in the range of 1-2 mV.

FIG. 5C shows an example in which the sensor device 250 comprises one or more sensor electrodes 251, 252, 253, 254 configured to measure a change in electrical impedance in the effector tissue in response to the stimulation signal. This approach may be referred to as electrical impedance myography (EIM). The electrodes may be arranged to apply an electric signal I, typically in the kHz to MHz frequency range, to the tissue 230 and measure the resulting voltages. The sensor device 250 may, in some examples, comprise a first electrode 251 and a second electrode 252 for applying the electric signal I and a third electrode 253 and a fourth electrode 254 for measuring the resulting voltage V. This information may be used to determine the impedance, which may be separated into a resistance and reactance. For a given resistance and reactance, a phase may be calculated. It has been found that all three parameters, i.e., resistance, reactance, and phase, may be indicative of the effector response caused by the stimulation signal delivered by the stimulation device. The impedance of the effector tissue 230 may, for example, be measured during the stimulation and when no stimulation signal is applied. Differences in impedance when the tissue 230 is stimulated and when not stimulated may be used to analyze the effects of the stimulation and control the operation of the stimulation device accordingly. In the present examples, the electrodes are arranged spaced apart on a surface of the effector tissue 230, with the third and fourth electrodes 253, 254 arranged between the first and second electrodes 251, 252. In some examples, a typical impedance over a muscle is about 1 kΩ and the measured changes in response to the stimulation are in the order of 1Ω. Thus, by measuring impedance variations of about 0.1%, information may be retrieved about the muscle's response to the applied stimulation signal.

Combinations of the EMG and EIM approaches are possible. Thus, in some examples, the sensor device 250 comprises an electromyographic sensor configured to measure an electric activity in the effector tissue 230 and an electric impedance sensor configured to measure a change in electrical impedance in the effector tissue 230. The control unit 240 may be configured to receive sensor signals from both the electromyographic sensor and the impedance sensor and control or adjust the application of the stimulation signal based on the received sensor signals. The combination of EMG and EIM may be beneficial because it may enhance the reliability of muscle contraction detection. In this example, EMG may provide detailed information on muscle activity, while EIM may offer insights into muscle composition and health. When used together, they may compensate for each other's limitations, improving the accuracy and robustness of effector response monitoring. This synergy may be particularly advantageous in environments with potential for mechanical disturbances to the electrodes, ensuring more consistent and reliable effector response readings.

The sensor electrode 251-254 of the sensor device 250 may be configured to be arranged at the effector tissue 230 or inserted into the effector tissue 230. The sensor electrode(s) 251 may in some examples be formed as one or more patch electrodes that can be attached to the effector tissue 230. In some examples, the sensor electrode(s) 251 may be formed as needle electrodes arranged to protrude at least partially into the effector tissue 230.

The sensor device 250 may further comprise a reference electrode allowing the sensor signal to be based on an electrical interaction between one or more sensor electrode 251 and the reference electrode. The reference electrode may be formed by a housing of the stimulation device and/or an electrode arranged at the effector tissue 230, spaced apart from the sensor electrode 211.

As mentioned above, the sensor device 250 may in some examples comprise one or more mechanical sensor elements for measuring a mechanical characteristics or response. The sensor device 250 may, for example, be configured to measure mechanical movement in the effector tissue 230. FIG. 5D shows an example of such a sensor device 250 which comprises a strain gauge for measuring a contraction or relaxation of effector tissue 230 in response to the stimulation signal. Thus, the effector tissue 230 may be muscle tissue, such as smooth tissue. The strain may be positive (due to elongation of the muscle tissue) or negative (compressive, due to contraction of the muscle tissue). The strain gauge may be arranged to convert a change in dimension to a change in electrical resistance. In the present example, the strain gauge comprises a wire or foil 256 arranged in a grid pattern. During operation, the electrical resistance of the strain gauge may change in proportion to the deformation (and thus strain) experienced by the wire or foil pattern 256. An excitation voltage may be applied to the strain gauge and a sense voltage may be measured as an output voltage. As the resistance changes due to induced strain, the output voltage also changes.

The present example comprises a metallic foil pattern 256 arranged on a flexible support 255, such as a thin silicone film 255. The flexible support or support patch 255, can be attached to an outer surface of the effector tissue 230 to be measured. Due to the flexible nature of the support 255, it may deform and contract as the effector tissue 230 deforms and contracts, thereby causing the metallic foil pattern 256 to deform accordingly.

The output from the sensor device 250 may be retrieved by the control unit 240, which may be configured to determine a response measure based on the sensor signal. The response measure may be understood as a measure indicative of the effector response. Hence, the response measure may be a certain voltage, impedance, phase, resistance, or degree of contraction or relaxation, depending on the principle of operation used by the sensor device. In case of the sensor device 250 being an EMG sensor, the response measure may be a voltage, in case of the sensor device 250 being an EIM sensor, the response measure may be an impedance and/or phase, and in case of the sensor device 250 being an mechanomyography (MMG) sensor, the response measure may be a resistance or degree of deformation.

The control unit 240 may be operable to compare the response measure with a predetermined reference measure and control the stimulation device based on the comparison in order to adjust or maintain a desired response in the effector tissue 230. The control unit 240 may, for example, increase an intensity of the stimulation signal in response to the response measure being below the reference measure and reduce the intensity of the stimulation signal in response to the response measure exceeding the reference measure. The control unit 240 may thus operate as a closed-loop controller, or feedback controller, using information carried by the sensor signal as feedback when controlling the operation of the stimulation device in a control loop. The control unit 240 may be configured to increase the intensity of the stimulation signal by increasing at least one of a frequency, current amplitude, and voltage amplitude of the stimulation signal. Further, the control unit 240 may be configured to reduce the intensity of the stimulation signal by reducing at least one of the frequency, current amplitude, and voltage amplitude of the stimulation signal.

The predetermined reference measure may be based on a previous measurement of the effector response in the patient and/or on previous measurements of effector responses in other patients.

The control unit 240 may be configured to monitor the level of effector response over time and control the stimulation device based on a change rate in the effector response over time. Thus, the control unit 240 may be arranged to calculate a time derivative of the effector response and control the operation of the stimulation device accordingly.

It will be appreciated that the response measure in some examples may be used to determine a calibration parameter of the stimulation device. The determination of the calibration parameter may form part of a calibration process, which may be performed in connection with implantation of the stimulation device. The calibration process may also be performed intermittently or on a regular basis, for example upon request by a healthcare professional. The calibration parameter may indicate an offset needed to adjust a characteristic of the stimulation signal, such as a voltage, frequency, or current, to achieve a desired level of effector response. The calibration process may hence be performed to ensure proper operation of the stimulation device and increase the prospects of a desired and predictable effect of the applied stimulation signal.

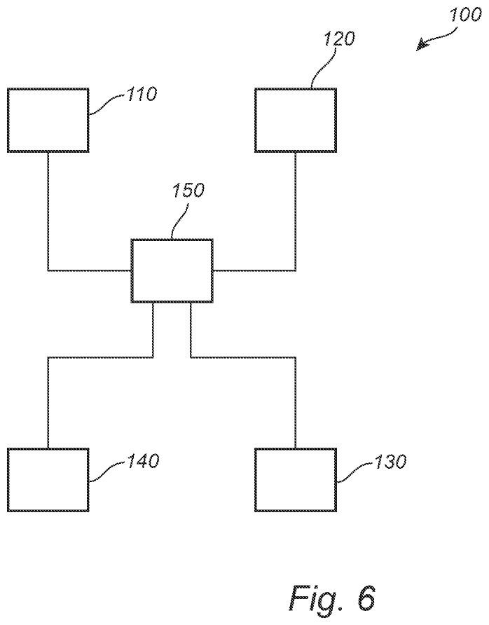

FIG. 6 is a schematic outline of a device, or system, for affecting an effector response in a patient. In a particular example, the system may be used for treating a patient with hypertension. The system may comprise an implantable stimulation device 110 and, optionally, an implantable signal damping device 120, which may be similarly configured as the stimulation and signal damping devices discussed above in connection with the previous examples. The system may further comprise an implantable source of energy, or energy storage unit 130, for energizing the stimulation device 110 and the signal damping device 120 and providing the electrical energy required for generating the electrical stimulation signal and the electric damping signal. Further, the system may comprise a control unit or controller 150 configured to control the generation of the stimulation signal and/or the damping signal, and a sensor configured to generate input that can be used for generating the damping signal.

Any of the above elements, such as the energy storage unit 130, the sensor 140, and the controller 150, or parts thereof, may be configured to be attached to a tissue wall of the body by means of a holding device.

The energy storage unit 130 may for instance be of a non-rechargeable type, such as a primary cell, or of a rechargeable type, such as a secondary cell. The energy storage unit 130 may be rechargeable by energy transmitted from outside the body, from an external energy storage unit, or be replaced by surgery when needed.

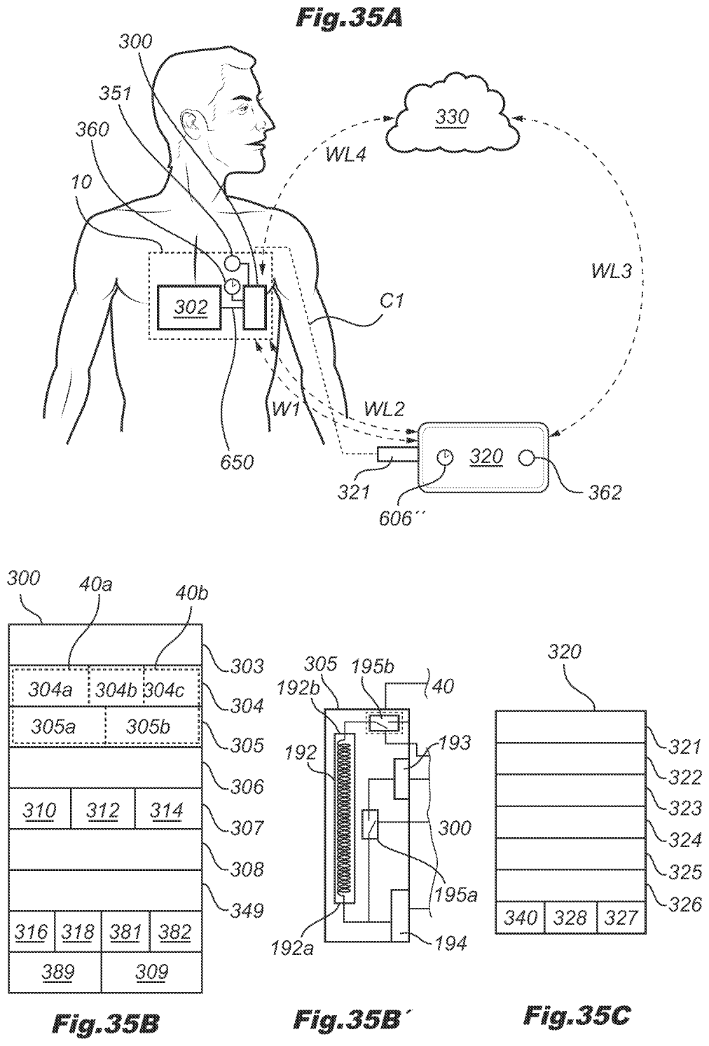

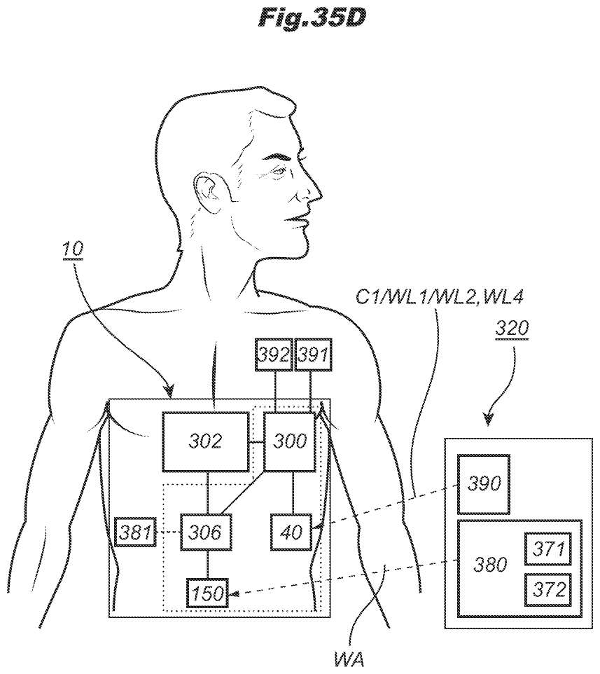

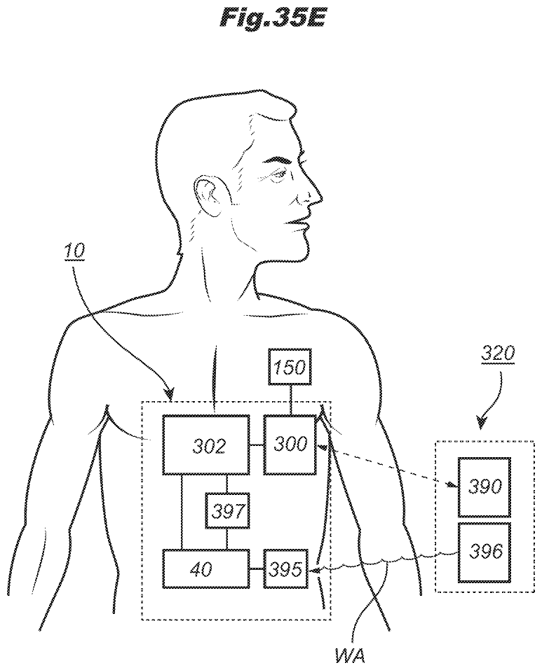

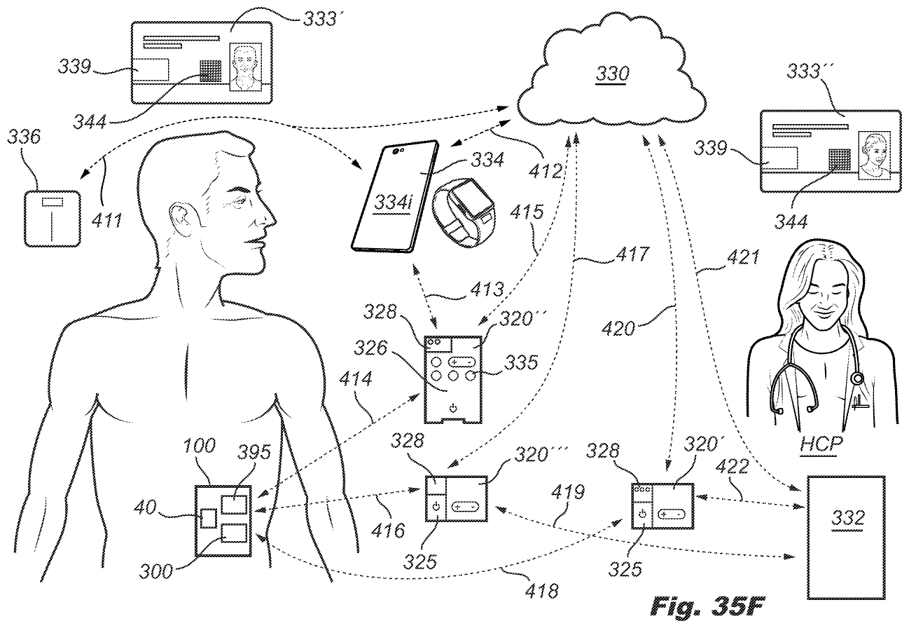

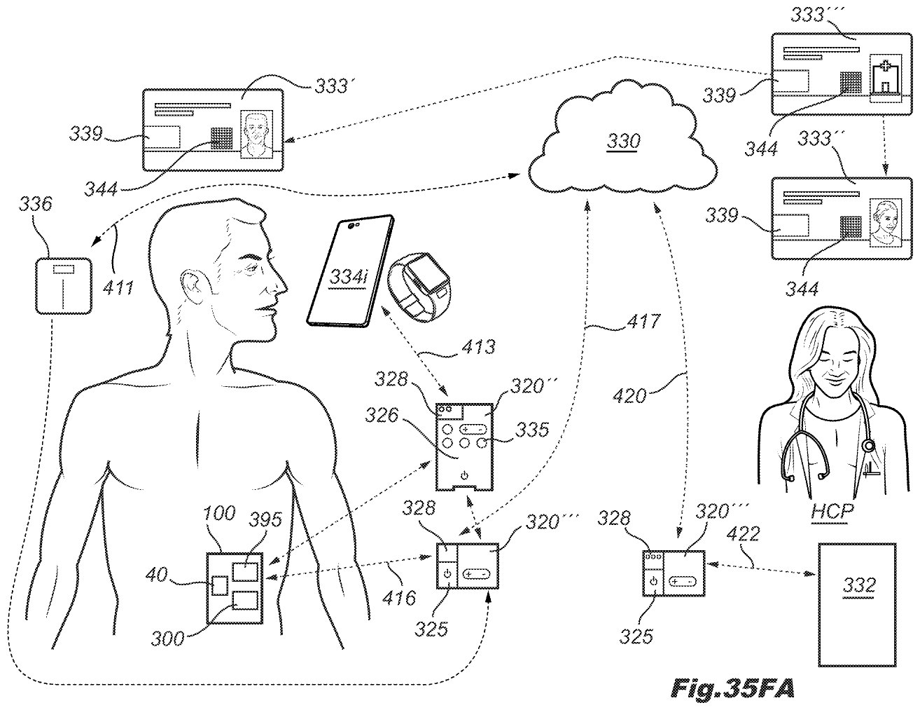

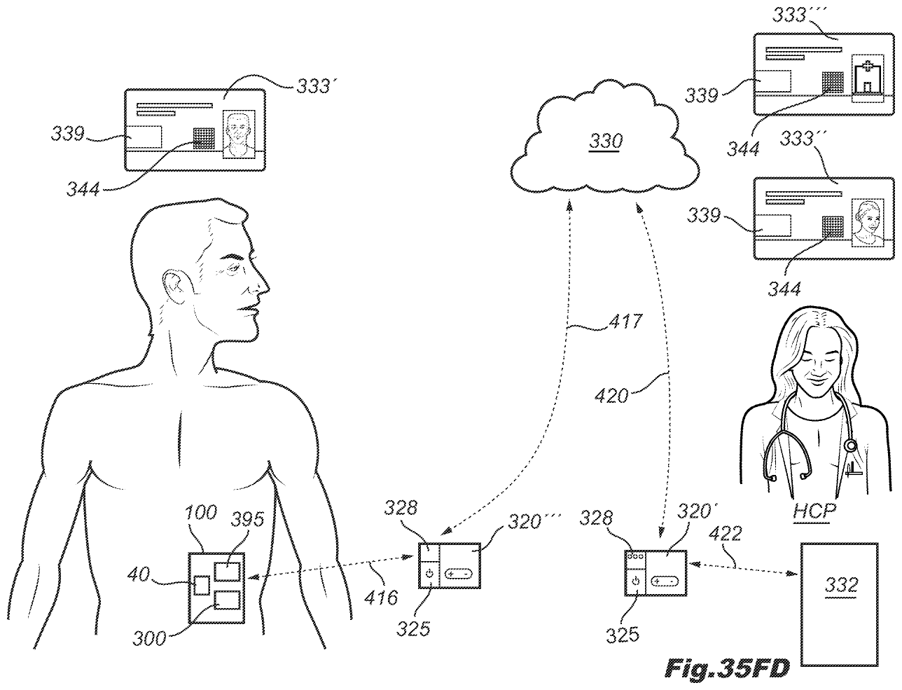

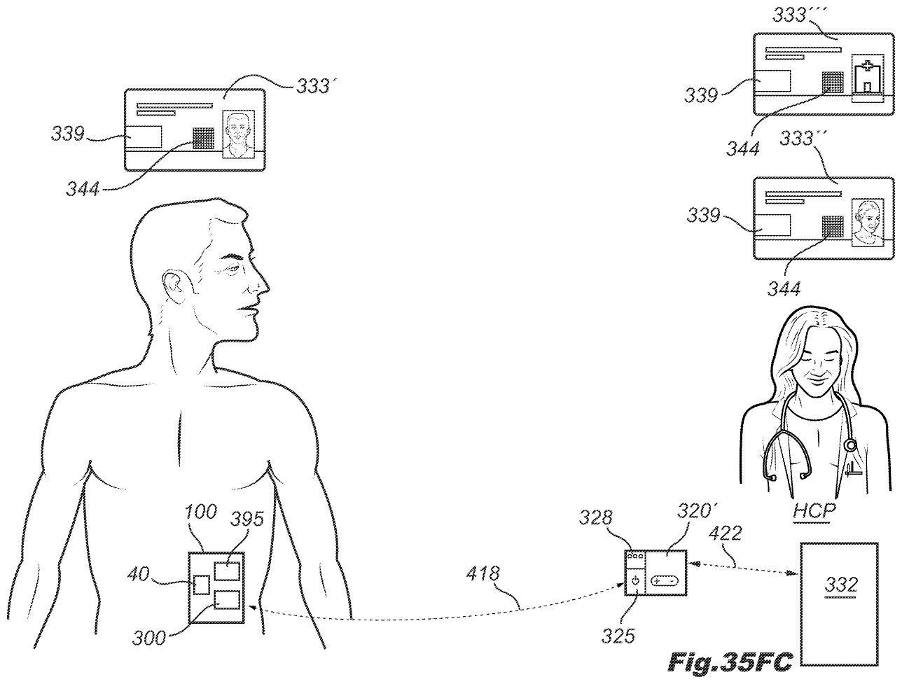

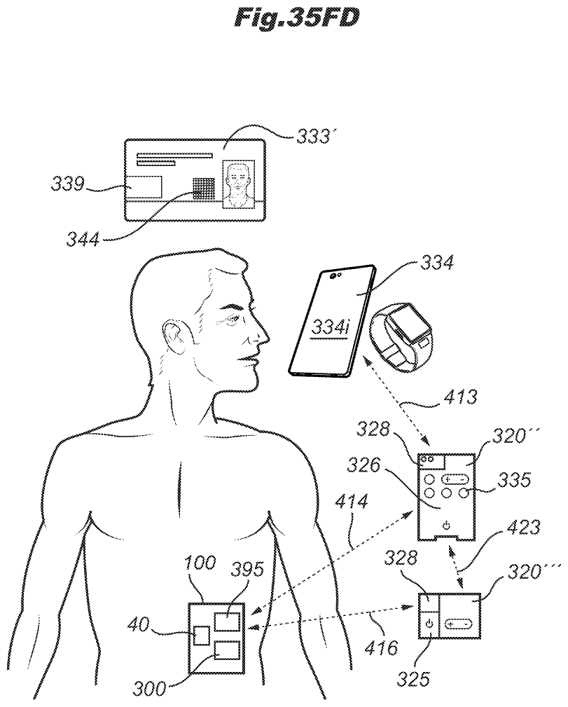

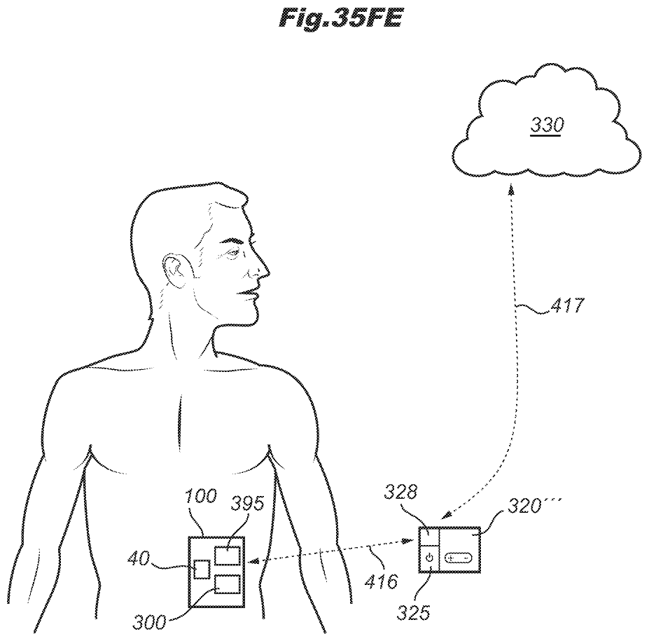

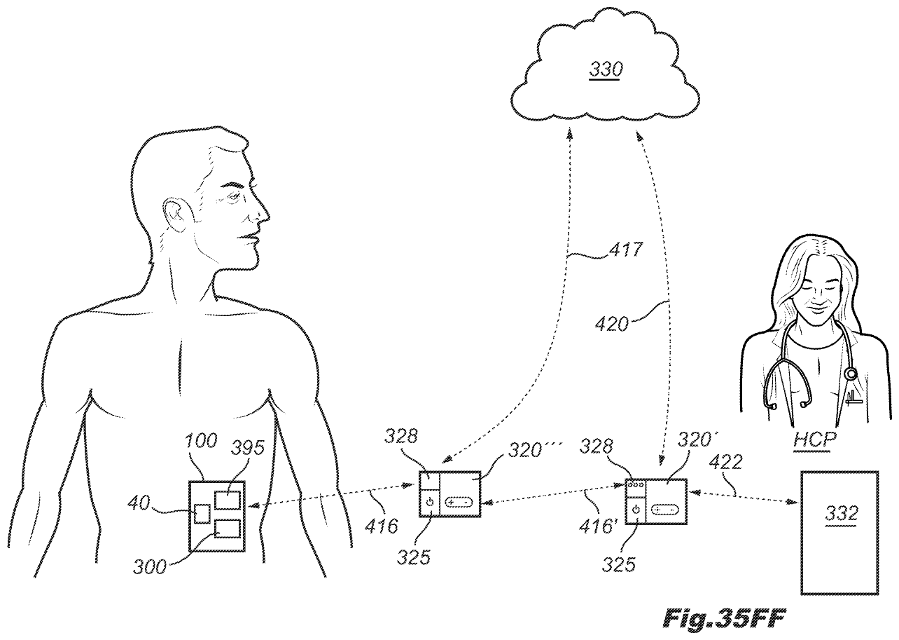

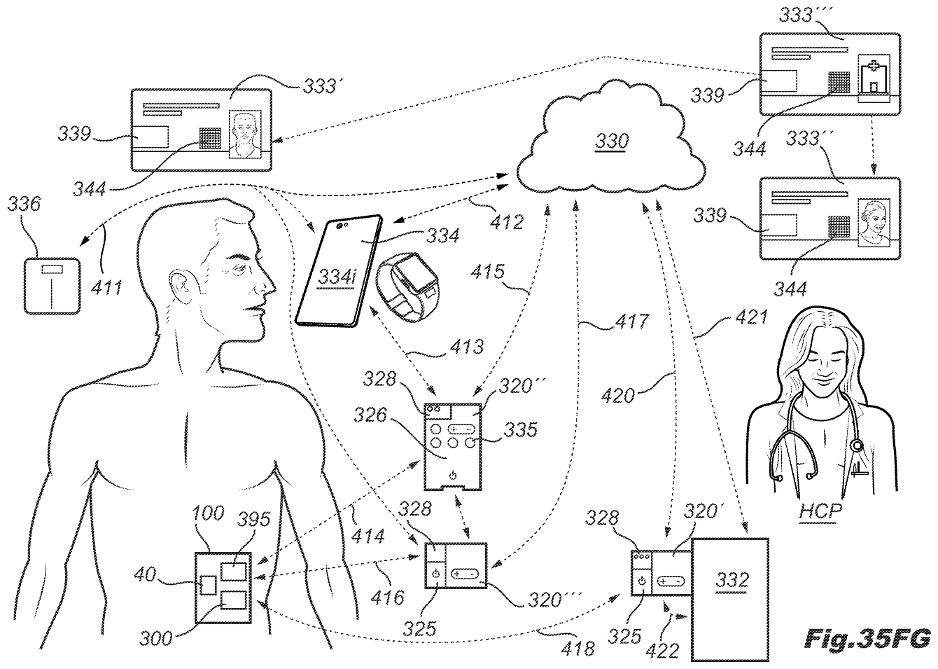

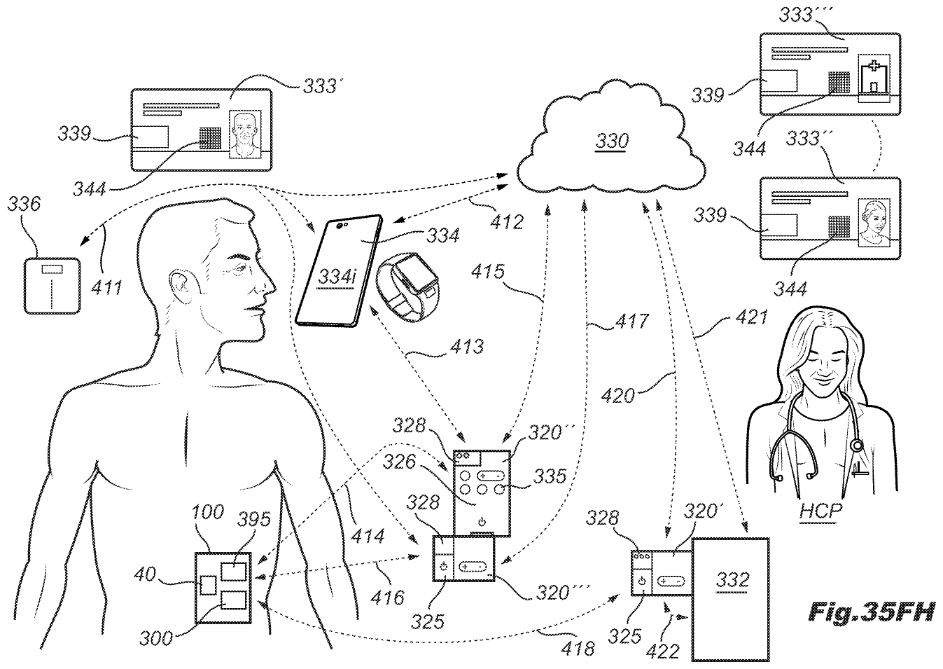

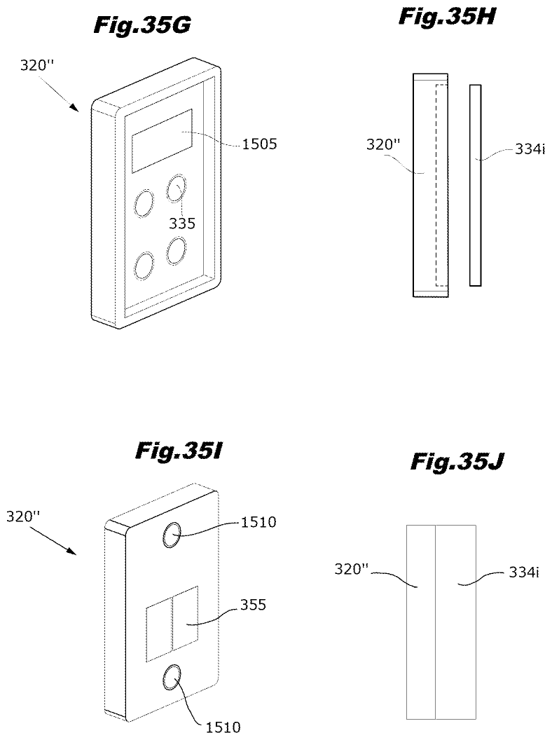

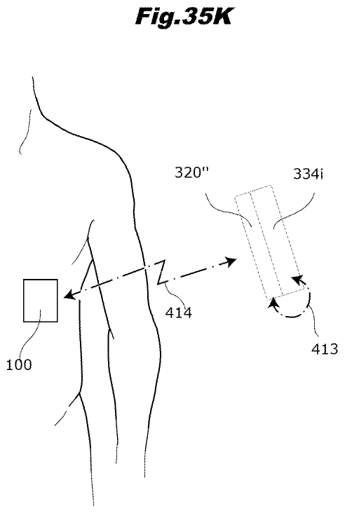

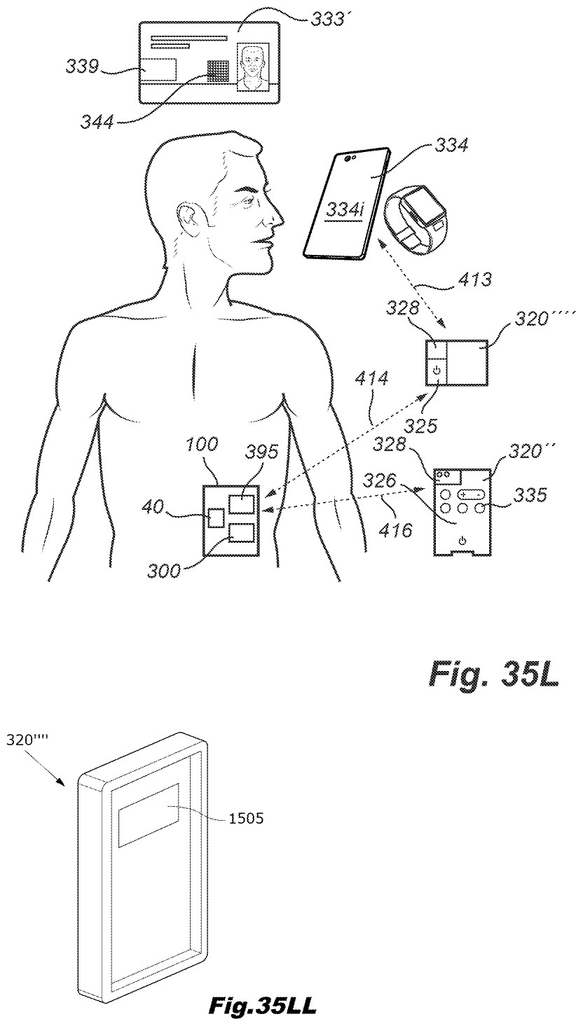

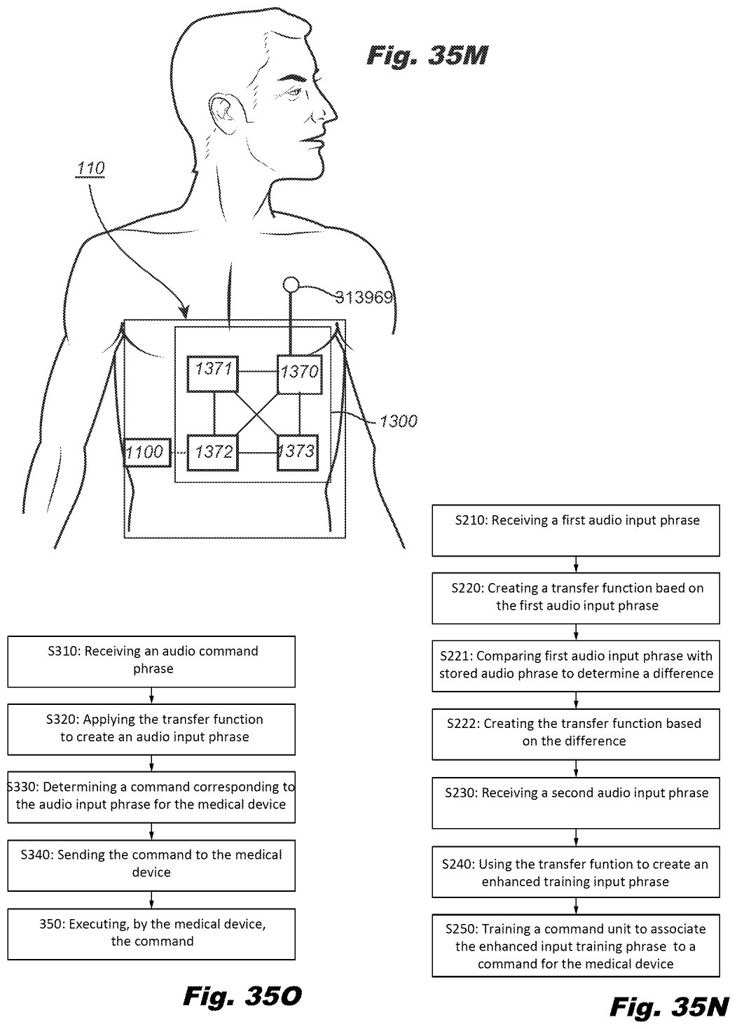

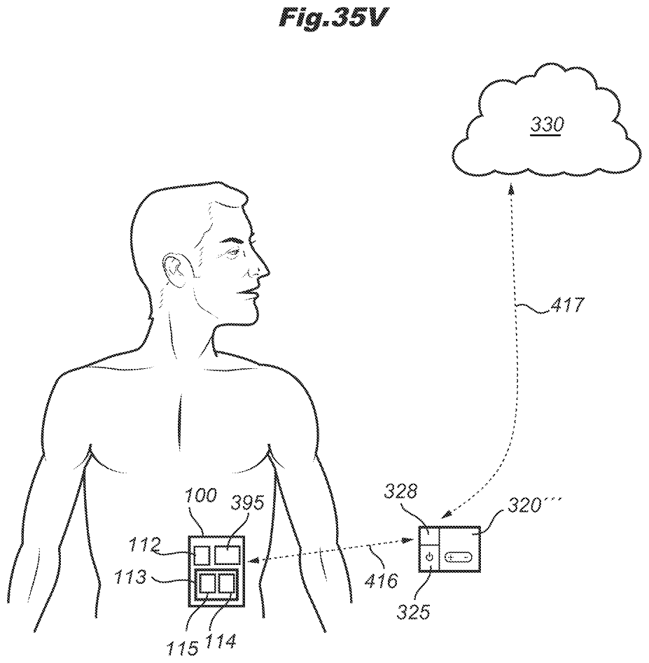

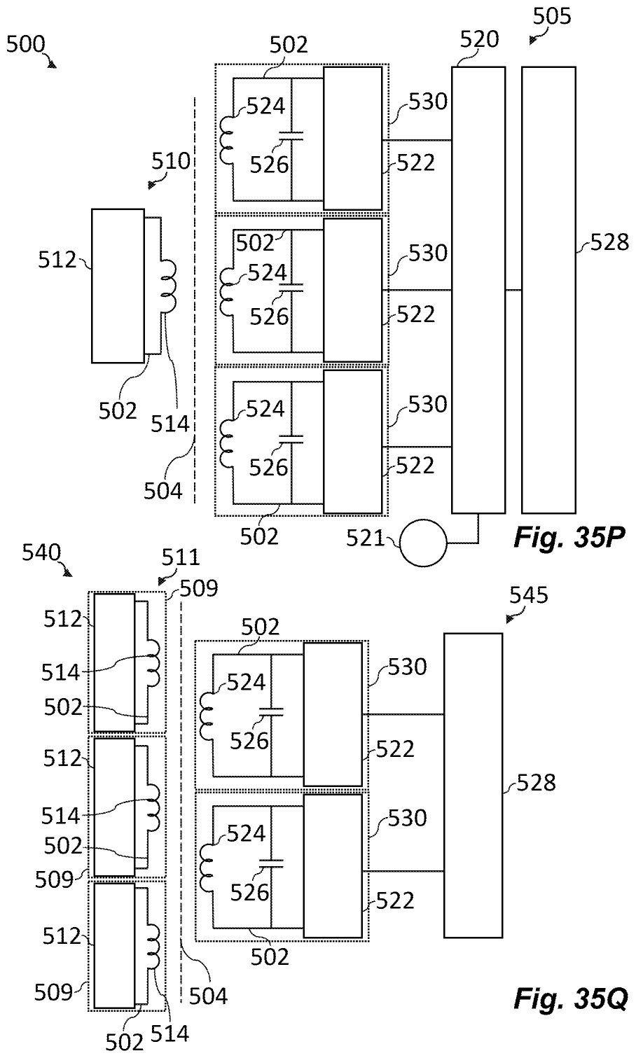

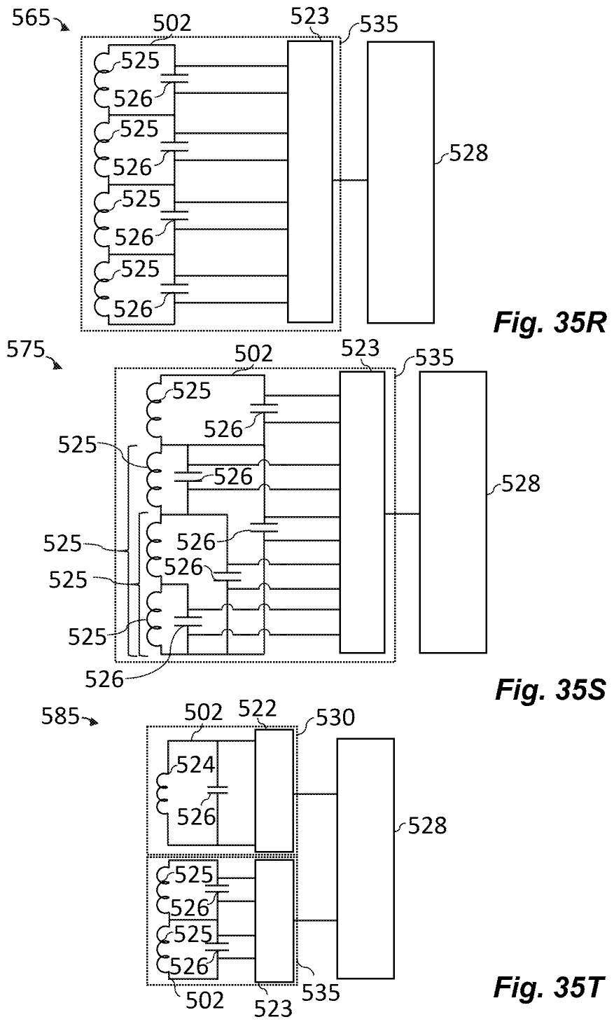

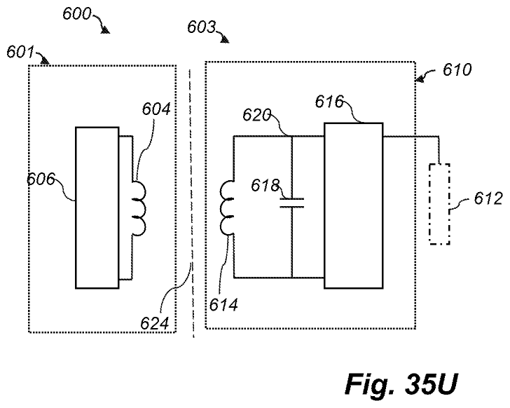

The controller 150 may comprise an electric pulse generator for generating electrical pulses to the stimulation signal and/or the damping signal. The controller 150 may be integrated with the energy storage unit 130 or provided as a separate, physically distinct unit which may be configured to be implanted in the body or operate from the outside of the body. In case of the latter, it may be advantageous to allow an external control unit to communicate wirelessly with the controller 150 for example by means of a communication unit of a more general controller (not shown). The external controller may for example be a wireless remote control, and the controller may in such cases advantageously comprise an internal signal transceiver configured to receive and transmit communication signals from/to an external signal transmitter. More detailed examples are disclosed in connection with FIGS. 35A-U.

In some examples, the controller 150 may be configured to generate a signal indicating a functional status of the source of energy 130, such as for instance a charge level or a temperature of the source of energy 130. Further, the control unit 150 may in some examples be configured to indicate a temperature of at least one of the stimulation device 110, the signal damping device 120 and tissue adjacent to the stimulation device 110 or the signal damping device 120.

In some cases, the system comprises a sensor 140, which may be configured to sense a physical parameter of the body and/or the implantable device. The sensor may be similarly configured as the sensors discussed below in connection with FIGS. 21A-D.

The sensor 140 may for example be employed to sense or detect an effector response in the form of a relaxation or contraction of muscle tissue. This may, for example, include a stretching or contraction of the outer wall of a renal artery 20, thereby allowing for the vasoconstriction and vasodilation of the renal artery 20 to be monitored. The sensor 140 may in this example comprise a strain gauge, as shown in FIG. 21D, configured to indicate a strain of the outer wall of the renal artery 20. In an example, the relaxation of the blood vessel may be verified by means of the sensor 140 and the stimulation device 110 controlled accordingly. The stimulation device 110 may for example modify the stimulation signal based on feedback from the sensor 140 pertaining to the muscular response to the stimulation signal, which advantageously may allow for the stimulation signal to be modified to improve or increase the vasodilation in the renal artery 20. In further examples, the sensor 140 may comprise a pressure sensor configured to generate a signal indicating a pressure in the renal artery 20. The signal indicating the pressure in the blood vessel may for instance be sent to the controller 150 and used as input for adjusting the electrical stimulation signal affecting the vasomotor tone of the smooth muscle tissue of the renal artery 20.

In further examples, the sensor 140 may be configured to generate a signal indicative of electrical properties of the signal propagating from the stimulation device 110, such as the signal propagating towards regions of the body which should not be stimulated by the stimulation signal. Examples of such regions may for instance include the aorta 22 and ganglia from which the nerves innervating the renal artery origin. The sensor 140 may for example include a voltage sensor and/or a current sensor and may be configured to deliver information to the controller 150 pertaining to for instance voltage, amplitude and frequency of signals propagating from the electrode elements 112a of the stimulation device 110.

The controller 150 may be configured to use this information to generate a damping signal which can be supplied to for instance the tissue of the renal artery, close to the bifurcation with the aorta, or at least reducing the tissue's muscular response to the propagated stimulation signal. The sensor may for example be structurally integrated with the signal damping device 120, or provided as a separate, structurally distinct unit. In some examples, the sensor may comprise one or several electrode elements or electrical probes, which may be arranged to engage the nerve or muscular tissue through which the signal from the stimulation device passes.

In some examples, the sensor 140 may be configured to sense or detect action potentials that are being transmitted to the muscle tissue. The action potentials may be registered by the sensor 140 and information relating to the action potentials be transmitted to the controller 150. The controller 150 may use the received information when controlling the signal damping device 120 to reduce the effect of the electric stimulation signal on tissue to which the electrical stimulation signal has propagated.

As mentioned above in connection with FIG. 6 it will be appreciated that any of the above embodiments, may include a sensor configured to generate a signal indicative of an effector response of the patient. In particular examples, the effector response may be a vasodilation or vasoconstriction, affecting a blood pressure (or vascular resistance) of the patient. The various examples and embodiments of sensors described herein may be combined with any of the systems and devices for delivering a stimulation signal as disclosed herein. Similar features and effects will therefore not be repeated in the following.

The inventive concept may utilize sensors of a transducer type, in which energy is converted from one form to another. The sensor may thus be configured to convert a pressure signal (measured directly in the blood or indirectly via an intermediate medium, such as the wall of the blood vessel) into for instance an electrical signal which thus may be considered to be a function of the pressure.

The sensor may be of a dynamic type, configured to capture or monitor the pressure over time and generate a signal indicating the pressure for each measurement point (or continuously, depending on sensor type). The control unit, to which the signal may be sent, may then analyses the signal and make the decision to initiate or stop the stimulation of the renal artery. Alternatively, the sensor may be of a switch type which is configured to turn on or off at a particular pressure. For example, the sensor may be configured to generate a trigger signal for blood pressures being above a certain threshold (or, in alternative configurations, for blood pressures being below a certain threshold). In such cases, the control unit may be configured to treat the signal as a trigger or ON signal, initiating the stimulation of the muscle tissue of the renal artery. In different words, the values of the signal from the sensor may either be (substantially) continuous (giving a substantially true representation of any changes in the measured quantity) or binary, indicating whether the measured quantity is above or below a given limit.

The sensor may be configured to measure the pressure relative to a reference pressure, such as perfect vacuum. This type of sensor may be referred to as an absolute pressure sensor. The sensor may also be a differential pressure sensor, configured to measure the difference between two pressures, such as the pressure inside the blood vessel compared to the pressure outside the blood vessel, or the atmospheric pressure. This type of sensor is sometimes referred to as a gauge pressure sensor.

The pressure sensor may be of a force collector type, using a force collector (such as a diaphragm, piston, bourdon type, or bellows) to measure strain (or deflection) due to applied force over an area (pressure). The sensor may for example utilize piezoelectric or piezoresistive effects to detect strain due to applied pressure or employ a variable capacitor technology to generate a signal as pressure deforms for instance a diaphragm. Pressure induced displacements of elements of the sensor (or parts of the patient's body) may also be measured by means of changes in inductance, Hall effect, eddy currents and the like. In further examples, electrically conductive strain gauges may be attached to an area which moves due to applied pressure and used for generating a signal indicative of the movement of the area. In yet further examples the sensor may operate based on an optical technique, including the use of the physical change of an optical fiber to detect strain due to applied pressure or optical coupling. Alternatively, or additionally, changes in the blood flow may be measured using optical methods, involving for instance radar or doppler effects, or by monitoring the optical coupling efficiency of light passing through the blood vessel. These principles may utilize the observations that light may behave differently depending on the pressure in the blood. Non-limiting details and examples will be discussed in further detail in the following.

The sensor may be arranged at the renal artery, preferably the same renal artery as the one to which the electrical muscle tissue stimulation is applied. A merit of this arrangement is that the sensor may deliver a signal indicating pressure changes resulting from vasodilation of the renal artery and can therefore be considered to provide a more direct feedback to the stimulation process. Put differently, a control loop may be achieved, which utilizes feedback data that are obtained from the same blood vessel as the one that is being electrically stimulated.

Alternatively, or additionally, a sensor may be arranged elsewhere, i.e., remote from the renal artery which is electrically stimulated. One or more sensors may hence be arranged at a blood vessel in another part of the patient's body, such as the aorta, or an artery in the abdomen or a limb of the body, to generate a signal indicative of a systemic blood pressure of the patient. It may be advantageous to arrange the sensor at a position which is easier to access than the renal artery, allowing for the sensor to be implanted in a less complicated and invasive surgical procedure.

The sensor may be configured for long-term implantation, or permanent implantation, in which the sensor is expected to be operating for several months or years without having to be replaced or physically accessed. This allows for the sensor to be operable continuously during the operation of the stimulation device. Alternatively, the sensor may be configured for a temporal use, for instance during a shorter period in which the stimulation device is calibrated. The sensor may thus be implanted for a few hours, days or weeks, for example during setup or calibration of the stimulation device, whereafter the sensor may be removed.

According to some embodiments, the sensor may be arranged to measure the pressure directly in the blood vessel. This may for example be achieved by arranging a probe inside the blood vessel, such as the renal artery, or another artery such as the radial artery, femoral, dorsalis pedis or brachial artery. The probe may thus be employed to generate a signal indicative of the pressure acting on the probe, thereby giving an indication of the blood pressure.

According to some embodiments, the pressure sensor may be arranged at an outer wall of the blood vessel of the patient. The sensor may for example be formed as a cuff at least partly enclosing the blood vessel or be arranged to abut at least a portion of the outer wall. By this arrangement, the sensor may be configured to measure pulse waves transmitted by the blood into the wall of the blood vessel. The pulse waves transmitted through the wall may be converted into a signal, such as an electrical signal, by means of a strain gauge reacting on a strain induced in the wall portion by the pressure pulses, or by means of a contact pressure sensor configured to react or monitor a contact pressure between the outer wall portion and the sensor. A pulse wave, transmitted through the blood, may hence give rise to an increased pressing force between the outer wall of the blood vessel and the pressure sensor, which in turn may be configured to convert the increased pressing force into a signal indicative of the pressure according to a technique mentioned above.

According to some embodiments, the sensor may comprise a light source configured to input light into the blood, such as through the wall portion of the blood vessel, and a light sensor configured to receive light transmitted from the light source. The light sensor may for instance be arranged outside the blood vessel, at a side opposing the light source. This may be referred to as an optical sensor, which in some examples may base the pressure measurements on a light coupling efficiency through the blood vessel. The light coupling efficiency may for instance be a function of a contact pressure between the light source and the wall portion of the blood vessel, and/or a contact pressure between the light sensor and a wall portion of the blood vessel and may therefore be used to indicate a characteristic of the pressure pulse generated by the heartbeats. Optical methods may also be used to measure a deflection, or movement, of a wall portion of the blood vessel in response to the pressure pulse wave travelling through the blood vessel. Such an optical method may for instance utilize the doppler radar effect to monitor a pulse wave causing a movement in the wall portion of the blood vessel.

According to some embodiments, the sensor may operate according to the auscultatory principle, in which a constrictive element, or constriction device, is placed around the blood vessel and operated to constrict the blood vessel until is occluded and the blood flow therein stopped. The constriction may then be gradually released, and the constrictive pressure registered as a function of the returning blood flow. In an example, the constriction device is used in an oscillometric method, in which oscillations in the constrictive pressure caused by oscillations in the blood flow, i.e., the pulse, are measured. The constriction device may for instance be operated to a pressure initially exceeding the systolic arterial pressure and then reduce to below the diastolic pressure. When blood flow is substantially nil (constrictive pressure exceeding systolic pressure) or substantially unimpeded (constrictive pressure below diastolic pressure), the constrictive pressure may be essentially constant. When blood flow is present, but restricted, the constrictive pressure, which may be monitored by the sensor, may vary periodically in synchrony with the cyclic expansion and contraction of the blood vessel, i.e., it will oscillate. Over the release period, in which the constrictive pressure is reduced, the recorded pressure waveform may form a signal from which the oscillometric pulses may be extracted using a bandpass filter. The extracted oscillometric pulses may form a signal referred to as the oscillometric waveform, OMW, which can be analyzed and processed to estimate the systolic, diastolic and mean arterial pressure.

The sensor may in some examples be configured to generate a signal indicative of a vascular resistance of a blood vessel of the patient. As the blood pressure may be understood as a function of (inter alia) the vascular resistance, this measure may be used when estimating the blood pressure. The sensor may for instance be configured to measure a flow of blood in the blood vessel, to measure a vasodilation or vasoconstriction of the blood vessel, and/or a size of the blood vessel (such as inner or outer diameter or cross-sectional size). The blood flow through the blood vessel may for instance be monitored by means of a light coupling method as indicated above, where the composition of the blood is monitored to estimate a flow of the blood. This may for example involve observing or estimating a number of red blood cells passing a certain region or volume of the blood cell per unit of time. An increase in blood flow may indicate a reduced vascular resistance, whereas a reduced blood flow may indicate an increased vascular resistance.

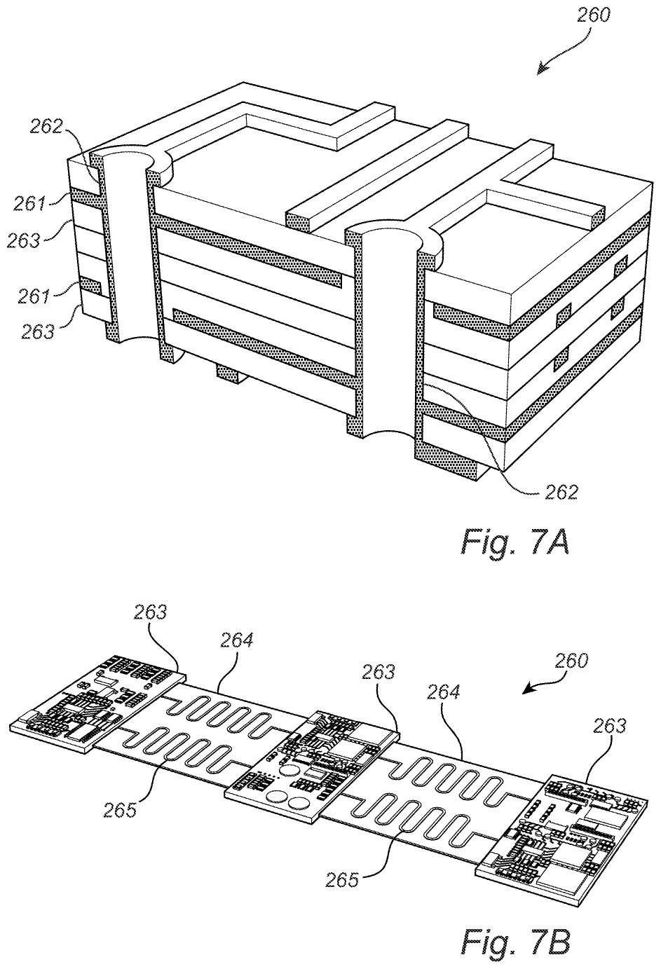

A printed circuit board (PCB) 260, may be employed to accommodate the circuitry and electrical components enabling the functionality of the systems described above. Accordingly, at least one of the stimulation device, the source of energy, and the control unit 240 may be supported by such a PCB. The PCB may be integrated in a housing or casing facilitating implantation in the body of the patient. Specific examples of PCBs 260 will now be discussed with reference to FIGS. 7A and B.

The PCB 260 serves as a physical platform for supporting and interconnecting electronic components of the system. The PCB 260 typically comprises a substrate 263 on which conductive paths 261 are etched or printed to establish electrical connections. Components such as resistors, capacitors, and integrated circuits such as ASICs may then be mounted on the substrate 263. The design and configuration of the PCB 260 depend on the intended application and site of implantation, with considerations for size and flexibility playing roles.

FIG. 7A shows an example of a multi-layer PCB 260, in which a plurality of substrate layers 263, each provided with its own set of conductive paths 261, are bonded together with layers of insulation. The multi-layer configuration reduces the footprint of the PCB 260, allowing for more functionality within the limited space of a medical implant, such as the stimulation device. The layers 263 are interconnected through vias 262 which may be through-holes filled or plated with conductive material. The relatively compact design of the PCB 260, with the reduced footprint enabled by the multi-layered configuration, makes it possible to implant the device in locations where the available space is relatively restricted.

The PCB 260 may also be of a flexible type and/or a stretchable type. Flexible PCBs are typically made using a flexible substrate, such as polyimide or polyester film, which allows the PCB to conform to a specific shape or flex during its use. This flexibility is particularly advantageous in medical implants that need to move or flex with the surrounding tissue, reducing the risk of damage to both the device and the tissue. Flexible PCBs can be single-layered or multi-layered and may, beneficially, be used in implants requiring adaptability to movement or specific anatomic contours.

Stretchable PCBs may be fabricated from materials that can withstand stretching, such as silicone-based substrates with conductive paths that can withstand stretching. The conductive paths may, for example, be formed of silver-filled silicone, or a conductive path or wire may be arranged in a ‘serpentine’ trace. The serpentine trace may be characterized by its zigzag or wave-like pattern, effectively distributing mechanical stress over a larger area and absorbing deformations caused by the substrate moving or stretching.

FIG. 7B shows a particular example of a PCB 260 which is both flexible and stretchable. The PCB 260 comprises one or more flexible portions, such as flexible substrate portions 263, as well as one or more flexible portions comprising a stretchable substrate 264 with a conductive path 265 arranged in a wave-like pattern. In the present example, the PCB 260 comprises three flexible portions 263 interconnected by two stretchable substrate portions 264. The resulting arrangement is a PCB 260 that can conform to the specific shape of the tissue to which it is attached and adapt to movements, such as contractions of the tissue. In further examples, two or more non-flexible portions (such as regular planar PCB portions or multi-layered PCB portions) may be interconnected by one or more flexible or stretchable portions to provide some flexibility/stretchability to the arrangement.

Various measures may be taken to ensure electrical safety and to comply with different regulatory frameworks. Direct current (DC) flowing through electrodes or other implanted parts of a system according to any of the aspects of the present disclosure may be a safety concern, as it may cause tissue damage. For example, it has been reported that DC levels as low as 2-3 μA may cause pathological changes in nerve tissue. It is therefore desirable to limit leakage current (DC) to 1 μA or less, such a 0.1 μA or less. This may be achieved by means of a capacitor, also referred to as a DC blocking capacitor, which may be arranged in any of the current pathways. Specifically, the capacitor may be connected in series with two or more electrodes of the implant, such as the ones employed to apply a stimulation signal or a measuring signal. Furthermore, the capacitor may be connected in series with a part of the implant (such as an electrode, an energy source, or a housing) and the body of the patient, thereby reducing any current that might flow between the implant and tissue of the patient.

A further advantage of the capacitor relates to prevention of charge accumulation on the electrodes. By coupling a capacitor to the electrodes, the capacitor may help dissipating accumulated charge from the electrodes, thereby allowing them to ‘slide back’ to their operating potential range.

The capacitor may be implemented in the circuitry of the medical device, such as the stimulation device discussed above. The capacitor may hence be provided as a component on any of the PCBs 260 or separate from the PCB 260.

Denervation is a process of interrupting the nerve supply to a particular organ or area in the body. This can be done for various medical reasons, such as to relieve pain, reduce muscle spasticity, or address certain pathological conditions or illness symptoms. Denervation typically involves cutting off or disrupting the nerve signals to specific part of the body. This can be achieved through various methods, such as surgical removal, the use of chemicals, or by applying thermal or electrical energy to destroy nerve tissue.

Surgical denervation may involve physically cutting or removing the nerve or part of it. It is often permanent and may be used in cases where other treatments have failed.

Chemical denervation involves injecting a substance (like alcohol or phenol) that destroys or blocks the nerve fibers. This method may often be used for spastic muscles or for pain management.

Radiofrequency (RF) ablation employs high-frequency electrical currents to heat up a small area of nerve tissue, thereby blocking further signaling through the nerve.

Cryoablation is an example where cold is employed to freeze and destroy the nerve tissue.

Denervation can be effective in providing a long term or even permanent effect on the treated tissue. However, it also comes with risks such as undesired side effects and denervation of the ‘wrong’ tissue. Incorrect placement of the ablation tool or administration of the nerve blocking or nerve destructing substance may lead to undesired damages and denervation of tissue not intended to be denervated.

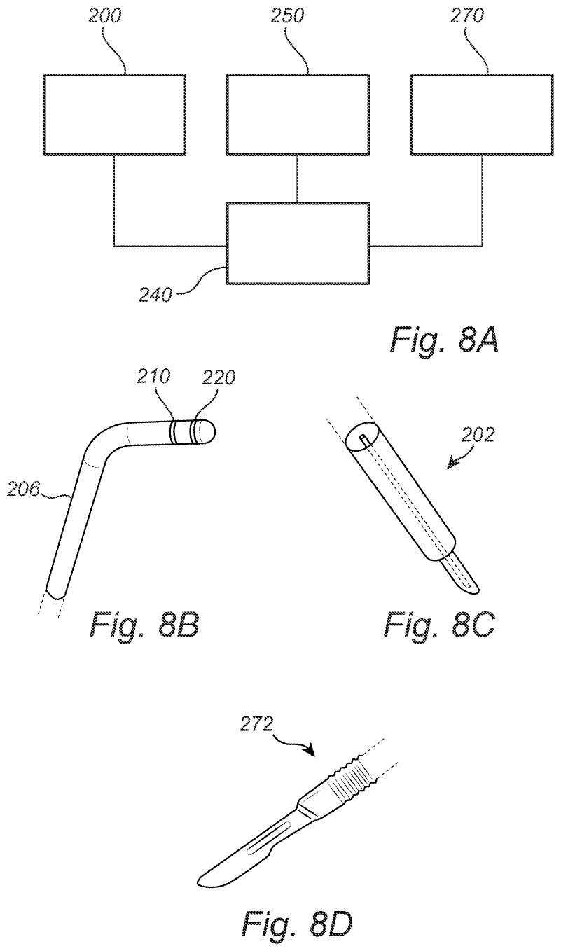

FIG. 8A shows a system comprising an inhibition device configured to temporarily inhibit a nerve innervating the effector tissue. A sensor 250 may be employed to generate a sensor signal indicative of an effector response in the effector tissue innervated by the nerve, wherein the effector response is at least partly induced by the inhibiting of the nerve. The effector response may be used to determine whether a desired effect is achieved by the inhibition of the nerve before the nerve is being denervated. The present system therefore makes it possible to verify whether the correct nerve is identified before the denervation is performed. This may be verified by determining a response measure based on the sensor signal, wherein the response measure is indicative of the effector response, in order to compare the response measure with a predetermined reference measure, and determining whether the desired effector response has been achieved.

The inhibition device may be a stimulation device according to any of the above-discussed examples, comprising one or more signal generating means, such as electrode arrangements 210, 220 or vibration arrangements for delivering an inhibition signal hindering action potentials from passing through the nerve. FIG. 8B shows an example of a probe 206 or catheter comprising a first electrode arrangement 210 and a second electrode arrangement 220. The probe 206 is configured to be inserted into the body and arranged such that the first and second electrode arrangements 210, 220 can be placed against the tissue that is to be ablated. In some examples, the probe 206 may be a combined inhibition and denervation device. In other words, the same probe 246 may be used both for temporarily inhibiting the nerve and for ablating it. This can be achieved by operating the electrode arrangements 210, 220 in different modes—an inhibition mode for inhibiting the nerve and a denervation mode for denervating the effector tissue. In the inhibition mode, the electrode arrangements 210, 220 may be operated to apply an inhibition signal as discussed above, whereas in the denervation mode they may be operated to apply an ablation signal for blocking further signaling through the nerve. While the inhibition signal is a relatively weak/low power signal intended not to cause permanent damage to the tissue, the ablation signal may be a relatively strong signal/high power signal. The inhibition signal may typically have a frequency in the range of 1-10 kHz, a voltage of about 1-15 V, and a current of about 1-50 mA (corresponding to a power of less than 1 W). The ablation signal, on the other hand, may comprise an alternating current in the range of 350-500 kHz, a voltage ranging from 500 to 400 V and, in some examples, a power of 50 to 40 W. This may be referred to as a type of radiofrequency ablation (RFA).

The inhibition device may in some examples comprise a cooling device configured to cool the nerve to cause a temporary inhibition of the nerve. The cooling device may be formed as a cooling element arranged on the probe 246, which hence may be referred to as a cryoprobe. The cryoprobe 246 may comprise a lumen or channel guiding the cooling gas to the tip of the probe 246, which tip may be placed against the nerve to induce a temporary inhibition. Similar to the electrical probe above, the cryoprobe 246 may be a combined inhibition and ablation device. The difference between the two functions may be determined by the temperature of the probe, more specifically by the degree of cooling of the tissue. A temporary inhibition may occur if the nerve is cooled to a temperature exceeding 0° C., whereas an ablation (or at least a long-term blocking) may be achieved by freezing the tissue (i.e., lowering the temperature below 0° C.). This allows for the placement of the probe 246 to be verified before the ablation is commenced.

In some examples, the inhibition device is operable to deliver a substance to the nerve, such as a toxin temporarily inhibiting the nerve. This may be achieved by means of a toxin administration device 242, of which a particular example is illustrated in FIG. 8C. The toxin administration device 242 may comprise a catheter or lumen that can be inserted into the body of the patient and arranged to deliver the substance directly to the relevant nerve. A relatively mild substance may be used to ensure a temporary inhibition. Should a stronger substance be used, a permanent or at least long-term effect may be achieved, and the process may then be referred to as a neurotoxin-induced ablation. By varying the types or concentrations of the administered substance, the toxin administration device 242 may be used both for an initial, temporary inhibition and a more permanent or long-term ablation. Examples of toxins include neurotoxins, such as botulinum toxin and tetanus toxin.

It will be appreciated that the denervation device 270 may comprise an ablation device configured to denervate the tissue by means of surgical ablation, RF ablation, cryoablation, laser ablation, heat ablation, electrocautery, and chemical ablation. FIG. 8D shows an example of a surgical ablation device 272, comprising a lancet for making incisions in the nerve tissue. The lancet may be understood as a small, sharp instrument that can be used for performing a catheter-based ablation of the nerve.

A control unit 240 may be provided for controlling the operation of one or more of the components of the system, i.e., one or more of the inhibition device, the sensor device 250, and the denervation device 270. The control unit 240 may be arranged to receive a sensor signal from the sensor device 250, indicating a physical response in the effector tissue innervated by the stimulated nerve. By detecting a response in the tissue caused by the signal delivered by the inhibition device, it can be assumed that the inhibition device has been inserted into the correct position or part of the patient's body.

Inhibition of a nerve—either temporarily by means of a suppression signal, or permanently by means of ablation—can also be employed to treat sensations of pain, such as phantom pain. Phantom pain is typically a neuropathic pain, relating to the nerves and often being a response to the loss of a limb or body part. Even though the body part is no longer present, nerve endings at the site of the amputation may continue to send pain signals to the brain, making the brain think the body part is still there and experiencing pain. The nerve may also be inhibited to address phantom limb sensation, which is the feeling that an amputated or missing part of the body is still present, but not necessarily painful.

Hence, according to some examples, a stimulation device 200 as shown in any of the previous examples may be provided to deliver, directly or indirectly, a suppression signal to a nerve 231, 232 of the patient. Further, the control unit 240 may be configured to control an operation of the stimulation device to suppress or block a propagation of action potentials in an afferent direction of the nerve, thereby reducing the sensation of pain originating from the action potentials. It will be appreciated that the suppression signal, or blocking signal, may be delivered in a similar way and by a similar system as discussed in connection with the unidirectional stimulation disclosed in FIG. 3A above. However, to block, inhibit, or at least reduce nerve signals generating a sensation of pain, such as phantom pain, or a sensation of a phantom limb, it may suffice to apply the suppression signal. There might not be a need for applying the stimulation signal as well, as the primary objective of the present example is to prevent action potentials from reaching the central nervous system and the brain.

Similar to the parameters used for generating the suppression signal for the unidirectional stimulation as discussed above, the control unit 240 may be configured to control an operation of the stimulation device 200 such that the suppression signal varies with a frequency in the range of 1-10 kHz. These frequencies have been observed to reduce the nerve's capability of transporting action potentials, effectively resulting in a blocking of the nerve.

In some examples, a sensor device 250 may be employed for generating a sensor signal indicating any action potentials propagating in the nerve. The sensor device 250 may be similarly configured as any of the sensor devices discussed above in connection with the previous figures and examples, and the sensor signal may hence be used as feedback when controlling the operation of the stimulation signal. The sensor device 250 may, for instance, comprise a sensor electrode configured to be arranged cranial to the suppression electrode. This allows for the suppression signal to be modified in response to the output from the sensor device, and more specifically for the intensity or frequency of the suppression signal to be increased in response to action potentials passing the suppression electrode in the cranial direction. Presence of action potentials cranial to the suppression electrode may indicate that the nerve is still capable of transporting action potentials and that a sufficient blocking is not yet achieved. By adjusting the parameters of the suppression signal accordingly, a more efficient blocking of the nerve may be achieved.

Beneficially, the suppression signal is delivered to a nerve via a suppression electrode, such as an electrode similar to the suppression electrodes 221, 222 shown in FIG. 3A. The location of the suppression electrode may vary depending on the type of pain and from where is originates. However, in general, the suppression signal may be delivered to a sensory nerve, also referred to as an afferent nerve, which is known to play a role in transmitting information from various sensory receptors to the CNS 233. Examples of sensory neurons include mechanoreceptors responding to mechanical stimuly such as pressure, touch, and vibrations; thermoreceptors sensitive to temperature changes; nocireceptors including A-delta fibers transmitting sharp, fast pain and C fibers transmitting dull, slow pain. Further examples include proprioreceptors, providing information about body position, movement, and muscle tension. The suppression signal may be applied to a sensory nerve to block transmission of action potentials from the sensory neurons to the spinal cord and further on to the brain. The suppression electrode 221, 222 may be arranged closer to the sensory receptor/neuron than to the spinal cord so as to ensure a more precise effect (reducing the risk of inadvertent blocking of other signals than the ones responsible for the pain/phantom sensation). In other examples, the suppression electrode 221, 222 may be arranged closer to the spinal cord than to the sensory receptor/neuron, depending on the available space and exact location of the sensory receptor.

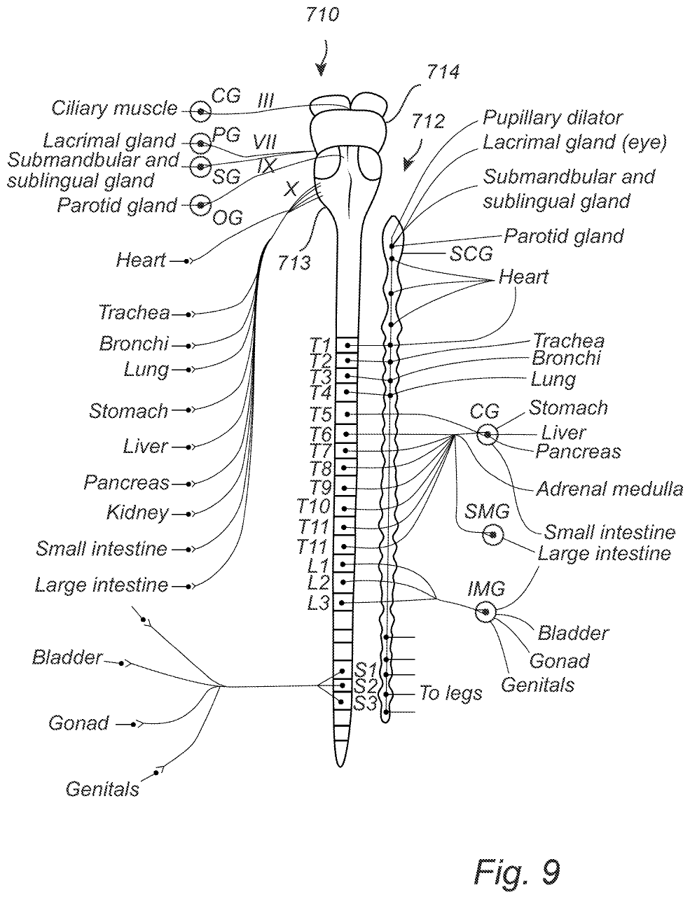

FIG. 9 is a schematic overview of the spinal nerves, and particularly of the sympathetic and parasympathetic nervous system. FIG. 9 illustrates the vertebral column 710 and one of the two sympathetic chains 712 (also called sympathetic trunks) located on each side of the vertebral column. Each sympathetic chain 712 comprises a ganglionated nerve trunk. These ganglia are referred to as paravertebral ganglia (25 pairs) and represent one of the sites wherein the sympathetic postganglionic neurons are located. Axons of the sympathetic preganglionic neurons typically synapse on many sympathetic postganglionic neurons in the sympathetic chain.

The course of preganglionic and postganglionic sympathetic fibers at different levels of the spinal cord 710 is shown in FIG. 9. The axons of sympathetic preganglionic neurons located at T1-L3 typically exit the spinal cord 710 via ventral roots, travel a short distance in the corresponding spinal nerves, and then enter the sympathetic chain via the white rami. Typically, the stimulation signal may be applied to the postganglionic part of the sympathetic nerves to address various symptoms, stimulate various effector tissue, and achieve a desired effector response.

It will be appreciated that the system according to the present disclosure, which may be used for applying one or more of an activation signal, an inhibition signal, and a suppression signal, may be arranged to deliver such signal(s) to any of the nerves originating from the spinal cord 710 or brainstem 714. In the following, some particular examples of possible stimulation sites and possible effects are discussed.

The superior cervical ganglion SCG forms a part of the SNS located in the neck region, near the base of the skull and received preganglionic sympathetic fibers from the thoracic spinal cord (specifically, spinal cord segments T1-T4). These preganglionic fibers synapse with postganglionic neurons in the SCG from where they then continue to their target organs. By applying a stimulation signal-such as an activation signal or an inhibition signal as previously discussed-effector responses such as increase or reduced pupillary dilation, sweating, vasoconstriction, and production can be achieved. Conditions such as Horner's syndrome, characterized by ptosis (drooping eyelid), miosis (constricted pupil), and anhidrosis (lack of sweating) on one side of the face can be addressed. Further, an effect on cluster headache has been observed. Overall, by stimulating neurons connected to the SCG the autonomic balance and coordination of sympathetic response in the head and neck region can be affected.

The sympathetic preganglionic fibers originating from spinal cord segments T1-T4 (or sometimes T5) synapse in the sympathetic trunk ganglia and commonly innervate the respiratory system. For example, the trachea, bronchi, and lungs receive innervation from these spinal cord segments T1-T4. Sympathetic fibers from T1-T4 typically contribute to the regulation of bronchi smooth muscle tone, blood flow, and glandular secretion in the lungs. Sympathetic activation or inhibition caused by application of a stimulation signal to nerve fibers originating from these cord segments may typically affect bronchodilation (for increased/reduced airflow), glandular secretion (increased/reduced mucus production), vasoconstriction (of blood vessels in the lungs), and increased/reduced heart rate (affecting oxygen delivery).

Sympathetic fibers from spinal cord segments T5-T12 typically contribute to the innervation of the stomach. These fibers regulate bodily functions such as gastric motility, blood flow, and secretion. However, the vagus nerve (cranial nerve X) is also known to play a significant role in stomach innervation.

The liver typically receives sympathetic innervation from spinal cord segments T5-T9. Stimulating these nerves may affect liver blood flow and glucose metabolism.

Sympathetic fibers from T5-T9 are known to innervate the pancreas. Stimulation of these nerve may influence pancreatic blood flow and secretion.

The adrenal medulla, which forms a part of the adrenal glands, is also known to be innervated by sympathetic fibers from cord segments T5-T12. Stimulating these nerves may result in an increased/reduced release of adrenaline.

The small and the large intestine is also innervated by nerves from cord segments T5-T12. Applying a stimulation signal to any of these nerves may affect motility and blood flow, which in turn may affect the digestion, the absorption of nutrients, and fecal consistency.

The bladder, gonads, and genitals are typically innervated by sympathetic nerves stemming from the lower end of the spinal cord 710, such as cord segments L1, L2, and L3. Stimulating these nerves may trigger bladder contraction during urination, urinal sphincter contraction, as well as genital sensation, gonadal activity, and motor control.

Turning to the parasympathetic nervous system, a major part of the nerves originate from the brainstem, such as the medulla 713 and pons 714. As shown in FIG. 9, cranial nerve III innervate the ciliary muscle, cranial nerve VII the lacrimal gland, the submandibular gland, and the sublingual gland, and cranial nerve IX the parotid gland. Activating a parasympathetic response in the cranial nerve III may cause the ciliary muscle to relax, whereas an inactivation or inhibition may cause it to contract. Similarly, parasympathetic activation of cranial nerves VII and IX may reduce production of tears and/or saliva, whereas a parasympathetic inhibition may increase the tear and/or saliva production.

Cranial nerve X is the vagus nerve, which also is discussed below with reference to FIG. 11. The vagus nerve can be stimulated on a “global” level, before it branches off to various organs in the body, “regionally” to address two or more branches innervating, for example, the digestive system, or “locally” to address specific organs such as the pancreas, kidney, small intestine, or large intestine. The exact location for the application of the stimulation signal may hence be selected based on the desired effector response to be achieved and the part of the body which is to be addressed. As shown in FIG. 9, branches of cranial nerve X innervate, inter alia, the heart, trachea, bronchi, lung, stomach, liver, pancreas, kidney, small intestine, and large intestine. A parasympathetic activation of these branches typically causes a muscle relaxation or reduced activity inter terms of, for example, glandular secretion. A parasympathetic inhibition, on the other hand, may cause a muscle contraction or increase glandular activity, as this may cause the autonomous tone (i.e., the balance between the PNS and SNS) to shift towards the sympathetic end of the scale. It will therefore be appreciated the stimulation of the PNS may be combined with stimulation of the SNS, as discussed above in connection with FIG. 1A, to achieve a complementary or synergistic effect adjusting the autonomic tone towards a more parasympathetic dominance or a sympathetic dominance.

The sacral nerves originating cord segments S1, S2, S3 are parasympathetic nerves contributing to the sacral plexus. These nerves innervate the lower limb, pelvis, and perineum and may take part in motor and sensory functions of the legs, feet, and pelvic organs. Stimulating these nerves may affect functions such as walking, urination, and sexual activity and may, just as mentioned above, be stimulated in combination with sympathetic complementary nerves originating from cord segments L1, L2, and L3.

In some examples, a method for affecting a balance between a sympathetic tone and a parasympathetic tone of the ANS is provided. According to the method, a first stimulation signal may be delivered by a first signal generating means, such as a first stimulation arrangement or first electrode arrangement 210 to a sympathetic nerve of the patient to increase a level of activity of the sympathetic nerve and move the balance towards a sympathetic dominance. The sympathetic nerve may be any of the nerves discussed above with reference to FIG. 9. Alternatively, a second stimulation signal may be delivered to a parasympathetic nerve to increase a level of activity of the parasympathetic nerve and move the balance towards a parasympathetic dominance. The parasympathetic nerve may be any of the nerves discussed above in connection with FIG. 9. Hence, the sympathetic and/or parasympathetic nerve may form part of at least one of a cardiovascular system, a respiratory system, a gastrointestinal system, a urinary system, an immune system, a sexual function system, and a stress response system. Adjusting the autonomic tone may affect a heart rate or blood pressure (cardiovascular system), a breathing rate or airway diameter (respiratory system), a sensation of satiety or hunger, or affect peristalsis or digestion (gastrointestinal system), a bladder function (urinary system), an inflammatory response (immune system), erection and ejaculation (sexual function system), and psychological health (a stress response system). The adjustment may be achieved by delivering at least one of the first and second stimulation signals to the respective plexuses, such as the sacral plexus, the cardiac plexus, the celiac plexus, the hypogastric plexus, and the pelvic plexus. In further examples, the stimulation may be delivered to the hypothalamus, or the brainstem (such as pons or medulla).

The present concept of nerve stimulation as discussed with reference to FIG. 9 may be combined with the unidirectional stimulation and the sensor feedback as discussed above in connection with FIGS. 3A-F and 5A-D. For brevity, common features and advantages are therefore not reiterated.

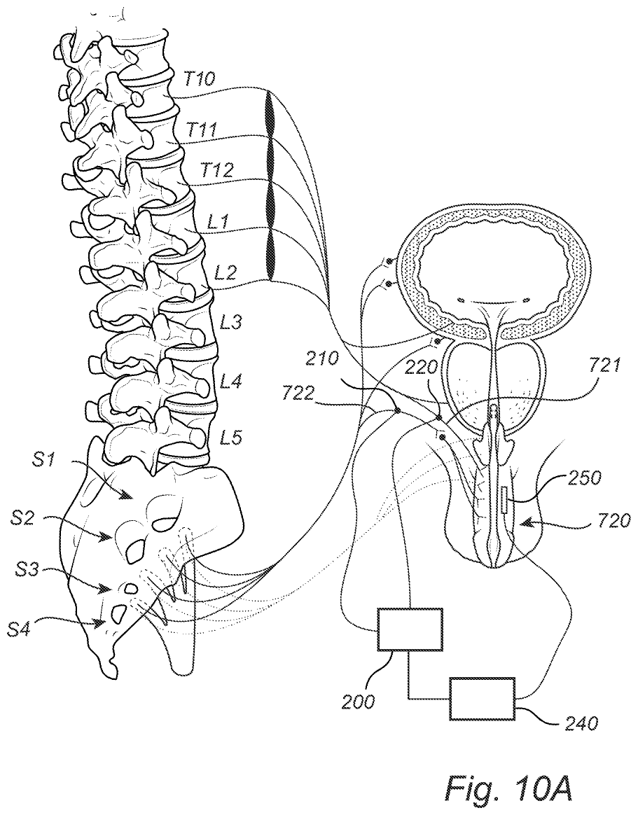

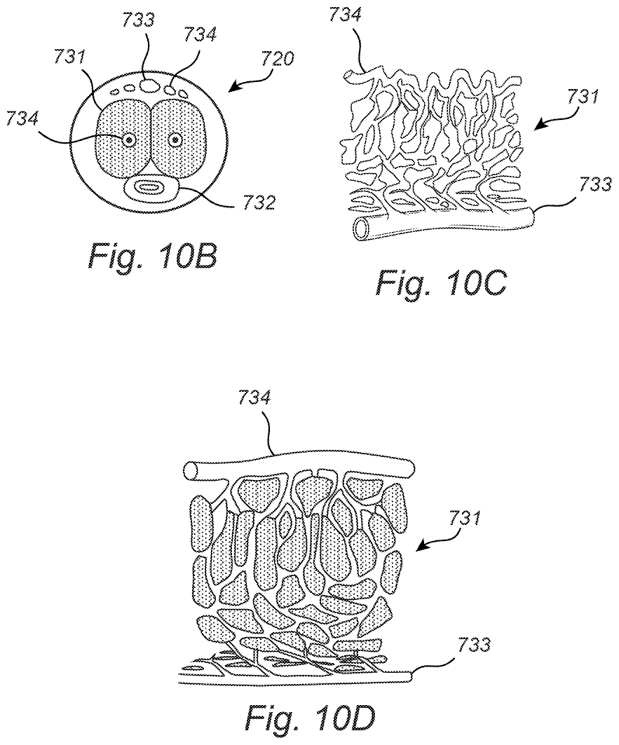

FIG. 10A is a schematic illustration of a system for treating erectile dysfunction. The system may be similar to the system for sympathetic and parasympathetic stimulation presented in FIGS. 1A-E, and may be combined with the technologies for unidirectional stimulation outlined in FIGS. 3A-F and the feedback mechanisms detailed in FIGS. 5A-F. To maintain brevity, common features and advantages are not repeated in detail here.

Erectile dysfunction, often referred to as ‘impotency’, is a common form of sexual dysfunction. The severity of erectile dysfunction can range from mild dysfunction, in which a man is occasionally unable to achieve and sustain an erection sufficient for intercourse, to frequent or moderate dysfunction, to severe or complete dysfunction, in which a man is never able to produce and sustain an erection sufficient for intercourse. The system according to the present disclosure may be employed to address erectile dysfunction over the full range, from mild to complete dysfunction.

In a healthy individual, penile erection is generated by increased blood flow into the penis 720 via arterial dilation and decreased blood flow from the penis 720 via venous occlusion. Arterial dilation may be caused by a parasympathetic activation of the smooth muscle cells of the erectile tissue in the penis 720. The cavernous nerve 722 in an example of such a nerve. The activation may include release of a vasodilator and smooth muscle relaxant, such as nitric oxide, which may cause the dilation of arteries such as the cavernous arteries and the helicine arterioles suppling the erectile tissue with blood. The parasympathetic activation may also cause the relaxation of the trabecular network, which is the smooth muscle tissue that surrounds the blood-filled spaces in the erectile tissue. This may lead to increased blood flow and decreased venous outflow in the penis 720, resulting in erection.

By applying a stimulation signal to a parasympathetic nerve innervating the erectile tissue, a parasympathetic activation may be triggered, which may result in erection. In the example shown in FIG. 10A, the stimulation signal may be applied to a branch of the parasympathetic cavernous nerve 722 by means of a stimulation arrangement 220, stimulation generating means, such as an electrode arrangement or vibrator as previously discussed.

Is has been observed that penile erection also may be generated by a sympathetic activation of nerves that innervate veins controlling the flow of blood leaving the penis. By applying a stimulation signal to such nerves, such as a branch of the hypogastric nerve plexus 721, a vasoconstrictor and smooth muscle contractant such as norepinephrine may be released. This may cause the constriction of the cavernous veins, which typically are the principal veins that drain the blood from the erectile tissue. The sympathetic activation may also cause the contraction of the deep dorsal vein, which typically is the main vein that drains the blood from the glans penis. These actions reduce the venous outflow from the penis, allowing for an increased intracavernous pressure and maintenance of erection.

By applying a stimulation signal to a sympathetic nerve innervating the erectile tissue, a sympathetic activation may be triggered, which may result in erection. In the example shown in FIG. 10A, the stimulation signal may be applied to a sympathetic branch of the hypogastric nerve plexus 721 by means of a stimulation arrangement 210, such as an electrode arrangement or vibrator as previously described.

It will be appreciated that the sympathetic stimulation and the parasympathetic stimulation may be performed individually and separately, or in combination. Combining the two may be particularly beneficial, as the venous vasoconstriction may help increasing the effectiveness of the increased blood supply provided by the arterial vasoconstriction.

Hence, in an example, a system for treating erectile dysfunction is provided. The system comprises a stimulation device 200 configured to deliver, directly or indirectly, a first stimulation signal to a sympathetic nerve innervating a penile vein, and a second stimulation signal to a parasympathetic nerve innervating a penile artery. The system further comprises a control unit 240 configured to control an operation of the stimulation device 200 such that the first stimulation signal stimulates an activity of the sympathetic nerve, thereby causing vasoconstriction of the penile vein, and such that the second stimulation signal stimulates an activity of the parasympathetic nerve, thereby causing vasodilation of the penile artery. By increasing the blood flow entering the erectile body and reducing the blood flow exiting the erectile body, engorgement of the erectile body may be induced.

The stimulation signals may be delivered in various ways. In some examples, a first stimulation arrangement, such as a first electrode arrangement 210, may be configured to be coupled to the sympathetic nerve at a position between a level of the T11-T2 vertebrae and the pelvic plexus. Further, a second stimulation arrangement, such as a second electrode arrangement 220, may be configured to be coupled to the parasympathetic nerve at a position between a level of sacral spinal cord segments S2-S4 and the pelvic plexus.

The control unit 240 may be configured to control the operation of the stimulation device 200 such that at least one of the first stimulation signal and the second stimulation signal is a periodic signal including a variable frequency component, a variable duty cycle component, a variable amplitude component, and a variable pause component. As previously mentioned, action potentials may be induced in the nerve by a relatively low frequency signal, such as in the range of 0.1-100 Hz, depending on the type of tissue.

In some examples, as sensor device 250 may be provided, which may be configured to generate a sensor signal indicating an effector response caused by the application of the stimulation signal(s). The sensor device 250 may be communicatively coupled with the control unit 240 so as to allow the control unit to use the sensor signal as feedback for controlling the operation of the stimulation device 200 and thus the characteristics of the applied stimulation signals. The sensor device 250 may be arranged to generate a signal indicating one or more of a vasoconstriction of the penile vein, a vasodilation of the penile artery, and an engorgement of the erectile body. Similar to what is disclosed in the examples of FIGS. 5B-D, various types of sensor devices may be employed to measure various physical responses. In some examples, the sensor device may comprise a sensor electrode configured to measure an electric activity and/or electrical impedance in smooth muscle tissue of the penile vein and/or artery. In further examples, the sensor signal may be generated by a strain gauge arranged to measure engorgement of the erectile body.

The stimulation signals may be mechanical vibration signals generated by, for example, one or more piezoelectric vibration elements. Alternatively, or additionally, the stimulation signals may be electric signals delivered to the nerves by one or more electrodes. The electrodes may be placed against the nerve, at least partly enclosing the nerve, or inserted into the nerve, as indicated in FIGS. 1B-F.

Further, a blocking signal, or suppression signal, may be employed to prevent action potentials, generated by a stimulation signal, from propagating in a certain direction. For example, the blocking signal may be applied caudal to the stimulation signal to reduce the risk of the stimulation signal propagating backwards towards the central nervous system. This may be referred to as unidirectional stimulation and may be achieved by the technology disclosed with reference to the examples of FIGS. 3A-F.

FIGS. 10B-D illustrate the above mechanisms, in which the penile arteries are dilated to increase blood flow to the erectile tissue and the penile veins are constricted to reduce the flow of blood leaving the erectile tissue.

FIG. 10B is a mid-shaft cross-section of penis, whereas FIGS. 10C and D show portions of the erectile tissue in collapsed and engorged state, respectively. The erectile tissue forms mainly three elongated bodies—two corpora cavernosa 731 and one corpus spongiosum 732. The corpora cavernosa 731 typically run along the length of the penis 751 on the left and right sides, above the corpus spongiosum 732 which runs along the underside of the penis 751, enclosing the urethra and preventing it from punching closed during erection. The blood supply to the erectile tissue 735, such as that of the corpora cavernosum 731, is typically provided by the internal pudendal artery, which branches into the penile arteries 734. Artificial nerve signals generated by the stimulation device 200 trigger the relaxation of smooth muscle cells in the arteries 734 and the erectile tissue 735, leading to the dilation of the arteries 734 and the filling of the erectile tissue 735 with blood. This process is supported by the restriction of venous return by the penile veins 733, such as the tunica albuginea, helping to maintain the erection. The restriction of venous return may be triggered by an artificial stimulation signal delivered by the stimulation device 200. As mentioned above, the arterial dilation may be caused by a parasympathetic activation, whereas the venous constriction may be caused by a sympathetic activation. FIG. 10C shows the erectile tissue 735 in a collapsed state and FIG. 10D shows the erectile tissue when filled with blood.

FIG. 11 is a schematic illustration of a system for controlling appetite in a patient. The system comprises a stimulation device 200, which may be similarly configured as any of the stimulation devices 200 discussed above. For example, the stimulation device 200 may be similarly configured as the stimulation devices disclosed with reference to FIGS. 1A, 3A, and 5A. The stimulation device 200 may be configured to deliver, directly or indirectly, one or more stimulation signals to the vagus nerve. In particular, the stimulation signal may be delivered to the anterior vagal trunk 752 and/or the posterior vagal trunk 753 of the vagus nerve, as shown in FIG. 11. The system may further comprise a control unit 240 configured to control an operation of the stimulation device 200. The control unit 240 may operate the stimulation device 200 to deliver a stimulation signal-either electric, or vibrational-controlling or modifying one or more bodily functions regulated at least in part by the vagus nerve. In particular, the stimulation signal may be delivered to affect the patient's feeling of satiety or hunger. The stimulation signal may be delivered to the vagus nerve by means of a first stimulation arrangement 210, which may be coupled to the anterior gastric division 756 and/or the posterior gastric division 757 of the vagal trunk(s) 752, 753, as indicated in the particular example shown in FIG. 11.

Controlling the patient's appetite has shown to an effective measure in the treatment of obesity. Obesity is known to be a major risk for many diseases, including cardiovascular disease, diabetes, kidney disease, various types of cancers, as well as musculoskeletal disorders. Traditionally, treatment of obesity imposes a heavy economic burden on the global healthcare system. Common approaches for treating obesity include non-surgical and surgical treatments such as gastric bypass and sleeve gastrectomy. However, there is a high risk of serious complications resulting from invasive procedures, as well as potential of weight rebound or side effects from drugs. Therefore, there is a need for alternative and more effective technologies for affecting and controlling the weight of the human body.

Neuromodulation, which typically may be considered a non-destructive and reversible therapeutic strategy, has shown effective in manipulating body functions by stimulating or influencing neurophysiological signals through the neural network to achieve therapeutic purposes. In this context, the vagus nerve (the tenth cranial nerve), may act as a signal bridge to transport information between the brain and various parts of the body. As discussed with reference to FIG. 9, the vagus nerve originates from the medulla oblongata in the brainstem and travels through the neck, chest, and abdomen, innervating various organs along the way. The vagus nerve is a mixed parasympathetic nerve carrying both sensory and motor information and plays an important role in the function of the digestive system.

As shown in the FIG. 11, the vagus nerve typically comprises two main branches innervating the stomach 751: the anterior vagal trunk 752 and the posterior vagal trunk 753. These typically enter the abdomen through the oesophageal hiatus. Hence, in some examples, the stimulation device 200 may be arranged to deliver the stimulation signal at this position, i.e., at or close to the oesophageal hiatus. The stimulation device 200 may, for instance, be combined with other types of medical devices and implants, such as devices arranged to stretch a portion of the stomach wall to trigger mechanoreceptors to induce a feeling of satiety, or devices invaginated in the stomach fundus to hinder the oesophageal sphincter from moving in relation to the oesophageal hiatus and thereby cause gastroesophageal reflux disease (GERD).

In the particular example shown the present figure, however, the stimulation signal may be delivered further down the nerve, after the branch sent to the celiac division 754. More specifically, the first stimulation arrangement 210, such as a first electrode arrangement, may be arranged to contact the gastric division 756, which provides parasympathetic innervation to the smooth muscle and glands of the stomach. Stimulating or inhibiting the gastric division 756 has shown to be effective in affecting gastric motility, secretion, and blood flow, as well as sensory information from the stomach to the brain, such as pain, distension, and acidity levels.

In the illustrated example, the first stimulation arrangement 210 delivers the stimulation signal to the anterior gastric division 756. This division typically runs along the lesser curvature of the stomach and innervates effector tissue of the anterior wall of the stomach 751. The first stimulation arrangement 210 may, however, additionally, or alternatively deliver a stimulation signal to the posterior gastric division 757, which typically runs along the lesser curvature of the stomach 751 and innervates effector tissue of the posterior wall of the stomach 751. It will be appreciated that other branches of the gastric divisions 756, 757 may be stimulated in additional or alternative implementations, such as divisions running along the greater curvature of the stomach 751 and innervate the fundus and body of the stomach 751. The stimulation device 200 may be arranged to deliver the stimulation signal to any of or all these branches, depending on the desired effect to be achieved.

In further examples, the stimulation device 200 may be arranged to deliver the stimulation signal to other branches and divisions of the anterior or posterior vagal trunks 752, 753, including the hepatic division 758, which innervates the liver and gallbladder, and the pyloric branch 759 which is a branch of the hepatic division 758 and comprises visceral sensory fibers originating in the pyloric canal, pyloric sphincter, and first part of the duodenum. The stimulation signal may be delivered to the pyloric branch 759 to reduce gastric acid secretion and impair the gastric motility, which may have an effect on obesity.

The stimulation device 200 may be operated in various modes depending on the desired effect to be achieved, or symptom to be treated. The stimulation device 200 may for example be operated to deliver an inhibition signal for inhibiting activity of the effector tissue innervated by the stimulated nerve. The stimulation device 200 may also be operated to deliver a suppression or blocking signal, preventing action potentials from propagating through the stimulated nerve. Further, the stimulation device 200 may be operated to deliver an activation signal, activating the effector tissue innervated by the stimulated nerve. Various combinations of these operation modes are possible, of which a few will be discussed in the following.

In an example, the control unit 240 is configured to control the operation of the stimulation device 200 such that the stimulation signal suppresses the propagation of action potentials in a cranial direction of the vagus nerve. This allows for a hunger signal, conveyed by sensor fibers of the vagus nerve, to be hindered from reaching the patient's brain. As mentioned above in connection with the unidirectional stimulation, the stimulation signal may be a time-varying signal with an amplitude varying in the range of 1-10 KHz. This type of signal, which also may be referred to as an inhibition signal, suppression signal, or blocking signal, may effectively impair the nerve's ability to transmit action potentials to a degree that reduces the patient's sensation of hunger.

The stimulation signal may typically be applied to nerve fibers that are part of the celiac branches 754 of the vagus nerve. The celiac branches 754 has been observed to detect the stretch and nutrient content of the stomach 751 and intestines, and to send signals to the brain to regulate the appetite or food intake. By blocking the celiac branch 754 of the anterior and/or posterior vagal trunks 752, 753—or at least reduce the signal transmission—the nerve signals indicating hunger may be prevented from reaching the brain- or at least reduced in signal strength—and the patient may be less hungry.

In some examples, the stimulation device 200 may be employed to activate mechanoreceptor cells that detect stomach stretch. Activating these cells may generate a nerve signal indicating that the stomach 751 is full, resulting in a reduced appetite of the patient. Put differently, the stimulation signal may be employed to generate artificial appetite-inhibiting signals to the brain.

There have also been observed a type of nerve fibers of the vagus nerve which affect the regulation of blood glucose levels. These nerve fibers are typically part of the celiac branches 754 of the vagus nerve and takes part in the regulation of the production or hormones that control hunger and satiety, such as ghrelin and leptin. By activating or inhibiting such nerve fibers, insulin secretion and glucose metabolism may be modulated, which in turn may have an effect on blood glucose levels and type 2 diabetes.

As mentioned above, a nerve blocking effect may be achieved by stimulation signals comprising frequencies in the range of 1-10 kHz. Generating artificial nerve signals, such as action potentials travelling towards the brain to induce a sensation of satiety, may on the other hand require a low-frequency signal as discussed in connection with FIG. 1A. Examples of such signals include frequencies in the range of 0.1-100 Hz.

In some examples, the stimulation signal may be applied to stimulate or suppress a stomach peristalsis. This may, for example, be achieved by inhibiting or stimulating an activity of parasympathetic nerve fibers of the vagus nerve that innervate muscle tissue of the stomach wall. By stimulating a parasympathetic response, for example with frequencies in the range of 0.1-100 Hz, movement of the smooth muscle tissue of the stomach wall (and, in some examples, the intestines), can be suppressed. The stimulation signal may be applied to the celiac division 754, as well as other branches that innervate the intestines, such as the anterior and posterior vagal trunks 752, 753, which has been observed to give off several intestinal nerves.

By activating or increasing a parasympathetic response in nerves that help regulate the peristalsis, i.e., the wave-like contraction of the muscles in the stomach 751 and the intestines, the movement of the food along the digestive tract can be slowed down and the stomach be emptied at a slower pace.

A similar effect may be achieved by applying a stimulation signal inhibiting a sympathetic response in the muscle tissue of the stomach wall. In some examples, combinations are employed, such that a parasympathetic activity is stimulated while a sympathetic activity is inhibited.

A reversed stimulation is also conceivable, in which parasympathetic and/or sympathetic responses in the muscles of the stomach 751 and/or intestines are modulated so as to increase the peristalsis and hence the movement of the food along the digestive tract. Increasing the pace with which the food travels may reduce the time for absorption of nutrients and water, thereby affecting nutritional uptake as well as fecal consistency. In some examples, the peristalsis may be varied over the length of the digestive channel, i.e., over different parts of the small/large intestine. For example, the food may be caused to pass through the small intestine at a relatively high pace to reduce the time for nutritional uptake, and at a slower pace by the end of the large intestine to promote absorption of water and improve fecal consistency.

In the particular example illustrated in FIG. 11, the stimulation device 200 further comprises a second stimulation arrangement 220 for applying a suppression signal suppressing action potentials propagating through the nerve in response to the first stimulation arrangement 210 applying the stimulation signal. The suppression signal, which may be an electric stimulation signal or a vibrational signal, may be employed to achieve a unidirectional stimulation, thereby reducing adverse side effects of the application of the signal. More specifically, the suppression signal may be employed to block action potentials travelling in certain directions, or at least reduce their intensity. Examples of adverse side effects include initiation of undesired or counter-productive feedback to the brain and can result in undesired sensations or activity of the patient.

The second stimulation arrangement 220 may be arranged caudal to the first stimulation arrangement 210 to prevent the applied stimulation signal from propagating towards the CNS and the brain. In other examples, the first stimulation arrangement 210 may be arranged caudal to the suppression arrangement 220, as this may prevent the stimulation signal from reaching effector tissue, such as muscle tissue of the stomach wall or sensory receptors. The relative orientation of the first stimulation arrangement 210 and the suppression arrangement 220 may hence vary depending on the specific result to be achieved. Put differently, the location at which the stimulation signal and the suppression signal, respectively, are delivered may be selected depending on where the response to the stimulation signal is to be achieved. The suppression signal may hence be applied at location beyond which the stimulation signal should not pass. In the example shown in FIG. 11, the suppression signal may be delivered to one or more of the vagal branches or divisions, such as the celiac division 754 or the pyloric branch 759 to reduce the risk of inadvertent stimulation thereof as the stimulation signal is delivered to the gastric division 756. The suppression arrangement 220 may thus be used to block any artificial nerve signals from spreading along nerve fibers which are not intended to be stimulated.

The stimulation signal and the suppression signal may be applied at the same time, such as concurrently or simultaneously. In some examples, the suppression signal may be applied with a delay timed to generally match a conduction velocity of the stimulation signal in the vagus nerve. This allows for the suppression signal to be applied for a slightly shorter time compared to a simultaneous application. Reducing the time of the application may reduce the risk for stimulation induced damages to the nervous tissue and fatigue.

In the present example, the stimulation signal and the suppression signal are applied by two separate and physically distinct stimulation arrangements 210, 220, such as a respective cuff electrode. However, it will be appreciated that the suppression or blocking capability may be integrated in the same structural arrangement, such as the same cuff electrode, as the stimulation capability. This may be achieved by integrating the means delivering the stimulation signal into the same housing or structural unit as the means delivering the suppression signal.

In some examples, the system may comprise a sensor device configured to generate a sensor signal indicating an effector response in the tissue innervated by the nerve to which the stimulation signal is applied. The sensor device may be similarly configured as the devices discussed above in connection with FIGS. 5A-D and similar features and effects are therefore not reiterated here.

In some examples, the sensor signal may be indicative of action potentials propagating in the nerve. This information, i.e., the effector response and/or the action potentials, may be used as feedback for controlling the operation of the stimulation device 200. The feedback may, for example, be used to adjust or modify parameters of the stimulation signal. The intensity of the stimulation signal may be increased in response to the sensor signal indicating a too low level of response in the effector tissue or reduced in response to the sensor signal indicating a too high level or response in the effector tissue. Furthermore, the intensity of the suppression signal may be increased in case the sensor response indicates that action potentials are still propagating past the suppression arrangement 220, or reduced in case the sensor response indicate that no action potentials travel past the suppression arrangement 220. Various parameters of the stimulation/suppression signal may be varied depending on the type of result intended to be achieved. The control unit 240 may, for example, use the sensor signal to adjust a frequency of the signal, an amplitude of the signal, or, in case of an electric signal, an electric current of the signal.

The sensor signal may be analyzed to retrieve a response measure, which may be indicative of the effector response or a strength of action potentials propagating through the nerve. The response measure may be compared with a reference measure to determine how to adjust one or more parameters of the signal. The reference measure may, for example, be based on one or more previous measurements of the response in the patient, or on previous measurements of responses in other patients. Further, the effector response may be monitored over time so as to detect any drifts or changes over time that may need to be taken into account when controlling the operation of the stimulation device 200.
the examples, features and advantages of the system for sympathetic and parasympathetic stimulation presented in FIGS. 1A-E, the unidirectional stimulation outlined in FIGS. 3A-F, and the sensors and feedback mechanisms detailed in FIGS. 5A-F are applicable to the present vagal stimulation system. To maintain brevity, common features and advantages are not repeated here.

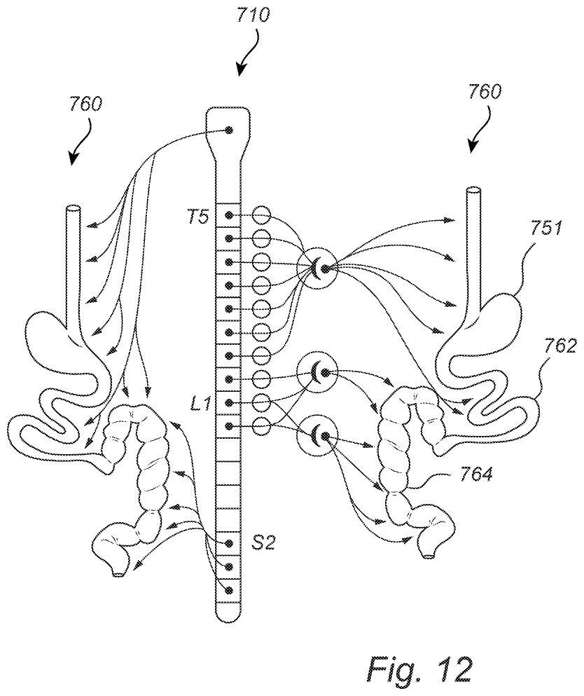

FIG. 12 is a schematic illustration of the alimentary canal 760, or digestive tract, of a typical human body. The walls of the alimentary canal, such as the stomach 751, the small intestine 762 and the large intestine 764, include layers of smooth muscle tissue controlled by the ANS. Alternating contraction and relaxation of these muscles is called peristalsis. Peristaltic waves push the swallowed bolus down the esophagus. In a healthy individual, the swallowed food may typically be treated in the following, exemplary way as described in the following.

In the stomach 751, peristalsis churns swallowed food, mixing it with gastric juices, these mechanical and chemical actions further break down food into a substance called chyme. This process, in which foodstuff is turned into chyme, typically takes a few hours.

A major part of the nutrient absorption typically occurs in the small intestine 762. When chyme passes from the stomach 751 into the small intestine 762, peristaltic waves shift it back and forth and mix it with digestive enzymes and fluids. The chyme is often pushed for about 3 to 6 hours in the small intestine 762 before it is passed to the large intestine 764 where any final absorption may take place. Peristaltic waves help compact and move waste and indigestible foodstuffs through the large intestine 764 for elimination.

The digestive tract 760 is typically endowed with its own, local nervous system, referred to as the enteric nervous system (ENS). An example of the innervation of the digestive tract 760 is shown in FIG. 12, with the parasympathetic nerves to the left in the figure (including the vagus nerve and the pelvic nerve) and the sympathetic nerves to the right. The ENS has been found to be capable of acting independently of the sympathetic and parasympathetic nervous systems, although it may be influenced by them. The neurons of the ENS typically control the motor functions of the system (i.e., the peristalsis), in addition to the secretion of gastrointestinal enzymes. These neurons communicate through various neurotransmitters similar to the CNS.

It has been found that by stimulating the ENS locally, either through application of a stimulation signal to one or more nerves innervating the digestive tract, or through application of a stimulation signal directly to the walls of the stomach 751, the small intestine 762, and/or the large intestine 764, the peristalsis may be controlled or at least affected. The stimulation signal, which may be provided by any of the systems discussed above, such as in connection with FIG. 1A, 3A, 5A or 11. In some examples, one or more stimulation signals are applied to a parasympathetic nerve (such as the vagus nerve) and/or a sympathetic nerve (e.g., via the prevertebral ganglia). By activating or inhibiting a nervous response in the ENS, the coordination of reflexes controlling the peristalsis and segmentation may be affected of modulated.

In an example, a system for affecting a flow of intestinal content in a gastrointestinal tract 760 of a patient is provided. The system comprises a stimulation device 200 configured to deliver a stimulation signal to a plurality of stimulation sites along the gastrointestinal tract 760 to induce an effector response in smooth muscles of a wall of the gastrointestinal tract. The stimulation device may be energized by a source of energy, which may be implanted in the patient or arranged external to the patient, or both. Further, a control unit 240 is provided, which is operably connected to the stimulation device 200 and configured to control an operation of the stimulation device 200 such that the stimulation signal causes at least one of promoting and suppressing peristalsis or segmentation of the gastrointestinal tract. Furthermore, the plurality of stimulation sites may be spaced apart along the gastrointestinal tract 760 by a predetermined spacing.

The stimulation signal may be applied in various modes, and to various types of tissue, to depending on the desired effect. In general, the stimulation device 200, delivering the stimulation, may be operated in an activation mode to trigger contraction or relaxation of the smooth muscle tissue, and an inhibition mode inhibiting contraction or relaxation. As discussed above, contraction may typically be triggered by stimulation signals having a relatively low frequency, such as less than 1 Hz, or a few Hz. Examples include 0.1-1 Hz, 1-10 Hz, and 10-100 Hz. Such a stimulation signal may be applied to trigger contraction of the smooth muscles of the walls of the gastrointestinal tract 760. The underlying mechanism is believed to involve parasympathetic activation, which stimulates the ENS to release acetylcholine, a neurotransmitter that binds to muscarinic receptors on the smooth muscle cells and causes them to contract. Additionally, or alternatively, the stimulation signal may be applied to trigger relaxation of the smooth muscles of the walls of the gastrointestinal tract 760. The underlying mechanism is believed to involve sympathetic activation, which stimulates the ENS to release norepinephrine, a neurotransmitter that binds to alpha and beta receptors on the smooth muscle cells and causes them to relax. A stimulation signal may hence be used both for contraction and relaxation, depending on which type of nervous response is triggered. A parasympathetic activation may result in a contraction, while a sympathetic activation may result in a relaxation. By alternating between these types of activations, and/or combining the two, peristalsis and segmentation may be affected.

Inhibition or suppression may be applied in a similar manner. Thus, by applying a relatively high frequency signal, such as in a range of 1-10 kHz, the smooth muscle tissue may be inhibited, i.e., contraction or relaxation of the muscle cells may be suppressed. By applying such a stimulation signal to a parasympathetic nerve, contraction of the muscle cells may be suppressed. By applying such a stimulation signal to a sympathetic nerve, relaxation of the muscle cells may be suppressed.

Various combinations of inhibition and activation may be applied to achieve the desired effect. In some examples, muscle cells may be activated to contract at the same time as relaxation may be inhibited. This may be alternated with activation of relaxation combined with inhibition of contraction. In other examples, activation signals may be employed in a sequence, in which triggering of contraction cycled with triggering of relaxation.

The stimulation signals may be applied at a plurality of locations along the alimentary canal 760. The stimulation signals may be delivered by stimulation arrangements, such as the stimulation arrangements 210, 220 discussed above in connection with FIGS. 1A, 3A, and 5A. The stimulation arrangements 210, 220 may comprise vibration generating means, such as piezoelectric elements, or one or more electrodes for delivering an electric signal, such as a pulsed electric signal. In the following, some examples of application of electric stimulation signals will be discussed. It should however be noted that the discussion applies equally well to other types of stimulation signals, such as vibrations.

A plurality of stimulation arrangements, such as electrodes, can be implanted along the alimentary canal 760 to trigger or inhibit a response in the smooth muscles of the walls of the canal 760. Various spacings between the location in which the stimulation signals are delivered are conceivable. The spacing may be selected based on the amount of tissue that can be affected by the applied signal, i.e., the area affected by the stimulation signal. As the response in the muscle tissue may reduce with increasing distance from the application point of the signal, it may be beneficial to place another stimulation arrangement at a position in which the muscular response has decreased below a certain limit. By applying the stimulation signal at regular intervals along at least a part of the alimentary canal 760, the muscular response to the stimulation may be distributed relatively evenly along the canal 760.

The spacing between the application points of the signal may also be selected based on a natural wave pattern of the peristalsis. The spacing may for example be selected to correspond with a wavelength of the natural wave pattern caused by the contraction and relaxation of the smooth muscle tissue during segmentation or peristalsis. Beneficially, this may allow the stimulation device 200 to be operated in a way that strengthens or amplifies the natural movement of the walls of the alimentary canal 760, or counteracts the same, depending on the timing or phase of the applied signals. A typical wavelength of the peristalsis, i.e., the distance between two consecutive peaks or throughs of the wave-like muscle contractions that move food along the digestive tract 760, varies with the location and function of the organ involved. For example, in the esophagus, the wavelength is typically about 15 cm and the wave speed about 3 cm/s. In the small intestine 762, the wavelength is about 10 cm and the wave speed about 0.5 cm/s. In the large intestine 764, the wavelength is typically about 20 cm and the wave speed is about 0.2 cm/s. Exemplary spacings of the applications points for the stimulation signal may hence be about 10 cm for the small intestine 762, such as 2-18 cm, such as 6-14 cm, such as 8-12 cm, or multiples thereof. For the large intestine 764, exemplary spacings between the points in which the stimulation signal is applied may include about 20 cm, such as 10-30 cm, such as 14-26 cm, such as 18-22 cm, or multiples thereof.

The control unit 240 may be configured to control the application of the stimulation signals based on a natural frequency of the wave pattern of the peristalsis or the segmentation. The stimulation signal may hence be delivered in a pulsed manner, in which a stimulation arrangement is switched between an OFF state and an ON state. In the OFF state, no stimulation signal (or a stimulation signal with a very low, negligible intensity) is delivered to the tissue. In the ON state, the stimulation signal is delivered to induce a response in the tissue. The pacing of the ON and OFF states may be determined based on a frequency of the wave pattern in the smooth muscle tissue. This wave pattern may be the result of a natural movement of the smooth muscle tissue, or a movement induced or controlled by the stimulation device 200. In an example, the stimulation signal may be applied to activate contraction of the muscle tissue when the muscle tissue at application point is about to contract anyway, according to the wave pattern in which the tissue moves. Similarly, the stimulation signal may be applied to cause the muscle tissue to relax at the application point when it is about to contract anyway, according to the wave pattern formed by the contracting and relaxing tissue. In this way, the stimulation signal may be employed to either amplify the movement of the muscle tissue or counteract the movement.

With the wavelength and speed examples above, the stimulation signal may be pulsed with a pulse separation or pulse train separation of about 20 s for the small intestine 762, such as 10-30 s, such as 14-26 s, such as 18-22 s. For the large intestine 764, the stimulation signal may be pulsed with pulse separation or pulse train separation of about 100 s, such as 40-160 s, such as 60-140 s, such as 80-120 s. The pulse length, or pulse duration, may be varied based on the strength or intensity of the stimulation signal. Typically, the pulse duration may be in the order of milliseconds, such as the range of 0.01-100 milliseconds, or 100-1000 ms, or in the order of seconds, such as a few seconds.

The system allows for the peristalsis to be affected or modulated, which in turn can be used to increase or slow down the pace with which the food moved along the digestive tract. This may be beneficial in various applications and for the treatment of various conditions. Controlling the movement of the food through the digestive tract may, for example, be of interest in connection with stoma, as this allows for a more controlled passage of fecal matter to the stoma. It may also have an effect on obesity, as discussed above, as well as on other types of diseases such as irritable bowel syndrome (IBS).

Vasodilation of a blood vessel, or dilation of the blood flow passageway of the blood vessel, is to be understood as an operation increasing a cross-sectional area of the inside space of the vessel. The renal artery is an example of a blood vessel, or luminary organ which can be filled with, and/or convey a flow of, a bodily fluid such as blood. The systems and stimulation devices in the present disclosure may be employed to affect the blood pressure and treat hypotension. In particular, the stimulation of the sympathetic and parasympathetic nervous system discussed above in connection with FIGS. 1A-E, the unidirectional stimulation discussed in connection with FIGS. 3A-F and the feedback mechanisms discussed in connection with FIGS. 5A-D may be employed as outlined below.

In the context of the present application, the term “renal artery” may be understood as any blood vessel providing a (main) supply of blood to a kidney. In case of a transplanted or artificial kidney, which often is placed in a location different from the original kidney, such as the iliac fossa, the renal artery may be connected to the external iliac artery. The present inventive concept may thus be applied also to such a blood vessel.

Blood pressure is generally referred to as the pressure of circulating blood against the walls of blood vessels. Most of this pressure results from the heart pumping blood through the circulatory system. In common language, the term ‘blood pressure’ often refers to the pressure in the larger arteries. Blood pressure is usually expressed in terms of the systolic pressure (maximum pressure during one heartbeat) over diastolic pressure (minimum pressure between two heartbeats). Blood pressure can be understood as being influenced by cardiac output, systemic vascular resistance and arterial stiffness and may vary depending on situation, emotional state, activity, and relative health/disease states.

Blood pressure that is too low is called hypotension, pressure that is consistently too high is called hypertension, and normal pressure is called normotension. Long-term hypertension is a risk factor for many diseases, including stroke, heart disease and kidney failure. The Task force for the management of arterial hypertension of the European Society of Cardiology (ESC) and the European Society of Hypertension (ESH) has provided the following definitions of hypertension:

| Category | Systolic BP, mmHg | Diastolic BP, mmHg |
|---|---|---|
| Optimal | <120 | <80 |
| Normal | 120-129 | 80-84 |
| High normal | 130-139 | 85-89 |
| Grade 1 hypertension | 140-159 | 90-99 |
| Grade 2 hypertension | 160-179 | 100-109 |
| Grade 3 hypertension | ≥180 | ≥110 |

The risk of cardiovascular disease is considered to increase progressively above 115/75 mmHg. Below this level there is limited evidence.

Vascular resistance is the resistance that must be overcome to push blood through the circulatory system and create flow. The resistance offered by the systemic circulation is known as the systemic vascular resistance (SVR). Vasoconstriction (i.e., decrease in inner blood vessel diameter) increases the SVR, whereas vasodilation (increase in inner diameter) decreases the SVR.

Many mechanisms have been proposed to account for the rise in SVR in hypertension. Most evidence implicates either disturbances in the kidneys' salt and water handling (particularly abnormalities in the intrarenal renin-angiotensin system, RAS) or abnormalities of the sympathetic nervous system. The mechanisms are not mutually exclusive, and it is likely that both contribute to some extent in hypertension. Excessive sodium or insufficient potassium in the diet may lead to excessive intracellular sodium, which may contract vascular smooth muscle tissue, restricting blood flow and so increases the blood pressure.

The renin-angiotensin system, RAS, is a hormone system that has been found to regulate blood pressure as well as systemic vascular resistance. When renal blood flow is reduced, which may be the case in for instance hemorrhage or dehydration, juxtaglomerular cells in the kidneys convert the precursor prorenin (already present in the blood) into renin and secrete it directly into circulation. This starts a chain reaction that eventually results in the release of angiotensin II, which has shown to be a potent vasoconstrictive peptide that may cause blood vessels to narrow and the blood pressure to increase accordingly. Angiotensin II is also known to be involved in an increase of extracellular fluid in the body, which also increases blood pressure.

The present invention is based on the realization that by causing an electrically induced vasodilation of the renal artery, a reaction that causes a reduction of the systemic vascular resistance may be triggered. The electrically induced vasodilation of the renal artery may be achieved by means of a stimulation device, which may be arranged to stimulate a nerve innervating the renal artery and/or to provide a direct or indirect stimulation of the smooth muscle tissue of the renal artery.

The stimulation device may be adapted to alter the vasomotor tone of the smooth muscle cells of the renal artery, causing the cells to relax. Sympathetic stimulation (norepinephrine) has been observed to constrict some blood vessels and dilate others, depending on whether the target cells (i.e., the smooth muscle cells) has alpha- or beta-adrenergic receptors. The sympathetic nervous system can also constrict or dilate vessels just by changing firing frequency. An increased firing frequency may cause the smooth muscle to contract and constrict the vessel, whereas a reduced firing frequency may cause the smooth muscle cells to relax, allowing blood pressure to dilate the vessel.

The inventor has realized that the electric stimulation device may be employed to affect the vasomotor tone of the smooth muscle cells to cause the lumen to relax, with the aim of triggering a reduction of the systemic vascular resistance. The electric stimulation device may thus form part of a system for treating a patient with hypertension.

While the focus of the present application may be laid on inducing vasodilation to trigger a bodily reaction to reduce the systemic blood pressure, it will be appreciated that the inventive concept of utilizing electrical stimulation for affecting the vasomotor tone of the renal artery may as well be employed for triggering a response increasing the systemic blood pressure. The present inventive concept may hence be applied also for treating patient suffering from hypotension. The aspects, embodiments and examples herein may be combined with implementations wherein electrically induced vasoconstriction is generated by electrical stimulation. The vasoconstriction may be achieved by controlling the electrical stimulation signal such that a contraction of the renal artery is achieved.

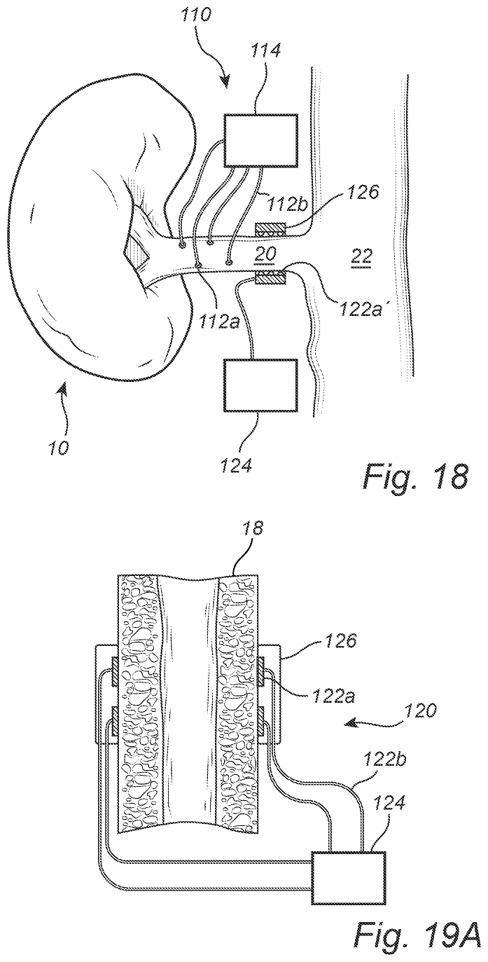

The inventor has further realized that a control, or regulation, of the electrically induced vasodilation may be achieved by providing a sensor, or sensor device, capable of generating a signal indicative of a blood pressure of the patient. The output signal from the sensor may then be supplied to a control unit, which is configured to control an operation of the stimulation device based on the signal generated by the sensor. The control unit may in some examples utilize the signal from the sensor as a trigger signal, indicating that the stimulation may be initiated and/or ceased. In further examples, the control unit may utilize the signal from the sensor as a feedback control signal, preferably driving the system (and hence the vasodilation or even systemic blood pressure) to a desired state (such as normotension. Exemplary embodiments, effects and advantages of using such an optional sensor is described in further detail in connection with FIGS. 18 and 21A-D.

Furthermore, the electrical stimulation signal used for causing the renal artery to relax may inadvertently progress towards the aorta and/or the spinal cord, thereby risking causing unwanted side effects and unpleasant experiences for the patient. Therefore, a signal damping device may according to some implementations of the inventive concept be provided to mitigate the effects of the electrical stimulation signal by damping, disturbing or at least partly cancelling the electrical stimulation signal, thereby limiting the spreading of the electrical stimulation signal to other parts of the patient's body, as previously discussed.

As an introduction to the field in which the present inventive concept can be applied, an exemplary description of the neurophysiology of the renal artery will be described in the following. It is to be noted that the following description of the neurophysiologic mechanisms affecting vasoconstriction of the renal artery is exemplary, simplified where needed, and based on the present knowledge in the art. The purpose of the following exemplary description of the bodily functions and responses is primarily not to limit or define the inventive concept, but to give an exemplary technical/physiological background and context of the inventive concept.

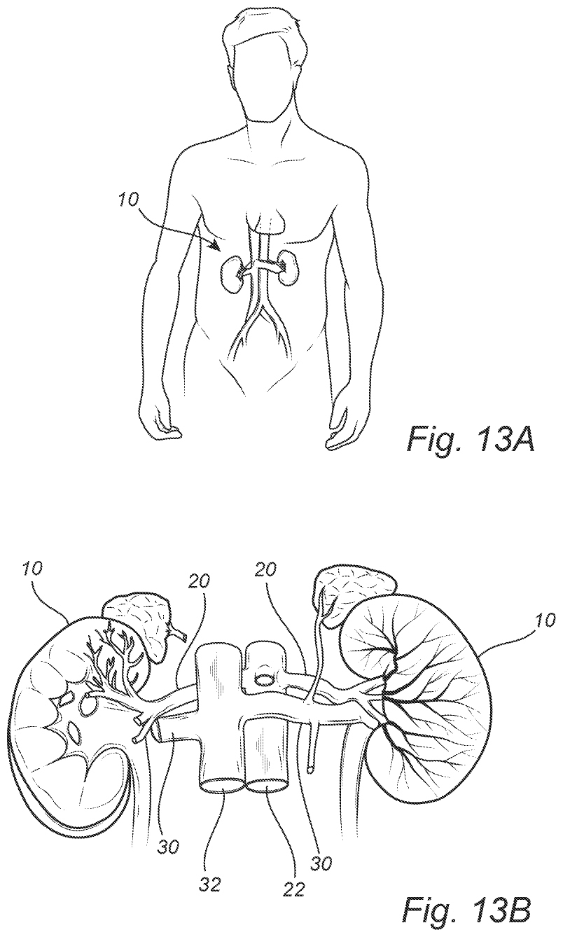

FIGS. 13a and b are schematic illustrations of the kidneys of an adult, human patient. It is common for a normal human to have two kidneys 10, each of which being connected to the circulatory system by means of a renal artery 20 that carries blood from the heart to the kidneys 10 via the aorta 22 and renal vein 30 that drains the kidney 10 and connects it to the inferior vena cava 32.

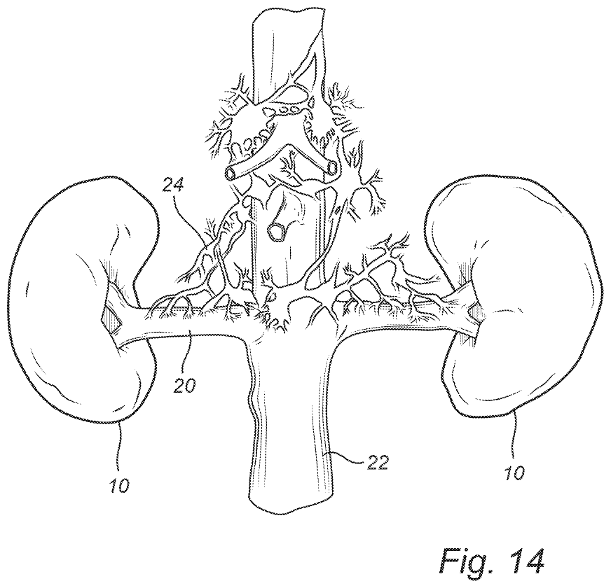

FIG. 14 shows the kidneys 10 and the main renal arteries (MRA) 20, which are identified as the renal main blood supply arteries arising from the aorta 22 and ending at its bifurcation split. Although the illustrations in the present application show a single renal artery 20 connecting a respective kidney 10, the inventive concept is equally applicable to patients wherein a kidney is supplied by multiple renal arteries, which may have a separate origin in the aorta 22. In case of multiple renal arteries, the electrical stimulation may be delivered to at least one of the renal arteries, such as the vessel with the greatest diameter (this may consequently be referred to as the MRA).

Renal nerves 24 may be identified as fiber structures originating from ganglia in the solar plexus or from the splanchnic nerve collection, forming the renal plexus. The renal nerve plexus may thus be understood as the network of nerve fibers 24 innervating the renal artery 20 as well as the kidney 10. It appears as a major part of the nerves are sympathetic nerves, but the renal plexus may according to some findings also comprise parasympathetic nerves. Beneficially, the stimulation device may be arranged to deliver the electric stimulation to a parasympathetic nerve at least in a branch of a spinal cord dispatching number 10 and along the Coccygeal nerves originating at vertebrae S2-S4, preferably S4.

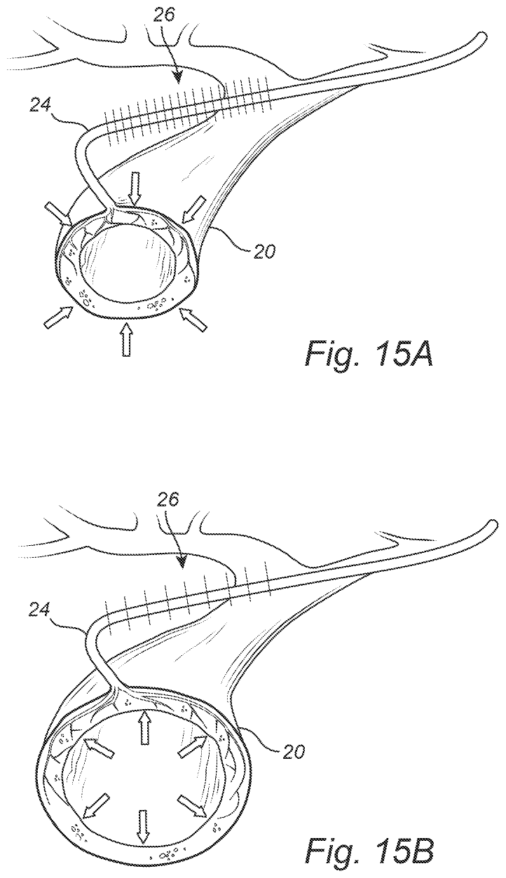

FIGS. 15a and b illustrate the concept of vasoconstriction and vasodilation. The open cross section of the lumen formed by the blood vessel, such as the renal artery 20 showed in FIGS. 3a and b, may be determined by the vasomotor tone of the smooth muscle cells. The smooth muscle cells of the wall of the renal artery 20 may be innervated by nerve fibers 24, such as for instance sympathetic nerve fibers 24. Sympathetic stimulation has been observed to constrict some blood vessels and dilate others, depending on whether the smooth muscle cells have alpha- or beta-adrenergic receptors. As mentioned above, the sympathetic nervous system can also constrict or dilate vessels just by changing frequency of the action potentials of the nerve fibers 24. In the present figures, an example is illustrated in which an increased action potential frequency (indicated by pulses 26) may cause the smooth muscle tissue to contract, leading to vasoconstriction as illustrated in FIG. 15a. Reducing the action potential frequency 26 may cause the smooth muscle tissue to relax, leading to vasodilation as illustrated in FIG. 15b. The stimulation device according to the present inventive concept may be employed to modify the action potential frequency to cause a relaxation of the smooth muscle tissue. Put differently, the stimulation device may be operated to change the vasomotor tone of the smooth muscle tissue of the vessel. The electrical stimulation may be delivered directly to the outer wall of the renal artery 20, or to the nerve fibers 24 innervating the wall of the renal artery 20.

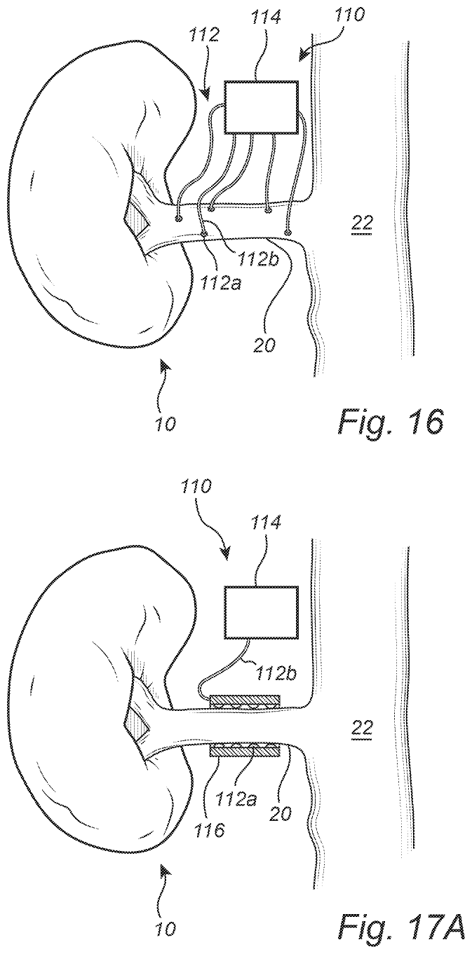

FIG. 16 shows a renal artery 20 connecting a kidney 10 to the aorta 22, and which may be similar to the renal arteries 20 disclosed in FIGS. 13a-15b. In order to treat hypertension, a stimulation device 110 may be implanted in the patient. The stimulation device 110 may comprise an electrode arrangement, such as a first electrode arrangement 112, configured to deliver an electric stimulation signal to tissue of the patient, thereby causing tissue of a wall portion of the renal artery 20 to relax and dilate a blood flow passageway of the renal artery 20. In the present example, the first electrode arrangement 112 is configured to be attached to the outer wall of the renal artery 20 to deliver an electric stimulation signal to the smooth muscle tissue of the renal artery wall 22. In this way, the smooth muscle tissue may be subject to an electrical stimulation that causes vasodilation.

The first electrode arrangement 112 may for example comprise a plurality of electrical electrodes 112a, 112b, each of which having a contacting portion, or electrode element 112a, configured to be arranged to engage the wall of the renal artery 20, and a lead portion 112b electrically connecting the contacting portion 112a to a control unit 114 of the stimulation device 110. The contacting portion 112a of the first electrode arrangement 112 may for example be attached to the wall of the renal artery 20 by means of stitches, for instance allowing for the contacting portion 112a to be at least partly inserted into the tissue on the outer surface of the wall. In further examples, the contacting portion 112a may be arranged on a surface portion, such as a patch (not shown), which in turn may be placed on the tissue of the wall of the renal artery 20.

The control unit 114 may be configured to be electrically connected to the electrode arrangement 112 to provide the contacting portions 112a with the electric stimulation signal. The control unit 114 may thus in turn be operatively connected to, or comprise, a power source energizing the control unit 144 and the electrode arrangement 112. Further, the device may according to some embodiments comprise an additional control unit, also referred to as a central control unit, which may be implanted in the body or be a remote unit, arranged outside the body. Further, the control unit 114 may in some examples be configured to transmit control instructions wirelessly to the stimulation device.

The number of contact points, in which the electric stimulation signal can be delivered to the smooth muscle tissue, may be selected based on the desired response and the characteristics of the stimulation signal used. Increasing the number of contact points may for example allow for a lower signal amplitude required to generate the desired response (i.e., a relaxation) of the muscle tissue. Conversely, an increase signal amplitude may be used for allowing a reduce in number of contact points. Further, it will be appreciated that some or all of the contacting portions 112a may be individually controlled with respect to the stimulation signal, such that the stimulation signal can be selectively and controllable delivered to one or several of the contact points at the time. The selective application at different contact points may for example be enabled by the control unit 114.

When a reduction in systemic vascular resistance is desired, the stimulation device 110 may be operated to generate an electrical stimulation signal that is transmitted from the control unit 114 through the leads 112b to the contacting portions, or electrode elements 112b, which deliver the electrical stimulation signal to the muscle tissue of the wall of the renal artery 20. The electrical stimulation signal may be configured, with respect to e.g. voltage, current or frequency, to trigger a vasodilation response in the renal artery. The vasodilation may in turn result in a systemic response as described above.

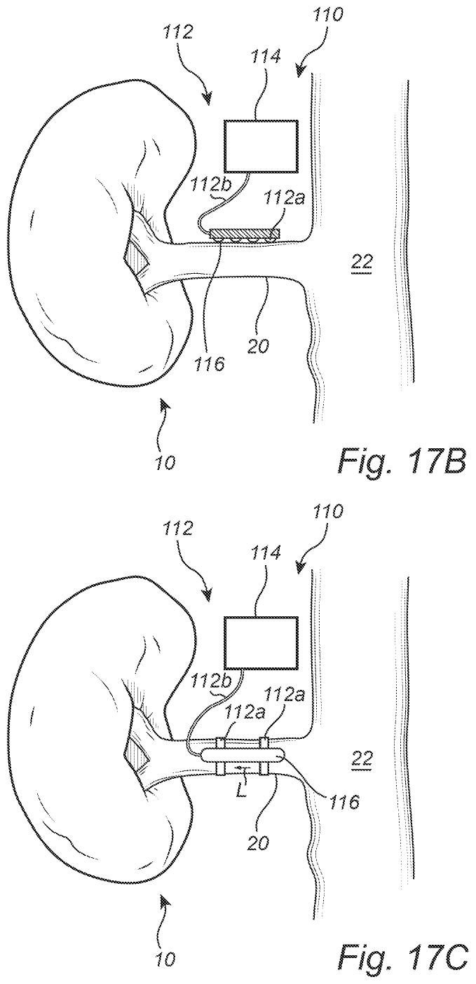

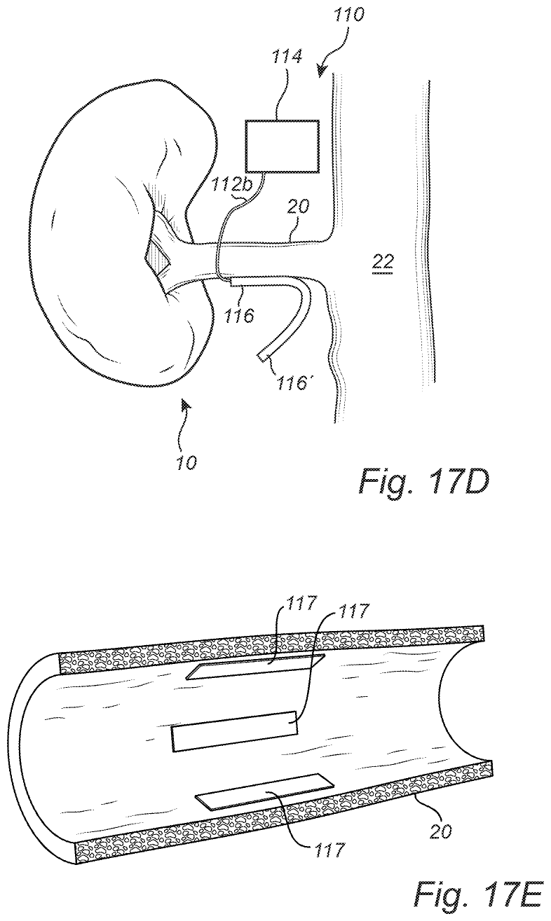

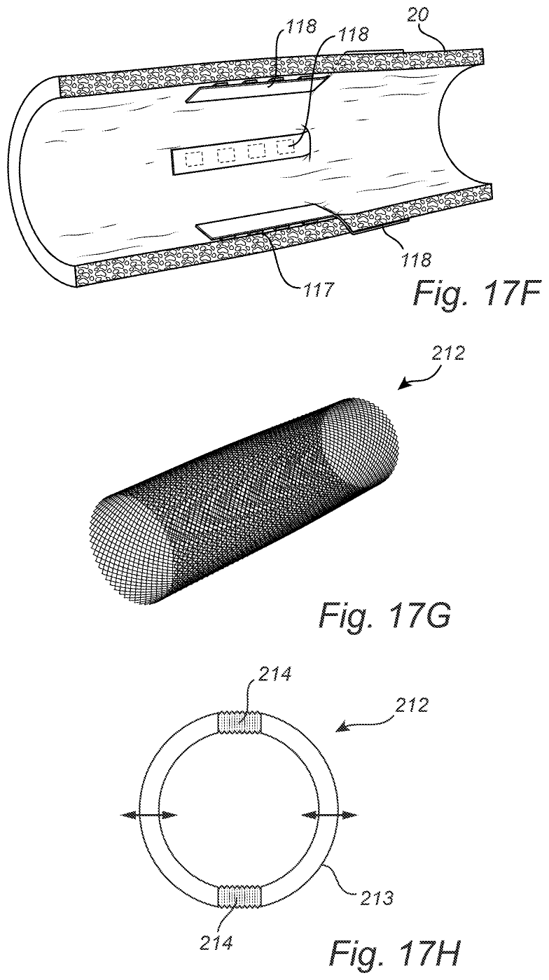

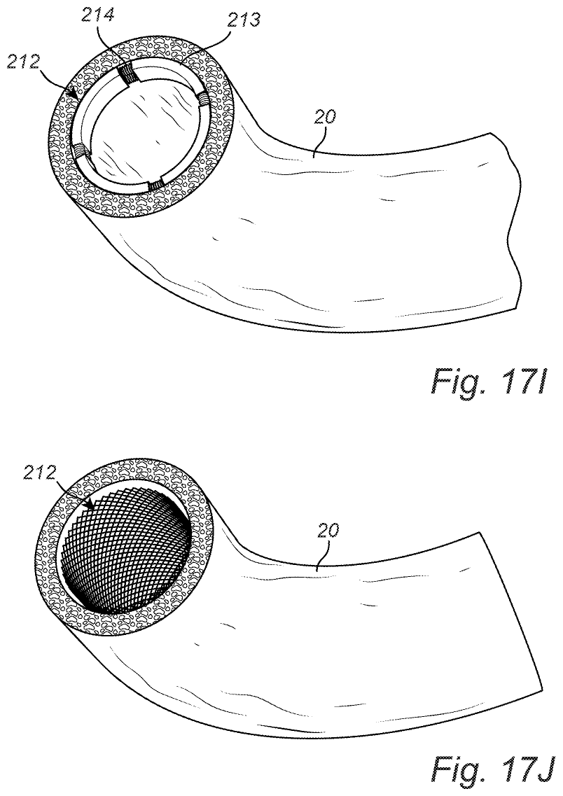

FIGS. 17a-h show a renal artery 20 which may be similar to the renal arteries disclosed in the previous figures. FIG. 17a-d further disclose a stimulation device which may be similarly configured as the one disclosed in connection with FIG. 16, and may thus comprise an electrode arrangement 112a, 112b configured to deliver an electric stimulation signal for affecting vasomotor tone in the renal artery 20. The stimulation device may comprise a plurality of contacting portions 112a, or electrode elements 112a, configured to mechanically engage, or be arranged to rest against, tissue of an outer wall of a portion of the renal artery 20 to transmit the electrical stimulation signal to the tissue. In the example in FIG. 17a, the electrode elements 112a are arranged on an inner surface of a cuff portion 116 configured to be arranged at least partly around the renal artery 20. The cuff portion 116 may in turn be electrically connected to the control unit 114 of the stimulation device 110 by means of a lead 112b. Further configurations are disclosed in FIGS. 17b-d, in which the electrode elements 112a are supported by an elongated holder 116 arranged to keep the electrode elements 112a in the desired position at the wall of the renal artery 20. The holder 116, also referred to as a holding device 116, is formed as an elongated device configured to be attached on the outer wall of the renal artery such that a length direction L of the holder 116 extends along a flow direction of the artery 20. Further, an attachment device may be provided to assist in fixating the holder 116 to the renal artery 20. The attachment device may for instance be formed of at least a part of the electrode element 112a, as shown in FIG. 17c, which may be arranged to at least partly encircle the renal artery 20 and thereby act as a clamp for fixating the holder 116 to the artery 20. Alternatively, or additionally, the attachment device may comprise a suture (not shown) configured to be sutured to the artery to assist in fixating the holder 116. In further examples, such as the configuration shown in FIG. 17d, the attachment device is configured to be attached to a tissue portion external to the renal artery 20. This may be realized by a supporting rod 116′ or lever adapted to extend from the holder 116 and to be attached to tissue surrounding the renal artery 20 or the kidney 10 by means of, for instance, sutures or staples. Beneficially, the supporting rod 116′ may eventually be embedded or encapsulated by fibrotic tissue assisting keeping the holder 116 and the electrode arrangement 112a in the correct position.

It will be appreciated that the holder 116 may be flexible to allow some movement of the stimulation device 110 when implanted. The movement may for instance be caused by the patient moving, or by vasodilation of the artery 20. Further, at least one of a source of energy and control unit of the system may be accommodated in the holder 116.

In the above, vasodilation induced by electrical stimulation of nerves have been discussed. Alternative or additional mechanisms for causing the renal artery to expand or contract are however possible, and can beneficially be combined with the inventive concept disclosed in the present application. Two examples of such mechanisms will now be discussed with reference to FIGS. 17e-i, namely thermally induced vasodilation and mechanically induced vasodilation.

FIG. 17e shows a portion of the renal artery 20 in FIGS. 17a-d, in which a stimulation device 110 having a plurality of heating members 117 have been implanted. In this embodiment, the control unit 114, 124 is configured to control an operation of the stimulation device such that heat is exchanged between the heating members 117 and the wall portion of the renal artery 20 to cause vasodilation thereof. The heat energy may be provided from a source of energy that is implanted inside the renal artery 20, for example integrated in the heating member, or transferred from outside the renal artery 20. In the latter case, the energy may be transferred by means of a wired connection or wirelessly, such as inductively.

While the present figure shows heating members 117 shaped as electrodes attached to the interior of the artery, it will be appreciated that they may as well have a tubular shape with an outer surface configured or rest against the inner surface of the artery, or be attached to such a tubular structure to facilitate insertion and possibly attachment in the vessel. An example of such a configuration is disclosed in FIG. 17f, in which a first and a second catheter 118 are inserted into the artery 20 through the arterial wall and arranged such that the heating members 117 are in thermal contact with the interior side of the artery 20.

In yet a further example, the heating member 117 may define a passage through which a blood flow of the renal artery 20 is allowed to pass. The heating member 117 may thus have a shape conforming to a stent abutting the inner surface of the artery. Beneficially, the heating member 117 may comprise a flexible or expandable portion configured to allow the heating member 117 to follow the change in width of the artery 20 such that a width of the passage increases with increased vasodilation and decreases with decreasing vasodilation. The heating member 117 may comprise a shape memory material configured to vary the width of the passage in response to a varying temperature of the heating member. Further, the heating member may comprise a biocompatible material configured to promote fibrotic tissue to promote fibrotic tissue growth thereon-especially on portions arranged outside the artery, such as the external portion of the catheter 118 shown in FIG. 17f. Preferably, the heating member may be configured to be secured to an inner surface of the artery, where it may be at least partly encapsulated by fibrotic tissue when implanted. Alternatively, or additionally the heating member may be secured to the inner surface by means of sutures or staples.

It will be appreciated that the heating member 117 in some examples may have a cooling capacity allowing it to cool the wall of the renal artery 20 to cause the artery to contract. The heating member 117 may thus also be referred to as a thermal member, having the capacity to transfer heat to the wall and/or transfer heat from the wall. The operating mechanism of the thermal member may be based on a resistive heating, or the Peltier effect. In further examples, the heat may be transferred by means of a carrier fluid, such as water, arranged to add or remove heat from the wall of the artery 20.

During operation, the control unit 114, 124 may operate the stimulation device 110 such that the thermal member 117 is heated, thereby heating the renal artery 20 locally at position of the thermal member 117. As a result, a dilation of the blood vessel 20 may be achieved, allowing the blood to flow more freely within the renal artery 20 and thereby increase the blood pressure in the kidney 10.

Mechanically induced vasodilation will now be discussed with reference to FIGS. 17g-h, in which the renal artery 20 may be expanded by means of dilation device having an expansion member 212 implanted inside the artery. The expansion member 212 is configured to engage at least a portion of a in inner circumferential surface of the renal artery 20 and exert and expanding pressure on the wall of the renal artery 20 to assist in the vasodilation. Thus, the expansion member 212 may be used instead of the thermal or electrical stimulation devices discussed above, or in combination with either of them. Similar to the previous stimulation devices, the operation of the dilation device may be controlled by the control unit 112, 124 and energized by a source of energy similarly configured as the previously discussed sources of energy. Thus, the source of energy may be configured to be implanted inside the renal artery, be integrated in the expansion member, or arranged outside the renal artery. In the latter case the energy may be transferred wirelessly, such as inductively, or by means of a wired connection. Further, the source of energy may be charged by energy wirelessly transferred from outside the renal artery, such as from an extraluminar source of energy which may be implanted elsewhere in the body or arranged outside the body of the patient.

The expansion member 212 may be understood as a device suitable for implantation inside the artery and possible to controllably expand and/or contract so as to cause vasodilation. The expansion may for example be caused by means of mechanic, hydraulic or thermal action as will be discussed in the following. Further, the expansion member may comprise a tubular shape having an outer surface configured to rest against the inner surface of the renal artery 20. The expansion member 212 may for instance define a passage through which a blood flow of the renal artery 20 is allowed to pass. The expansion member 212 may be secured at its position by means of sutures or staples, and/or by means of fibrotic tissue at least partly covering or encapsulating the expansion member 212. Preferably, the expansion member 212 comprises a biocompatible material promoting fibrotic tissue growth.

In the example shown in FIG. 17g the expansion member may be a tubular structure, such as a stent-like structure, configured to be fitted within the inner walls of the artery 20. The tubular structure may be formed by a net-like structure, and preferably by a shape-memory materials that varies its shape with the temperature. This allows for the passageway defined by the expansion member to vary its cross-sectional area with the temperature, such that a heating of the tubular structure may cause the structure to expand and thereby induce vasodilation in the renal artery 20. Correspondingly, a cooling of the tubular structure may result in the structure contracting, reducing the pressure on the arterial wall and allowing it to contract again.

The heating may for instance be achieved by resistive heating of the shape-memory material, either directly or indirectly, or by means of additional heating elements (such as the ones disclosed in connection with FIGS. 17e-d).

An alternative principle of operation of the expansion member 212 is shown in FIGS. 17h-i, in which the expansion member 212 comprises at least one hydraulic expansion means, or bellows 214, operable to cause the expansion member 212 to increase its circumference. In the present example, the expansion member 212 is cylindrical or at least ring-shaped and comprises a first and a second abutment element 213 configured to be arranged to rest against the inner surface of the artery 20. The abutment elements 213 are interconnected by a first and a second bellows 214, which are hydraulically operated via a hydraulic reservoir (not shown) to cause the first and second abutment elements 213 to expand the arterial wall. The hydraulic reservoir may be implanted at a location different from the renal artery, and a motor or pump may be employed to move hydraulic fluid between the bellows and the reservoir to control the expansion and contraction. The motor or pump may be controlled by the control unit 114, 124 as discussed above.

Other operation principles are also possible, such as a mechanical expansion means instead of the bellows 214. A threaded, rotating bolt is an example of such a mechanical expansion means, wherein the bolt may be moved into and out from a nut to cause the expansion member 212 to increase or reduce its width.

FIG. 17i illustrates the hydraulic expansion member 212 in FIG. 17h when implanted in the renal artery 20, whereas FIG. 17j shows the stent-like expansion member 212 in FIG. 17g when implanted.

FIG. 18 shows a similar renal artery 20 as in FIGS. 17a-j, in which a signal damping device 120 has been implanted to at least partly enclose a portion of the renal artery 20. The signal damping device 120 may comprise a second electrode arrangement 122a, 122b configured to deliver an electric signal for damping or disturbing the electrical stimulation signal generated by the stimulation device 110, which may be similar to the ones disclosed in FIGS. 16 and 17. Alternatively, the signal damping device 120 is configured to divert the electrical stimulation signal, for instance by connecting a portion of the renal artery to ground or at least to a lower electrical potential, allowing the electric stimulation signal to travel towards the reduced potential rather than towards the spinal cord of the patient. The damping device 120 may be provided and operated with the purpose of reducing the effect of the electric stimulation signal on parts of the body other than the renal artery 20.

The utilization of the signal damping device 120 relies on the insight that the electrical stimulation signal used for causing the renal artery 20 to relax inadvertently may progress towards the spinal aorta 22 and/or the spinal cord, thereby risking causing unwanted side effects and unpleasant experiences for the patient. The signal damping device 120 may hence be provided to mitigate the effects of the electrical stimulation signal by damping, disturbing or at least partly cancelling the electrical stimulation signal on its way away from the renal artery 20 and the kidney 10. The signal damping device 120 may hence be arrange to at least partly intercept the electrical stimulation signal during its progress through the tissue towards the aorta 22/spinal cord. These mechanisms are discussed in greater details below.

The functionality of the medical device 110 generating the electric stimulation signal, i.e., the control unit 124 and the electrode arrangement 112a, 112b may also be referred to as a stimulation device 110. The stimulation device 110 and the signal damping device 120 may hence be operated at the same time, or simultaneously, to treat hypertension. The stimulation device 110 may be operated to deliver the stimulation signal and cause vasodilation, while the signal damping device 120 is operated to damp or disturb the stimulation signal propagating towards tissue for which electrical stimulation is unwanted.

The signal damping device 120 may be arranged to engage tissue of the renal artery 20, or a nerve innervating the renal artery 20, at a position allowing the stimulation device 110 to be arranged between the kidney 10 and the signal damping device 120. By this placement, the signal damping device 120 may be employed to prevent or at least partly hinder the electrical stimulation signal from propagating ‘upstream’ the nerve or renal artery 20, that is, towards the spinal cord or aorta.

In the present example, the stimulation device 110 may comprise a first electrode arrangement 112 comprising a plurality of electrode elements 112a configured to engage the wall of the renal artery 20. The electrode elements 112a may be connected to a control unit 114 by means of electrical lead portions 112b and configured to deliver an electric stimulation signal generated by the control unit 114. The contacting portions 112a may for example be attached to the wall of the renal artery 20 by means of stitches, for instance allowing for the contacting portion 112a to be at least partly inserted into the tissue on the outer surface of the wall, or arranged on a surface portion, such as a patch (not shown), which in turn may be placed on the tissue of the wall of the renal artery 20.

The signal damping device 120 may comprise a plurality of contacting portions 122a, or electrode elements 122a, configured to mechanically engage, or be arranged to rest against, a portion of the renal artery 20 to transmit the electrical stimulation signal to the tissue. In the present example, the electrode elements 122a are arranged on an inner surface of a cuff portion 126 configured to be arranged at least partly around the renal artery 20. The cuff portion 126 may in turn be electrically connected to the control unit 124 of the stimulation device 110 by means of a lead 122b.

During operation, when a reduction of blood pressure, or systemic vascular resistance, is desired, the signal damping device 120 may be caused to deliver an electrical damping signal preventing or at reducing propagation of the stimulation signal delivered to the renal artery 20. The electrical damping device 120 may for instance be configured to counteract or damping the action potentials that may be generated by the stimulation signal, thereby reducing the reaction from muscle cells or nerve cells in the vicinity of the electrode elements 122a of the signal damping device 120. Additionally, or alternatively, the electrical damping device 120 may be configured to deliver an electrical damping signal which is configured to cancel or damp the electrical stimulation signal by means of amplitude cancellation or by scrambling the signal (e.g., in terms of frequency contents) into a signature which cannot be ‘read’ by the muscle tissue or the nervous tissue. In different words, the electrical stimulation signal may be modified in a way that reduces its effect on tissue. Combinations are conceivable: the signal damping device 120 may be arranged to counteract or damp the action potentials affecting tissue such as smooth muscle cells and nerve cells and to damp or disturb the electrical stimulation signal before it propagates past the electrode elements 122b of the signal damping device 120.

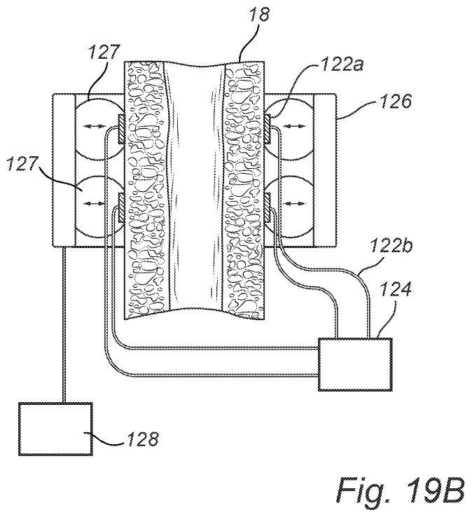

FIGS. 19a-b are cross sections of devices for delivering an electric signal to tissue of the patient. The devices may for example be a stimulation device 110 or a signal damping device 120, similarly configured as any of the embodiments described above with reference to FIGS. 16-18. In the following description of FIG. 19a the device will be exemplified as a signal damping device 120. However, it will be appreciated that the description may equally well apply to a stimulation device 110.

In the present example, the signal damping device 120 may comprise a support structure 126, such as for example a cuff 126, which may be formed to two or more support elements that are hingedly connected to each other and movable to allow the support structure 126 to be arranged around the renal artery 20. The support structure 126 may thus be arranged to at least partly, or completely, surround the renal artery 20, such that an inner surface portion of the support structure 126 faces or abuts an outer wall surface of the renal artery 20 when implanted. The support structure 126 may for instance be positioned by the surgeon attaching two or more interconnecting support elements to each other, when the support structure 126 is positioned around the renal artery 20. The inner circumference of the support structure 126 may be adapted to fit snuggly around the renal artery 20, and may either be adjustable, for instance by varying an overlap of the elements forming the support structure 126 upon attachment to the renal artery 20, or by selecting a support structure 126 (out of a plurality of different support structures) having a suitable circumference.

The inner surface may be adapted to support one or several electrode elements, or contacting portions 122a, for delivering an electrical damping signal to the tissue of the renal artery 20, or for connecting the tissue of the renal artery 20 to a lower electrical potential, such as ground, to divert the electrical stimulation signal from the tissue to the lower electrical potential. In this way, the signal damping device 120 may be capable of preventing the electrical stimulation signal from propagating past the signal damping device 120, or at least of reducing the impact of the electrical stimulation signal otherwise may have on tissue in the vicinity of, or upstream, the signal damping device 120. The electrode elements 122a may be electrically connected to a ground potential, or at least to a lower electric potential, by means of electrical leads 122b. Alternatively, the electric leads 122b connect the electrode elements 122a to a control unit 124 configured to generate a signal for damping or disturbing the electric stimulation signal, as described above.

The electrode elements 122a may preferably be arranged at the interface or contact surface between the support structure 126 and the tissue. The electrode elements 122a may for instance be plate electrodes, comprising a plate-shaped part forming contact with the tissue (as already stated, this applies both to signal damping devices as well as stimulation devices). In other examples, the electrode elements 122a may be a wire electrode or a lead, formed of a conducting wire that can be attached to the inner surface of the support structure 126 and brought in electrical contact with the tissue. Further examples may include needle- or pin-shaped electrodes, having a point at the end which can protrude from the inner surface of the support structure 126 and be inserted in the tissue of the wall, at which the signal damping device 120 or stimulation device 110 may be arranged to rest.

The control unit 124 may be operably connected to the electrode elements 122a for controlling the electric damping (or stimulation) signal provided to the tissue of the renal artery 20. The control unit 124 may be structurally integrated in the stimulation device shown in for example FIG. 18 and may be configured to receive input from a sensor arranged to sense or measure the electric stimulation signal generated by the stimulation device 110. In some examples, the sensor may be integrated with the control unit 124.

The sensor (not shown in FIG. 19) may be arranged in close vicinity of the portion of the renal artery 20 at which the electrode elements 122a contact the tissue of the renal artery 20. This advantageously may allow for the characteristics of the electrical stimulation signal to be determined close to the location of the signal damping device so that the damping device more efficiently can generate a damping or counteracting signal.

In a further configuration the cuff 126 of the stimulation device 110 and/or signal damping device 120 may be configured to adapt its shape, and more specifically its inner cross section, to the vasodilation so as to maintain a certain contact or abutment with the outer surface of the renal artery 20. This may for instance be realized by means of a hydraulically or pneumatically operated cuff 126 configured to maintain a substantially constant contact pressure between the artery 20 and the cuff 126, or by means of a mechanically operated adjustment mechanism configured to adjust the inner circumference of the cuff 126 according to the vasodilation.

An example is shown in FIG. 19b, disclosing a holding device 126, such as a cuff, configured to support the electrode arrangement 122a at the outer wall 18 of the artery and to define a passage through which the artery passes. The cuff 126 further comprises a plurality of abutment elements 127 having a varying volume and being configured to rest against the outer wall portion 18 of the artery. The varying volume allows a width of the passage, through which the artery passes, to increase with increased vasodilation and decrease with decreasing vasodilation. In the example shown in FIG. 19b the abutment elements comprises inflatable elements 127 varying their volume in response to the width of the artery varying. The abutments elements 127 are hydraulic or pneumatic elements fluidly connected to a fluid reservoir 128. The control unit 124 may be configured to cause fluid to be transported between the fluid reservoir 128 and the inflatable elements 127 based on a contact pressure between the holding device 126 and the outer wall 18 of the artery so as to control the volume of the inflatable elements 127 and thus the contact pressure accordingly. The contact pressure may be determined by means of a pressure sensor communicatively connected to the control unit 124. Further, in some examples the control unit 124 is configured to control an operation of the stimulation device, and thus the electric stimulation signal causing vasodilation, based on the signal generated by the sensor device.

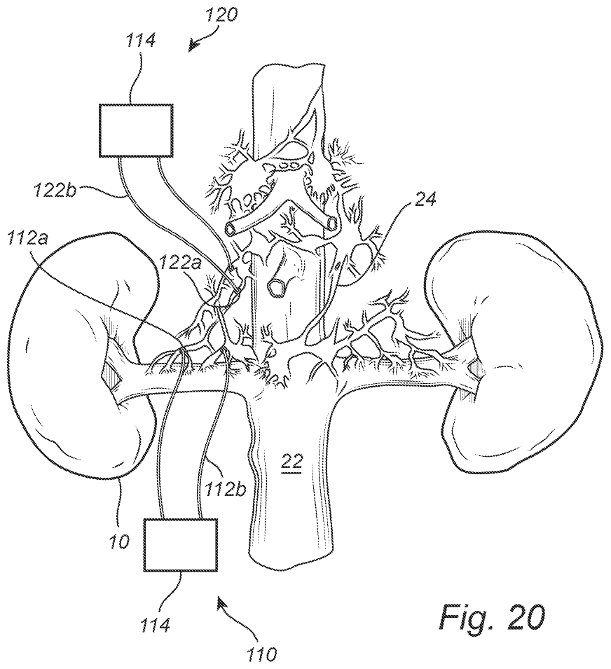

FIG. 20 schematically illustrates an example of the innervation of the renal artery 20, and shows the kidney 10, the aorta 22 and the renal artery 20 connecting the two. As mentioned above, the renal artery 20 may be innervated by renal sympathetic fibres 24 originating from ganglia in the solar plexus or from the splanchnic nerve collection and connecting the renal artery 20 as well as the kidney 10 forming the renal plexus. It is believed that a major part of the renal plexus comprises sympathetic nerves 24, but the presence of parasympathetic nerves may not be excluded.

While the stimulation device 110 and the signal damping device 120 in the above examples are described as configured to deliver electric signals, such as the electrical stimulation signal and the electric damping signal, directly to tissue of the wall of the renal artery 20, it will be appreciated that the electrical stimulation device 20 and the signal damping device 120 also may be arranged to act on nerves instead (or in addition). Thus, the stimulation device 110 may in some examples be configured to engage and electrically stimulate the nerves 24 innervating the renal artery 20 to cause vasodilation thereof, thereby promoting a reduction in systemic vascular resistance. The electrical stimulation device 110 may be similarly configured as any of the stimulation devices described above with reference to the previous figures and may for instance comprise one or several electrode elements 112a or contacting portions that can be attached directly to the nerve 24, or arranged on a supporting structure, such as a cuff of the like, which can be attached to the nerve that that is at least partly encloses the nerve 24. The stimulating electrode elements may thus be attached directly to the nerve, at a position between the innervated muscle tissue and the spinal cord 20 from which the nerve may origin. It will be understood that while the electrode elements can be attached directly onto a wall of the renal artery, for instance by means of a patch or stitching, the electrode elements may need to be slightly differently configured to be able to engage a nerve instead. Mostly due to the differences in diameter between the renal artery and a nerve. In the latter case, the electrode elements may be arranged on the inner surface of a support device, such as a cuff, which is dimensioned to be fit snugly around the nerve. Thus, while electric stimulation of a wall, or an entire blood vessel, may require the electrode elements to be arranged on the wall, the electrode elements may be arranged around the nerve in case a nerve stimulation is desired. In some examples the electrode elements 112a are arrange at a position closer to the innervated tissue than to the ganglia in the solar plexus or the splanchnic nerve collection, from which the nerve 24 may origin. In the present figure, an exemplary configuration is illustrated in sympathetic renal fibers 24 are stimulated at two positions by a respective electrode element 112a, each connected to a control unit 124 by means of a lead 112b.

When submitting an electrical stimulation signal to a nerve 24, there is a risk that the signal propagates to surrounding tissue which should not be stimulated. The stimulation signal may for instance propagate not only to the renal artery wall (which should be stimulated), but also ‘upstream’ towards the ganglia from which the nerve 24 origins. This may cause unwanted reactions in other parts of the nervous system, and an unpleasant experience for the patient. To address this issue, a signal damping device 120 may be arranged to engage the nerve 24 upstream of the stimulation device 110, at a position between the stimulation device 110 and the ganglia from which the nerve 24 origins. In the present figure, a signal damping device 120 has been arranged to engage nerves 124 at two different locations ‘upstream’ the electrode elements 112a of the stimulation device 110. The signal damping device 120 may be similarly configured as any of the signal damping devices described above with reference to the previous figures and may for instance comprise one or several electrode elements 122a or contacting portions that can be attached directly to the nerve 24, or arranged on a supporting structure, such as a cuff of the like, which can be attached to the nerve that that is at least partly encloses the nerve 24. The stimulating electrode elements may thus be attached directly to the nerve, at a position between the electrode elements 112a of the stimulation device 110 and the spinal cord 20 from which the nerve 24 may origin. The signal damping device 120 may be configured to hinder, damp or scramble the electrical signal propagating from the electrode elements 112a of the stimulation device 110. Alternatively, the signal damping device 120 may be configured to divert the propagating stimulation signals to a lower potential, such as ground.

Alternative, or additional approaches to address potential issues originating from unintended or unwanted propagation of the electric stimulation signal may involve supplying an additional signal to the tissue in which the electric stimulation signal propagates, thereby reducing or at least partly counteracting the tissue's reaction of the stimulation signal. Such an approach may rely on a mechanism relating to phase cancellation of the signal, in which the signal damping device 120 may be employed to deliver an electric signal to the muscle tissue of the renal artery 20 or the nervous tissue of the nerve 24, wherein the electric signal is configured to cancel or at least reduce the amplitude of the electric stimulation signal that has propagated from the electrode elements 112a of the electrical stimulation device 110. Generally, muscle cells are adapted to react on nerve fibre action potential waveforms, which in their natural state are biphasic (negative-positive) with a duration in the order of milliseconds. By adding a slightly phase shifted or reversed signal damping signal to the muscle tissue, the signal damping signal may be timed to position its negative peaks at the time of the positive peaks of the action potential originating from the stimulation signal propagating from the stimulation device 110. With a correct timing, a significant phase cancellation and lowering of the action potential waveform may be achieved, resulting in a reduced response from the muscle cells.

FIGS. 21A-D show examples of electrode arrangements according to some embodiments, which may be implemented in any of the signal damping devices and stimulation devices discussed above in connection with for instance FIG. 3A.

Various examples of sensors, which all may operate as outlined above, will now be discussed with reference to FIGS. 21a-d. 22a-b and 23. It will be appreciated that the below sensors may be combined with any of the above arrangements disclosed in for instance FIGS. 16-20 for causing electrically induced vasodilation of the renal artery. The description of such systems and devices will not be repeated in the following.

FIGS. 21a-d show examples of sensors 140 for generating a signal indicative of a blood pressure, a vascular flow, or a vascular resistance, in a blood vessel 20 of a patient. The blood vessel 20 may for instance be a renal artery of the patient, or another artery such as the radial artery, femoral, dorsalis pedis or brachial artery. In some examples the blood vessel may be a vein. The sensors 140 may be for instance be configured to convert a pressure signal into another signal, such as an electrical signal, indicative of the pressure in the blood vessel 20. This signal may be transmitted to a control unit (not shown) configured to control an operation of a stimulation device as previously discussed in the present disclosure. The transmission between the sensor 140 and the control unit may take place over a wired or wireless communication channel, which for example may be formed of one or more electrical leads interconnecting the control unit and the sensor.

FIG. 21a shows an example of a sensor 140 configured to be arranged to measure the pressure directly in the blood vessel 20. The sensor 140 may hence comprise a probe 142 configured to penetrate a wall portion of the blood vessel 20 and be arranged within the lumen, or blood passageway defined by the interior of the blood vessel 20. The sensor may further comprise a body part configured to be arranged on the outside of the blood vessel 20. The body part may for example be configured to be attached or secured to the outer surface of the wall. The probe 142 may be provided at an underside of the body part so as to allow the probe to extend into the interior of the blood vessel 20 when the body part is attached to the wall part of the blood vessel 20.

FIG. 21b shows an example of an optical sensor 140 for measuring blood pressure pulses transmitted through the walls of the blood vessel 20. The sensor 140 may comprise a light source 141 and a light sensor 143 configured to be arranged on opposite sides of the blood vessel such that light from the light source 141 can be transmitted through the blood vessel 20 and the blood flowing therethrough. In an example, the light is transmitted from the light source 141 through a light transmitting body 141′ towards the blood vessel 20. The light transmitting body 141′, or light guide 141′, may comprise a convex surface being curved towards the outer wall of the blood vessel 20. The curved surface may be arranged to abut the outer wall, such that a contact area is provided at the interface between the blood vessel 20 and the light transmitting body 141′. It has been observed that the size of the contact area may vary with the contact force between the blood vessel 20 and the light transmitting body 141′ (i.e., the force with which the surfaces of the blood vessel 20 and the light transmitting body 141′ abuts each other), such that an increased contact area is achieved when the wall of the blood vessel 20 is pushed against the light transmitting body 141′ and a reduced contact area is achieved when the contact force between the wall of the blood vessel and the light transmitting body 141′ is reduced. By arranging the sensor 140 such that it abuts the outside of the wall of the blood vessel 20, the contact area between the two may be caused to vary in size with the pressure pulse waves transmitted through the blood vessel 20. It has further been observed that the light coupling efficiency through the blood vessel 20, for example measured as the percentage of the light generated by the light source 141 that is registered by the sensor 143, may vary with the size of the contact area between the light transmitting body 141′ and the wall of the blood vessel 20. Hence, the variations in the signal at the light sensor 143 may be analyzed to calculate a corresponding variation in contact pressure and thus get an indication of the pressure in the blood vessel 20. As indicated in the present figure, there may alternatively, or additionally, be provided a light transmitting body 143′ arranged at the light sensor side, which may be configured and function in a similar way as described above.

The light transmitting body 141′, 143′ may in some examples be rigid so that the variations in contact area are caused by the wall of the blood vessel 20 deforming rather than the light transmitting body 141′, 143′ deforming. In further examples the light transmitting body 141′, 143′ may be flexible, allowing it to deform with an applied contact force between the blood vessel and the light transmitting body 141′, 143′.

The sensor 140 may comprise a holding structure, such as a cuff 144, configured to at least partly enclose the blood vessel 20 and push the light transmitting body (or bodies) 141′, 143′ against the outer wall of the blood vessel 20.

Another embodiment, which may be similar to the one illustrated in FIG. 21b, may operate based on acoustic waves instead of optical principles. Such a sensor 140 may hence comprise an acoustic transducer instead of the light source 141 and an acoustic sensor instead of the light sensor 143. Still, the coupling efficiency through the blood vessel may be determined as a function of a varying contact area between the sensor 140 and the outer wall of the blood vessel 20, allowing for a signal to be generated which is indicative of the pulse waves travelling through the blood vessel 20. Similar to the optic version above, the coupling efficiency of the sound may increase with increasing contact area and decrease with decreasing contact area, as the pulse wave passes by.

FIG. 21c show an example of a sensor 140 operating by means of a doppler radar principle, in which a beam of electromagnetic (or acoustic) waves is sent from a transmitter 147 and reflected from the outer wall of the blood vessel. Assuming that the wall moves slightly back and forth along a radial direction of the vessel as the pulse wave passes through the vessel, the movement may be determined based on a slight change in frequency of the reflected waves. These changes may be observed by the transmitter 147 and used as a basis for generating a signal indicative of the pressure in the blood vessel 20.

FIG. 21d shows a further example of a sensor 140, which may be configured as a strain sensor generating a signal in response to strain induced in the wall by the pressure variations caused by the patient's pulse beats. The sensor 140 may for example operate based on a capacitive principle, in which the capacitance between two electrodes 145, 146 may vary with varying structural dimensions between the electrodes 145, 146. The electrodes 145, 146 may for instance comprise a first and a second interdigitated finger electrodes, having a separation which may vary with induced strain in the wall of the blood vessel 20. An increased separation between the electrodes 145, 146 may be observed as a reduced capacitance, indicating an increased strain in the wall, whereas a reduced separation may be observed as an increased capacitance, indicating a reduced strain in the wall. Further, the strain sensor 140 may be used to measure a vasodilation and/or vasoconstriction in the blood vessel 20.

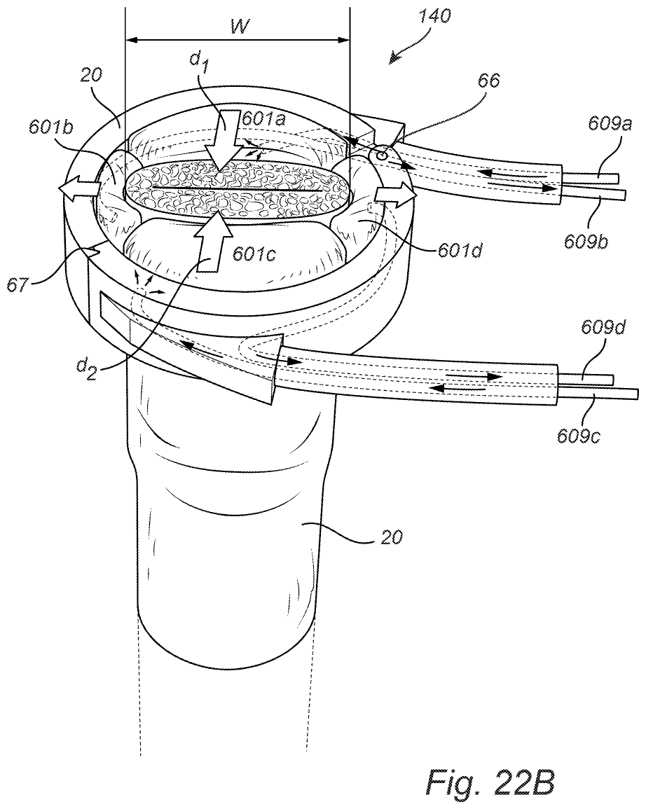

FIGS. 22a and b show an example of a sensor 140 formed of a constriction device configured to at least partly constrict, or at least push against the outer wall of, the blood vessel 20. The sensor 140 in some examples operate according to the auscultatory principle, in which the blood pressure is measured by constricting the blood vessel until it is occluded, and the blood flow substantially stopped, and in other examples according to an oscillometric method in which oscillations in a constrictive pressure applied by the constriction device (which hence not necessarily is operated to fully close the blood flow passageway) are measured.

The constriction device 140 in FIG. 22a may hence be configured to constrict a wall portion of the of a blood vessel 20, such as the renal artery or another artery which may be more easily accessed for the implantation procedure. The constriction device 116 may comprise a surrounding structure, or support structure, having a periphery arranged to surround the blood vessel 20 when implanted. The surrounding structure may be configured to support one or several constriction elements configured to expand inwards, towards an opposing wall of the surrounding structure, to abut the outer wall of the blood vessel 20 and thereby allowing pressure pulses induced by the blood pressure in the vessel to be transmitted into the constriction elements. Further, in some examples, the constriction elements may be operable to close the passage through the blood vessel to allow the blood pressure to be measured using the auscultatory principle.

In the example shown in the present figure, the surrounding structure comprises two support elements 64a. 64b connected to each other for forming the surrounding structure. The first support element 64a may be configured to support a first operable hydraulic constriction element 601a and a second operable hydraulic constriction element 601b. The second support element 604b may be configured to support a third operable hydraulic constriction element 601c and a fourth operable hydraulic constriction element 601d. The first, second, third and fourth operable hydraulic constriction elements 101a-d may be configured to constrict the blood vessel 20 for restricting the flow and configured to release the constriction when so desired.

The first and second support elements 64a, 64b each comprises a curvature C adapted to follow the curvature of the portion of the blood vessel 20 at which the pressure sensor 140 is arranged, such that the pressure sensor 140 snuggly fits around the blood vessel 20 and the distance which the operable hydraulic constriction elements 604a-d needs to expand to abut or even constrict the blood vessel 20 is reduced.

The first and second support elements 64a, 64b may be hingedly connected to each other such that a periphery of the surrounding structure is possible to open, thereby allowing the surrounding structure to be placed around the blood vessel 20. A first end of the first and second support elements 64a, 64b may comprise a hinge 66, whereas the other ends of the first and second support elements 64a, 64b may comprise portions of a locking member 67′, 67″, each comprising protruding snap-lock locking members materially integrated in the first and second support elements 64a, 64b and configured to be snapped together for closing the periphery of the surrounding structure, thereby allowing the surrounding structure to partially or completely encircle the blood vessel 20.

The constriction elements may be hydraulically connected to a pressure sensor configured to register pressure pulses induced in the constriction elements by the pressure pulses travelling through the blood vessel. The registered pressure pulses may then be converted in to a signal that is indicative of the blood pressure in the blood vessel and transmitted to the control unit as discussed above.

In the embodiment shown in FIG. 22a, each of the first and second support elements 64a. 64b comprises fluid conduits 609a-d partially integrated in the support elements 64a, 64b. In the first support element 64a a first conduit 609a comprises a first portion in the form of a first tubing which enters a tubing fixation portion 65a fixated to, or materially integrated with, the first support element 64a. In the tubing fixation portion 65a the fluid conduit 109a is transferred into a first integrated channel 23a in the first support element 24a. The first support element 64a may comprise an inner surface 68a which configured to be oriented to face the wall of the blood vessel 20, when the sensor 140 is implanted. The inner surface 68a of the first support element 64a may comprise a fixation surface for fixating the first and second operable hydraulic constriction elements 601a. 601b. The fixation surface also comprises an outlet from the first integrated channel 63a into the first operable hydraulic constriction element 601a, such that fluid can be transferred from the first tubing to the first integrated channel 63a and into the first operable hydraulic constriction element 601a for expanding the first operable hydraulic constriction element 601a.

A second tubing of the second fluid conduit 609b may also enter the tubing fixation portion 65a fixated to, or materially integrated with, the first support element 64a. In the tubing fixation portion 65a the second fluid conduit 609b is transferred into a second integrated channel 63b in the first support element 64a. The fixation surface also comprises an outlet from the second integrated channel 63b into the second operable hydraulic constriction element 601b, such that fluid can be transferred from the second tubing to the second integrated channel 63b and into the second operable hydraulic constriction element 601b for expanding the second operable hydraulic constriction element 601b.

The second support element 64b may, similar to the first support element 64a, comprise a fixation surface for fixating a third and fourth operable hydraulic constriction element 601c, 601d operated by fluid supplied by third and fourth fluid conduits 609c, 609d as illustrated above with reference to the first and second constriction elements 601a, 601b.

The tubing portion of the fluid conduits 109a-d is preferably made from a biocompatible material such as silicone and/or polyurethane.

Integrating the fluid conduit(s) in the support element(s) enables the fluid entry to the operable hydraulic constriction elements 601a-d to be protected and encapsulated by the support element(s) which reduces the space occupied by the constriction device 116. Further, it may reduce the amount of protruding portions, thereby reducing the risk of damaging the tissue of the blood vessel 20 around which it is implanted.

The first, second, third and fourth operable hydraulic constriction element 601a-d may be connected to a shared hydraulic system, such that the abutment against the wall of the blood vessel as well as any potential constriction of the blood flow passageway may be regulated by pumping the hydraulic fluid to and from the constriction elements 601a-d. Alternatively, one or several of the hydraulic constriction elements 601a-d are individually controllable, such that for instance the first and third hydraulic construction elements 601a, 601c share a first hydraulic system and the second and fourth hydraulic constriction elements 601b, 601d share a second hydraulic system, separate from the first hydraulic system. This advantageously allows for the first and third constriction elements 601a, 601c to be inflated at the same time as the second and fourth constrictions elements 601b, 601d are deflated. Further, one or several of the hydraulic constriction elements may be connected to a hydraulic pressure sensor for measuring pressure variations in the constriction element(s) caused by the pulse waves in the blood vessel 20.

The first and third constriction elements 601a, 601c may have a respective volume that is larger than a respective volume of the second and fourth constriction elements 601b, 601d. In the embodiment of FIGS. 22a and b, the first and third constriction elements 601a, 601c may have a respective volume that is more than 1.5 times larger than the respective volumes of the second and fourth constriction elements 601b, 601d. However, it is also conceivable that the first and third constriction elements 601a, 601c have a respective volume that is more than 2 times as large as the volume of the second and fourth constriction elements 601b, 601d.

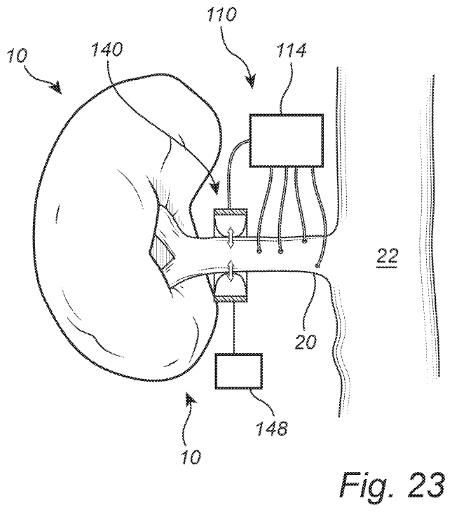

The sensors shown in FIGS. 21a-d and 22a-b may be connected to, or form part of, a stimulation device 110 for causing electrically induced vasodilation in a renal artery of a patient. As example of such an implementation is illustrated in FIG. 23, wherein a stimulation device 110 similar to the one discussed with reference to FIGS. 1A and 16 is implanted together with a sensor 140 similar to the one disclosed in connection with FIGS. 5A and 22a-b. As shown in FIG. 23, both the stimulation device 110 and the sensor 140 may be arranged to act on the renal artery 20 of the patient, with the stimulation device 110 configured to electrically stimulate smooth muscle tissue of a wall portion of the renal artery 20 and the sensor 140 arranged as a cuff at least partly enclosing the renal artery 20. The cuff comprises at least two inflatable elements arranged to abut the outer wall of the renal artery. The inflatable elements are connected to an operation device 148 for varying a contact pressure between the inflatable elements and the renal artery. The operation device 148 may for instance be a hydraulic device configured to move a hydraulic fluid to and from the inflatable elements to adjust their inflation. The sensor 140 may be communicatively connected to a control unit, or controller 114, of the stimulation device 110 to provide the control unit 114 with a signal indicative of a pressure in the renal artery 20. This signal may be used by the control unit 114 for controlling the stimulation and hence the vasodilation of the renal artery 20.

The present disclosure also relates to implantable vibration devices configured to deliver vibration stimulation to human tissue at an implanted position in the human body. In particular, the implantable vibration device may comprise a vibrating generating unit as described in greater detail in the following. It should be noted that the vibrating generating unit also may be referred to as a signal generating means or stimulation arrangement, and may hence be similarly configured as any of the stimulation arrangements 210, 220 disclosed above in connection with, for example, FIGS. 1A, 3A, 10A, 11, and 12. Hence, in those embodiments of the present disclosure where the system comprises a stimulation device 200 for affecting or generating an effector response in effector tissue, the implantable vibration device may be provided in order to cause vibration of the nerve innervating the effector tissue or of the effector tissue as such.

FIG. 24a schematically illustrates an implantable vibration device 910 which is suitable for operation in the human body according to any one of the embodiments described with reference to FIGS. 1-23 comprising a casing 920. Contained in the casing 920, a piezoelectric vibration generating unit VGU is provided. The VGU is capable of causing the implantable vibration device 910 to vibrate in a suitable manner. In FIG. 24a, the implantable vibration device 910 is also provided with wireless energy receiver R for receiving wireless energy for the operation of the vibration generating unit, and an internal controller C_{I} configured to control the operation of the vibration generating unit. By providing a wireless energy receiver R and a controller C_{I} within the casing 920, a physically independent vibration generating device which can be implanted in the human body is obtained. Thus, leads and cables for controlling and/or providing energy to the vibration device can be omitted. The internal controller C_{I} may correspond to the control unit 240 discussed above with reference to, e.g., FIGS. 1A, 3A, and 5A. In some examples, the internal controller C_{I} may form part of such as control unit 240.

In alternative embodiments, the wireless energy receiver R and the internal controller may be provided in a different casing, separate from the casing 920, and connected to the vibration generating unit via lead or cable. Preferably, such lead or cable flexible and short, typically having a length of less than 10 cm, such as of less than 5 cm, such as less than 1 cm. This may be advantageous as it allows for the casing 920 to be implanted via invagination in the tissue of the patient, whereas a smaller casing containing the wireless energy receiver and/or internal control C_{I} can be implanted in a different manner.

In some examples, the VGU is arranged in a holder suitable for attachment at or to a nerve 231, 232. Examples of such holders include a cuff 215 as shown in, e.g., FIG. 1F. The VGU may be attached to an exterior of the cuff 215 to bring the entire cuff to vibrate, thereby allowing vibrations to be imparted to the nerve around which the cuff 215 is arranged. In other examples, the VGU may be integrated into the cuff, such as in a wall of the cuff 215. Further configurations and designs are conceivable, depending on the available space at the implantation site. However, it should be noted that the VGU can be implemented without the particular casing 920 shown in the present figures.

The casing is shown in FIG. 24A to have a substantially cylindrical outer shapes, but other shapes such a sphere can also be contemplated, which is shown in FIG. 24B. The casing may be made of any suitable biocompatible material known to the person skilled in the art. In order to mitigate fibrin creation caused by contact between the implantable vibration device 910 and the tissue or flowing blood of a patient, the implantable casing may comprise a specific coating arranged on the outer surface of the casing. The coating may comprise at least one layer of a biomaterial. The biomaterial is preferably fibrin-based. The coating may comprise at least one drug or substance with antithrombotic and/or antibacterial and/or antiplatelet characteristics. The drug or substance may be encapsulated in a porous material.

There may be provided a second coating arranged on the first coating. The second coating may be a different biomaterial than said first coating. In particular, the first coating may comprise a layer of perfluorocarbon chemically attached to the surface and the second coating may comprise a liquid perfluorocarbon layer.

Furthermore, the surface may comprise a micro pattern, wherein the micro pattern may be etched into the surface prior to insertion into the body. The layer of a biomaterial may be coated on the micro pattern.

According to an embodiment, the implantable vibration device may be configured to be invaginated when placed on the outside of the stomach wall. In another embodiment, the vibration device may be configured to be invaginated when placed on the inside of the stomach wall. Invaginating the implantable vibration device in the stomach wall may be an effective way of delivering a vibrational stimulation signal to effector tissue, such as receptors, glands, or smooth muscle cells, as well as delivering a stimulation signal to nerves innervating tissue of the stomach wall. In some examples, this may be employed to deliver a stimulation signal to branches of the vagus nerve innervating the stomach or other parts of the gastrointestinal tract.

In some embodiments, the implantable vibration device 910 is configured to be at least partially invaginated by the tissue of the stomach wall using stomach-to-stomach sutures or staplers. In some embodiments, the system comprises the stomach-to-stomach sutures or staplers. Thus, the implantable vibration device can be kept in a partly invaginated position, preferably on the outside of the stomach or intestine wall. Consequently, the vibration device can be kept in a position where it abuts the tissue of the stomach wall.

The vibration device may be adapted to be placed in the stomach cavity. To this end, the vibration device may be adapted to be inserted into the stomach cavity via a gastroscope or intraluminar instrument, and be adapted to be invaginated in the stomach or intestine wall by surgery.

In some embodiments, the implantable vibration device is configured to abut the tissue of the stomach wall on the outside thereof, preferably be being invaginated by the tissue of the stomach wall.

In some embodiments the implantable vibration device is configured to abut the tissue of the intestine wall on the outside thereof, preferably be being invaginated by the tissue of the intestine wall.

In some embodiments, the implantable vibration device is configured to be implanted in or near the sexually responsive tissue near the vulva of a female patient, the erectile tissue of a male patient, or nerves innervating such tissue.

In some embodiments, the implantable vibration device has a volume of from 0.3 cm³-6.6 cm³, or in the range 0.5 cm³-7.3 cm³, or in the range 3 cm³-8 cm³, or in the range 2.5 cm³-6.6 cm³, or in the range 4 cm³-7.3 cm³.

The wireless energy receiver R is configured to receive wireless energy for the operation of the vibration generating unit. In some embodiments, the wireless energy receiver R of the implantable vibration device includes a secondary coil, configured to receive wireless energy from a wireless energy transmitter, preferably comprising a primary coil configured to induce a voltage in the secondary coil of the vibration device. This way, energy can be transmitted wirelessly from the energy transmitter to the energy receiver via the primary and secondary coils. A suitable transmitter is typically implanted at a second, distant position in the body of the patient. Suitable transmitters are described in relation to FIGS. 35B, F, L, and V.

In some embodiments, RFID technology is used to transfer the energy wirelessly from an energy transmitter to the energy receiver R. RFID technology is widely known, and transfer of energy via the aforementioned primary and secondary coils is a well-known way of transferring energy by RFID technology. More specifically, the wireless energy receiver R may be configured to receive the energy via RFID pulses.

In some embodiments, the internal controller C_{I} further comprises a feedback unit configured to provide feedback pertaining to an amount of energy received by the wireless energy receiver (R) via the RFID pulses. Based on this feedback an amount of transmitted and/or received energy can be controlled based on the feedback. More specifically, the amount of RFID pulse energy that is being received may be adjusted based on the feedback such that the pulse frequency is successively raised until a satisfying level is reached.

In some embodiments, the implantable vibration device comprises a rechargeable energy storage unit for temporarily storing at least part of the wirelessly received energy. The rechargeable energy storage unit may be a rechargeable battery or a capacitor. The rechargeable energy storage unit may be charged over time so that an energy amount required by the vibration device or vibration devices is available when needed.

The internal controller C_{I} may serve various functions, the main function consisting in controlling the timing and amount of energy applied to vibration device for controlling the vibrations. Another important function consists in controlling and possibly storing away the amount of energy that is received via the wireless energy receiver. In some embodiments, the reachable energy storage unit discussed above is part of the internal controller. The internal controller may further serve to communicate with an external controller and/or with a remote controller. For instance, such communication may relate, inter alia, to the energy transfer via the energy receiver and/or to the timing and/or amount of energy to be applied to the vibration device.

The internal controller C_{I} may further be adapted to control whether received energy should be utilized to charge the rechargeable energy storage device or to operate the vibration generating unit.

In some embodiments, the internal controller C_{I} is configured to wirelessly receive vibration control data for controlling the vibration of the implantable vibration device.

In some embodiments, the internal controller is configured to receive the vibration control data wirelessly via the wireless energy receiver R. The control data may be used to for controlling the vibration of the implantable vibration devices, such that the relevant response in the patient can be activated. Thus, not only the energy transfer but also data transfer is carried out wirelessly in order for the vibration device to be physically independent other parts of the system. Such data may be received either from an implanted external controller or from a remote controller outside the patient's body.

Preferably, the internal controller receives the vibration control data wirelessly via the wireless energy receiver. In other words, the same port may be used to receive both energy and data. In particular, the energy transferred to and received by the vibration device via the wireless energy receiver may be appropriately modulated, the modulation defining and, thus, carrying a signal which may be decoded by the internal controller and interpreted as data. This is a well-known technique, which is particularly known and used within the RFID technology. That is, an RFID signal may be used to transport both energy and information.

In some embodiments, the internal controller includes an individual code by which it is individually addressable by an external controller or remote controller. If more than one vibration devices are provided in the patient, each vibration device may be addressable individually by an external controller or remote controller using an individual code, i.e. a code which is specific to the respective internal controller. This is particularly useful where one external controller or remote controller is used to control more than one vibration device and/or where one wireless transmitter is used to transmit energy wirelessly to the wireless energy receivers of more than one vibration device. For instance, when vibration devices are to be activated sequentially, e.g. for stimulating the stomach or intestine in a wave-like manner, the respective vibration device may be addressed individually using the individual code of the corresponding internal controller. Typically, such individual code is placed at the beginning of the data transmitted to the internal controller. This way, only one or more desired vibration device may be instructed at a given time to vibrate and/or only one or more desired vibration devices will receive and possibly store energy received through the wireless energy receiver.

Modes of transferring data and energy to the implantable vibration device 910 are described in further detail with reference to below figures.

The vibration generating unit VGU is configured to cause the implantable vibrator to vibrate, so that it is capable of temporarily displacing tissue of the patient when the vibrator is in its implanted position in the body.

The vibrations provided by the vibration device can generally be defined by their frequency, their period and by their amplitude. The frequency denotes the number of complete cycles of vibration occurring per period of time.

The vibration generating unit VGU can be configured to cause the implantable vibrator to vibrate with a frequency in the range of from 0.01 Hz to 10 000 Hz.

It has been found that a vibration frequency in the range of 1-200 Hz, such as in the range of 1-150 Hz, such as in the range of 30-150 Hz, preferably in the range of 35-150 Hz, such as in the range of 35-100 hz, such as in the range of 60-100 Hz has proven advantageous for activating at least some of the mechanoreceptors responsible for at least part of the control of satiety in the patient.

In some embodiments, the implantable vibration device is configured to vibrate with a period of 0.01-1 seconds, such as of 0.05-1 seconds. The period of the vibration is defined as the time it takes for the vibration to complete its cycle.

Further, it has been found that a vibration frequency in the range of 0.1-100 Hz, such as 1-50 Hz, may be advantageous for activating effector tissue, i.e., to generate an artificial nerve signal that triggers, for example, muscle contraction or increase secretion.

It has also been found that a vibration frequency in the range of 1-10 KHz, such a 2-5 kHz, can be used to inhibit propagation of action potentials in a nerve. Hence, applying such a signal to a nerve may result in the nerve signals being blocked or at least heavily reduced. In some embodiments, vibration generating unit VGU is configured to cause the implantable vibrator 910 to vibrate at an amplitude of at least 0.01 mm. In some embodiments the vibration generating unit VGU is configured to cause the implantable vibrator 910 to vibrate at an amplitude of from 0.01 mm-30 mm.

The amplitude is defined as the maximum displacement of the mass center of the vibration device 910 from its resting position. In case of the vibration device 910 being invaginated, the vibration device can displace tissue of the stomach or intestine wall a distance approximately corresponding to the amplitude of the vibrations. In other applications, the vibration device 910 can be arranged to displace the effector tissue or the nerve, to which the stimulation signal is delivered, a distance approximately corresponding to the amplitude of the vibrations.

A displacement of tissue of at least 1 mm has been found sufficient to activate the relevant mechanoreceptors in the stomach, whereas less than 1 mm may suffice to generate action potentials in a nerve.

Preferably, the implantable vibration device is configured to vibrate at an amplitude of at 0.1 mm, such as at least 1 mm, such as of at least 2 mm, such as of at least 3 mm, such as at of at least 4 mm, such as at least 5 mm, such as at least 6 mm. In some embodiments, the vibration generating unit VGU is configured to cause the implantable vibrator 910 to vibrate at an amplitude in the range of 1-10 mm, such in the range of 1-5 mm, preferably 2-4 mm.

This means that tissue which abuts the vibration device in the implanted position can displaced a corresponding length by each vibration cycle.

In some embodiments, the vibration device has a mass of at least 0.1 g, such as at least 1 g. A sufficient mass is preferred such that a sufficient force can be delivered to the tissue for activating at least some of the mechanoreceptors or axons of the nerve.

The vibration generating unit is preferably operated by a piezoelectric motor. Depending on the piezoelectric motor type, the vibrations may be caused by various mechanisms, such as those exemplified in the following.

The piezoelectric effect is a property of certain solid materials to generate an electrical voltage in response to an applied mechanical stress (so-called direct piezoelectric effect) and to deform elastically in response to an applied electrical voltage (so-called inverse piezoelectric effect). The piezoelectric effect is a reversible process, meaning that materials exhibiting the direct piezoelectric effect also exhibit the inverse piezoelectric effect.

Materials exhibiting the piezoelectric effect are denoted as piezoelectric materials. Examples of piezoelectric materials comprise: crystalline materials, such as lithium niobate, lithium tantalate and quartz; ceramics, such as lead zirconate titanate, potassium niobate and barium titanate; polymers, such as polyvinylidene fluoride.

Piezoelectric coefficients are a fundamental property of piezoelectric materials. A given piezoelectric material is characterized by a set of piezoelectric coefficients, wherein a piezoelectric coefficient is a measure of the relationship between the applied mechanical stress along a first direction and the generated electric charge along a second direction. Piezoelectric coefficients are usually expressed in units of picocoulombs per newton (pC/N). The value of piezoelectric coefficients may strongly vary depending on the piezoelectric material and piezoelectric coefficient being considered. For example, the d₃₃ piezoelectric coefficient is commonly reported for piezoelectric materials and quantifies the electric charge generated along a given direction in response to the mechanical stress applied along the same direction.

A piezoelectric motor or piezo motor is a type of electric motor that uses the inverse piezoelectric effect to generate mechanical motion, typically linear or rotatory motion. Piezo motors are often used in applications where precise positioning and fine control of movement are required. Piezo motors have the advantage of providing high motion accuracy, being possible to miniaturize and being relatively immune to interference, such as electromagnetic interference. Piezoelectric motors can also be manufactured without magnetic and/or metallic parts, and instead be manufactured from ceramics or certain composites. This feature is particularly advantageous in medical and biotechnology applications with strong magnetic fields. Piezoelectric motors can thus be made MRI-safe, meaning that the patient can undergo Magnetic Resonance Imaging (MRI) while having the piezo motor implanted. MRI is a medical imaging technique used to form pictures of the anatomy and the physiological processes of the body using strong magnetic fields. Conventional implantable electromagnetic motors prevent the use of MRI as the strong magnetic field risks damaging both the patient and the implant.

Also, compared to classical electromagnetic motors, piezo motors may have a simpler structure and smaller footprint. Piezo motors may offer improved positioning accuracy and simpler design, as linear motion may be obtained directly, without the need of mechanical coupling elements otherwise required to convert the rotary motion of classical electromagnetic motors to linear motion. That linear motion can be obtained directly may improve the positioning accuracy.

An additional advantage of piezoelectric motors is that they usually feature higher energy-efficiency and less power consumption compared to conventional electromagnetic motors.

As discussed in more detail in the sections below, inchworm motors, inertial motors, walk-drive and ultrasonic motors are three common types of piezoelectric motors.

FIG. 25 shows an embodiment of an inchworm motor MO configured to generate linear motion. The inchworm motor is configured to acts as at least part of a vibration generating unit VGU. The inchworm motor MO comprises a first lateral piezoelectric actuator 801a and second lateral piezoelectric actuator 801b. The first lateral piezoelectric actuator 801a is laterally connected to a first clutching actuator 802a′ and a second clutching actuator 802a″. The second lateral piezoelectric actuator 801b is laterally connected to a third clutching actuator 802b′ and a fourth clutching actuator 802b″. The inchworm motor MO is configured to impart a linear motion to a movable member 805. The movable member 805 is configured to be attached to a load or to the casing of the implantable vibrator, so that it can cause the implantable vibrator to vibrate.

FIG. 26 illustrates an operation cycle of the piezoelectric inchworm motor MO wherein the movable member 805 is linearly moved in a direction to the right in the illustration by sequentially controlling the first and second piezoelectric actuators 801a, 801b and the clutching actuators 802a′, 802a″, 802b′, 802b″. In an initial relaxation state, the movable member 805 is detached from all the clutching actuators 802a′, 802a″, 802b′, 802b″. The inchworm motor MO is subsequently brought into an initialization state by electrically activating the second and fourth clutching actuators 802a″, 802b″. As a result, the clutching actuators 802a″, 802b″ extend and clutch the movable member 805.

In step 1, the first and second lateral actuators 801a, 801b extend in response to an applied electrical voltage. As a result, the movable member 805 undergoes a first linear displacement with a distance equal to half the distance of the extension of the lateral actuators 801a, 801b.

In step 2, the first and third clutching actuators 802a′, 802b′ are electrically activated. As a result, the first and third clutching actuators 802a′, 802b′ extend and clutch the movable member 805.

In step 3, the electrical voltage applied to the third and fourth clutching actuators 802a″, 802b″ is decreased as compared to the initialization state. As a result, the third and fourth clutching actuators 802a″, 802b″ detach from the movable member 805.

In step 4, the electrical voltage applied to the first and second lateral actuators 801a, 801b is decreased as compared to step 1. As a result, the first and second lateral actuators 801a, 801b contract and the movable member 805 undergoes a second linear displacement with a distance equal to half the distance of the contraction of the lateral actuators 801a, 801b.

In step 5, the second and fourth clutching actuators 802a″, 802b″ are electrically activated such that they extend and clutch the movable member 805.

In step 6, the electrical voltage applied to the first and second clutching actuators 802a′, 802b′ is decreased as compared to step 2. As a result, the first and second clutching actuators 802a′, 802b′ contract and detach from the movable member 805.

A reversal of the applied electrical voltages will reverse the steps 1 to 6. This will cause the moveable member to move in the opposite directions, towards its starting position. Thus, by alternating between a first voltage and a second, reversed voltage, a back- and forth movement of the moveable member can be obtained. By coupling the moveable member to a load, or by connecting the moveable member to the casing of the implantable vibrator, the back-and-forth motion of the moveable member can cause the implantable vibrator to vibrate.

In alternative embodiments, the movable member 805 may be replaced with a rotary module (not shown) such that the inchworm motor can be configured to generate rotary motion. Such an inchworm motor could then be used to rotate an eccentric mechanism to thereby cause a rotation in the implantable vibrator. Such a configuration is described in greater detail in relation to FIGS. 30A and B.

The inchworm motor is configured to generate a linear motion at a speed in the range 1 mm/s to 10 mm/s, a stroke length in the range 1 mm-30 mm and a force in the range 2 N-30 N.

FIG. 27 illustrates an embodiment of a piezoelectric inertial motor MO configured to generate linear motion. The piezoelectrical inertial motor is configured to acts as at least part of a vibration generating unit VGU. The motor MO comprises a movable member 805, a piezoelectric actuator 801, a recoiling member 804 and a base 806. The movable member 805 is attached to the piezoelectric actuator 801. The piezoelectric actuator 801 is attached to the recoiling member 804. The movable member 805 is in contact with the base 806. The movable member 805 is configured to be attached to the load or mechanism to be moved.

An operation mode of the piezoelectric inertial motor MO described in FIG. 27 comprises two steps. In step 1, the piezoelectric actuator 801 slowly elongates in response to an applied voltage. Due to the static friction between the movable member 805 and the base 806, the movable member 805 does not move. In step 2, the piezoelectric actuator 801 rapidly contracts in response to a change in the applied voltage. As a result of the recoil produced by the recoiling member 804, the movable member 805 moves by a distance Ax to the left in the illustration of FIG. 27.

By reversing the operation cycle, the piezoelectric inertial motor MO generates motion in the opposite direction. Thus, a back-and-forth movement of the moveable member 805 can be obtained. By coupling the moveable member to a load or to the casing of the implantable vibration device, the implantable vibration device can be caused to vibrate.

In the embodiment shown in FIG. 27, the piezoelectric inertial motor MO is configured to generate linear motion may feature a speed in the range 1 mm/s to 10 mm/s, a stroke length in the range 1 mm-30 mm and a force in the range 2 N-30 N.

In alternative embodiments of piezoelectric inertial motors (not shown), the movable member 805 may be replaced with a with a rotary module such that the piezoelectric inertial motor is configured to generate rotary motion. Piezoelectric inertial motors configured to generate rotary motion may have a rotational speed in the range 1 mrad/s-100 mrad/s and a torque in the range 100 Nmm-900 Nmm. In such embodiments, the rotating piezoelectric inertial motor could be used as the rotational motor described with reference to FIGS. 30A-B.

Yet another design of a piezoelectric motor suitable for use in the implantable medical device described herein is the walk-drive motor. Walk-drive motors take their name from the fact that their working principle essentially resembles a walk. Linear motion is achieved through the coordinated and sequential action of a number of piezoelectric actuators acting as legs.

FIG. 28 shows an embodiment of a piezoelectric walk-drive motor MO configured to acts as at least part of a vibration generating unit VGU. The piezoelectric walk-drive motor MO comprises a number of piezoelectric actuators 801a-801d attached to a supporting member 800. The piezoelectric actuators 801a-801d may be divided into a first set 801a, 801c and a second set 801b, 801d. The first and second set 801a, 801c and 801b, 801d of piezoelectric actuators may be controlled independently. The piezoelectric walk-drive motor MO is configured to impart a linear motion to a movable member 805 by sequentially controlling the piezoelectric actuators 801a-801d. The movable member 805 is configured to be attached to the load or mechanism to be moved.

A piezoelectric walk-drive motor 805 may be operated in various operation modes, each offering specific advantages in terms of performance.

FIG. 29 illustrates an operation cycle of a piezoelectric walk-drive motor MO according to one embodiment. In this embodiment, the first set of piezoelectric actuators 801a, 801c is controlled by a first electrical voltage V₁. The second set of piezoelectric actuators 801b, 801d is controlled by a second electrical voltage V₂. The cycle comprises a number of steps.

In step 1, in response to a change in V₁, the first set of piezoelectric actuators 801a, 801c stretch and make contact with the movable member 805. When in contact, the first set of piezoelectric actuators 801a. 801c are bended sideways in a direction opposite to the motion direction D. Conversely, the second set of piezoelectric actuators 801b, 801d detach from the movable member 805 in response to a change in V₂.

In step 2, the first set of piezoelectric actuators 801a, 801c maintain contact with the movable member 805 and bend in the motion direction D in response to a change in V₁. The second set of piezoelectric actuators 801b, 801d remain detached from the movable member 805. As a result of the friction between the first set of piezoelectric actuators 801a, 801c and the movable member 805, the movable member 805 is moved in the motion direction D.

In step 3, in response to a change in V₂, the second set of piezoelectric actuators 801b, 801d stretch and make contact with the movable member 805. When in contact, the second set of piezoelectric actuators 801b, 801d are bended in a direction opposite to the motion direction D. Conversely, the first set of piezoelectric actuators 801a, 801c detach from the movable member 805 in response to a change in V₁.

In step 4, the second set of piezoelectric actuators 801b, 801d maintain contact with the movable member 805 and bend in the motion direction D in response to a change in V₂. The first set of piezoelectric actuators 801a, 801c remain detached from the movable member 805. As a result of the friction between the second set of piezoelectric actuators 801b, 801d and the movable member 805, the movable member 805 is moved in the motion direction D.

The piezoelectric actuators 801a-801d in FIG. 27 and FIG. 29 may be bimorph piezoelectric actuators. A bimorph actuator comprises at least two piezoelectric layers bonded together and oppositely responding to a given applied voltage. E.g., one layer extends and the other contracts. As a result, the bimorph actuator may stretch and bend. Alternatively, any of piezoelectric actuators 801a-801d in FIG. 28 and FIG. 29 may comprise a top part (illustrated as 801a′ in FIG. 28), configured to be attached to the supporting member 800, and a bottom part (exemplified by illustration as 801a″ in FIG. 27). The top part 801a′ is configured to deform perpendicularly to the motion direction D in response to the voltage applied to the piezoelectric actuator 801a. The bottom part 801a″ is configured to deform parallelly to the motion direction D in response to the voltage. As a result, the piezoelectric actuator 801a may stretch and bend.

By reversing the operation cycle, the piezoelectric walk-drive motor MO generates motion in the opposite direction. Thus, a back-and-forth movement of the moveable member 805 can be obtained. By coupling the moveable member to a load or to the casing of the implantable vibration device, the implantable vibration device can be caused to vibrate.

In the embodiment shown in FIGS. 28 and 29, the piezoelectric walk-drive motor MO is configured to generate linear motion at a speed in the range 1 mm/s to 10 mm/s and a force in the range 2 N-30 N. As the maximum stroke is limited by the length of the movable member 805 (also called a runner), there is no set limit for the maximum stroke.

In alternative embodiments of the piezoelectric walk-drive motor (not shown), the movable member 805 may be replaced with a with a rotary module such that the piezoelectric walk-drive motor is configured to generate rotary motion. Piezoelectric walk-drive motors configured to generate rotary motion may have a rotational speed in the range 0.5 mrad/s to around 70 mrad/s and a torque ranging from around 100 Nmm to around 900 Nmm. In such embodiments, the rotating piezoelectric walk-drive motor could be used as the rotational motor described with reference to FIGS. 30A-B.

FIGS. 30A-B schematically shows an implantable vibration device 910. FIGS. 30A and 30B differ in the shape of the casing 920. FIG. 30A shows a substantially cylindrical casing. FIG. 30B shows a substantially spherical casing. Of course, other casing shapes suitable for implantation in the human body are also contemplated.

The implantable vibration device comprises a casing 910 which contains a vibration generating unit VGU. Herein, the vibration generating unit VGU is based on an eccentric mechanism for causing the implantable vibration device to vibrate. The vibration generating unit comprises motor 604, a first motor axis 606, an eccentric element 608 eccentrically mounted to the first motor axis 606, a second axis 610 which suitably is supported by a bearing mounted to the casing 920. The vibration generating unit VGU may comprise a gear box 611 that transforms the speed of rotation of the motor 604 to a suitable speed.

Upon operation of the motor 201, the eccentric element 608 will rotate eccentrically about the first axis 606, to thereby cause the implantable vibration device to vibrate. The operation of the motor is preferably operated by the internal control unit Cr. The motor is preferably powered by energy received wirelessly by the energy transmitter T.

Another option for causing vibrations in the implantable vibration device it to mount a weight on the motor 604 via an axis 606, wherein said axis is attached to the weight at a position offset from the center of the weight. Upon rotation of the weight, the implantable vibration device will be made to vibrate.

All parts of the vibration generating unit VGU, including the motor 201 are preferably made of materials compatible with MRI scanning. Consequently, the VGU does not comprise any metallic and/or magnetic parts, and can instead by manufactured by polymeric and/or ceramic materials.

Alternatively, in other embodiments, the motor 604 can be an electromagnetic motor, such as a brushless DC motor.

In some embodiments, the motor 604 may be an inchworm motor with a rotatable module.

In some embodiments, the motor 604 may be a piezoelectric inertial motor with a rotary module.

In some embodiment, the motor 604 may be a walk-drive motor with a rotary module.

In some embodiments, the motor device 604 is a rotary ultrasonic motor, such as a traveling wave ultrasonic motor (TWUSM) shown in greater detail in FIG. 31 or a standing wave ultrasonic motor (SWUSM) shown in greater detail in FIG. 32.

A rotary ultrasonic motor is a piezoelectric ultrasonic motor configured to generate rotary motion. Rotary ultrasonic motors comprise traveling wave ultrasonic motors (TWUSM) and standing wave ultrasonic motor (SWUSM). In TWUSMs the stator vibrates according to a travelling wave pattern. In SWUSMs the stator vibrates according to a standing wave pattern.

FIG. 31 shows an embodiment of a TWUSM MO, configured to acts as at least part of a vibration generating unit VGU. The TWUSM MO comprises a ring-shaped stator 810 with a top and a bottom surface. The stator 810 is configured to engage with a ring-shaped rotor 811. The stator 810 comprises a ring-shaped member 810′, a first number of piezoelectric actuators 801a and a second number of piezoelectric actuators 801b. The piezoelectric actuators 801a, 801b are attached to the member 810′. An alternating electrical voltage V_{A} may be applied from a first voltage generator 812a (typically a controller connected to an energy source) to the piezoelectric actuators 801a. An alternating electrical voltage V_{B}, phase-shifted with respect to V_{A}, may be applied to the from a second voltage generator 812b to the second number of piezoelectric actuators 801b. The ring-shaped member 810′ may comprise a number of teeth 813. The rotor 811 is configured to be attached to the load or mechanism to be moved.

The first number of piezoelectric actuators 801a deform in response to the voltage V_{A} such that they induce a first vibration pattern in the stator 810. The second number of piezoelectric actuators 801b deform in response to the voltage V_{B} such that they induce a second vibration pattern in the stator 810. The interference of the first and second vibration pattern excites a travelling wave 814 in the stator 810. The travelling wave 814 has a given propagation direction D₁, either clock-wise or counter-clockwise. The regions of maximum displacement—so-called antinodes—and regions of no displacement—so-called nodes—of the travelling wave pattern oscillate transversely with respect to the top and bottom surface of the stator 810, but they also travel circumferentially along the stator 810 perimeter.

The propagation of the travelling wave 814 makes the stator vibrate accordingly. As a result, the stator 810 imparts a rotatory motion to the rotor 811 in a rotation direction D₂, opposite to the travelling wave 814 propagation direction D₁. The teeth 813 facilitate the motion transmission from the stator 810 to the rotor 811 by enhancing the friction between the rotor 811 and the stator 810.

The frequency and amplitude of the applied electrical voltages may be controlled and adjusted to tune the performance of the TWUSM MO, including speed, direction and accuracy of motion.

In contrast to TWUSMs, a standing wave ultrasonic motor (SWUSM) requires only a single alternating electrical voltage to operate. In response to this applied voltage, the piezoelectric actuators 801a, 801b of the stator 810 make the stator 810 vibrate according to a standing wave pattern. A standing wave is characterized by antinodes and nodes that do not travel in space. As a result, a standing wave does not have a propagation direction. The stator 810 vibrates in a way that antinodes and nodes oscillate transversely with respect to the top and bottom surface of the stator. However, antinodes and nodes do not travel circumferentially along the stator.

FIG. 32 shows an embodiment of a SWUSM, configured to acts as at least part of a vibration generating unit VGU. The SWUSM MO comprises a ring-shaped stator 810 with a top and a bottom surface. The stator 810 is configured to engage with a ring-shaped rotor 811. The stator 810 comprises a first set of piezoelectric actuators 801a and a second set of piezoelectric actuators 801b. An alternating electrical voltage may be selectively and exclusively applied to either set of piezoelectric actuators 801a, 801b while the other set is left floating. In such a case, the set to which the voltage is applied is referred to as active, while the other set is referred to as free.

The stator 810 comprises a number of protrusions 815. The stator 810 is configured to engage with the rotor 811 via the protrusions 815. The rotor 811 is configured to be attached to the load or mechanism to be moved.

A standing wave vibration pattern may be excited in the stator 810 in response to the applied voltage. As a result, the protrusions 815 oscillate at a first angle with respect to the top surface of the stator 810 when the piezoelectric actuators 801a are active. The protrusions 815 oscillate at a second angle with respect to the top surface of the stator 810 when the piezoelectric actuators 801b are active, with the second angle different from the first angle. The first angle is such that the stator 810 imparts a clockwise rotary motion to the rotor 811. The second angle is such that the stator 810 imparts a counter-clockwise rotary motion to the rotor 811.

In the embodiment shown in FIG. 32, the rotary ultrasonic motor MO has a rotational speed in the range 10 mrad/s-10000 mrad/s. and produces a torque in the range 20 Nmm-450 Nmm.

Rotary ultrasonic motors, such as the SWUSM or TWUSM configured to generate rotary motion could be used as the rotational electrical motor described with reference to FIGS. XX-YY

FIG. 33 shows an embodiment of a linear ultrasonic motor MO, configured to acts as at least part of a vibration generating unit VGU. The linear ultrasonic motor MO comprises a piezoelectric actuator 801, a pushing member 816, and a movable member 805 (or slider). The piezoelectric actuator 801 vibrates at its resonance frequency in response to an applied alternating voltage. The piezoelectric actuator 801 is attached to the pushing member 816. As a result of the vibrations in the piezoelectric actuator 801, the pushing member 816 alternatively contacts the movable member 805 and makes it move linearly by frictional coupling.

By reversing the operation cycle the moveable member can be caused to move in the opposite direction. Thus, a back-and-forth motion can be obtained by alternating the direction of the operation cycle. Back-and-forth motion of the moveable member 805 can be utilized to generate vibrations in an implantable vibration device, by attaching the moveable member 805 to a casing in an implantable vibration device as shown herein, to thereby transfer the vibrations to the casing.

In the embodiment shown in FIG. 33, the linear ultrasonic piezo motor MO is configured to generate linear motion with a speed in the range 4 mm/s to 100 mm/s and a force in the range 0.5 N-30 N. As the maximum stroke is limited by the length of the movable member 805 (also called a slider), there is no set limit for the maximum stroke.

FIG. 34 illustrates an alternative mechanism suitable for a vibration generating unit, which is also based on a piezoelectric motor. In FIG. 34, there is a shown a schematic side view of vibration generating unit VGU, suitable to be placed in an implantable vibration device 910 according to the present invention. FIG. 34 shows a simple design of a vibration generating mechanism which comprises a piezoelectric material 101 sandwiched between a respective electrode layers 902a, b, preferably comprised of copper foils. Each electrode layer comprises a tab which extends outside the footprint of the piezoelectric material, which allows for further electrical connections in the form of leads connected to the wireless energy receiver R and/or controller C_{I}. The energy transfer from the energy receiver R to the vibration generating unit is typically controlled by an internal controller C_{I}.

The piezoelectric material 101 is configured to extend in response to an applied electrical voltage controllable by the internal controller C_{I}, or by an external controller. Once the voltage is removed or reversed, the piezoelectric material 101 contracts to its relaxed state. Consequently, by alternatingly applying and removing (or reversing) a voltage over the piezoelectric material, the material can be made to expand and contract at a frequency which corresponds to the frequency of the alternating voltage. If this is performed at a certain frequency, the alternating expansion and contraction can cause an implantable vibration device to vibrate at a corresponding frequency.

The vibration generating unit is attached to the casing 920 of the implantable vibration device 910 via attachment means 904, such that the expansion and contraction movement of the piezoelectric material in the vibration generating unit can be transferred to the casing and thereby cause the implantable vibration device 910 to vibrate.

The electrode-piezoelectric material-electrode configuration is sandwiched between a pair of insulators 903a, b of e.g. alumina.

As mentioned above, the system shown in FIGS. 1-23 may comprise an energy source for providing the energy required to energize the stimulation device 200 and the stimulation arrangement, such as the electrode arrangement or vibration device, and thereby allow the stimulation signal to be applied to the nerve or effector tissue. Further, the system may comprise a control unit, or controller 240, as discussed above.

An energy receiver, which for example may comprise a coil arrangement configured to receive energy inductively, may also be provided. The energy receiver may be implanted in the body of the patient. The energy receiver may be integrated in one or more control units, or controllers. However, other arrangements are also possible, in which the energy receiver for example is arranged as a separate element that can be implanted at a different location than the control unit(s). In the latter case, the received energy may be transmitted to the control units(s), or stimulation device, or stimulation arrangement, by a wired connection extending between the energy receiver and the control unit/electrode arrangement.

The energy source may be implantable in the body or arranged outside the body. Similar to the energy receiver, the energy source may be configured to be operated on an inductive basis, in which the energy is transferred from the energy source to the energy receiver wirelessly. Hence, the energy source may comprise a coil arrangement enabling the inductive coupling to the energy receiver. It will be appreciated that the energy source further may comprise an energy storage, such as a primary or secondary cell, for storing electrical energy for transfer upon request. In case the energy source is implanted in the body, a non-rechargeable battery may require a surgical procedure for replacement, whereas a rechargeable battery may be recharged wirelessly/inductively from a charging source arranged outside the body. Beneficially, the latter allows the energy source to be recharged without requiring any surgical procedures.

Additionally, or alternatively, the system may comprise a control unit which is configured to transmit control instructions wirelessly to the stimulation device. The control unit may comprise an external part configured to be arranged outside the body of the patient, and an internal part configured to be implanted in the patient. The internal part and the external part may be configured to communicate wirelessly with each other, for example by means of radiofrequency signals or inductive signals. It will be appreciated that the internal part as well as the external part may form a control unit similar to what has been described above in connection with previous embodiments, and that they in some embodiments may be structurally integrated in the above described energy receiver and energy source. The wireless transmission of data from the internal external part to the external parts may, for instance, relate to sensor values indicating functional or status parameters of the implant or the patient. Examples of such parameters may for example include temperature of an implanted energy source, or another part of the implanted system or the body of the patient. Further examples include information indicating an effector response, such as a muscle contraction or an activity of a gland, or a nervous reaction triggered by the stimulation signal provided by the stimulation device. The internal and external parts may further be configured to transmit data relating to a status of an implanted energy source of the system, such as charging capacity, charge status, and the like.

A controller, or control unit, may thus be provided for controlling the implantable device. The control may require transmission of data, such as sensor values, operational parameters and ditto instructions, to and/or from the implanted devices and functions. In the following, various aspects and examples of such communication will be discussed.

The function and features of the controller comprised in the system for controlling the operation of the stimulation device 200 will now described with reference to FIGS. 35A-35F. The features of the controller described with reference to FIGS. 35A-35F may be implemented and combined with any of the embodiments of systems and devices disclosed herein. The features may for example be implemented in the controllers (300) shown and described with reference to FIGS. 35A-U. Any controller 300 may comprise an internal computing unit, also called a processor or processing unit, and it may comprise a communication unit and implement methods for communication, including verification, authentication and encryption of data, as described in the following.

The controller may comprise a collection of communication related sub-units such as a wired transceiver, a wireless transceiver, energy storage unit, an energy receiver, a computing unit, a memory, or a feedback unit. The sub-units of the controller may cooperate with each other or operate independently with different purposes. The sub-units of the controller may inherit the prefix “internal”. This is to distinguish these sub-units from the sub-units of the external devices as similar sub-units may be present for both the implanted controller and the external devices. The sub-units of the external devices may similarly inherit the prefix “external”.

A wireless transceiver may comprise both a wireless transmitter and a wireless receiver. The wireless transceiver may also comprise a first wireless transceiver and a second wireless transceiver. In this case, the wireless transceiver may be part of a first communication system (using the first wireless transceiver) and a second communication system (using the second wireless transceiver).

In some embodiments, two communication systems may be implemented using a single wireless transceiver in e.g. the implant and a single wireless transceiver in e.g. an external device (i.e. one antenna at the implant and one antenna at the external device), but where for example the network protocol used for data transmission from the external device to the implant is different from the network protocol used for data transmission from the implant to the external device, thus achieving two separate communication systems.

Alternatively, the wireless transceiver may be referred to as either a wireless transmitter or a wireless receiver as not all embodiments of secure wireless communication discussed herein require two-way communication capability of the wireless transceiver. The wireless transceiver may transmit or receive wireless communication via wireless connections. The wireless transceiver may connect to both the implant and to external devices, i.e. devices not implanted in the patient.

The wireless connections may be based on radio frequency identification (RFID), near field charge (NFC), Bluetooth, Bluetooth low energy (BLE), or wireless local area network (WLAN). The wireless connections may further be based on mobile telecommunication regimes such as 1G, 2G, 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

A wired transceiver may comprise both a wired transmitter and a wired receiver. The wording wired transceiver aims to distinguish between it and the wireless transceiver. It may generally be considered a conductive transceiver. The wired transceiver may transmit or receive conductive communication via conductive connections. Conductive connections may alternatively be referred to as electrical connections or as wired connections. The wording wired however, does not imply there needs to be a physical wire for conducting the communication. The body tissue of the patient may be considered as the wire. Conductive connection may use the body of the patient as a conductor. Conductive connections may still use ohmic conductors such as metals to at least some extent, and more specifically at the interface between the wired transceiver and the chosen conductor.

Communication, conductive or wireless may be understood as digital or analogue. In analogue communication, the message signal is in analogue form i.e., a continuous time signal. In digital communication, usually digital data i.e., discrete time signals containing information is transmitted.

The controller may comprise a sensation generator. A sensation generator is a device or unit that generates a sensation. The sensation generated may be configured to be experienceable by the patient such that the patient may take actions to authenticate a device, connection or communication. The sensation generator may be configured to generate a single sensation or a plurality of sensation components. The sensation or sensation components may comprise a vibration (e.g., a fixed frequency mechanical vibration), a sound (e.g. a superposition of fixed frequency mechanical vibrations), a photonic signal (e.g. a non-visible light pulse such as an infra-red pulse), a light signal (e.g. a visual light pulse), an electric signal (e.g. an electrical current pulse) or a heat signal (e.g. a thermal pulse). The sensation generator may be implanted, configured to be worn in contact with the skin of the patient or capable of creating sensation without being in physical contact with the patient, such as a beeping alarm.

The sensations generated by the sensation generator may be configured to be experienceable by a sensory function or a sense of the patient from the list of tactile, pressure, pain, heat, cold, taste, smell, sight, and hearing. Sensations may be generated of varying power or force as to adapt to sensory variations in the patient. Power or force may be increased gradually until the patient is able to experience the sensation. Variations in power or force may be controlled via feedback. Sensation strength or force may be configured to stay within safety margins. The sensation generator may be connected to the implant. The sensation generator may be comprised within the implant or be a separate unit.

A motor, e.g., of the active device or unit of the implant, for controlling a physical function in the body of the patient may provide a secondary function as a sensation generator, generating a vibration or sound. Generation of vibrations or sounds of the motor may be achieved by operating the motor at specific frequencies. When functioning as to generate a sensation the motor may operate outside of its normal ranges for frequency controlling a physical function in the body. The power or force of the motor when operating to generate a sensation may also vary from its normal ranges for controlling a physical function in the body. The motor for use as an active device and a sensation generator could for example be an implantable brushless DC motor with integrated gear box, such as the motors provided by Maxon group or Dr. Fritz Faulhaber.

An external device is a device which is external to the patient in which the implant is implanted in. The external device may also be enumerated (first, second, third, etc.) to separate different external devices from each other. Two or more external devices may be connected by means of a wired or wireless communication as described above, for example through IP (internet protocol), or a local area network (LAN). The wired or wireless communication may take place using a standard network protocol such as any suitable IP protocol (IPv4, IPv6) or Wireless Local Area Network (IEEE 802.11), Bluetooth, NFC, NFMI, RFID etc. The wired or wireless communication may take place using a proprietary network protocol. Any external device may also be in communication with the implant using wired or wireless communication according to the above. Communication with implanted devices may be thus accomplished with a wired connection, with wireless radiofrequency (RF) telemetry or near field magnetic induction (NFMI) technologies. Other methods of wireless communication may be used to communicate with implants, including optical and ultrasound. Alternatively, the concept of intrabody communication may be used for wireless communication, which uses the conductive properties of the body to transmit signals, i.e., conductive (capacitive or galvanic) communication with the implant. Means for conductive communication between an external device and an implant may also be called “electrical connection” between an external device and an implant. The conductive communication may be achieved by placing a conductive member of the external device in contact with the skin of the patient. By doing this, the external device and/or the implant may assure that it is in direct electrical connection with the other device. The concept relies on using the inherent conductive or electrical properties of a human body. Signals may preferably be configured to affect the body or body functions minimally. For conductive communication this may mean using low currents. A current may flow from an external device to an implant or vice versa. Also, for conductive communication, each device may have a transceiver portion for transmitting or receiving the current. These may comprise amplifiers for amplifying at least the received current. The current may contain or carry a signal which may carry e.g., an authentication input, implant operation instructions, or information pertaining to the operation of the implant.

Alternatively, conductive communication may be referred to as electrical or ohmic or resistive communication.

The conductive member may be an integrated part of the external device (e.g., in the surface of a smartwatch that is intended to be in contact with the wrist of the person wearing it), or it may be a separate device which can be connected to the external device using a conductive interface such as the charging port or the headphone port of a smartphone.

A conductive member may be considered any device or structure set up for data communication with the implant via electric conductive body tissue. The data communication to the implant may be achieved by e.g., current pulses transmitted from the conductive member through the body of the patient to be received by a receiver at the implant. Any suitable coding scheme known in the art may be employed. The conductive member may comprise an energy storage unit such as a battery or receive energy from e.g., a connected external device.

The term conductive interface is representing any suitable interface configured for data exchange between the conductive member and the external device. The conductive member may in an alternative configuration receive and transmit data to the external device through a radio interface, NFC, and the like.

An external device may act as a relay for communication between an implant and a remote device, such as e.g., second, third, or other external devices. Generally, the methods of relaying communication via an external device may be preferable for a large number of reasons. The transmission capabilities of the implant may be reduced, reducing its technical complexity, physical dimensions, and medical effects on the patient in which the implant is implanted. Communication may also be more efficient as direct communication, i.e., without a relaying device, with an implant from a remote device may require higher energy transmissions to account for different mediums and different rates of attenuation for different communication means. Remote communication with lower transmission energy may also increase the security of the communication as the spatial area or volume where the communication may be at all noticeable may be made smaller. Utilizing such a relay system further enables the use of different communication means for communication with the implant and communication with remote devices that are more optimized for their respective mediums.

An external device may be any device having processing power or a processor to perform the methods and functions needed to provide safe operation of the implant and provide the patient or other stakeholders (caregiver, spouse, employer etc.) with information and feedback from the implant. Feedback parameters could include battery status, energy level at the controller, number of operations that the stimulation device has performed, properties, version number etc. relating to functionality of the implantable medical device. The external device may for example be a handset such as a smartphone, smartwatch, tablet etc. handled by the patient or other stakeholders. The external device may be a server or personal computer handled by the patient or other stakeholders. The external device may be cloud based or a virtual machine. In the drawings, the external device handled by the patient is often shown as a smart watch, or a device adapted to be worn by the patient at the wrist of the patient. This is merely by way of example and any other type of external device, depending on the context, is equally applicable.

Several external devices may exist such as a second external device, a third external device, or another external device. The above listed external devices may e.g., be available to and controllable by a patient, in which an implant is implanted, a caregiver of the patient, a healthcare professional of the patient, a trusted relative of the patient, an employer or professional superior of the patient, a supplier or producer of the implant or its related features. By controlling the external devices may provide options for e.g., controlling or safeguarding a function of the implant, monitoring the function of the implant, monitoring parameters of the patient, updating or amending software of the implant etc.

An external device under control by a supplier or producer of the implant may be connected to a database comprising data pertaining to control program updates and/or instructions. Such database may be regularly updated to provide new or improved functionality of the implant, or to mitigate for previously undetected flaws of the implant. When an update of a control program of an implant is scheduled, the updated control program may be transmitted from the database in a push mode and optionally routed via one or more further external devices before received by the implanted controller. In another embodiment, the update is received from the database by request from e.g., an external device under control by the patient having the implant implanted in his/her body, a pull mode.

The external device may require authentication to be operated in communication with other external devices or the implant. Passwords, multi-factor authentication, biometric identification (fingerprint, iris scanner, facial recognition, etc.) or any other way of authentication may be employed.

The external device may have a user interface (UI) for receiving input and displaying information/feedback from/to a user. The UI may be a graphical UI (GUI), a voice command interface, speaker, vibrators, lamps, etc.

The communication between external devices, or between an external device and the implant may be encrypted. Any suitable type of encryption may be employed such as symmetric or asymmetric encryption. The encryption may be a single key encryption or a multi-key encryption. In multi-key encryption, several keys are required to decrypt encrypted data. The several keys may be called first key, second key, third key, etc. or first part of a key, second part of the key, third part of the key, etc. The several keys are then combined in any suitable way (depending on the encryption method and use case) to derive a combined key which may be used for decryption. In some cases, deriving a combined key is intended to mean that each key is used one by one to decrypt data, and that the decrypted data is achieved when using the final key.

In other cases, the combination of the several key result in one “master key” which will decrypt the data. In other words, it is a form of secret sharing, where a secret is divided into parts, giving each participant (external device(s), internal device) its own unique part. To reconstruct the original message (decrypt), a minimum number of parts (keys) is required. In a threshold scheme this number is less than the total number of parts (e.g., the key at the implant and the key from one of the two external device are needed to decrypt the data). In other embodiments, all keys are needed to reconstruct the original secret, to achieve the combined key which may decrypt the data.

In should be noted that it is not necessary that the generator of a key for decryption is the unit that in the end sends the key to another unit to be used at that unit. In some cases, the generator of a key is merely a facilitator of encryption/decryption, and the working on behalf of another device/user.

A verification unit may comprise any suitable means for verifying or authenticating the use (i.e., user authentication) of a unit comprising or connected to the verification unit, e.g. the external device. For example, a verification unit may comprise or be connected to an interface (UI, GUI) for receiving authentication input from a user. The verification unit may comprise a communication interface for receiving authentication data from a device (separate from the external device) connected to the device comprising the verification unit. Authentication input/data may comprise a code, a key, biometric data based on any suitable techniques such as fingerprint, a palm vein structure, image recognition, face recognition, iris recognition, a retinal scan, a hand geometry, and genome comparison, etc. The verification/authentication may be provided using third party applications, installed at or in connection with the verification unit.

The verification unit may be used as one part of a two-part authentication procedure. The other part may e.g., comprise conductive communication authentication, sensation authentication, or parameter authentication.

The verification unit may comprise a card reader for reading a smart card. A smart card is a secure microcontroller that is typically used for generating, storing and operating on cryptographic keys. Smart card authentication provides users with smart card devices for the purpose of authentication. Users connect their smart card to the verification unit. Software on the verification unit interacts with the keys material and other secrets stored on the smart card to authenticate the user. In order for the smart card to operate, a user may need to unlock it with a user-PIN. Smart cards are considered a very strong form of authentication because cryptographic keys and other secrets stored on the card are very well protected both physically and logically, and are therefore hard to steal.

The verification unit may comprise a personal e-ID that is comparable to, for example, passport and driving license. The e-ID system comprises is a security software installed at the verification unit, and a e-ID which is downloaded from a web site of a trusted provided or provided via a smart card from the trusted provider. The e-ID may comprise a hardware or a software key. The verification unit may comprise software for SMS-based two-factor authentication. Any other two-factor authentication systems may be used. Two-factor authentication requires two things to get authorized: something you know (your password, code, etc.) and something you have (an additional security code from your mobile device (e.g., a SMS, or a e-ID) or a physical token such as a smart card).

Other types of verification/user authentication may be employed. For example, a verification unit which communicate with an external device using visible light instead of wired communication or wireless communication using radio. A light source of the verification unit may transmit (e.g., by flashing in different patterns) secret keys or similar to the external device which uses the received data to verify the user, decrypt data or by any other means perform authentication. Light is easier to block and hide from an eavesdropping adversary than radio waves, which thus provides an advantage in this context. In similar embodiments, electromagnetic radiation is used instead of visible light for transmitting verification data to the external device.

Parameters relating to functionality of the implant may comprise for example a status indicator of the implant such as battery level, version of control program, properties of the implant, status of a motor of the implant, etc.

Data comprising operating instructions sent to the implant may comprise a new or updated control program, parameters relating to specific configurations of the implant, etc. Such data may for example comprise instructions how to operate the body engaging portion of the implantable medical device, instructions to collect patient data, instructions to transmit feedback, etc.

The expressions “confirming the electrical connection between an implant and an external device” or “authenticating a connection between an implant and an external device”, or similar expressions, are intended to encompass methods and processes for ensuring or be reasonably sure that the connection has not been compromised. Due to weaknesses in the wireless communication protocols, it is a simple task for a device to “listen” to the data and grab sensitive information, e.g., personal data regarding the patient sent from the implant, or even to try to compromise (hack) the implant by sending malicious commands or data to the implant. Encryption may not always be enough as a security measure (encryption schemes may be predictable), and other means of confirming or authenticating the external device being connected to the implant may be needed.

The expression “network protocol” is intended to encompass communication protocols used in computer networks, a communication protocol is a system of rules that allow two or more entities of a communications system to transmit information via any kind of variation of a physical quantity. The protocol defines the rules, syntax, semantics and synchronization of communication and possible error recovery methods. Protocols may be implemented by hardware, software, or a combination of both. Communication protocols have to be agreed upon by the parties involved. In this field, the term “standard” and “proprietary” is well defined. A communication protocol may be developed into a protocol standard by getting the approval of a standards organization. To get the approval the paper draft needs to enter and successfully complete the standardization process. When this is done, the network protocol can be referred to a “standard network protocol” or a “standard communication protocol”. Standard protocols are agreed and accepted by whole industry. Standard protocols are not vendor specific. Standard protocols are often, as mentioned above, developed by collaborative effort of experts from different organizations.

Proprietary network protocols, on the other hand, are usually developed by a single company for the devices (or Operating System) which they manufacture. A proprietary network protocol is a communications protocol owned by a single organization or individual. Specifications for proprietary protocols may or may not be published, and implementations are not freely distributed. Consequently, any device may not communicate with another device using a proprietary network protocol, without having the license to use the proprietary network protocol, and knowledge of the specifications for proprietary protocol. Ownership by a single organization thus gives the owner the ability to place restrictions on the use of the protocol and to change the protocol unilaterally.

A control program is intended to define any software used for controlling the implant. Such software may comprise an operating system of the implant, of parts of an operating system or an application running on the implant such as software controlling a specific functionality of the implant (e.g., the active unit of the implant, feedback functionality of the implant, a transceiver of the implant, encoding/decoding functionality of the implant, etc.). The control program may thus control the medical function of the implant, for example the pressure applied by a member or the power of the electrical stimulation device. Alternatively, or additionally, the control program may control internal hardware functionality of the implant such as energy usage, transceiver functionality, etc.

The systems and methods disclosed hereinabove may be implemented as software, firmware, hardware or a combination thereof. In a hardware implementation, the division of tasks between functional units referred to in the above description does not necessarily correspond to the division into physical units; to the contrary, one physical component may have multiple functionalities, and one task may be carried out by several physical components in cooperation. Certain components or all components may be implemented as software executed by a digital signal processor or microprocessor or be implemented as hardware or as an application-specific integrated circuit. Such software may be distributed on computer readable media, which may comprise computer storage media (or non-transitory media) and communication media (or transitory media). As is well known to a person skilled in the art, the term computer storage media includes both volatile and non-volatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information, and which can be accessed by a computer. Further, it is well known to the skilled person that communication media typically embodies computer readable instructions, data structures, program modules or other data in a modulated data signal such as a carrier wave or other transport mechanism and includes any information delivery media.

A controller 300 for controlling the medical device according to any of the embodiments herein and for communicating with devices external to the body of the patient and/or implantable sensors will now be described with reference to FIGS. 35A-35C. FIG. 35A shows a patient when a medical device 10 comprising a controller 300 has been implanted. The medical device 10 comprises an active unit 302, which is the part of the medical device which comprises the one or more members and operation device for operating the members etc. The active unit is directly or indirectly connected to the stomach wall of the patient for stretching the stomach wall for creating a sensation of satiety. The active unit 302 is connected to the controller 300 via an electrical connection C2. The controller 300 (further described with reference to FIG. 35B) is configured to communicate with an external device 320 (further described with reference to FIG. 35C). The controller 300 can communicate wirelessly with the external device 320 through a wireless connection WL1, and/or through an electrical connection C1.

Referring now to FIG. 35B, one embodiment of the controller 300 will be describe in more detail. The controller 300 comprises an internal computing unit 306 configured to control the function performed by the implantable medical device 10. The computing unit 306 comprises an internal memory 307 configured to store programs thereon. In the embodiment described in FIG. 35B, the internal memory 307 comprises a first control program 310 which can control the function of the medical device 10. The first control program 310 may be seen as a program with minimum functionality to be run at the medical device only during updating of the second control program 312. When the medical device is running with the first control program 310, the medical device may be seen as running in safe mode, with reduced functionality. For example, the first control program 310 may result in that no sensor data is stored in the medical device while being run, or that no feedback is transmitted from the medical device while the first control program 310 is running. By having a low complexity first control program, memory at the medical device is saved, and the risk of failure of the medical device during updating of the second control program 312 is reduced.

The second control program 312 is the program controlling the medical device in normal circumstances, providing the medical device with full functionality and features.

The memory 307 can further comprise a second, updatable, control program 312. The term updatable is to be interpreted as the program being configured to receive incremental or iterative updates to its code, or be replaced by a new version of the code. Updates may provide new and/or improved functionality to the implant as well as fixing previous deficiencies in the code. The computing unit 306 can receive updates to the second control program 312 via the controller 300. The updates can be received wirelessly WL1 or via the electrical connection C1. As shown in FIG. 35B, the internal memory 307 of the controller 300 can possibly store a third program 314. The third program 314 can control the function of the implantable medical device 10 and the computing unit 306 may be configured to update the second program 312 to the third program 314. The third program 314 can be utilized when rebooting an original state of the second program 312. The third program 314 may thus be seen as providing a factory reset of the controller 300, e.g., restore it back to factory settings. The third program 314 may thus be included in the implant 300 in a secure part of the memory 307 to be used for resetting the software (second control program 312) found in the controller 300 to original manufacturer settings.

The controller 300 may comprise a reset function 316 connected to or part of the internal computing unit 306 or transmitted to said internal computing unit 306. The reset function 316 is configured to make the internal computing unit 306 switch from running the second control program 312 to the first control program 310. The reset function 316 could be configured to make the internal computing unit 306 delete the second control program 312 from the memory 307. The reset function 316 can be operated by palpating or pushing/put pressure on the skin of the patient. This could be performed by having a button on the implant. Alternatively, the reset function 316 can be invoked via a timer or a reset module. Temperature sensors and/or pressure sensors can be utilized for sensing the palpating. The reset function 316 could also be operated by penetrating the skin of the patient. It is further plausible that the reset function 316 can be operated by magnetic means. This could be performed by utilizing a magnetic sensor and applying a magnetic force from outside the body. The reset function 316 could be configured such that it only responds to magnetic forces applied for a duration of time exceeding a limit, such as 2 seconds. The time limit could equally plausible be 5 or 10 seconds, or longer. In these cases, the implant could comprise a timer. The reset function 316 may thus include or be connected to a sensor for sensing such magnetic force.

In addition to or as an alternative to the reset function described above, the implant may comprise an internal computing unit 306 (comprising an internal processor) comprising the second control program 312 for controlling a function of the implantable medical device, and a reset function 318. The reset function 318 may be configured to restart or reset said second control program 312 in response to: i.e., a timer of the reset function 318 has not been reset, or ii. a malfunction in the first control program 310.

The reset function 318 may comprise a first reset function, such as, for example, comprise a computer operating properly, COP, function connected to the internal computing unit 306. The first reset function may be configured to restart or reset the first or the second control program 312 using a second reset function. The first reset function comprises a timer, and the first or the second control program is configured to periodically reset the timer.

The reset function 318 may further comprise a third reset function connected to the internal computing unit and to the second reset function. The third reset function may in an example be configured to trigger a corrective function for correcting the first 310 or second control program 312, and the second reset function is configured to restart the first 310 or second control program 312 sometime after the corrective function has been triggered. The corrective function may be a soft reset or a hard reset.

The second or third reset function may, for example, configured to invoke a hardware reset by triggering a hardware reset by activating an internal or external pulse generator which is configured to create a reset pulse. Alternatively, the second or third reset function may be implemented by software.

The controller 300 may further comprise an internal wireless transceiver 308. The transceiver 308 communicates wirelessly with the external device 320 through the wireless connection W1. The transceiver may further communicate with an external device 320, 300 via wireless connection WL2 or WL4. The transceiver may both transmit and receive data via either of the connections C1, WL1, WL2 and WL4. Optionally, the external devices 320 and 300, when present, may communicate with each other, for example via a wireless connection WL3.

The controller 300 can further be electrically connected C1 to the external device 320 and communicate by using the patient's body as a conductor. The controller 300 may thus comprise a wired transceiver 303 or an internal transceiver 303 for the electrical connection C1.

The confirmation/authentication of the electrical connection can be performed as described herein in the section for confirmation and/or authentication. In these cases, the implanted medical device and/or external device(s) 320 comprises the necessary features and functionality (described in the respective sections of this document) for performing such confirmation/authentication. By authenticating according to these aspects, security of the authentication may be increased as it may require a malicious third party to know or gain access to either the transient physiological parameter of the patient or detect randomized sensations generated at or within the patient.

In FIGS. 35A-35C the patient is a human, but other mammals are equally plausible. It is also plausible that the communication is performed by inductive means. It is also plausible that the communication is direct.

The controller 300 of the implantable medical device 10 according to FIG. 35B further comprises a feedback unit 349. The feedback unit 349 provides feedback related to the switching from the second control program 312 to the first control program 310. The feedback could for example represent the information on when the update of the software, i.e., the second control program 312, has started, and when the update has finished. This feedback can be visually communicated to the patient, via for example a display on the external device 320. This display could be located on a watch, or a phone, or any other external device 320 coupled to the controller 300. Preferably, the feedback unit 349 provides this feedback signal wirelessly WL1 to the external device 320. Potentially, the words “Update started”, or “Update finished”, could be displayed to the patient, or similar terms with the same meaning. Another option could be to display different colors, where green for example could mean that the update has finished, and red or yellow that the update is ongoing. Obviously, any color is equally plausible, and the user could choose these depending on personal preference. Another possibility would be to flash a light on the external device 320. In this case the external device 320 comprises the light emitting device(s) needed. Such light could for example be a LED. Different colors could, again, represent the status of the program update. One way of representing that the update is ongoing and not yet finished could be to flash the light, i.e., turning the light on and off. Once the light stops flashing, the patient would be aware of that the update is finished. The feedback could also be audible, and provided by the implantable medical device 300 directly, or by the external device 320. In such cases, the implantable medical device 10 and external device 320 comprises means for providing audio. The feedback could also be tactile, for example in the form of a vibration that the user can sense. In such case, either the implantable medical device 10 or external device 320 comprises means for providing a tactile sensation, such as a vibration and/or a vibrator.

As seen in FIG. 35B, the controller 300 can further comprise a first energy storage unit 40A. The first energy storage unit 40A runs the first control program 310. The controller 300 further comprises a second energy storage unit 40B which runs the second control program 312. This may further increase security during update, since the first control program 310 has its own separate energy storage unit 40A. The energy storage unit 40A can comprise a first energy storage 304a and/or a first energy receiver 305a. The second energy storage unit 40B can comprise a second energy storage 304b and/or a second energy receiver 305b. The energy can be received wirelessly by inductive or conductive means. An external energy storage unit can for example transfer an amount of wireless energy to the energy receiver 305a, 305b inside the patient's body by utilizing an external coil which induces a voltage in an internal coil (not shown in figures). It is plausible that the first energy receiver 305a receives energy via a RFID pulse. The feedback unit 349 can provide feedback pertaining to the amount of energy received via the RFID pulse. The amount of RFID pulse energy that is being received can be adjusted based on the feedback, such that the pulse frequency is successively raised until a satisfying level is reached.

The controller 300 of the medical device 10 according to FIG. 35B further comprises a feedback unit an electrical switch 309. The electrical switch 309 could be mechanically connected to a member of the medical device configured to exert a force on the stomach wall of the patient and being configured to be switched as a result of the force exerted on the stomach wall of the patient exceeding a threshold value. The switch 309 could for example be bonded to one of the members being connected to the stomach wall, in any of the embodiments herein, or to a portion of a fluid conduit, reservoir or hydraulic operation device, such as a pump, being in fluid connection with the member and be switched by the expansion, movement or bending of the member. The switch 309 could alternatively be electrically connected to the operation device and being configured to be switched as a result of the current supplied to the operation device exceeding a threshold value. The switch 309 could for example be connected to the motor and be configured to be switched if the current to the motor exceeds a threshold value. Such a switch could for example be a switch 309 configured to switch if exposed to a temperature exceeding a threshold value, such as a bimetal switch which is switched by the heat created by the flow of current to e.g., the motor. In the alternative, the switch 309 configured to switch if exposed to a temperature exceeding a threshold value could be placed at a different location on the medical device 10 to switch in case of exceeding temperatures, thereby hindering the medical device from overheating which may cause tissue damage.

The switch 309 could either be configured to cut the power to the operation device or to generate a control signal to the processor 306 of the implantable controller 300, such that the controller 300 can take appropriate action, such as reducing power or turning off the operation device.

The external device 320 is represented in FIG. 35C. The external device 320 can be placed anywhere on the patient's body, preferably on a convenient and comfortable place. The external device 320 could be a wristband, and/or have the shape of a watch. It is also plausible that the external device is a mobile phone or other device not attached directly to the patient. The external device as shown in FIG. 35C comprises a wired transceiver 323, and an energy storage 324. It also comprises a wireless transceiver 328 and an energy transmitter 325. It further comprises a computing unit 326 and a memory 327. The feedback unit 322 in the external device 320 is configured to provide feedback related to the computing unit 326. The feedback provided by the feedback unit 322 could be visual. The external device 320 could have a display showing such visual feedback to the patient. It is equally plausible that the feedback is audible, and that the external device 320 comprises means for providing audio. The feedback given by the feedback unit 322 could also be tactile, such as vibrating. The feedback could also be provided in the form of a wireless signal WL1, WL2, WL3, WL4.

The second, third or fourth communication methods WL2, WL3, WL4 may be a wireless form of communication. The second, third or fourth communication method WL2, WL3, WL4 may preferably be a form of electromagnetic or radio-based communication. The second, third and fourth communication method WL2, WL3, WL4 may be based on telecommunication methods. The second, third or fourth communication method WL2, WL3, WL4 may comprise or be related to the items of the following list: Wireless Local Area Network (WLAN), Bluetooth, Bluetooth 5, BLE, GSM or 2G (2nd generation cellular technology), 3G, 4G or 5G.

The external device 320 may be adapted to be in electrical connection C1 with the medical device 10, using the body as a conductor. The electrical connection C1 is in this case used for conductive communication between the external device 320 and the medical device 10.

In one embodiment, the communication between controller 300 and the external device 320 over either of the communication methods WL2, WL3, WL4, C1 may be encrypted and/or decrypted with public and/or private keys, now described with reference to FIGS. 35A-35C. For example, the controller 300 may comprise a private key and a corresponding public key, and the external device 320 may comprise a private and a corresponding public key.

The controller 320 and the external device 320 may exchange public keys and the communication may thus be performed using public key encryption. The person skilled in the art may utilize any known method for exchanging the keys.

The controller may encrypt data to be sent to the external device 320 using a public key corresponding to the external device 320. The encrypted data may be transmitted over a wired, wireless or electrical communication channel C1, WL1, WL2, WL3 to the external device. The external device 320 may receive the encrypted data and decode it using the private key comprised in the external device 320, the private key corresponding to the public key with which the data has been encrypted. The external device 320 may transmit encrypted data to the controller 300. The external device 320 may encrypt the data to be sent using a public key corresponding to the private key of the controller 300. The external device 320 may transmit the encrypted data over a wired, wireless or electrical connection C1, WL1, WL2, WL3, WL4, directly or indirectly, to the controller of the implant. The controller may receive the data and decode it using the private key comprised in the controller 300.

In an alternative to the public key encryption, described with reference to FIGS. 35a-35c, the data to be sent between the controller 300 of the implantable medical device 10 and an external device 320, 330 or between an external device 320, 330 and the controller 300 may be signed. In a method for sending data from the controller 300 to the external device 320, 330, the data to be sent from the controller 300 may be signed using the private key of the controller 300. The data may be transmitted over a communication channel or connection C1, WL1, WL2, WL3, WL4. The external device 320, 330 may receive the message and verify the authenticity of the data using the public key corresponding to the private key of the controller 300. In this way, the external device 320, 330 may determine that the sender of the data was sent from the controller 300 and not from another device or source.

A method for communication between external devices and the controller 300 of the implantable medical device 10 using a combined key is now described with reference to FIGS. 35a-35c. A first step of the method comprises receiving, at the implant, by a wireless transmission WL1, WL2, WL3, WL4 or otherwise, a first key from an external device 320, 330. The method further comprises receiving, at the implant, by a wireless transmission WL1, WL2, WL3, a second key. The second key may be generated by a second external device, separate from the external device 320, 330 or by another external device being a generator of the second key on behalf of the second external device 320, 330. The second key may be received at the medical device from anyone of, the external device 320, the second external device 330, and the generator of the second key. The second external device may be controlled by a caretaker, or any other stakeholder. Said another external device may be controlled by a manufacturer of the implant, or medical staff, caretaker, etc.

In case the controller 300 is receiving the second key from the external device 320, this means that the second key is routed through the external device from the second external device 330 or from another external device (generator). The routing may be performed as described herein under the tenth aspect. In these cases, the implanted medical device and/or external device(s) comprises the necessary features and functionality (described in the respective sections of this document) for performing such routing. Using the external device 320 as a relay, with or without verification from the patient, may provide an extra layer of security as the external device 320 may not need to store or otherwise handle decrypted information. As such, the external device 320 may be lost without losing decrypted information. The controller 300 a computing unit 306 configured for deriving a combined key by combining the first key and the second key with a third key held by the controller 300, for example in memory 307 of the controller 300. The third key could for example be a license number of the implant or a chip number of the implantable medical device. The combined key may be used for decrypting, by the computing unit 306, encrypted data transmitted by a wireless transmission WL1 from the external device 320 to the controller 300. Optionally, the decrypted data may be used for altering, by the computing unit 306 an operation of the implantable medical device. The altering an operation of the implantable medical device may comprise controlling or switching an active unit 302 of the implantable medical device. In some embodiments, the method further comprises at least one of the steps of, based on the decrypted data, updating a control program running in the controller 300, and operating the implantable medical device 10 using operation instructions in the decrypted data.

Methods for encrypted communication between an external device 320 and the controller 300 are provided. These methods may comprise:
receiving, at the external device 320, by a wireless transceiver 328, a first key, the first key being generated by a second external device 330, separate from the external device 320 or by another external device being a generator of the second key on behalf of the second external device 330, the first key being received from anyone of the second external device 330 and the generator of the second key,
receiving, at the external device 320 by the wireless transceiver 328, a second key from the controller 300,
deriving a combined key, by a computing unit 326 of the external device 320, by combining the first key and the second key with a third key held by the external device 320 (e.g., in memory 307),
transmitting encrypted data from the implant to the external device and receiving the encrypted data at the external device by the wireless transceiver 328, and decrypting, by the computing unit 326, the encrypted data, in the external device 320, using the combined key.

As described above, further keys may be necessary to decrypt the data. Consequently, the wireless transceiver 328 is configured for:
receiving a fourth key from a third external device,
wherein the computing unit 326 is configured for:
deriving a combined key by combining the first, second and fourth key with the third key held by the external device, and
decrypting the encrypted data using the combined key.

These embodiments further increase the security in the communication. The computing unit 326 may be configured to confirm the communication between the implantable medical device and the external device, wherein the confirmation comprises:
measuring a parameter of the patient, by the external device 320,
receiving a measured parameter of the patient, from the implantable medical device 10,
comparing the parameter measured by the implantable medical device 10 to the parameter measured by the external device 320,
performing confirmation of the connection based on the comparison, and
as a result of the confirmation, decrypting the encrypted data, in the external device, using the combined key.

The keys described in this section may in some embodiments be generated based on data sensed by sensors described herein under the twelfth or thirteenth aspect, e.g., using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

Further, increased security for communication between an external device(s) and the implantable medical device is provided.

A method of communication between an external device 320 and an implantable medical device 10 is now described with reference to FIGS. 35a-35c, when the implantable medical device 10 is implanted in a patient and the external device 320 is positioned external to the body of the patient. The external device 320 is adapted to be in electrical connection C1 with the controller 300, using the body as a conductor. The electrical connection C1 is used for conductive communication between the external device 320 and the implantable medical device 10. The implantable medical device 10 comprises the controller 300. Both the controller 300 and the external device 320 comprises a wireless transceiver 308, 208 for wireless communication C1 between the controller 300 and the external device 320. The wireless transceiver 308 (included in the controller 300) may in some embodiments comprise sub-transceivers for receiving data from the external device 320 and other external devices, e.g., using different frequency bands, modulation schemes etc.

In a first step of the method, the electrical connection C1 between the controller 300 and the external device 320 is confirmed and thus authenticated. The confirmation and authentication of the electrical connection may be performed as described herein under the fifth, thirteenth and fifteenth aspect. In these cases, the implant and/or external device(s) comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

The implant may comprise a first transceiver 303 configured to be in electrical connection C1 with the external device, using the body as a conductor. The implantable medical device may comprise a first external transmitter 203 configured to be in electrical connection C1 with the implantable medical device, using the body as a conductor, and the wireless transmitter 208 configured to transmit wireless communication W1 to the controller 300. The first transmitter 323 of the external device 320 may be wired or wireless. The first transmitter 323 and the wireless transmitter 208 may be the same or separate transmitters. The first transceiver 303 of the controller 300 may be wired or wireless. The first transceiver 303 and the wireless transceiver 102 may be the same or separate transceivers.

The controller 300 may comprise a computing unit 306 configured to confirm the electrical connection between the external device 320 and the internal transceiver 303 and accept wireless communication WL1 (of the data) from the external device 320 on the basis of the confirmation.

Data is transmitted from the external device 320 to the controller 300 wirelessly, e.g., using the respective wireless transceiver 308, 208 of the controller 300 and the external device 320. Data may alternatively be transmitted through the electrical connection C1. As a result of the confirmation, the received data may be used for instructing the implantable medical device 10. For example, a control program 310 running in the controller 300 may be updated, the controller 300 may be operated using operation instructions in the received data. This may be handled by the computing unit 306.

The method may comprise transmitting data from the external device 320 to the controller 300 wirelessly comprises transmitting encrypted data wirelessly. To decrypt the encrypted data (for example using the computing unit 306), several methods may be used.

In one embodiment, a key is transmitted using the confirmed conductive communication channel C1 (i.e., the electrical connection) from the external device 320 to the controller 300. The key is received at the controller (by the first internal transceiver 303). The key is then used for decrypting the encrypted data.

In some embodiments the key is enough to decrypt the encrypted data. In other embodiments, further keys are necessary to decrypt the data. In one embodiment, a key is transmitted using the confirmed conductive communication channel C1 (i.e., the electrical connection) from the external device 320 to the controller 300. The key is received at the controller 300 (by the first internal transceiver 303). A second key is transmitted (by the wireless transceiver 208) from the external device 320 using the wireless communication WL1 and received at the controller 300 by the wireless transceiver 308. The computing unit 306 is then deriving a combined key from the key and second key and uses this for decrypting the encrypted data.

In yet other embodiments, a key is transmitted using the confirmed conductive communication channel C1 (i.e., the electrical connection) from the external device 320 to the controller 300. The key is received at the controller (by the first internal transceiver 303). A third key is transmitted from a second external device 330, separate from the external device 320, to the implant wirelessly WL2. The third key may be received by a second wireless receiver (part of the wireless transceiver 308) of the controller 300 configured for receiving wireless communication WL2 from second external device 330.

The first and third key may be used to derive a combined key by the computing unit 306, which then decrypts the encrypted data. The decrypted data is then used for instructing the implantable medical device 10 as described above.

The second external device 330 may be controlled by for example a caregiver, to further increase security and validity of data sent and decrypted by the controller 300.

It should be noted that in some embodiments, the external device is further configured to receive WL2 secondary wireless communication from the second external device 330, and transmit data received from the secondary wireless communication WL2 to the implantable medical device. This routing of data may be achieved using the wireless transceivers 308, 208 (i.e., the wireless connection WL1, or by using a further wireless connection WL4 between the controller 300 and the external device 320. In these cases, the medical device and/or external device(s) comprises the necessary features and functionality for performing such routing. Consequently, in some embodiments, the third key is generated by the second external device 330 and transmitted WL2 to the external device 320 which routes the third key to the controller 300 to be used for decryption of the encrypted data. In other words, the step of transmitting a third key from a second external device, separate from the external device, to the implant wirelessly, comprises routing the third key through the external device 320. Using the external device 320 as a relay, with or without verification from the patient, may provide an extra layer of security as the external device 320 may not need to store or otherwise handle decrypted information. As such, the external device 320 may be lost without losing decrypted information.

In yet other embodiments, a key is transmitted using the confirmed conductive communication channel C1 (i.e., the electrical connection) from the external device 320 to the controller 300. The key is received at the implant (by the first internal transceiver 303). A second key is transmitted from the external device 320 to the controller 300 wirelessly WL1, received at the at the controller 300. A third key is transmitted from the second external device, separate from the external device 320, to the controller 300 wirelessly WL4. Encrypted data transmitted from the external device 320 to the controller 300 is then decrypted using a derived combined key from the key, the second key and the third key. The external device may be a wearable external device.

The external device 320 may be a handset. The second external device 330 may be a handset. The second external device 330 may be a server. The second external device 330 may be cloud based.

In some embodiments, the electrical connection C1 between the external device 320 and the controller 300 is achieved by placing a conductive member 201, configured to be in connection with the external device 200, in electrical connection with a skin of the patient for conductive communication C1 with the medical device. In these cases, the medical device and/or external device(s) comprises the necessary features and functionality (described in the respective sections of this document) for performing such conductive communication. The communication may thus be provided with an extra layer of security in addition to the encryption by being electrically confined to the conducting path e.g., external device 320, conductive member 201, conductive connection C1, controller 300, meaning the communication will be excessively difficult to be intercepted by a third party not in physical contact with, or at least proximal to, the patient.

The keys described in this section may in some embodiments be generated based on data sensed by sensors described herein, e.g., using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

Increased security for communication between an external device(s) and an implanted medical device is provided, now described with reference to FIGS. 35a-35c.

In these embodiments, a method for communication between an external device 320 and the implantable controller 300 is provided. The wireless transceiver 308 (included in the controller 300) may in some embodiments comprise sub-transceivers for receiving data from the external device 320 and other external devices 330, e.g., using different frequency bands, modulation schemes etc.

A first step of the method comprises receiving, at the implanted medical device, by a wireless transmission WL1 or otherwise, a first key from an external device 320. The method further comprises receiving, at the implanted medical device, by a wireless transmission WL1. WL2, WL3, a second key. The second key may be generated by a second external device 330, separate from the external device 320 or by another external device being a generator of the second key on behalf of the second external device 330. The second key may be received at the implanted medical device from anyone of, the external device 320, the second external device 330, and a generator of the second key. The second external device 330 may be controlled by a caretaker, or any other stakeholder. Said another external device may be controlled by a manufacturer of the medical device, or medical staff, caretaker, etc.

In case the medical device is receiving the second key from the external device 320, this means that the second key is routed through the external device from the second external device 330 or from the another external device (generator). In these cases, the medical device and/or external device(s) comprises the necessary features and functionality (described in the respective sections of this document) for performing such routing. Using the external device 320 as a relay, with or without verification from the patient, may provide an extra layer of security as the external device 320 may not need to store or otherwise handle decrypted information. As such, the external device 320 may be lost without losing decrypted information.

The controller 300 comprises a computing unit 306 configured for deriving a combined key by combining the first key and the second key with a third key held by the controller 300, for example in memory 307 of the controller. The combined key may be used for decrypting, by the computing unit 306, encrypted data transmitted by a wireless transmission WL1 from the external device 320 to the controller 300. Optionally, the decrypted data may be used for altering, by the computing unit 306 an operation of the implantable medical device 10. The altering an operation of the implantable medical device may comprise controlling or switching an active unit 302 of the medical device. In some embodiments, the method further comprises at least one of the steps of, based on the decrypted data, updating a control program running in the implant, and operating the implantable medical device 10 using operation instructions in the decrypted data.

In some embodiments, further keys are necessary to derive a combined key for decrypting the encrypted data received at the controller 300. In these embodiments, the first and second key are received as described above. Further, the method comprises receiving, at the implanted medical device, a fourth key from a third external device, the third external device being separate from the external device, deriving a combined key by combining the first, second and fourth key with the third key held by the controller 300, and decrypting the encrypted data, in the controller 300, using the combined key. Optionally, the decrypted data may be used for altering, by the computing unit 306, an operation of the implanted medical device as described above. In some embodiments, the fourth key is routed through the external device from the third external device.

In some embodiments, further security measures are needed before using the decrypted data for altering, by the computing unit 306, an operation of the implantable medical device. For example, an electrical connection C1 between the implantable medical device and the external device 320, using the body as a conductor, may be used for further verification of validity of the decrypted data. The electrical connection C1 may be achieved by placing a conductive member 201, configured to be in connection with the external device, in electrical connection with a skin of the patient for conductive communication C1 with the implantable medical device. The communication may thus be provided with an extra layer of security in addition to the encryption by being electrically confined to the conducting path e.g. external device 320, conductive member 201, conductive connection C1, controller 300, meaning the communication will be excessively difficult to be intercepted by a third party not in physical contact with, or at least proximal to, the patient.

Accordingly, in some embodiments, the method comprising confirming the electrical connection between the controller 300 and the external device 320, and as a result of the confirmation, altering an operation of the implantable medical device based on the decrypted data. The confirmation and authentication of the electrical connection may be performed as described herein under the general features section. In these cases, the implantable medical device and/or external device(s) 320 comprises the necessary features and functionality (described in the respective sections of this document) for performing such authentication. By authenticating according to these aspects, security of the authentication may be increased as it may require a malicious third party to know or gain access to either the transient physiological parameter of the patient or detect randomized sensations generated at or within the patient.

In some embodiments, the confirmation of the electrical connection comprises: measuring a parameter of the patient, by e.g. a sensor of the implantable medical device 10, measuring the parameter of the patient, by the external device 320, comparing the parameter measured by the implantable medical device to the parameter measured by the external device 320, and authenticating the connection based on the comparison. As mentioned above, as a result of the confirmation, an operation of the implantable medical device may be altered based on the decrypted data.

Further methods for encrypted communication between an external device 320 and an implantable medical device 10 are provided. These methods comprise:
receiving, at the external device 320 by a wireless transceiver 328, a first key, the first key being generated by a second external device 330, separate from the external device 320 or by another external device being a generator of the second key on behalf of the second external device 320, the first key being received from anyone of the second external device 330 and the generator of the second key,
receiving, at the external device 320 by the wireless transceiver 328, a second key from the controller 300,
deriving a combined key, by a computing unit 326 of the external device 320, by combining the first key and the second key with a third key held by the external device 320 (e.g. in memory 327),
transmitting encrypted data from the implant to the external device and receiving the encrypted data at the external device by the wireless transceiver 328, and
decrypting, by the computing unit 326, the encrypted data, in the external device 320, using the combined key.

As described above, further keys may be necessary to decrypt the data. Consequently, the wireless transceiver 328 is configured for:
receiving a fourth key from a third external device,
wherein the computing unit 326 is configured for:
deriving a combined key by combining the first, second and fourth key with the third key held by the external device, and
decrypting the encrypted data using the combined key.

In some embodiments, the communication between the controller 300 and the external device 320 needs to be confirmed (authenticated) before decrypting the data. In these cases, the implantable medical device and/or external device(s) comprises the necessary features and functionality (described in the respective sections of this document) for performing such authentication.

These embodiments further increase the security in the communication. In these embodiments the computing unit 326 is configured to confirm the communication between the implantable medical device and the external device, wherein the confirmation comprises: measuring a parameter of the patient, by the external device 320,
receiving a measured parameter of the patient, from the implantable medical device 10,
comparing the parameter measured by the implantable medical device 320 to the parameter measured by the external device 320,
performing confirmation of the connection based on the comparison, and
as a result of the confirmation, decrypting the encrypted data, in the external device, using the combined key.

One or more of the first, second and third key may comprise a biometric key.

The keys described in this section may in some embodiments be generated based on data sensed by sensors, e.g. using the sensed data as seed for the generated keys. A seed is an initial value that is fed into a pseudo random number generator to start the process of random number generation. The seed may thus be made hard to predict without access or knowledge of the physiological parameters of the patient which it is based on, providing an extra level of security to the generated keys.

Further, increased security for communication between an external device(s) 320, 330 and an implantable medical device is provided, described with reference to FIGS. 35a-35c. The system being configured for enabling communication between an external device 320 and the controller 300 implanted in a patient. The system comprises a conductive member 321 configured to be in connection (electrical/conductive or wireless or otherwise) with the external device, the conductive member 321 being configured to be placed in electrical connection with a skin of the patient for conductive communication C1 with the implantable medical device 10. By using a conductive member 321 as defined herein, an increased security for communication between the external device and the implantable medical device may be achieved. For example, when a sensitive update of a control program of the controller 300 is to be made, or if sensitive data regarding physical parameters of the patient is to be sent to the external device 320 (or otherwise), the conductive member 321 may ensure that the patient is aware of such communication and actively participate in validating that the communication may take place. The conductive member may, by being placed in connection with the skin of the patient, open the conductive communication channel C1 between the external device and the controller to be used for data transmission.

Electrical or conductive communication, such as this or as described under the other embodiments, may be very hard to detect remotely, or at least relatively so, in relation to wireless communications such as radio transmissions. Direct electrical communication may further safeguard the connection between the implantable medical device 10 and the external device 320 from electromagnetic jamming i.e. high-power transmissions other a broad range of radio frequencies aimed at drowning other communications within the frequency range. Electrical or conductive communication will be excessively difficult to be intercepted by a third party not in physical contact with, or at least proximal to, the patient, providing an extra level of security to the communication.

In some embodiments, the conductive member comprises a conductive interface for connecting the conductive member to the external device.

In some embodiments, the conductive member 201 is a device which is plugged into the external device 200, and easily visible and identifiable for simplified usage by the patient. In other embodiments, the conductive member 321 is to a higher degree integrated with the external device 320, for example in the form of a case of the external device 320 comprising a capacitive area configured to be in electrical connection with a skin of the patient. In one example, the case is a mobile phone case (smartphone case) for a mobile phone, but the case may in other embodiments be a case for a personal computer, or a body worn camera, or any other suitable type of external device as described herein. The case may for example be connected to the phone using a wire from the case and connected to the headphone port or charging port of the mobile phone.

The conductive communication C1 may be used both for communication between the controller 300 and the external device 320 in any or both directions. Consequently, according to some embodiments, the external device 320 is configured to transmit a conductive communication (conductive data) to the controller 300 via the conductive member 321.

According to some embodiments, the controller 300 is configured to transmit a conductive communication to the external device 320. These embodiments start by placing the conductive member 321, configured to be in connection with the external device 320, in electrical connection with a skin of the patient for conductive communication C1 with the controller 300. The conductive communication between the external device 320 and the controller 300 may follow an electrically/conductively confined path comprising e.g. the external device 320, conductive member 321, conductive connection C1, controller 300.

For the embodiments when the external device 320 transmits data to the controller, the communication may comprise transmitting a conductive communication to the controller 300 by the external device 320.

The transmitted data may comprise instructions for operating the implantable medical device 10. Consequently, some embodiments comprise operating the implantable medical device 10 using operation instructions, by an internal computing unit 306 of the controller 300, wherein the conductive communication C1 comprises instructions for operating the implantable medical device 10. The operation instruction may for example involve adjusting or setting up (e.g. properties or functionality of) the active unit 302 of the implantable medical device 10.

The transmitted data may comprise instructions for updating a control program 310 stored in memory 307 of the controller 300. Consequently, some embodiments comprise updating the control program 310 running in the controller 300, by the internal computing unit 306 of the implantable medical device, wherein the conductive communication comprises instructions for updating the control program 310.

For the embodiments when the controller 300 transmits data to the external device 320, the communication may comprise transmitting conductive communication C1 to the external device 320 by the controller 300. The conductive communication may comprise feedback parameters. Feedback parameters could include battery status, energy level at the controller, the effector response induced by the stimulation signal, number of operations or cycles that the vibration device has performed, properties, version number etc. relating to functionality of the implantable medical device 10. In other embodiments, the conductive communication C1 comprises data pertaining to least one physiological parameter of the patient, such as blood pressure etc. The physiological parameter(s) may be stored in memory 307 of the controller 300 or sensed in prior (in real time or with delay) to transmitting the conductive communication C1. Consequently, in some embodiments, the implantable medical device 10 comprises a sensor 150 for sensing at least one physiological parameter of the patient, wherein the conductive communication comprises said at least one physiological parameter of the patient.

To further increase security of the communication between the controller 300 and the external device 320, different types of authentication, verification and/or encryption may be employed. In some embodiments, the external device 320 comprises a verification unit 340. The verification unit 340 may be any type of unit suitable for verification of a user, i.e. configured to receive authentication input from a user, for authenticating the conductive communication between the implantable medical device and the external device. In some embodiments, the verification unit and the external device comprises means for collecting authentication input from the user (which may or may not be the patient). Such means may comprise a fingerprint reader, a retina scanner, a camera, a GUI for inputting a code, a microphone, device configured to draw blood, etc. The authentication input may thus comprise a code or any be based on a biometric technique selected from the list of: a fingerprint, a palm vein structure, image recognition, face recognition, iris recognition, a retinal scan, a hand geometry, and genome comparison. The means for collecting the authentication input may alternatively be part of the conductive member which comprise any of the above examples of functionality, such as a fingerprint reader or other type of biometric reader. In some embodiments, the security may thus be increased by receiving an authentication input from a user by the verification unit 340 of the external device 320, and authenticating the conductive communication between the controller 300 and the external device using the authentication input. Upon a positive authentication, the conductive communication channel C1 may be employed for comprising transmitting a conductive communication to the controller 300 by external device 320 and/or transmitting a conductive communication to the external device 320 by the controller 300. In other embodiments, a positive authentication is needed prior to operating the implantable medical device 10 based on received conductive communication, and/or updating a control program running in the controller 300 as described above.

FIGS. 35a-35c further shows an implantable medical device 10 implanted in a patient and being connected to a sensation generator 381.

The sensation generator 381 may be configured to generate a sensation. The sensation generator 381 may be contained within the implantable medical device 10 or be a separate unit. The sensation generator 381 may be implanted. The sensation generator 381 may also be located so that it is not implanted as such but still is in connection with a patient so that only the patient may experience sensations generated. The controller 300 is configured for storing authentication data, related to the sensation generated by the sensation generator 381.

The controller 300 is further configured for receiving input authentication data from the external device 320. Authentication data related to the sensation generated may by stored by a memory 307 of the controller 300. The authentication data may include information about the generated sensation such that it may be analyzed, e.g. compared, to input authentication data to authenticate the connection, communication or device. Input authentication data relates to information generated by a patient input to the external device 320. The input authentication data may be the actual patient input or an encoded version of the patient input, encoded by the external device 320. Authentication data and input authentication data may comprise a number of sensations or sensation components.

The authentication data may comprise a timestamp. The input authentication data may comprise a timestamp of the input from the patient. The timestamps may be a time of the event such as the generation of a sensation by the sensation generator 381 or the creation of input authentication data by the patient. The timestamps may be encoded. The timestamps may feature arbitrary time units, i.e. not the actual time. Timestamps may be provided by an internal clock 360 of the controller 300 and an external clock 362 of the external device 320. The clocks 360, 362 may be synchronized with each other. The clocks 360, 362 may be synchronized by using a conductive connection C1 or a wireless connection WL1 for communicating synchronization data from the external device 320, and its respective clock 362, to the controller 300, and its respective clock 360, and vice versa. Synchronization of the clocks 360, 362 may be performed continuously and may not be reliant on secure communication.

Authentication of the connection may comprise calculating a time difference between the timestamp of the sensation and the timestamp of the input from the patient, and upon determining that the time difference is less than a threshold, authenticating the connection. An example of a threshold may be 1 s. The analysis may also comprise a low threshold as to filter away input from the patient that is faster than normal human response times. The low threshold may e.g. be 50 ms. Authentication data may comprise a number of times that the sensation is generated by the sensation generator, and wherein the input authentication data comprises an input from the patient relating to a number of times the patient detected the sensation. Authenticating the connection may then comprise: upon determining that the number of times that the authentication data and the input authentication data are equal, authenticating the connection.

A method of authenticating the connection between an implantable medical device 10 implanted in a patient, and an external device 320 according includes the following steps.

Generating, by a sensation generator 381, a sensation detectable by a sense of the patient. The sensation may comprise a plurality of sensation components. The sensation or sensation components may comprise a vibration (e.g. a fixed frequency mechanical vibration), a sound (e.g. a superposition of fixed frequency mechanical vibrations), a photonic signal (e.g. a non-visible light pulse such as an infra-red pulse), a light signal (e.g. a visual light pulse), an electric signal (e.g. an electrical current pulse) or a heat signal (e.g. a thermal pulse). The sensation generator may be implanted, configured to be worn in contact with the skin of the patient or capable of creating sensation without being in physical contact with the patient, such as a beeping alarm.

Sensations may be configured to be consistently felt by a sense of the patient while not risking harm to or affecting internal biological processes of the patient.

The sensation generator 381, may be contained within the controller 300 or be a separate entity connected to the controller 300. The sensation may be generated by a motor (denoted as M in several embodiments shown herein) of the implantable medical device 10, wherein the motor being the sensation generator 381. The sensation may be a vibration, or a sound created by running the motor. The sensation generator 381 may be located close to a skin of the patient and thus also the sensory receptors of the skin. Thereby the strength of some signal types may be reduced.

Storing, by the controller 300, authentication data, related to the generated sensation.

Providing, by the patient input to the external device, resulting in input authentication data. Providing the input may e.g. comprise an engaging an electrical switch, using a biometric input sensor or entry into digital interface running on the external device 320 to name just a few examples.

Transmitting the input authentication data from the external device to the controller 300. If the step was performed, the analysis may be performed by the controller 300.

Transmitting the authentication data from the implantable medical device 10 to the external device 320. If the step was performed, the analysis may be performed by the external device 320. The wireless connection WL1 or the conductive connection C1 may be used to transmit the authentication data or the input authentication data.

Authenticating the connection based on an analysis of the input authentication data and the authentication data e.g. by comparing a number of sensations generated and experienced or comparing timestamps of the authentication data and the input authentication data. If step was performed, the analysis may be performed by the implantable medical device 10.

Communicating further data between the controller 300 and the external device 320 following positive authentication. The wireless connection WL1 or the conductive connection C1 may be used to communicate the further data. The further data may comprise data for updating a control program 310 running in the controller 300 or operation instructions for operating the implantable medical device 10. The further data may also comprise data sensed by a sensor 150 connected to the controller 300. The controller may comprise at least one unit having a sleep mode and an active mode, and the unit consumes less energy in the sleep mode than in the active mode. The unit is configured to switch from the sleep mode to the active mode on the basis of at least one signal from the sensor. The unit could for example be a DSP (Digital Signal Processor), another type of processor or a wake-up circuit of the controller, which in turn activates the functions of the controller. The unit may be configured to switch from the sleep mode to the active mode on the basis of a signal from the sensor related to the patient swallowing a number of times and/or on the basis of a signal from the sensor related to the patient swallowing a number of times during a time period. The number of times the patient swallows and the time could be counted/measured and compared with a pre-set or moving threshold value. The controller could further comprise at least one filtering unit configured to filter signals related to at least one of: speech, the swallowing of saliva and chewing. The filter could be a digital filter implemented as hardware or software in the controller and could have the filter characteristics of a high, low or bandpass filter.

If the analysis was performed by the controller 300, the external device 320 may continuously request or receive, information of an authentication status of the connection between the controller 300 and the external device 320, and upon determining, at the external device 320, that the connection is authenticated, transmitting further data from the external device 320 to the controller 300.

If the analysis was performed by the external device 320, the controller 300 may continuously request or receive, information of an authentication status of the connection between the controller 300 and the external device 320, and upon determining, at the controller 300, that the connection is authenticated, transmitting further data from the controller 300 to the external device 320.

A main advantage of authenticating a connection according to this method is that only the patient may be able to experience the sensation. Thus, only the patient may be able to authenticate the connection by providing authentication input corresponding to the sensation generation.

The sensation generator 381, sensation, sensation components, authentication data, input authentication data, and further data may be further described herein. In these cases, the implantable medical device 10 and/or external device(s) comprises the necessary features and functionality (described in the respective sections of this document). Further information and definitions can be found in this document in conjunction with the other aspects.

The method may further comprise transmitting further data between the controller 300 and the external device, wherein the further data is used or acted upon, only after authentication of the connection is performed.

The analysis or step of analyzing may be understood as a comparison or a step of comparing.

In one method, increased security for communication between an external device(s) and an implanted controller is provided. FIGS. 35a-35c show an implantable medical device 10 comprising a controller 300 and an external device 320 which may form a system.

The controller 300 comprises a transceiver 308, 303 configured to establish a connection with an external device 320, i.e. with a corresponding transceiver 328, 323. The connection may be an electrical connection C1 using the transceivers 303, 323, or a wireless connection WL1 using the transceivers 308, 328. The controller 300 further comprises a computing unit 306 configured to verify the authenticity of instructions received at the transceiver 308, 303 from the external device 320. In this aspect, the concept of using previously transmitted instructions for verifying a currently transmitted instructions are employed. Consequently, the transmitting node (in this case the external device) need to be aware of previously instructions transmitted to the implantable medical device, which reduces the risk of a malicious device instructing the implant without having the authority to do so.

In an embodiment, the computing unit 306 is configured to verify the authenticity of instructions received at the transceiver 308, 303 by extracting a previously transmitted set of instructions from a first combined set of instructions received by the transceiver. The external device 320 may thus comprise an external device comprising a computing unit 326 configured for: combining a first set of instructions with a previously transmitted set of instructions, forming a combined set of instructions, and transmitting the combined set of instructions to the implantable medical device. The previously transmitted set of instructions, or a representation thereof, may be stored in memory 327 of the external device 320.

The combined set of instructions may have a data format which facilitates such extraction, for example including metadata identifying data relating to the previously transmitted set of instructions in the combined set of instructions. In some embodiments, the combined set of instructions comprises the first set of instructions and a cryptographic hash of the previously transmitted set of instructions. Consequently, the method comprises combining, at the external device, a first set of instructions with a previously transmitted set of instructions, forming a first combined set of instructions. A cryptographic hash function is a special class of hash function that has certain properties which make it suitable for use in cryptography. It is a mathematical algorithm that maps data of arbitrary size to a bit string of a fixed size (a hash) and is designed to be a one-way function, that is, a function which is infeasible to invert. Examples include MD5, SHA1, SHA 256, etc. Increased security is thus achieved.

The first combined set of instructions is then transmitted to the implanted controller 300, where it is received by e.g. the transceiver 303, 308. The first combined set of instructions may be transmitted to the implantable medical device using a proprietary network protocol. The first combined set of instructions may be transmitted to the controller 300 using a standard network protocol. In these cases, the controller 300 and/or external device(s) comprises the necessary features and functionality (described in the respective sections of this document) for performing transmission of data. By using different communication protocols, at the external device 320, for communication with the controller 300 and with a second external device 330, an extra layer of security is added as the communication between controller 300 and the external device 320 may be made less directly accessible to remote third parties.

At the controller 300, the computing unit 306 verifies the authenticity of the received first combined set of instructions, by: extracting the previously transmitted set of instructions from the first combined set of instructions, and comparing the extracted previously transmitted set of instructions with previously received instructions stored in the implantable medical device. Upon determining that the extracted previously transmitted set of instructions equals the previously received instructions stored in the controller 300, the authenticity of the received first combined set of instructions may be determined as valid, and consequently, the first set of instructions may be safely run at the controller 300, and the first combined set of instructions may be stored in memory 307 of the controller 300, to be used for verifying a subsequent received set of instructions. In some embodiments, upon determining by the internal computing unit 306 that the extracted previously transmitted set of instructions differs from the previously received instructions stored in the controller 300, feedback related to an unauthorized attempt to instruct the implantable medical device 10 may be provided. For example, the transceiver 308, 303 may send out a distress signal to e.g. the external device 320 or to any other connected devices. The controller 300 may otherwise inform the patient that something is wrong by e.g. vibration or audio. The implantable medical device 10 may be run in safe mode, using a preconfigured control program which is stored in memory 307 of the controller 300 and specifically set up for these situations, e.g. by requiring specific encoding to instruct the implantable medical device 10, or only allow a predetermined device (e.g. provided by the manufacturer) to instruct the implantable medical device 10. In some embodiments, when receiving such feedback at the external device 320, the external device 320 retransmits the first combined set of instructions again, since the unauthorized attempt may in reality be an error in transmission (where bits of the combined set of instructions are lost in transmission), and where the attempt to instruct the implantable medical device 10 is indeed authorized.

The step of comparing the extracted previously transmitted set of instructions with previously received instructions stored in the controller 300 may be done in different ways. For example, the step of comparing the extracted previously transmitted set of instructions with previously received instructions stored in the controller 300 comprises calculating a difference between the extracted previously transmitted set of instructions with previously received instructions stored in the controller 300, and comparing the difference with a threshold value, wherein the extracted previously transmitted set of instructions is determined to equal the previously received instructions stored in the controller 300 in the case of the difference value not exceeding the threshold value. This embodiment may be used when received instructions is stored in clear text, or a representation thereof, in the controller 300, and where the combined set of instructions, transmitted from the external device also includes such a representation of the previously transmitted instructions. This embodiment may be robust against error in transmission where bits of information are lost or otherwise scrambled.

In other embodiments, the combined set of instructions comprises the first set of instructions and a cryptographic hash of the previously transmitted set of instructions, wherein the method further comprises, at the controller 300, calculating a cryptographic hash of the previously received instructions stored in the controller 300 and comparing the calculated cryptographic hash to the cryptographic hash included in the first combined set of instructions. This embodiment provides increased security since the cryptographic hash is difficult to decode or forge.

The above way of verifying the authenticity of received instructions at the controller 300 may be iteratively employed for further sets if instructions.

To further increase security, the transmission of a first set of instructions, to be stored at the controller 300 for verifying subsequent sets of combined instructions, where each set of received combined instructions will comprise data which in some form will represent, or be based on, the first set of instruction, may be performed.

In one example, the external device 320 may be adapted to communicate with the controller 300 using two separate communication methods. A communication range of a first communication method WL1 may be less than a communication range of a second communication method WL2. A method may comprise the steps of: sending a first part of a key from the external device 320 to the controller 300, using the first communication method WL1 and sending a second part of the key from the external device 320 to the controller 300, using the second communication method WL2. The method may further comprise deriving, in the controller 300, a combined key from the first part of the key and the second part of the key and decrypting the encrypted data, in the controller 300, using the combined key. The encrypted data may also be sent from the external device 320 to the controller 300 using the second communication method WL2. The method may then further comprise confirming an electrical connection C1 between the controller 300 and the external device 320 and as a result of the confirmation, decrypting the encrypted data in the controller 300 and using the decrypted data for instructing the controller 300.

The method may also comprise placing a conductive member 321, configured to be in connection with the external device 320, in electrical connection with a skin of the patient for conductive communication with the controller 300. By means of the electrical connection an extra layer of security is added as a potential hacker would have to be in contact with the patient to access or affect the operation of the implantable medical device 10.

Using a plurality of communication methods, may increase the security of the authentication and the communication with the implantable medical device 10 as more than one channel for communication may need to be hacked or hijacked by an unauthorized entity to gain access to the implantable medical device 10 or the communication.

The electrical connection C1 the conductive member 321 and conductive communication may be further described herein in the general definitions section. In these cases, the controller 300 and/or external device 320 comprise the necessary features and functionality (described in the respective sections of this document).

It should also be noted that any one of the first and second communication methods WL1. WL2 may be needed to be confirmed in order to decrypt the encrypted data in the controller 300 and using the decrypted data for instructing the implantable medical device 10.

The method may further comprise the step of wirelessly receiving, at the controller 300, a third part of the key from the second external device 330. In this case, the combined key may be derived from the first part of the key, the second part of the key and the third part of the key.

The first communication method WL1 may be a wireless form of communication. The first communication method WL1 may preferably be a form of electromagnetic or radio-based communication however, other forms of communication are not excluded. The first communication method WL1 may comprise or be related to the items of the following list: Radio-frequency identification (RFID), Bluetooth, Bluetooth 5, Bluetooth Low Energy (BLE), Near Field Communication (NFC), NFC-V, Infrared (IR) based communication, Ultrasound based communication.

RFID communication may enable the use of a passive receiver circuit such as those in a RFID access/key or payment card. IR based communication may comprise fiber optical communication and IR diodes. IR diodes may alternatively be used directly, without a fiber, such as in television remote control devices. Ultrasound based communication may be based on the non-invasive, ultrasound imaging found in use for medical purposes such as monitoring the development of mammal fetuses.

The first communication method WL1 may use a specific frequency band. The frequency band of the first communication method WL1 may have a center frequency of 13.56 MHz or 27.12 MHz. These bands may be referred to as industrial, scientific and medical (ISM) radio bands. Other ISM bands not mentioned here may also be utilized for the communication methods WL1, WL2. A bandwidth of the 13.56 MHz centered band may be 14 kHz and a bandwidth of the 27.12 MHz centered band may be 326 kHz.

The communication range of the first communication method WL1 may be less than 10 meters, preferably less than 2 meters, more preferably less than 1 meter and most preferably less than 20 centimeters. The communication range of the first communication method WL1 may be limited by adjusting a frequency and/or a phase of the communication. Different frequencies may have different rates of attenuation. By implementing a short communication range of the first communication method, security may be increased since it may be ensured or made probable that the external device is under control of the patient (holding the external device close to the implant)

The communication range of the first communication method WL1 should be evaluated by assuming that a patient's body, tissue, and bones present the propagation medium. Such a propagation medium may present different attenuation rates as compared to a free space of an air-filled atmosphere or a vacuum.

By restricting the communication range, it may be established that the external device communicating with the implanted controller 300 is in fact on, or at least proximal to, the patient. This may add extra security to the communication.

The second communication method WL2 may be a wireless form of communication. The second communication method WL2 may preferably be a form of electromagnetic or radio-based communication. The second communication method WL2 may be based on telecommunication methods. The second communication method WL2 may comprise or be related to the items of the following list: Wireless Local Area Network (WLAN), Bluetooth, Bluetooth 5, BLE, GSM or 2G (2nd generation cellular technology), 3G, 4G, 5G.

The second communication method WL2 may utilize the ISM bands as mentioned in the above for the first communication method WL1.

A communication range of the second communication method WL2 may be longer than the communication range of the first communication method WL1. The communication range of the second communication method WL2 may preferably be longer than 10 meters, more preferably longer than 50 meters, and most preferably longer than 100 meters.

Encrypted data may comprise instructions for updating a control program 310 running in the implantable medical device 10. Encrypted data may further comprise instructions for operating the implantable medical device 10.

In one embodiment, the implantable medical device 10 may transmit data to an external device 320 which may add an additional layer of encryption and transmit the data to a second external device 330, described with reference to FIGS. 35a-35c. By having the external device add an additional layer of encryption, less computing resources may be needed in the implanted controller 300, as the controller 300 may transmit unencrypted data or data encrypted using a less secure or less computing resource requiring encryption. In this way, data can still be relatively securely transmitted to a third device. The transmission of data can be performed using any of the method described herein in addition to the method or in the system described below.

Thus, in an embodiment, a system is provided. The system comprises an implantable medical device 10 comprising a controller 300 configured to transmit data from the body of the patient to an external device 320, and an encryption unit 382 for encrypting the data to be transmitted. The system further comprises an external device 320 configured to receive the data transmitted by the controller 300, encrypt the received data using a first key and transmit the encrypted received data to a third external device 330. The encryption can be performed using any of the keys described above or below. In some embodiments, the external device 320 is configured to decrypt the data received from the controller 300 before encrypting and transmitting the data. Alternatively, the external device 320 may encrypt and transmit the data received from the controller 300 without decrypting it first.

In one example, the encryption unit 382 is configured to encrypt the data to be transmitted using a second key. The first key or the second key may, for example, information specific to the implantable medical device 10, a secret key associated with the external device 320, an identifier of the implantable medical device 10 or an identifier of the controller 300. The second key could be a key transmitted by the external device 320 to the controller 300. In some examples, the second key is a combined key comprising a third key received by the controller 300 from the external device 320.

The first key may be a combined key comprising a fourth key, wherein the fourth key is received by the external device 320 from a fourth device. The fourth device may be a verification unit, either comprised in the external device, or external to the external device and connected to it. The verification unit may have a sensor 350 for verification, such as a fingerprint sensor. More details in regard to this will be described below. Alternatively, the verification unit may be a generator, as described above.

The system may be configured to perform a method for transmitting data using a sensed parameter. The method may comprise transmitting a parameter measured by the external device 320 from the external device 320 to the controller 300. In this case, the comparison of the parameter of the patient measured by the external device 320 and the parameter of the patient measured by the controller 300 may be performed by the controller 300. The implantable medical device 10 may comprise a first sensor 150 for measuring the parameter of the patient at the implantable medical device 10. The external device 320 may comprise an external sensor 350 for measuring the parameter of the patient at the external device 320.

Authentication of the connection between the controller 300 and the external device 320 may be performed automatically without input, authentication, or verification from a user or patient. This is because the comparison of parameters measured internally and externally, by the internal and external sensors 351, 350 respectively may be enough to authenticate the connection. This may typically be the case when the parameter of the patient is related to an automatically occurring physiological function of the patient such as e.g. a pulse of the patient. Certain types of authentication may however require actions from the patient, e.g. having the patient perform specific movements.

In the embodiments described herein, the controller 300 may comprise or be connected to a sensation generator 381 as described above. In response to an event in the implantable medical device, such as a reset, a restart, receipt of new instructions, receipt of a new configuration or update, installation or activation of new instructions or configuration or update, the controller 300 may be configured to cause the sensation generator 381 to generate a sensation detectable by the patient in which the implantable medical device 10 is implanted. In some examples, the user may after the sensation verify an action, for example via a user interface of an external device 320. The implantable medical device 10 may further implement a method for improving the security of the data transmitted from the controller 300. The method, for encrypted communication between a controller 300, when implanted in a patient's body, and an external device 320, comprises encoding or encrypting, by the controller 300 or a processor 306 comprised in or connected to the controller 300, data relating to the implantable medical device 10 or the operation thereof; transmitting, by the controller 300, the data; receiving, by a second communication unit comprised the external device 320, the data; encrypting, by the external device 320, the data using an encryption key to obtain encrypted data; and transmitting the encrypted data to a third external device 330. In this way, the external device 320 may add or exchange the encryption, or add an extra layer of encryption, to the data transmitted by the controller 300. When the controller 300 encodes the data to be transmitted it may be configured to not encrypt the data before transmitting, or only using a light-weight encryption, thus not needing as much processing power as if the controller were to fully encrypt the data before the transmission.

The encrypting, by the controller 300, may comprise encrypting the data using a second key. The encryption using the second key may be a more light-weight encryption than the encryption performed by the external device using the second key, i.e. an encryption that does not require as much computing resources as the encryption performed by the external device 320. The first or the second key may comprise a private key exchanged as described above with reference to encryption and authentication, or the first or the second key may comprise an information specific to the implantable medical device 10, a secret key associated with the external device, an identifier of the implantable medical device 10 or an identifier of the controller 300. They may be combined keys as described in this description, and the content of the keys, any combination of keys, and the exchange of a key or keys is described in the encryption and/or authentication section.

In an embodiment, the implantable medical device 10 comprises at least one sensor for sensing at least one physiological parameter of the patient or a functional parameter of the implantable medical device 10, now described with reference to FIGS. 35a-35c. The sensor 351 may, for example, be a pressure sensor, an electrical sensor, a clock, a temperature sensor, a motion sensor, an optical sensor, an acoustic sensor, an ultrasonic sensor. The sensor 351 is configured to periodically sense the parameter and the controller 300 is configured to, in response to the sensed parameter being above a predetermined threshold, wirelessly broadcast information relating to the sensed parameter. The controller 300 may be configured to broadcast the information using a short to mid-range transmitting protocol, such as a Radio Frequency type protocol, a RFID type protocol, a WLAN type protocol, a Bluetooth type protocol, a BLE type protocol, an NFC type protocol, a 3G/4G/5G type protocol, or a GSM type protocol.

The controller of the implant may be connected to the sensor 351 and be configured to anonymize the information before it is transmitted. The transmission of data may also be called broadcasting of data.

In addition to or as an alternative to transmitting the data when the sensed parameter is above a predetermined threshold, the controller 300 may be configured to broadcast the information periodically. The controller 300 may be configured to broadcast the information in response to a second parameter being above a predetermined threshold. The second parameter may, for example, be related to the controller 300 itself, such as a free memory or free storage space parameter, or a battery status parameter. When the implantable medical device 10 comprises an implantable energy storage unit and an energy storage unit indicator, the energy storage unit indicator is configured to indicate a functional status of the implantable energy storage unit and the indication may be comprised in the transmitted data. The functional status may indicate at least one of charge level and temperature of the implantable energy storage unit.

In some embodiments the external device 320 is configured to receive the broadcasted information, encrypt the received information using an encryption key and transmit the encrypted received information. In this way, the external device 320 may add an additional layer of encryption or exchange the encryption performed by the controller 300.

In an embodiment, the controller 300 is configured to transmit the data using the body of the patient as a conductor C1, and the external device 320 is configured to receive the data via the body. Alternatively, or in combination, the controller 300 of the implant is configured to transmit the data wirelessly to the external device WL2.

Thus, the controller 300 may implement a method for transmitting data from the controller 300 comprising a processor 306, comprising: obtaining sensor measurement data via a sensor 150 connected to or comprised in the controller 300, the sensor measurement relating to at least one physiological parameter of the patient or a functional parameter of the implantable medical device 10, and transmitting by the controller 300 the sensor measurement data in response to the sensor measurement being above a predetermined threshold, wherein the sensor 150 is configured to periodically sense the parameter. The method may further comprise broadcasting the sensor measurement data, to be received by an external device 320. The transmitting or broadcasting may comprise using at least one of a Radio Frequency type protocol, RFID type protocol, WLAN type protocol, Bluetooth type protocol, BLE type protocol, NFC type protocol, 3G/4G/5G type protocol, or a GSM type protocol.

The method may further comprise, at the processor 306, anonymizing, by the processor, the sensor measurement data before it is transmitted, or encrypting the sensor measurement data, using an encryptor 382 comprised in the processing unit 306, before it is transmitted. The transmitting of the data may further comprise to encode the data before the transmitting. The type of encoding may be dependent on the communication channel or the protocol used for the transmission.

The transmitting may be performed periodically, or in response to a signal received by the processor, for example, by an internal part of the implantable medical device 10 such as a sensor 150, or by an external device 320.

The parameter may, for example, be at least one of a functional parameter of the implantable medical device 10 (such as a battery parameter, a free memory parameter, a temperature, a pressure, an error count, a status of any of the control programs, or any other functional parameter mentioned in this description) or a parameter relating to the patient (such as a temperature, a blood pressure, or any other parameter mentioned in this description). In one example, the implantable medical device 10 comprises an implantable energy storage unit 40 and an energy storage unit indicator 304c, and the energy storage unit indicator 304c is configured to indicate a functional status of the implantable energy storage unit 40, and the sensor measurement comprises data related to the energy storage unit indicator.

In one example, the transmitting comprises transmitting the sensor measurement to an internal processor 306 configured to cause a sensation generator 381 to cause a sensation detectable by the patient in which the implantable medical device 100 is implanted.

The method may be implemented in a system comprising the implantable medical device 100 and an external device 320, and further comprise receiving the sensor measurement data at the external device 320, and, at the external device 320, encrypting the sensor measurement data using a key to obtain encrypted data, and, transmitting the encrypted data. The transmitting may, for example, be performed wirelessly WL3 or conductively C1.

In the examples or embodiments transmitting data from or to the implantable medical device 10, the following method may be implanted in order to verify the integrity of the data, described with reference to FIGS. 35a-35b. By verifying the integrity of the data, an external device 320 or a processor 306 comprised in the controller 300 may verify that the data has not been corrupted or tampered with during the transmission. In some examples, data integrity for data communicated between a controller 300 and an external device 320 or between an external device 320 and the controller 300 may be performed using a cyclic redundancy check.

Thus, in a first example, a method for evaluating a parameter of a controller 300 implanted in a patient is described. The controller 300 comprises a processor 306 and a sensor 150 for measuring the parameter. The method comprises measuring, using the sensor 150, the functional parameter to obtain measurement data; establishing a connection between the internal controller 300 and an external device 320 configured to receive data from the implant; determining, by the processor 306, a cryptographic hash or a metadata relating to the measurement data and adapted to be used by the external device 320 to verify the integrity of the received data; transmitting the cryptographic hash or metadata; and transmitting, from the controller 300, the measurement data.

The parameter may, for example, be a parameter of the controller 300, such as a temperature, a pressure, a battery status indicator, a time period length, an effector response, or a physiological parameter of the patient, such as a pulse, a blood pressure, or a temperature. In some examples, multiple parameters may be used.

The method may further comprise evaluating the measurement data relating to the functional parameter. By evaluating it may be meant to determine if the parameter is exceeding or less than a predetermined value, to extract another parameter from the measurement data, compare the another parameter to a predetermined value, or displaying the another parameter to a user. For example, the method may further comprise, at the external device 320, to determining, based on the evaluating, that the implantable medical device 10 is functioning correctly, or determining based on the evaluating that the implantable medical device 10 is not functioning correctly.

If it is determined that the implantable medical device 10 is not functioning correctly, the method may further comprise sending, from the external device 320, a corrective command to the controller 300, receiving the corrective command at the controller 300, and by running the corrective command correcting the functioning of the implantable medical device 10 according to the corrective command.

The method may further comprise, at the external device 320, receiving the transmitted cryptographic hash or metadata, receiving the measurement data, and verifying the integrity of the measurement data using the cryptographic hash or metadata. The cryptographic hash algorithm be any type of hash algorithm, i.e. an algorithm comprising a one-way function configured to have an input data of any length as input and produce a fixed-length hash value. For example, the cryptographic hash algorithm may be MD5, SHA1, SHA 256, etc.

In some examples, the cryptographic hash is a signature obtained by using a private key of the controller 300, and wherein the verifying, by the external device 320, comprises verifying the signature using a public key corresponding to the private key.

When using a cryptographic hash, the method may further comprise calculating a second cryptographic hash for the received measurement data using a same cryptographic hash algorithm as the processor, and determining that the measurement data has been correctly received based on that the cryptographic hash and the second cryptographic hash are equal (i.e. have the same value).

When using a metadata the verifying the integrity of the data may comprises obtaining a second metadata for the received measurement data relating to the functional parameter, and determining that the data has been correctly received based on that metadata and the second metadata are equal. The metadata may, for example, be a length of the data or a timestamp. In some examples the measurement data is transmitted in a plurality of data packets. In those examples, the cryptographic hash or metadata comprises a plurality of cryptographic hashes or metadata each corresponding to a respective data packet, and the transmitting of each the cryptographic hashes or metadata is performed for each of the corresponding data packets.

A similar method may be utilized for communicating instructions from an external device 320 to a controller 300 implanted in a patient. The method comprises establishing a first connection between the external device 320 and the controller 300, establishing a second connection between a second external device 330 and the controller 300, transmitting, from the external device 320, a first set of instructions to the controller 300 over the first connection, transmitting, from the second external device 330, a first cryptographic hash or metadata corresponding to the first set of instructions to the controller 300, and, at the controller 300, verifying the integrity of the first set of instructions and the first cryptographic hash or metadata, based on the first cryptographic hash or metadata. The external device 320 may be separate from the second external device 330.

The first connections may be established between the controller 300 and a transceiver of the external communication unit 323. In some examples, the communication using the second connection is performed using a different protocol than a protocol used for communication using the first communication channel. In some examples, the first connection is a wireless connection and the second connection is an electrical connection. The second connection may, for example, be an electrical connection using the patient's body as a conductor (using 321). The protocols and ways of communicating may be any communication protocols described in this description with reference to C1, and WL1-WL4. The establishing of the first and second connections are performed according to the communication protocol used for each of the first and the second connections.

When using a cryptographic hash, the verifying the integrity of the first set of instructions may comprise calculating a second cryptographic hash for the received first set of instructions using a same cryptographic hash algorithm as the processor 306, and determining that the first set of instructions has been correctly received based on that the cryptographic hash and the second cryptographic hash are equal. The cryptographic hash may, for example, be a signature obtained by using a private key of the implantable medical device 10, and wherein the verifying comprises verifying the signature using a public key corresponding to the private key. In some examples, the cryptographic hash is a signature obtained by using a private key of the implantable medical device 10, and wherein the verifying comprises verifying the signature using a public key corresponding to the private key. The private keys and public keys, as well as the exchange or transmittal of keys have been described in this description. Alternatively, other well-known methods can be used for transmitting or exchanging a key or keys between the external device 320 and the controller 300. When using a metadata, and wherein the verifying the integrity of the data may comprise obtaining a second metadata for the received first set of instructions, and determining that the first set of instructions has been correctly received based on that metadata and the second metadata are equal. The metadata may, for example, be any type of data relating to the data to be transmitted, in this example the first set of instructions. For example, the metadata may be a length of the data to be transmitted, a timestamp on which the data was transmitted or retrieved or obtained, a size, a number of packets, or a packet identifier.

In some examples, the controller 300 may transmit data to an external device 320 relating to the data information in order to verify that the received data is correct. The method may thus further comprise, transmitting, by the controller 300, information relating to the received first set of instructions, receiving, by the external device 320, the information, and verifying, by the external device 320, that the information corresponds to the first set of instructions sent by the external device 320. The information may, for example, comprise a length of the first set of instructions. The method may further comprise, at the controller 300, verifying the authenticity of the first set of instructions by i. calculating a second cryptographic hash for the first set of instructions, ii. comparing the second cryptographic hash with the first cryptographic hash, iii. determining that the first set of instructions are authentic based on that the second cryptographic hash is equal to the first cryptographic hash, and upon verification of the authenticity of the first set of instructions, storing them at the controller 300.

In some examples, the first set of instructions comprises a cryptographic hash corresponding to a previous set of instruction, as described in other parts of this description. In some examples, the first set of instructions may comprise a measurement relating to the patient of the body for authentication, as described in other parts of this description.

A system and a method for communication of instructions or control signals between an external device 320 and an implantable medical device 10 will now be described with reference to FIGS. 35a-35c.

The system shown in FIGS. 35a-35c comprises an implantable medical device 10, a first external device 320, and a second external device 330. The implantable medical device a controller 300. The controller 300 is adapted to receive an instruction from an external device 320 over the communication channel WL1, C1 and run the instruction to control a function of the medical device 10. The communication channel WL1. C1 may be any type of communication channel, such as a wireless connection WL1 or a conductive connection C1 described herein. For example, the wireless connection may comprise at least one of the following protocols: Radio Frequency type protocol, RFID type protocol, WLAN type protocol, Bluetooth type protocol, a BLE type protocol, an NFC type protocol, a 3G/4G/5G/6G type protocol, a GSM type protocol, and/or Bluetooth 5.

The first external device 320 is adapted to receive, such as through a user interface, or determine an instruction to be transmitted to the implantable medical device 10. The determination of the instruction may, for example, be based on received data from the implantable medical device 10, such as measurement data or data relating to a state of the implantable medical device 10, such as a battery status or a free memory status. The first external device 320 may be any type of device capable of transmitting information to the implantable medical device and capable of determining or receiving an instruction to be transmitted to the implantable medical device 10. In a preferred embodiment, the first external device 320 is a hand-held device, such as a smartphone, smartwatch, tablet etc. handled by the patient, having a user interface for receiving an instruction from a user, such as the patient or a caregiver.

The first external device 320 is further adapted to transmit the instruction to a second external device 330 via communication channel WL3. The second external device 320 is adapted to receive the instruction, encrypt the instruction using an encryption key, and then transmit the encrypted instruction to the implantable medical device 10. The implantable medical device 10 is configured to receive the instruction at the controller 300. The controller 300 thus comprises a wired transceiver or a wireless transceiver for receiving the instruction. The implantable medical device 10 is configured to decrypt the received instruction. The decryption may be performed using a decryption key corresponding to the encryption key. The encryption key, the decryption key and methods for encryption/decryption and exchange of keys may be performed as described in the “general definition of features” or as described with reference to FIGS. 35a-35c. Further, there are many known methods for encrypting data which the skilled person would understand to be usable in this example.

The second external device 330 may be any computing device capable of receiving, encrypting and transmitting data as described above. For example, the second external device 320 may be a network device, such as a network server, or it may be an encryption device communicatively coupled to the first external device.

The instruction may be a single instruction for running a specific function or method in the implantable medical device 10, a value for a parameter of the implantable medical device 10, or a set of sub-steps to be performed by the controller 300 comprised in the implantable medical device 10.

In this way, the instruction for controlling a function of the implantable medical device 10 may be received at the first external device 320 and transmitted to the implantable medical device 10 via the second external device 330. By having a second external device 330 encrypting the instruction before transmitting it to the implantable medical device 10, the instruction may be verified by the second external device 330 and the first external device 320 may function so as to relay the instruction. In some alternatives, the second external device 330 may transmit the instruction directly to the implantable medical device 10. This may provide an increased security as the instruction sent to the implantable medical device 10 may be verified by the second external device 330, which, for example, may be a proprietary device managed by the medical professional responsible for the implantable medical device 10. Further, by having the second external device 330 verifying and encrypting the instruction, the responsibility authenticity and/or correctness of the instruction may lie with the second external device 330, which may be beneficial for regulatory purposes, as the first external device 320 may not be considered as the instructor of the implantable medical device 10.

Further, the second external device 330 may verify that the instruction is correct before encrypting or signing and transmitting it to the implantable medical device 10. The second external device 330 may, for example, verify that the instruction is correct by comparing the instruction with a predetermined set of instructions, and if the instruction is comprised in the predetermined set of instructions determine that the instruction is correct. If the instruction comprises a plurality of sub-steps, the second external device 330 may determine that the instruction is correct if all the sub-steps are comprised in the predetermined set of instructions. If the instruction comprises a value for a parameter of the implantable medical device 10, the second external device 330 may verify that the value is within a predetermined range for the parameter. The second external device 320 may thus comprise a predetermined set of instructions, or a predetermined interval or threshold value for a value of a parameter, stored at an internal or external memory.

The second external device 330 may be configured to reject the instruction, i.e. to not encrypt and transmit the instruction to the implantable medical device 10, if the verification of the instruction would fail. For example, the second external device 330 determines that the instruction or any sub-step of the instruction is not comprised in the predetermined set of instructions, or if a value for a parameter is not within a predetermined interval, the second external device 330 may determine that the verification has failed.

In some embodiments, the implantable medical device 10 may be configured to verify the instruction. The verification of the instruction may be performed in the same way as described with reference to FIGS. 35a-35c. If the verification is performed by comparing the instruction or any sub-steps of the instruction with a predetermined set of instructions, the controller 300 may comprise a predetermined set of instructions. The predetermined set of instructions may, for example, be stored in an internal memory of the controller 300. Similarly, the controller 300 may store predetermined reference intervals for any parameter that can be set, and the controller 300 may be configured to compare a received value for a parameter to such a predetermined reference interval. If the verification of the instruction would fail, the controller 300 may be configured to reject the instruction, i.e. not run the instruction.

In an alternative to encrypting and decrypting the instruction, the instruction may be signed by the second external device 330 using a cryptographic hash, and the controller 300 may be configured to verify that the signature is correct before running the instruction.

A corresponding method for transmitting an instruction will now be described with reference to FIGS. 35a-35c. The instruction may relate to a function of the implantable medical device, such as an instruction to run a function or method of the implantable medical device, or to set a value of a parameter of the implantable medical device. The method comprises: transmitting an instruction for the implantable medical device from the first external device 300 to a second external device 320, the instruction relating to a function of the implantable medical device 10, encrypting, at the second external device 330 using a first encryption key, the instruction into an encrypted instruction, and transmitting the encrypted instruction from the second external device 330 to the implantable medical device 10, decrypting, at the implantable medical device, the instructions using a second encryption key corresponding to the first encryption key. The steps performed by or at the implantable medical device may be executed by the controller 300.

The instruction may be any type of instruction for controlling a function of the implantable medical device. For example, the instruction may be an instruction to run a function or method of the implantable medical device 10 or controller 300, an instruction comprising a plurality of sub-steps to be run at the controller 300, or a value for a parameter at the controller 300. The first external device 320 may, for example, receive the instruction from a user via a user interface displayed at or connected to the first external device 320. In another example, the first external device 320 may determine the instruction in response to data received from the implantable medical device 10, such as measurement data, or from another external device. Thus, in some examples, the method may further comprise receiving, at the first external device 320, an instruction to be transmitted to the implantable medical device 10. The method may further comprise displaying a user interface for receiving the instruction. In another example, the method comprises determining, at the first external device 320, an instruction to be transmitted to the implantable medical device 10.

In some embodiments, the transmitting of the encrypted instruction from the second external device 330 to the implantable medical device 10 comprises transmitting the encrypted instruction from the second external device 330 to the first external device 320, and transmitting the encrypted instruction from the first external device 320 to the controller 300 of the implantable medical device 10. In other words, the first external device 320 may relay the encrypted instruction from the second external device 330 to the controller 300, preferably without decrypting the instruction before transmitting it.

The method may further comprise to, at the controller 300, running the instruction or performing the instruction. The running of the instruction may be performed by an internal computing unit or a processor 306 comprised in the controller 300, and may, for example, cause the internal computing unit or processor 306 to instruct the implantable medical device 302 to perform an action.

The method may further comprise verifying, at the second external device 330, that the instructions are correct. The verifying may be performed as described above with reference to the corresponding system.

The method may further comprise verifying, at the controller 300, that the instructions are correct. The verifying may be performed as described above with reference to the corresponding system.

The method may further comprise authenticating the connection between the first external device 320 and the controller 300 over which the encrypted instruction is to be transmitted. The authentication may be performed as described herein.

As described above, a control program of the controller 300 may be updatable, configurable or replaceable. A system and a method for updating or configuring a control program of the controller 300 is now described with reference to FIGS. 35a-35c. The controller may comprise an internal computing unit 306 configured to control a function of the implantable medical device 10, the internal computing unit 306 comprises an internal memory 307 configured to store: i. a first control program 310 for controlling the internal computing unit, and ii. a second, configurable or updatable, with predefined program steps, control program 312 for controlling said function of the implantable medical device 10, and iii. a set of predefined program steps for updating the second control program 312. The controller 300 is configured to communicate with an external device 320. The internal computing unit 306 is configured to receive an update to the second control program 312 via the controller 300, and a verification function of, connected to, or transmitted to the controller 300. The verification function is configured to verify that the received update to the second control program 312 comprises program steps comprised in the set of predefined program steps. In this way, the updating or programming of the second control program may be performed using predefined program steps, which may decrease the risk that the new or updated control program is incorrect or comprises malicious software, such as a virus, spyware or a malware.

The predefined program steps may comprise setting a variable related to a pressure, a time, a minimum or maximum temperature, a current, a voltage, an intensity, a frequency, an amplitude of electrical stimulation, a feedback mode (sensorics or other), a post-operative mode or a normal mode, a catheter mode, a fibrotic tissue mode (for example semi-open), a time open after urination, a time open after urination before bed-time.

The verification function may be configured to reject the update in response to the update comprising program steps not comprised in the set of predefined program steps and/or be configured to allow the update in response to the update only comprising program steps comprised in the set of predefined program steps.

The internal computing unit 306 may be configured to install the update in response to a positive verification, for example by a user using an external device, by a button or similarly pressed by a user, or by another external signal.

The authentication or verification of communications between the implant and an external device has been described above.

When updating a control program of the controller 300, it may be beneficial to transmit a confirmation to a user or to an external device or system. Such a method is now described with reference to FIGS. 35a-35b.

The method for updating a control program of a controller 300 comprised in the implantable medical device 10 according to any of the embodiments herein. The controller 300 is adapted for communication with a first external device 320 and a second external device 330, which may comprise receiving, by the internal computing unit, an update or configuration to the control program from the first external device, wherein the update is received using a first communication channel; installing, by the internal computing unit 306, the update; and transmitting, by the internal computing unit, logging data relating to the receipt of the update or configuration and/or logging data relating to an installation of the update to the second external device 330 using the second communication channel; wherein the first and the second communication channels are different communication channels. By using a first and a second communication channels, in comparison to only using one, the security of the updating may be improved as any attempts to update the control program will be logged via the second communication channel, and thus, increasing the chances of finding incorrect or malicious update attempts.

The update or configuration comprises a set of instructions for the control program, and may, for examples comprise a set of predefined program steps as described above. The configuration or update may comprise a value for a predetermined parameter.

In some examples, the method further comprises confirming, by a user or by an external control unit, that the update or configuration is correct based on the received logging data.

The logging data may be related to the receipt of the update or configuration, and the controller 300 is configured to install the update or configuration in response to receipt of a confirmation that the logging data relates to a correct set of instructions. In this way, the controller 300 may receive data, transmit a logging entry relating to the receipt, and then install the data in response to a positive verification that the data should be installed.

In another example, or in combination with the one described above, the logging data is related to the installation or the update or configuration. In this example the logging data may be for information purposes only and not affect the installation, or the method may further comprise activating the installation in response to the confirmation that the update or configuration is correct.

If the update or configuration is transmitted to the controller 300 in one or more steps, the verification as described above may be performed for each of the steps.

The method may further comprise, after transmitting the logging data to the second external device, verifying the update via a confirmation from the second external device 330 via the second communication channel.

With reference to FIGS. 35a-35c there may further be provided an implantable controller 300. The controller 300 is connected to a sensor 351 wherein the sensor 351 is at least one microphone sensor 351 configured to record acoustic signals. For instance, the controller 300 may be configured to register a sound related to at least one of a bodily function of the patient and a function of the implantable medical device 10. The controller 300 comprises a computing unit 306 configured to derive at least one of a pulse of the patient from the registered sound related to a bodily function, such as information related to the patient swallowing, from the registered sound related to a bodily function. In the alternative, the controller 300 could be configured to derive information related to a functional status of the implantable medical device 10 from the registered sound, such as RPM of the motor. To this end the computing unit 306 may be configured to perform signal processing on the registered sound (e.g. on a digital or analog signal representing the registered sound) so as to derive any of the above mentioned information related to a bodily function of the patient or a function of the implantable medical device 10. The signal processing may comprise filtering the registered sound signals of the microphone sensor 351.

The implantable controller is placed in an implantable housing for sealing against fluid, and the microphone sensor 351 is placed inside of the housing. Accordingly, the controller and the microphone sensor 351 do not come into contact with bodily fluids when implanted which ensures proper operation of the controller and the microphone sensor 351.

In some implementations, the computing unit 306 is configured to derive information related to the functional status of an active unit 302 of the implantable medical device 10, from the registered sound related to a function of the implantable medical device 10. Accordingly, the computing unit 306 may be configured to derive information related to the functional status of at least one of: a motor, a pump and a transmission of the active unit 302 of the implantable medical device 10, from the registered sound related to a function of the implantable medical device 10.

The controller may comprise a transceiver 303,308 configured to transmit a parameter derived from the sound registered by the at least one microphone sensor 351 using the transceiver 303,308. For example, the transceiver 303,308 is a transceiver configured to transmit the parameter conductively (303) to an external device 320 or wirelessly (308) to an external device 320.

A method of authenticating the implantable medical device 10, the external device 320 or a communication signal or data stream between the external device 320 and the implantable medical device 10 is also described with reference to FIGS. 35a-35c. The method comprises the steps of registering a sound related to at least one of a bodily function and a function of the implantable medical device 10, using the at least one microphone sensor 351, connected to the controller 300. The method could in a first authentication embodiment comprise transmitting a signal derived from the registered sound, using the transceiver 303,308, receiving the signal in the external device 320, using the receiver 323,328 and comparing, in the external device 320, a parameter derived from the received signal with a reference parameter, using the computing unit 306. The method could in a second authentication embodiment comprise receiving a signal in the controller 300, from the external device 320, using the transceiver 323,328 and deriving a reference parameter from the received signal, using the computing unit 306 of the controller 300, and comparing, in the controller 300, a parameter derived from the received signal with the derived reference parameter, using the computing unit 306 of the controller 300. The methods further comprise the steps of the implantable controller 300 authenticating the external device 320, or the external device 320 authenticating the implantable controller 300, on the basis of the comparison. The registered sound could for example be related to the patient eating.

According to one embodiment described with reference to FIG. 35A-35C, the communication unit or internal controller 300 or control unit 300 comprises a wireless transceiver 308 for communicating wirelessly with an external device, a security module 389, and a central unit, also referred to herein as a computing unit 306, which is to be considered as equivalent. The central unit 306 is configured to be in communication with the wireless transceiver 308, the security module 389 and the active unit 302 i.e. the medical device for stretching the stomach. The wireless transceiver 308 is configured to receive communication from the external device 320 including at least one instruction to the implantable system 10 and transmit the received communication to the central unit or computing unit 306. The central unit or computing unit 306 is configured to send secure communication to the security module 389, derived from the received communication from the external device 200, and the security module 389 is configured to decrypt at least a portion of the secure communication and verify the authenticity of the secure communication. In one embodiment, the security module is further configured to transmit a response communication to the central unit or computing unit 306 and the central unit or computing unit is configured to communicate the at least one instruction to the active unit 302. In another embodiment, the security module is configured to communicate the at least one instruction to the active unit 302 directly. In the embodiment shown in FIG. 35A-35C, the at least one instruction is based on the response communication, or a combination of the response communication and the received communication from the external device 320.

In the embodiment shown in FIG. 35A-35C, the security module 389 comprises a set of rules for accepting communication from the central unit or computing unit 306. In the embodiment shown in FIG. 35A-35C, the wireless transceiver 308 is configured to be able to be placed in an off-mode, in which no wireless communication can be transmitted or received by the wireless transceiver 308. The set of rules comprises a rule stipulating that communication from the central unit or computing unit 306 to the security module 389 or to the active unit 302 is only accepted when the wireless transceiver 308 is placed in the off-mode.

In the embodiment shown in FIG. 35A-35C, the set of rules comprises a rule stipulating that communication from the central unit or computing unit 306 is only accepted when the wireless transceiver 308 has been placed in the off-mode for a specific time period.

In the embodiment shown in FIG. 35A-35C, the central unit or computing unit 306 is configured to verify a digital signature of the received communication from the external device 320. The digital signature could be a hash-based digital signature which could be based on a biometric signature from the patient or a medical professional. The set of rules further comprises a rule stipulating that communication from the central unit 306 is only accepted when the digital signature of the received communication has been verified by the central unit 306. The verification could for example comprise the step of comparing the digital signature or a portion of the digital signature with a previously verified digital signature stored in the central unit 306. The central unit 306 may be configured to verify the size of the received communication from the external device and the set of rules could comprise a rule stipulating that communication from the central unit 306 is only accepted when the size of the received communication has been verified by the central unit 306. The central unit could thus have a rule stipulating that communication above or below a specified size range is to be rejected.

In the embodiment shown in FIG. 35A-35C, the wireless transceiver is configured to receive a message from the external device 320 being encrypted with at least a first and second layer of encryption. The central unit 306 the decrypts the first layer of decryption and transmit at least a portion of the message comprising the second layer of encryption to the security model 389. The security module 389 then decrypts the second layer of encryption and transmits a response communication to the central unit 306 based on the portion of the message decrypted by the security module 389 or transmits the decrypted data to the active unit 302.

In the embodiment shown in FIG. 35A-35C, the central unit 306 is configured to decrypt a portion of the message comprising a digital signature, such that the digital signature can be verified by the central unit 306, also the central unit 306 is configured to decrypt a portion of the message comprising message size information, such that the message size can be verified by the central unit 306.

In the embodiment shown in FIG. 35A-35C, the central unit 306 is configured to decrypt a first and second portion of the message, and the first portion comprises a checksum for verifying the authenticity of the second portion.

In the embodiment shown in FIG. 35A-35C, the response communication transmitted from the security module 389 comprises a checksum, and the central unit 306 is configured to verify the authenticity of at least a portion of the message decrypted by the central unit 306 using the received checksum, i.e. by adding portions of the message decrypted by the central unit 306 and comparing the sum to the checksum.

In the embodiment shown in FIG. 35A-35C, the set of rules further comprise a rule related to the rate of data transfer between the central unit 306 and the security module 389. The rule could stipulate that the communication should be rejected or aborted if the rate of data transfer exceeds a set maximum rate of data transfer, which may make it harder for unauthorized persons to inject malicious code or instructions to the medical implant.

In the embodiment shown in FIG. 35A-35C, the security module 389 is configured to decrypt a portion of the message comprising the digital signature being encrypted with the second layer of encryption, such that the digital signature can be verified by the security module 389. The security module 389 then transmits a response communication to the central unit 306 based on the outcome of the verification, which can be used by the central unit 306 for further decryption of the message or for determining if instructions in the message should be communicated to the active unit 302. Alternatively, the security module determines at least one instruction for the active unit 302 based on the message, and transmits the message to the active unit 203 directly.

In the embodiment shown in FIG. 35A-35C, the central unit 306 is only capable of decrypting a portion of the received communication from the external device 320 when the wireless transceiver 308 is placed in the off-mode. In the alternative, or as an additional layer of security, the central unit 306 may be limited such that the central unit 306 is only capable of communicating instructions to the active unit 302 of the implantable medical device 10 when the wireless transceiver 308 is placed in the off-mode. This ensures that no attacks can take place while the central unit 306 is communicating with the active unit 301.

In the embodiment shown in FIG. 35A-35C, the implantable controller 300 is configured to receive, using the wireless transceiver 308, a message from the external device 320 comprising a first un-encrypted portion and a second encrypted portion. The implantable controller 300 (e.g. the central unit 306 or the security module 389) then decrypts the encrypted portion, and uses the decrypted portion to verify the authenticity of the un-encrypted portion. As such, computing power and thereby energy can be saved by not encrypting the entire communication, but rather only the portion required to authenticate the rest of the message (such as a checksum and/or a digital signature)

In the embodiment shown in FIG. 35A-35C, the central unit 306 is configured to transmit an encrypted portion to the security module 389 and receive a response communication from the security module 389 based on information contained in the encrypted portion being decrypted by the security module. The central unit 306 is then configured to use the response communication to verify the authenticity of the un-encrypted portion. The un-encrypted portion could comprise at least a portion of the at least one instruction to the implantable medical device 306. Alternatively, the central unit 306 is configured to transmit an encrypted portion and an unencrypted portion to the security module 389 and the security module 398 decrypts the encrypted portion and, using the decrypted portion, verifies the authenticity of the un-encrypted portion.

In the embodiment shown in FIG. 35A-35C, the implantable controller 300 is configured to receive, using the wireless transceiver 308, a message from the external device 320 comprising information related to at least one of: a physiological parameter of the patient and a physical or functional parameter of the implanted system 10, and use the received information to verify the authenticity of the message. The physiological parameter of the patient could be a parameter such as a parameter based on one or more of: a temperature, a heartrate and a saturation value. The physical or functional parameter of the implanted system 10 could comprise at least one of a current setting or value of the active unit 302, a prior instruction sent to the implantable system 10 or an ID of the implanted system 10.

The portion of the message comprising the information related to the physiological parameter of the patient and/or physical or functional parameter of the implanted system 10 could be encrypted, and the central unit 306 may be configured to transmit the encrypted portion to the security module 389 and receive a response communication from the security module 389 based on the information having been decrypted by the security module 389.

In the embodiment shown in FIG. 35A-35C, the security module 389 is a hardware security module comprising at least one hardware-based key. The security module 389 may have features that provide tamper evidence such as visible signs of tampering or logging and alerting. It may also be so that the security module 389 is “tamper resistant”, which makes the security module 389 inoperable in the event that tampering is detected. For example, the response to tampering could include deleting keys is tampering is detected. The security module 389 could comprise one or more secure crypto processor chip. The hardware-based key(s) in the security module 389 could have a corresponding hardware-based key placeable in the external device 320. The corresponding external hardware-based key could be placed on a key-card connectable to the external device 320.

In one embodiment, the security module 387 and the central unit 309 are both comprised in a multi-processor, wherein the security module 387 runs on a first processor and the central unit runs on second processor, different from the first.

In alternative embodiments, the security module 389 is a software security module comprising at least one software-based key, or a combination of a hardware and software-based security module and key. The software-based key may correspond to a software-based key in the external device 320. The software-based key may correspond to a software-based key on a key-card connectable to the external device 320.

In the embodiment shown in FIG. 35A-35C, the external device 320 is a handheld external device, however, in alternative embodiments, the external device may be a remote external device or a cloud based external device

In the embodiment shown in FIG. 35A-35C, the at least one instruction to the implantable system 10 comprises an instruction for changing an operational state of the implantable system 10, including an operational state of the active unit 302, i.e. medical device for stretching the stomach.

In the embodiment shown in FIG. 35A-35C, the wireless transceiver 308 is configured to communicate wirelessly with the external 320 device using electromagnetic waves at a frequency below 100 kHz, or more specifically below 40 kHz. The wireless transceiver 308 is thus configured to communicate with the external device 320 using “Very Low Frequency” communication (VLF). VLF signals have the ability to penetrate a titanium housing of the implantable system 10, such a titanium housing of the remote unit, such that the electronics of the implantable system 10 can be completely encapsulated in a titanium housing.

The wireless transceiver 308 is configured to communicate wirelessly with the external device 320 using a first communication protocol and the central unit 306 is configured to communicate with the security module 389 using a second, different, communication protocol. This adds an additional layer of security as security structures could be built into the electronics and/or software in the central unit 106 enabling the transfer from a first to a second communication protocol. The wireless transceiver 308 may be configured to communicate wirelessly with the external device using a standard network protocol, which could be one of an RFID type protocol, a WLAN type protocol, a Bluetooth (BT) type protocol, a BLE type protocol, an NFC type protocol, a 3G/4G/5G type protocol, and a GSM type protocol. In the alternative, or as a combination, the wireless transceiver 308 could be configured to communicate wirelessly with the external device 320 using a proprietary network protocol. The wireless transceiver 308 could comprises an Ultra-Wide Band (UWB) transceiver and the wireless communication between the implantable controller 300 and the external device 320 could thus be based on UWB. The use of UWB technology enables positioning of the external device 320 which can be used by the implanted system 10 as a way to establish that the external device 320 is at a position which the implanted system 10 and/or the patient can acknowledge as being correct, e.g. in the direct proximity to the implanted system 10 and/or the patient, such as within reach of the patient and/or within 1 or 2 meters of the implanted system 10. In the alternative, a combination of UWB and BT could be used, in which case the UWB communication can be used to authenticate the BT communication, as it is easier to transfer large data sets using BT.

Embodiments relating to an implantable medical device 10 having a controller 300 having a processor 306 with a sleep mode and an active mode will now be described with reference to FIG. 35d. The implant, the internal communication unit and the external device(s) may have the features described above with reference to FIGS. 35a-35c.

In an embodiment in which the controller 300 comprises a processor 306 having a sleep mode and an active mode, the controller 300 comprises or is connected to a sensor 150 and a processing unit 306 having a sleep mode and an active mode. The sensor 150 is configured to periodically measure a physical parameter of the patient, and the controller 300 is further configured to, in response to a sensor measurement preceding a predetermined value, setting the processing unit 306 in an active mode. That is, the controller 300 may “wake up” or be set in an active mode in response to a measurement from, for example, the body. A physical parameter of the patient could for example be a local or systemic temperature, saturation/oxygenation, blood pressure or a parameter related to an ischemia marker such as lactate.

By sleeping mode, it is meant a mode with less battery consumption and/or processing power used in the processing unit 306, and by “active mode” it may be meant that the processing unit 306 is not restricted in its processing.

The sensor 150 may, for example, be a pressure sensor. The pressure sensor may be adapted to measure a pressure in an organ of a patient, a reservoir of the implant or a pressure exerted by at least one member. The sensor 150 may be an analog sensor or a digital sensor, i.e., a sensor 150 implemented in part in software. In some examples, the sensor is adapted to measure one or more of a battery or energy storage status of the implantable medical device 10 and a temperature of the implantable medical device 10. In this way, the sensor 150 may periodically sense a pressure of the implantable medical device 10 or of the patient and set the processing unit 306 in an active mode if the measured pressure is above a predetermined value. Thus, less power, i.e., less of for example a battery or energy storage comprised in the implant, may be used, thereby prolonging the lifetime of the implantable medical device 10 or increasing the time between charging occasions of the implantable medical device 10.

In some examples, the processor 306, when in set in the active mode, may cause a sensation generator 381 connected to the implant, comprised in the implantable medical device 10 or comprised in an external device 320, 330, to generate a sensation detectable by a sense of the patient. For example, the processor may cause the sensation generator to generate a sensation in response to a measure battery status, for example that the battery is above or below a predetermined level, that a measured pressure is above or below a predetermined level, or that another measured parameter has an abnormal value, i.e., less than or exceeding a predetermined interval or level. The sensation generator has been described in further detail earlier in this description.

The processing unit 306 may be configured to perform a corrective action in response to a measurement being below or above a predetermined level. Such a corrective action may, for example, be increasing or decreasing a pressure, increasing or decreasing electrical stimulation, increasing or decreasing power.

The controller 300 may comprise a signal transmitter 320 connected to the processing unit, and wherein the processing unit is configured to transmit data relating to the measurement via the transceiver 308 of the controller 300 or an additional internal signal transmitter 392. The transmitted data may be received by an external device 320.

The external device may have an external communication unit 390. The external device 320 may comprise a signal provider 380 for providing a wake signal to the controller 300. In some examples, the signal provider comprises a coil or magnet 371 for providing a magnetic wake signal.

The controller 300 may implement a corresponding method for controlling an implantable medical device 10 when implanted in a patient. The method comprises measuring, with a sensor of the controller 300 connected to or comprised in the controller 300, a physiological parameter of the patient or a parameter of the implantable medical device 10, and, in response to a sensor measurement having an abnormal value, setting, by the controller 300, a processor 306 of the controller 300 from a sleep mode to an active mode. The measuring may be carried out periodically. By “abnormal value” it may be meant a measured value exceeding or being less than a predetermined value, or a measured value being outside a predetermined interval. The method may further comprise generating, with a sensation generator 381 as described above, a sensation detectable by the patient. In some examples, the generating comprises requesting, by the processor, the sensation generator 381 to generate the sensation.

The method may further comprise to perform a medical intervention in response to a sensor measurement having an abnormal value, preferably after the processing unit has been set in the active mode.

A system comprising an implantable medical device 10 having a controller 300 having a sleep mode and an active mode will now be described with reference to FIG. 35d. In one embodiment, the controller 300 comprises a sensor 150 adapted to detect a magnetic field and a processing unit 306 having a sleep mode and an active mode, now described with reference to FIGS. 35a-35c. The external control unit 320 comprises a signal provider 380 adapted to provide a magnetic field detectable by the internal sensor 150. The controller 300 is further configured to, in response to a detected magnetic field exceeding a predetermined value, setting the processing unit 306 in an active mode. In this way, the external device 320 may cause a sleeping controller 300 or processor 306 to “wake up”.

The sensor 150 may, for example, be a hall effect sensor, a fluxgate sensor, an ultra-sensitive magnetic field sensor, a magneto-resistive sensor, an AMR or GMR sensor, or the sensor may comprise a third coil having an iron core.

The magnetic field provider 380 may have an off state, wherein it does not provide any magnetic field, and an on state, wherein it provides a magnetic field. For example, the magnetic field provider 380 may comprise a magnet 371, a coil 371, a coil having a core 371, or a permanent magnet 371. In some embodiments, the magnetic field provider 380 may comprise a shielding means for preventing a magnet 371 or permanent magnet 371 from providing a magnetic field in the off state. In order to provide a substantially even magnetic field, the magnetic field provider may comprise a first and a second coil arranged perpendicular to each other.

After the processing unit 306 has been set in an active mode, i.e., when the processing unit 306 has been woken, the implant may determine a frequency for further communication between the controller 300 and the external device 320. The controller 300 may thus comprise a frequency detector 391 for detecting a frequency for communication between the controller 300 and the second communication unit 390. The frequency detector 391 is, for example, an antenna. The external device 320 may comprise a frequency indicator 372, for transmitting a signal indicative of a frequency. The frequency indicator 372, may, for example, be a magnetic field provider capable of transmitting a magnetic field with a specific frequency. In some examples the frequency indicator is comprised in or the same as the magnetic field provider 371. In this way, the frequency signal is detected using means separate from the sensor, and can, for example, be detected using a pin on a chip.

Alternatively, the controller 300 and the external device 320 may communicate using a predetermined frequency or a frequency detected by means defined by a predetermined method according to a predetermined protocol to be used for the communication between the controller 300 and the external device 320.

In some embodiments, the sensor 150 may be used for the communication. The communication may in these embodiments be performed with such that a frequency of the magnetic field generated by the coil is 9-315 kHz, or the magnetic field generated by the coil is less than or equal to 125 kHz, preferably less than 58 kHz. The frequency may be less than 50 Hz, preferably less than 20 Hz, more preferably less than 10 Hz, in order to be transmittable through a titanium box.

In some embodiments, the controller 300 comprises a receiver unit 392, and the internal control unit and the external control unit are configured to transmit and/or receive data via the receiver unit 392 via magnetic induction. The receiver unit 392 may comprise a high-sensitivity magnetic field detector, or the receiver unit may comprise a fourth coil for receiving the magnetic induction.

The system may implement a method for controlling a medical implant implanted in a patient. The method comprises monitoring for signals by a sensor 150 comprised in the controller 300 communicatively coupled to the active unit 302, providing, from a signal provider 380 comprised in an external device 320, a wake signal, the external device 320 being adapted to be arranged outside of the patient's body, and setting, by the controller 300 and in response to a detected wake signal WS, a mode of a processing unit 306 comprised in the internal control unit from a sleep mode to an active mode.

The method may also comprise detecting, using a frequency detector 391, a frequency for data communication between the controller 300 and a second communication unit 390 being associated with the external device 320. The frequency detector 391 is communicatively coupled to the controller 300 or the external device 320. The detection may be performed using a detection sequence for detecting the frequency. This detection sequence may, for example, be a detection sequence defined in the protocol to be used for communication between the controller 300 and the second communication unit 390. Potential protocols that may be used for communication between the controller 300 and the external device 320 has been described earlier in this description. Thus, the method may comprise determining, using the frequency detector 391, the frequency for data communication, and initiating data communication between the controller 300 and the second communication unit 390. The data communication can, for example, comprise one or more control instructions for controlling the implantable medical device 10 transmitted from the external device 320, or, for example, comprise data related to the operation of the implantable medical device 10 and be transmitted from the controller 300.

In some examples, the implantable medical device may comprise or be connected to a power supply for powering the implantable medical device 10. This will now be described with reference to FIG. 35e. The medical device, the internal control unit, and the external device(s) may comprise all elements described above with reference to FIGS. 35a-35c and FIG. 35d. The power supply may comprise an implantable energy storage unit 40 for providing energy to the medical device, an energy provider 397 connected to the implantable energy storage unit 40 and connected to an energy consuming part of the implantable medical device 10, the energy provider 397 being configured to store energy to provide a burst of energy to the energy consuming part, wherein the energy provider 397 is configured to be charged by the implantable energy storage unit 40 and to provide the energy consuming part with electrical power during startup of the energy consuming part.

Alternatively, the implantable medical device 10 may comprise a first implantable energy storage unit 40 for providing energy to an energy consuming part of the implantable medical device 10, a second implantable energy storage unit 397 connected to the implantable energy storage unit 40 and connected to the energy consuming part, wherein the second implantable energy storage unit 397 is configured to be charged by the implantable energy storage unit 40 and to provide the energy consuming part with electrical power during startup of the energy consuming part. The second implantable energy storage unit 397 has a higher energy density than the first implantable energy storage unit 40. By having a “higher energy density” it may be meant that the second implantable energy storage unit 397 has a higher maximum energy output per time unit than the first implantable energy storage unit 40. The second energy storage 397 may be an energy provider as discussed below.

The energy consuming part may be any part of the implantable medical device 10, such as a motor for powering the hydraulic pump, a valve, a processing or computing unit, a communication unit, a device for providing electrical stimulation to a tissue portion of the body of the patient, a CPU for encrypting information, a transmitting and/or receiving unit for communication with an external unit (not shown as part of the energy consuming part in the drawings, that is, the communication unit may be connected to the energy storage unit 40 and to the energy provider 397), a measurement unit or a sensor, a data collection unit, a solenoid, a piezoelectrical element, a memory metal unit, a vibrator, a part configured to operate a valve comprised in the medical device, or a feedback unit.

In this way, an energy consuming part requiring a quick start or an energy consuming part which requires a high level or burst of energy for a start may be provided with sufficient energy. This may be beneficial as instead of having an idle component using energy, the component may be completely turned off and quickly turned on when needed. Further, this may allow the use of energy consuming parts needing a burst of energy for a startup while having a lower energy consumption when already in use. In this way, a battery or an energy storage unit having a slower discharging (or where a slower discharging is beneficial for the lifetime or health of the battery) may be used for the implant, as the extra energy needed for the startup is provided by the energy provider.

Energy losses may occur in a battery or energy storage unit of an implant if the battery or energy storage unit is discharged too fast. These energy losses may for example be in the form of heat, which may damage the battery or energy storage unit. By the apparatus described in these examples, energy may be provided from the battery or energy storage unit in a way that does not damage the battery or energy storage unit, which may improve the lifetime of the battery or energy storage unit and thereby the lifetime of the medical device.

In some examples, the discharging from the implantable energy storage unit 40 during startup of the energy consuming part is slower than the energy needed for startup of the energy consuming part, i.e., the implantable energy storage unit 40 is configured to have a slower discharging than the energy needed for startup of the energy consuming part. That is, there is a difference between the energy needed by the energy consuming part and the energy the implantable energy storage unit 40 is capable of providing without damaging the implantable energy storage unit 40. In other words, a maximum energy consumption of the energy consuming part may be higher than the maximum energy capable of being delivered by the implantable energy storage unit 40 without causing damage to the implantable energy storage unit, and the energy provider 397 may be adapted to deliver an energy burst corresponding to difference between the required energy consumption and the maximum energy capable of being delivered by the implantable energy storage unit 40. The implantable energy storage unit 40 may be configured to store a substantially larger amount of energy than the energy burst provider 397 but may be slower to charge.

The implantable energy storage unit 40 may be any type of energy storage unit suitable for an implant, such as a re-chargeable battery or a solid-state battery, such as a tionyl-chlorid battery. The implantable energy storage unit 40 may be connected to the energy consuming part and configured to power the energy consuming part after it has been started using the energy provider 397.

The energy provider 397 may be any type of part configured to provide a burst of energy for the energy consuming part. In some examples, the energy provider 397 is a capacitor, such as a start capacitor, a run capacitor, a dual run capacitor or a supercapacitor. The energy provider 397 may be connected to the implantable energy storage unit 40 and be adapted to be charged using the implantable energy storage unit 40. In some examples, the energy provider may be a second energy provider 397 configured to be charged by the implantable energy storage unit 40 and to provide the energy consuming part with electrical energy. The implantable medical device 10 may further comprising a temperature sensor for sensing a temperature of the capacitor and the temperature sensor may be integrated or connected to the controller 300 such that the sensed temperature can be used as input for controlling the implantable medical device 10 or as feedback to be sent to an external device 320.

A corresponding method for powering a medical device may also be contemplated. The method comprises the steps of initiating an energy consuming part 302 of the implant, the energy consuming part being connected to an implantable energy storage unit 40, providing an initial burst of energy to the energy consuming part using an energy provider 397 connected to the implantable energy storage unit 40 and to the energy consuming part 302, the energy provider 397 being adapted to provide a burst of energy to the energy consuming part, and subsequently powering the energy consuming part 302 using the implantable energy storage unit 40.

In some examples, a maximum energy consumption of the energy consuming part is higher than the maximum energy capable of being delivered by the implantable energy storage unit 40 without causing damage to the implantable energy storage unit 40, and the energy provider 397 is adapted to deliver an energy burst corresponding to difference between the required energy consumption and the maximum energy capable of being delivered by the implantable energy storage unit 40.

The method may further comprise the step of charging the energy provider 397 using the implantable energy storage unit 40.

Initiating an energy consuming part 302 may comprise transitioning a control unit of the medical device from a sleep mode to an operational or active mode.

The implantable energy storage unit 40 may be adapted to be wirelessly charged and the implantable energy storage unit may be connected to an internal charger 395 for receiving wireless energy from an external device 320 via an external charger 396, and the method may comprise wirelessly charging the implantable energy storage unit 40. In some examples, the method comprises controlling a receipt of electrical power from an external energy storage unit at the internal charger 395. The internal energy storage unit 40 may be charged via the receipt of a transmission of electrical power from an external energy storage unit 396 by the internal charger 395.

The embodiments described herein may advantageously be combined. For example, all the embodiments relating to the communication and controlling of the medical device may be combined with the embodiments relating to the programming of the implant, the methods and systems for improving energy consumption or the power supply. The embodiments relating to the programming of the medical device may be combined with any of the embodiments relating to improving the energy consumption or the power supply. The embodiments relating to the power supply maybe combined with the methods and systems for improving the energy consumption.

A computer program product of, or adapted to be run on, an internal computing unit or an external device is also provided, which comprises a computer-readable storage medium with instructions adapted to make the internal computing unit and/or the external device perform the actions as described in any embodiment or example above.

FIG. 35f and FIG. 35fa shows one embodiment of a system for charging, programming and communicating with the controller 300 of the implanted medical device 100. FIGS. 35f and 35fa further describes the communication and interaction between different external devices which may be devices held and operated by the patient, by the health care provider (HCP) or by the Dedicated Data Infrastructure (DDI), which is an infrastructure supplier for example by the manufacturer of the implanted medical device 100 or the external devices 320′,320″,320′″. The system of the embodiment of FIGS. 35f and 35fa comprises three external devices 320′,320″,320′″ capable of communicating with the controller 300. The basic idea is to ensure the security of the communication with, and the operation of, the medical device 100 by having three external devices 320′,320″,320′″ with different levels of authority. The lowest level of authority is given to the patient operated remote control 320″. The remote control external device 320″ is authorized to operate functions of the implanted medical device 100 via the implanted controller 300, on the basis of patient input. The remote control 320″ is further authorized to fetch some necessary data from the controller 300. The remote control 320″ is only capable of operating the controller 300 by communicating with the software currently running on the controller 300, with the currently settings of the software. The next level of authority is given to the Patient External Interrogation Device (P-EID) 320′″, which is a charging and communication unit which is held by the patient but is partially remotely operated by the Health Care Provider (HCP) (Usually a medical doctor with the clinic providing the treatment with help of the implanted medical device 100). The P-EID 320′″ is authorized to make setting changes by selecting pre-programmed steps of the software or hardware running on the controller 300 of the implanted medical device 100. The P-EID is remotely operated by the HCP, and receives input from the HCP, via the DDI. The highest level of authority is given to the HCP-EID 320′ and its controller the HCP Dedicated Display Device (DDD). The HCP-EID 320′ is a charging and communication unit which is held by the HCP physically at the clinic of the HCP. The HCP-EID 320′ is authorized to freely alter or replace the software running on the controller 300, when the patient is physically in the clinic of the HCP. The HCP-EID 320′ is controlled by the HCP DDD, which either acts on a “webview” portal from the HCP-EID or is a device closed down to any activities (which may include the absence of an internet connection) other than controlling and communicating with the HCP-EID. The webview portal does not necessarily mean internet based or HTML-protocol and the webview portal may be communicated over other communicating protocols such as Bluetooth or any other type of standard or proprietary protocol. The HCP DDD may also communicate with the HCP-EID over a local network or via Bluetooth or other standard or proprietary protocols.

Starting from the lowest level of authority, the patient remote control external device 320″ comprises a wireless transceiver 328 for communicating with the implanted medical device 100. The remote control 320″ is capable of controlling the operation of the implanted medical device 100 via the controller 300, by controlling pre-set functions of the implantable medical device 100, e.g., for operating an active portion of the implanted medical device 100 for performing the intended function of the implanted medical device 100. The remote control 320″ is able communicate with implanted medical device 100 using any standard or proprietary protocol designed for the purpose. In the embodiment shown in FIGS. 35f and 35fa, the wireless transceiver 328 comprises a Bluetooth (BT) transceiver, and the remote control 320″ is configured to communicate with implanted medical device 100 using BT. In an alternative configuration, the remote control 320″ communicates with the implanted medical device 100 using a combination of Ultra-Wide Band (UWB) wireless communication and BT. The use of UWB technology enables positioning of the remote control 320″ which can be used by the implanted medical device 100 as a way to establish that the remote control 320″ is at a position which the implanted medical device 100 and/or the patient can acknowledge as being correct, e.g. in the direct proximity to the medical device 100 and/or the patient, such as within reach of the patient and/or within 1 or 2 meters of the implanted medical device 100.

UWB communication is performed by the generation of radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses. A UWB radio system can be used to determine the “time of flight” of the transmission at various frequencies. This helps overcome multipath propagation, since some of the frequencies have a line-of-sight trajectory, while other indirect paths have longer delay. With a cooperative symmetric two-way metering technique, distances can be measured to high resolution and accuracy. UWB is useful for real-time location systems, and its precision capabilities and low power make it well-suited for radio-frequency-sensitive environments, such as health care environments.

In embodiments in which a combination of BT and UWB technology is used, the UWB technology may be used for location-based authentication of the remote control 320″, whereas the communication and/or data transfer could take place using BT or any other way of communicating different from the UWB. The UWB signal could in some embodiments also be used as a wake-up signal for the controller 300, or for the BT transceiver, such that the BT transceiver in the implanted medical device 100 can be turned off when not in use, which eliminates the risk that the BT is intercepted, or that the controller 300 of the implanted medical device 100 is hacked by means of BT communication. In embodiments in which a BT (or alternatives)/UWB combination is used, the UWB connection may be used also for the transmission of data. In the alternative, the UWB connection could be used for the transmission of some portions of the data, such as sensitive portions of the data, or for the transmission of keys for the unlocking of encrypted communication sent over BT.

The remote control 320″ comprises computing unit 326 which runs a software application for communicating with the implanted medical device 100. The computing unit 326 can receive input directly from control buttons 335 arranged on the remote control 320″ or may receive input from a control interface 334i displayed on a patient display device 334 operated by the patient. In the embodiments in which the remote control 320″ receives input from a control interface 334i displayed on the patient display device 334 operated by the patient, the remote control 320″ transmits the control interface 334i in the form of a web-view portal, i.e., a remote interface that run in a sandbox environment on the patient's display device 334. A sandbox environment means that it runs on the display device 334 but only displays what is presented from the remote control and can only use a tightly controlled set of commands and resources, such as storage and memory space as well as network access, the ability to inspect the host system and read or write from other input devices connected to the display device 334 is extremely limited. Any action or command generated by the patient display device is like controlling a webpage. All acting software is located on the remote control that only displays its control interface onto the patient display unit. The computing unit 326 is further configured to encrypt the control interface before transmission to the patient display device 334, and encrypt the control commands before transmission to the implanted medical device 100. The computing unit 326 is further configured to transform the received user input into control commands for wireless transmission to the implantable medical device 100.

The patient's display device 334 could for example be a mobile phone, a tablet or a smart watch. In the embodiment shown in FIGS. 35f and 35fa, the patient's display device 334 communicates with the remote control 320″ by means of BT. The control interface 334i in the form of a web-view portal is transmitted from the remote control 320″ to the patient's display device 334 over BT. Control commands in the form of inputs from the patient to the control interface 334i is transmitted from the patient's display device 334 to the remote control 320″, providing input to the remote control 320″ equivalent to the input that may be provided using the control buttons 335. The control commands created in the patient's display device 334 is encrypted in the patient's display device 334 and transmitted to the remote control 320′ using BT or any other communication protocol.

The remote control is normally not connected to the DDI or the Internet to increase security. In addition, the remote control 320″ may in one embodiment have its own private key and in a specific embodiment the remote control 320″ is activated by the patient's private key for a certain time period. This may activate the function of the patient's display device and the remote wed-view display portal supplied by the remote control to the patient's display device.

The patient's private key is supplied in a patient private key device compromising a smartcard that may be inserted or provided close to the remote control 320″ to activate a permission to communicate with the implant 100 for a certain time period.

The patient's display device 334 may (in the case of the display device 334 being a mobile phone or tablet) comprise auxiliary radio transmitters for providing auxiliary radio connection, such as Wi-Fi or mobile connectivity (e.g., according to the 3G, 4G or 5G standards). The auxiliary radio connection(s) may have to be disconnected to enable communication with the remote control 320″. Disconnecting the auxiliary radio connections reduces the risk that the integrity of the control interface 334i displayed on the patient's display device 334 is compromised, or that the control interface 334i displayed on the patient's display device 334 is remote controlled by an unauthorized device.

In alternative embodiments, control commands are generated and encrypted by the patient's display device and transmitted to the DDI 330. The DDI 330 could either alter the created control commands to commands readable by the remote control 320″ before further encrypting the control commands for transmission to the remote control 320″ or could simply add an extra layer of encryption before transmitting the control commands to the remote control 320″, or could simply act as a router for relaying the control commands from the patients' display device 334 to the remote control 320″. It is also conceivable that the DDI 330 adds a layer of end-to-end encryption directed at the implanted medical device 100, such that only the implanted medical device 100 can decrypt the control commands to perform the commands intended by the patient. In the embodiments above, when the patient remote display device 334 is communicating with the DDI, the patient's display device 334 may be configured to only display and interact with a web-view portal provided by a section of the DDI and it is conceivable that the web-view portal is a view of a back-end provided on the DDI 330, and in such embodiments, the patient interacting with the control interface on the patient's display device 334 is equivalent to the patient interacting with an area of the DDI 330.

The patient's display device 334 could have a first and second application related to the implanted medical device 100. The first application is the control application displaying the control interface 334i for control of the implanted medical device 100, whereas the second application is a general application for providing the patient with general information of the status of the implanted medical device 100 or information from the DDI 330 or HCP, or for providing an interface for the patient to provide general input to the DDI 330 or HCP related to the general wellbeing of the patient, the lifestyle of the patient or related to general input from the patient concerning the function of the implanted medical device 100. The second application, which do not provide input to the remote control 320″ and/or the implanted medical device 100 thus handles data which is less sensitive. As such, the general application could be configured to function also when all auxiliary radio connections are activated, whereas switching to the control application which handles the more sensitive control commands and communication with the implanted medical device 100 could require that the auxiliary radio connections are temporarily de-activated. It is also conceivable that the control application is a sub-application running within the general application, in which case the activation of the control application as a sub-application in the general application could require the temporary de-activation of auxiliary radio connections. In the embodiment shown in FIG. 35f, access to the control application requires the use of the optical and/or NFC means of the hardware key 333′ in combination with biometric input to the patient's display device, whereas accessing the general application only requires biometric input to the patient's display device and/or a pin code. In the alternative, a two-factor authentication solution, such as a digital key in combination with a pin code could be used for accessing the general application and/or the control application.

In general, a hardware key is needed to activate the patient display device 334 for certain time period to control the web-view portal of the remote control 320″, displaying the control interface 334i for control of the implanted medical device 100.

In the embodiments in which the patients display device 334 is configured to only display and interact with a web-view provided by another unit in the system, it is conceivable that the web-view portal is a view of a back-end provided on the DDI 330, and in such embodiments, the patient interacting with the control interface on the patient's display device is equivalent to the patient interacting with an area of the DDI 330.

Moving now to the P-EID 320′″. The P-EID 320′″ is an external device used by the patient, patient external device, which communicates with, and charges, the implanted medical device 100. The P-EID 320″′ can be remotely controlled by the HCP to read information from the implanted medical device 100. The P-EID 320″′ controls the operation of the implanted medical device 100, control the charging of the medical device 100, and adjusts the settings on the controller 300 of the implanted medical device 100 by changing pre-defined pre-programed steps and/or by the selection of pre-defined parameters within a defined range., e.g. Just as the remote control 320″, the P-EID 320″′ could be configured to communicate with the implanted medical device 100 using BT or UWB communication or any other proprietary or standard communication method. Since the device may be used for charging the implant, the charging signal and communication could be combined. Just as with the remote control 320″, it is also conceivable to use a combination of UWB wireless communication and BT for enabling positioning of the P-EID 320″ as a way to establish that the P-EID 320″ is at a position which the implanted medical device 100 and/or patient and/or HCP can acknowledge as being correct, e.g. in the direct proximity to the correct patient and/or the correct medical device 100. Just as for the remote control 320″, in embodiments in which a combination of BT and UWB technology is used, the UWB technology may be used for location-based authentication of the P-EID 320″, whereas the communication and/or data transfer could take place using BT. The P-EID 320″ comprises a wireless transmitter/transceiver 328 for communication and also comprises a wireless transmitter 325 configured for transferring energy wirelessly, which may be in the form of a magnetic field or any other signal such as electromagnetic, radio, light, sound or any other type of signal to transfer energy wirelessly to a wireless receiver 395 of the implanted medical device 100. The wireless receiver 395 of the implanted medical device 100 is configured to receive the energy in the form of the magnetic field and transform the energy into electric energy for storage in an implanted energy storage unit 40, and/or for consumption in an energy consuming part of the implanted medical device 100 (such as the operation device, controller 300 etc.). The magnetic field generated in the P-EID 320″′ and received in the implanted medical device 100 is denoted charging signal. In addition to enabling the wireless transfer of energy from the P-EID 320″′ to the implanted medical implant 10, the charging signal may also function as a means of communication. E.g., variations in the frequency of the transmission, and/or the amplitude of the signal may be uses as signaling means for enabling communication in one direction, from the P-EID 320″′ to the implanted medical device 100, or in both directions between the P-EID 320″′ and the implanted medical device 100. The charging signal in the embodiment shown in FIGS. 35a and 35fa is a signal in the range 10-65 kHz or 115-140 kHz and the communication follow a proprietary communication signaling protocol, i.e., it is not based on an open standard. In alternative embodiments, BT could be combined with communication using the charging signal, or communication using the charging signal could be combined with an UWB signal. The energy signal could also be used as a carrying signal for the communication signal.

Just as for the remote control 320″, the UWB signal could in some embodiments also be used as a wake-up signal for the controller 300, or for the BT transceiver, such that the BT transceiver in the implanted medical device 100 can be turned off when not in use, which eliminates the risk that the BT is intercepted, or that the controller 300 of the implanted medical device 100 is hacked by means of BT communication. In the alternative, the charging signal could be used as a wakeup signal for the BT, as the charging signal does not travel very far. Also, as a means of location-based authentication, the effect of the charging signal or the RSSI could be assessed by the controller 300 in the implanted medical device 100 to establish that the transmitter is within a defined range. In the BT/UWB combination, the UWB may be used also for transmission of data. In some embodiments, the UWB and/or the charging signal could be used for the transmission of some portions of the data, such as sensitive portions of the data, or for the transmission keys for unlocking encrypted communication sent by BT. Wake-up could be performed with any other signal.

UWB could also be used for waking up the charging signal transmission, to start the wireless transfer of energy or for initiating communication using the charging signal. As the signal for transferring energy has a very high effect in relation to normal radio communication signals, the signal for transferring energy cannot be active all the time, as this signal may be hazardous e.g., by generating heat.

The P-EID 320′″ communicates with the HCP over the Internet by means of a secure communication, such as over a VPN. The communication between the HCP and the P-EID 320″′ is preferably encrypted. Preferably, the communication is sent via the DDI, which may only be relying on the information. The communication from the HCP to the implanted medical device 100 may be performed using an end-to-end encryption, in which case the communication cannot be decrypted by the P-EID 320″′. In such embodiments, the P-EID 320″′ acts as a router, only passing on encrypted communication from the HCP to the controller 300 of the implanted medical device 100 (without full decryption). This solution further increases security as the keys for decrypting the information rests only with the HCP and with the implanted medical device 100, which reduces the risk that an unencrypted signal is intercepted by an unauthorized device. The P-EID 320′″ may add own encryption or information, specifically for security reasons. The P-EID 320′″ may hold its own private key and may be allowed to communicate with the implant 100 based on confirmation from the patient's private key, which may be provided as a smartcard to be inserted in a slot of the P-EID 320′″ or hold in close proximity thereto to be read by the P-EID 320′″. These two keys will add a high level of security to the performed communication between the Implant 100 and the P-EID 320′″ since the patient's hardware key in this example on the smartcard may activate and thereby allow the communication and action taken in relation to the implant. The P-EID 320′″ may as previously described change the treatment setting of the implant by selecting pre-programmed steps of the treatment possibilities. Such pre-programmed treatment options may include for example to change:
at least one of the level and time of stretching and when such stretching occurs in relation to food intake of a patient for an operable implant for stretching the stomach wall of the patient for creating satiety,
parameters of an implant able to be programmed from outside the body,
parameters of an implant able to be programmed from outside the body with a wireless signal,
parameters of an implant adapted to move fluid inside the body of the patient, such as volume, pumping parameters,
parameters of an implant configured to sense a parameter related to the patient swallowing,
parameters of an implant configured to exercise a muscle with electrical or mechanical stimulation, such as stimulation parameters, amplitude frequency time period etc.,

When the implanted medical device 100 is to be controlled and/or updated remotely by the HCP, via the P-EID 320′″, an HCP Dedicated Device (DD) 332 displays an interface in which predefined program steps or setting values are presented to the HCP. The HCP provides input to the HCP DD 332 by selecting program steps, altering settings and/or values or by altering the order in which pre-defined program steps is to be executed. The instructions/parameters inputted into the HCP DD 332 for remote operation is in the embodiment shown in FIG. 35f routed to the P-EID 320′″ via the DDI 330, which may or may not be able to decrypt/read the instructions. The DDI 330 may store the instructions for a time period to later transfer the instructions in a package of created instructions to the P-EID 320′″. It is also conceivable that an additional layer of encryption is provided to the package by the DDI 330. The additional layer of encryption may be a layer of encryption to be decrypted by the P-EID 330, or a layer of encryption which may only be decrypted by the controller 300 of the implanted medical device 100, which reduces the risk that unencrypted instructions or packages are intercepted by unauthorized devices. The instructions/parameters are then provided to the P-EID 320″, which then loads the instructions/parameters into the during the next charging/energy transfer to the implanted medical device 100 using any of the signal transferring means (wireless or conductive) disclosed herein.

The Health Care Provider EID (HCP EID) 320′ have the same features as the P-EID 320″ and can communicate with the implanted medical device 100 in the same alternative ways (and combinations of alternative ways) as the P-EID 320′″. However, in addition, the HCP EID 320′ also enables the HCP to freely reprogram the controller 300 of the implanted medical device 100, including replacing the entire program code running in the controller 300. The idea is that the HCP EID 320′ always remain with the HCP and as such, all updates to the program code or retrieval of data from the implanted medical device 100 using the HCP EID 320′ is performed with the HCP and patient present (i.e., not remote). The physical presence of the HCP is an additional layer of security for these updates which may be critical to the function of the implanted medical device 100.

In the embodiment shown in FIGS. 35f and 35fa, the HCP communicates with the HCP EID 320′ using an HCP Dedicated Display Device 332 (HCP DDD), which is an HCP display device comprising a control interface for controlling and communicating with the HCP EID 320′. As the HCP EID 320′ always stays physically at the HCP's clinic, communication between the HCP EID 320′ and HCP DDD 332 does not have to be sent over the Internet. Instead, the HCP DDD 332 and the HCP EID 320′ can communicate using one or more of BT, a proprietary wireless communication channel, or a wired connection. The alteration to the programming is then sent to the implanted medical device 100 directly via the HCP EID 320′. Inputting into the HCP DDD 332 for direct operation by means of the HCP EID 320′ is the same as inputting directly into the HCP EID 320′, which then directly transfers the instructions into the implanted medical device 100.

In the embodiment shown in FIGS. 35f and 35fa, both the patient and the HCP has a combined hardware key 333′,333″. The combined keys 333′,333″ comprises a hardware component comprising a unique circuitry (providing the highest level of security), a wireless NFC-transmitter 339 for transmitting a specific code (providing mid-level security), and a printed QR-code 344 for optical recognition of the card (providing the lowest level of security). The HCP private key is supplied by an HCP private key device 333″ adapted to be provided to the HCP EID external device via at least one of; a reading slot or comparable for the HCP private key device 333″, an RFID communication or other close distance wireless activation communication to both the HCP EID 320′ and the HCP DDD 332 if used. The HCP DDD 332 will be activated by such HCP private key device 333″, which for example may comprise at least one of, a smartcard, a keyring device, a watch an arm or wrist band a neckless or any shape device.

The HCP EID external device may comprise at least one of;
a reading slot or comparable for the HCP private key device,
an RFID communication and
other close distance wireless activation communication means

The HCP external device 320′ may further comprise at least one wireless transceiver 328 configured for communication with a data infrastructure server, DDI, through a first network protocol.

A dedicated data infrastructure server, DDI, is in one embodiment adapted to receive commands from said HCP external device 320′ and may be adapted to relay the received commands without opening said commands directed to the patient external device 320″, the DDI 330 comprising one wireless transceiver configured for communication with said patient external device 320″.

The patient EID external device 320″ is in one embodiment adapted to receive the commands relayed by the DDI, and further adapted to send these commands to the implanted medical device 100, which is adapted to receive commands from the HCP, Health Care Provider, via the DDI 330 to change the pre-programmed treatment steps of the implanted medical device 100. The patient EID is adapted to be activated and authenticated and allowed to perform the commands by the patient providing a patient private key device 333′. The patient's private key device is in one embodiment adapted to be provided to the patient external device by the patient via at least one of: a reading slot or comparable for the patient private key device 333′, an RFID communication or other close distance wireless activation communication.

The patient EID external device, in one or more embodiments, comprises at least one of;
a reading slot or comparable for the HCP private key device,
an RFID communication, or
other close distance wireless activation communication

The patient EID external device may in one or more embodiments comprise at least one wireless transceiver configured for communication with the implanted medical device through a second network protocol.

The patient's key 333′ is in the embodiment shown in FIGS. 35f and 35fa in the form of a key card having an interface for communicating with the P-EID 320′″, such that the key card could be inserted into a key card slot in the P-EID 320″. The NFC-transmitter 339 and/or the printed QR-code 344 can be used as means for accessing the control interface 334i of the display device 334. In addition, the display device 334 may require a pin-code and/or a biometric input, such as face recognition or fingerprint recognition.

The HCP's key 333″, in the embodiment shown in FIGS. 35f and 35fa is in the form of a key card having an interface for communicating with the HCP-EID 320′, such that in one embodiment the key card could be inserted into a key card slot in the HCP-EID 320′. The NFC-transmitter 339 and/or the printed QR-code 344 can be used as means for accessing the control interface of the HCP DDD 332. In addition, the HCP DDD 332 may require a pin-code and/or a biometric input, such as face recognition or fingerprint recognition.

In alternative embodiments, it is however conceivable that the hardware key solution is replaced by a two-factor authentication solution, such as a digital key in combination with a PIN code or a biometric input (such as face recognition and/or fingerprint recognition). The key could also be a software key, holding similar advance key features, such as the Swedish Bank ID being a good example thereof.

In the embodiment shown in FIGS. 35f and 35fa, communication over the Internet takes place over a Dedicated Data Infrastructure (DDI) 330, running on a cloud service. The DDI 330 in this case handles communication between the HCP DDD 332 and the P-EID 320′″. however, the more likely scenario is that the HCP DDD 332 is closed down, such that only the necessary functions of the control application can function on the HCP DDD 332. In the closed down embodiment, the HCP DDD 332 is only able to give the necessary commands to HCP EID 320′ to further update the pre-programmed treatment steps of the Implant 100 via the P-EID 320′″ in direct contact, or more likely indirect contact via the DDI 332. If the patient is present locally, the HCP EID may communicate and act directly on the patient's implant. However, before anything is accepted by the implant, a patient private key device 333′ has to be presented to the P EID 320′″ or HCP EID 320′ for maximum security.

The DDI 330 is logging information of the contact between the HCP and the remote control 320″ via implant feedback data supplied from the implant to P-EID 320′″. Data generated between the HCP and the patient's display device 334, as well as between the HCP and auxiliary devices 336 (such as tools for following up the patient's treatments e.g., a scale in obesity treatment example or a blood pressure monitor in a blood pressure treatment example) are logged by the DDI 330. In some embodiments, although less likely, the HCP DDD 332 may also handle the communication between the patient's display device 334 and the remote control 320″. In FIG. 35fa, the auxiliary devices 336 is connected to the P-EID as well and can thus provide input from the auxiliary devices 336 to the P-EID which can be used by the P-EID for altering the treatment or for follow up.

In all examples, the communication from the HCP to: the P-EID 320″′, the remote control 320″, the patient's display device 334 and the auxiliary devices 336 may be performed using an end-to-end encryption. In embodiments with end-to-end encryption, the communication cannot be decrypted by the DDI 330. In such embodiments, the DDI 330 acts as a router, only passing on encrypted communication from the HCP to various devices. This solution further increases security as the keys for decrypting the information rests only with the HCP and with the device sending or receiving the communication, which reduces the risk that an unencrypted signal is intercepted by an unauthorized device. The P-EID 320″′ may also only pass on encrypted information.

In addition to acting as an intermediary or router for communication, the DDI 330 collects data on the implanted medical device 100, on the treatment and on the patient. The data may be collected in an encrypted form, in an anonymized form or in an open form. The form of the collected data may depend on the sensitivity of the data or on the source from which the data is collected. In the embodiment shown in FIGS. 35f and 35fa, the DDI 330 sends a questionnaire to the patient's display device 334. The questionnaire could comprise questions to the patient related to the general health of the patient, related to the way of life of the patient, or related specifically to the treatment provided by the implanted medical device 100 (such as for example a visual analogue scale for measuring pain). The DDI 330 could compile and/or combine input from several sources and communicate the input to the HCP which could use the provided information to create instructions to the various devices to be sent back over the DDI 330. The data collection performed by the DDI 330 could also be in the form a log to make sure that all communication between the units in the system can be back traced. Logging the communication ensures that all alterations to software or the settings of the software, as well as the frequency and operation of the implanted medical device 100 can be followed. Following the communication enables the DDI 330 or the HCP to follow the treatment and react it something in the communication indicates that the treatment does not provide the intended results or if something appears to be wrong with any of the components in the system. If patient feedback from the patient display device 334 indicates that a new treatment step of the implant is needed, such information must be confirmed by direct contact between HCP and patient.

In the specific embodiment disclosed in FIGS. 35f and 35fa, the wireless connections between the different units are as follows. The wireless connection 411 between the auxiliary device 336 and the DDI 330 is based on WiFi or a mobile telecommunication regime or may be sent to the DDI 330 via the P-EID 320′″ and the wireless connection 411 between the auxiliary device 336 and the patient's display device 334 is based on BT or any other communication pathway disclosed herein. The wireless connection 412 between the patient's display device 334 and the DDI 330 is based on WiFi or a mobile telecommunication regime. The wireless connection 413 between the patient's display device 334 and the remote control 320″ is based on BT or any other communication pathway disclosed herein. The wireless connection 414 between the patient remote control 320″ and the implanted medical device 100 is based on BT and UWB or any other communication pathway disclosed herein. The wireless connection 415 between the remote control 320″ and the DDI 330 is likely to not be used, and if present be based on WiFi or a mobile telecommunication regime. The wireless connection 416 between the P-EID 320′″ and the implanted medical device 100 is based on BT, UWB and the charging signal or any other communication or energizing pathway disclosed herein. The wireless connection 417 between the P-EID 320′″ and the DDI 330 is based on WiFi or a mobile telecommunication regime. The wireless connection 418 between the HCP-EID 320′ and the implanted medical device 100 is based on at least one of the BT, UWB and the charging signal. The wireless connection 419 between the P-EID 320′″ and the HCP DD 332 is based on BT or any other communication path disclosed herein. The wireless connection 420 between the HPC-EID 320′ and the DDI 330 is based on WiFi or a mobile telecommunication regime. The wireless connection 421 between the HPC DD 332 and the DDI 330 is normally closed and not used and if so based on WiFi or a mobile telecommunication regime. The wireless connection 422 between the HCP-EID 320′ and the HCP DD 332 is based on at least one of BT, UWB, local network or any other communication path disclosed herein.

The wireless connections specifically described in the embodiment shown in FIGS. 35f and 35fa may however be replaced or assisted by wireless connections based on radio frequency identification (RFID), near field communication (NFC), Bluetooth, Bluetooth low energy (BLE), or wireless local area network (WLAN). The mobile telecommunication regimes may for example be 1G, 2G, 3G, 4G, or 5G. The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves.

FIG. 35fa also discloses a master private key 333′″ device that allow issuance of new private key device wherein the HCP or HCP admin have such master private key 333′″ device adapted to be able to replace and pair a new patient private key 333′ device or HCP private key device 333″ into the system, through the HCP EID external device 320′.

A system configured for changing pre-programmed treatment settings of an implantable medical device, when implanted in a patient, from a distant remote location in relation to the patient, the system comprising:

FIG. 35fa also discloses a scenario in which at least one health care provider, HCP, external device 320′ is adapted to receive a command from the HCP to change said pre-programmed treatment settings of an implanted medical device 100, further adapted to be activated and authenticated and allowed to perform said command by the HCP providing a HCP private key device 333″. The HCP EID external device 320′ further comprising at least one wireless transceiver 328 configured for communication with a patient EID external device 320″′, through a first network protocol. The system comprises the patient EID external device 320″′, the patient EID external 320′″ device being adapted to receive command from said HCP external device 320′, and to relay the received command without modifying said command to the implanted medical device 100. The patient EID external device 320′″ comprising one wireless transceiver 328. The patient EID 320′″ is adapted to send the command to the implanted medical device 100, to receive a command from the HCP to change said pre-programmed treatment settings of the implanted medical device 100, and further to be activated and authenticated and allowed to perform said command by the patient providing a patient private key 333′ device comprising a patient private key.

Although wireless transfer is primarily described in the embodiment disclosed with reference to FIGS. 35f, 35fa the wireless communication between any of the external device may be substituted for wired communication. Also, some or all of the wireless communication between an external device and the implanted medical device 100 may be substituted for conductive communication using a portion of the human body as conductor.

FIG. 35fb shows a portion of FIG. 35f, in which some of the components have been omitted to outline a specific scenario. In the scenario outlined in FIG. 35fb, the system is configured for changing pre-programmed treatment settings of an implantable medical device 100, when implanted in a patient, from a distant remote location in relation to the patient. The system if FIG. 35fb comprises at least one HCP EID 320′ external device adapted to receive commands from the HCP to change said pre-programmed treatment settings of an implanted medical device 100. The HCP EID 320′ external device is further adapted to be activated and authenticated and allowed to perform said command by the HCP providing a HCP private key device 333″ adapted to be provided to the HCP EID external device 320′. The private key device 333″ is adapted to be provided to the HCP EID external device 320′ via at least one of: a reading slot or comparable for the HCP private key device 333″, and an RFID communication or other close distance wireless activation communication. The HCP EID external device 320′ comprises at least one of: a reading slot or comparable for the HCP private key device 333″, an RFID communication, and other close distance wireless activation communication or electrical direct contact. The HCP EID external device 320′ further comprises at least one wireless transceiver 328 configured for communication with a dedicated data infrastructure server (DDI) 330, through a first network protocol. The system further comprises a dedicated data infrastructure server (DDI) 330, adapted to receive command from said HCP EID external device 320′, adapted to relay the received commands without modifying said command to a patient EID external device 320″′. The dedicated data infrastructure server (DDI) 330 further comprises a wireless transceiver 328 configured for communication with said patient external device. The system further comprises a patient EID external device 320′″ adapted to receive the command relayed by the dedicated data infrastructure server (DDI) 330 and further adapted to send commands to the implanted medical device 100 and further adapted to receive commands from the HCP EID external device 320′ via the dedicated data infrastructure server (DDI) 330 to change said pre-programmed treatment settings of the implanted medical device 100. The patient EID external device 320″′, and further adapted to be activated and authenticated and allowed to perform said command by the patient providing a patient private key device 333′ adapted to be provided to the patient EID external device 320″′ by the patient via at least one of: a reading slot or comparable for the patient private key device 333′, an RFID communication or other close distance wireless activation communication or electrical direct contact. The patient EID external device 320″′ further comprises at least one of: a reading slot or comparable for the HCP private key device, an RFID communication and other close distance wireless activation communication or electrical direct contact. The patient EID external device 320″′ further comprises at least one wireless transceiver 328 configured for communication with the implanted medical device 100 through a second network protocol. The implanted medical device 100 is in turn configured to treat the patient or perform a bodily function.

The scenario described with reference to FIG. 35fb may in alternative embodiments be complemented with additional units or communication connections, or combined with any of the scenarios described with reference to FIGS. 35fc-35fe.

FIG. 35fc shows a portion of FIG. 35f, in which some of the components have been omitted to outline a specific scenario. In the scenario outlined in FIG. 35fc, system configured for changing pre-programmed treatment settings of an implantable medical device 100 is disclosed. The changing pre-programmed treatment settings are performed by a health care provider (HCP) in the physical presence of the patient. The system comprises at least one HCP EID external device 320′ adapted to receive commands from the HCP, directly or indirectly, to change said pre-programmed treatment settings in steps of an implantable medical device 100, when implanted. The HCP EID external device 320′ is further adapted to be activated, authenticated, and allowed to perform said command by the HCP providing a HCP private key device 333″ comprising a HCP private key. The HCP private key device in the embodiment of FIG. 35fc, comprises at least one of: a smart card, a keyring device, a watch, an arm or wrist band, a necklace, and any shaped device. The HCP EID external device 320′ is adapted to be involved in at least one of: receiving information from the implant 100, receiving information from a patient remote external device 336, actuating the implanted medical device 100, changing pre-programmed settings, and updating software of the implantable medical device 100, when implanted. The HCP EID external device 320′ is adapted to be activated, authenticated, and allowed to perform said command also by the patient, the system comprises a patient private key device 333′ comprising a patient private key. The patient private key device 333′ comprising at least one of: a smart card, a keyring device, a watch, an arm or wrist band, a necklace, and any shaped device. The HCP private key 333″ and the patients private key are required for performing said actions by the HCP EID external device 320′ to at least one of: receive information from the implant 100, to receive information from a patient remote external device 336, to actuate the implanted medical device 100, to change pre-programmed settings, and to update software of the implantable medical device 100, when the implantable medical device is implanted.

FIG. 35c also outlines a scenario in which the system is configured for changing pre-programmed treatment settings in steps of an implantable medical device, when implanted in a patient, by a health care provider, HCP, with the patient on remote on distance, the system comprising: at least one HCP EID external device 320′ adapted to receive a command from the HCP direct or indirect, to change said pre-programmed treatment settings in steps of an implantable medical device, when implanted, wherein the HCP EID external device 320′ is further adapted to be activated, authenticated, and allowed to perform said command by the HCP. The said action by the HCP EID external device 320′ to change pre-programmed settings in the implant 100 and to update software of the implantable medical device 100, when the implantable medical device 100 is implanted, is adapted to be authenticated by a HCP private key device 333″ and a patient private key device 333′.

The scenario described with reference to FIG. 35fc may in alternative embodiments be complemented with additional units or communication connections, or combined with any of the scenarios described with reference to FIG. 35fb, or 35fd-35fc.

FIG. 35fd shows a portion of FIG. 35f, in which some of the components have been omitted to outline a specific scenario. In the scenario outlined in FIG. 35fd, a system configured to change pre-programmed and pre-selected treatment actions of an implantable medical device 100 by command from the patient is described. The system comprises an implantable medical device 100, a patient remote external device 320″, and a wireless transceiver 328 configured for communication with the implantable medical device 100, when the medical device is implanted, through a second network protocol. The system further comprises a remote display portal interface 334i configured to receive content delivered from the patient remote external device 320″ to expose buttons to express the will to actuate the functions of the implanted medical device 100 by the patient through the patient remote external device 320″. The remote external device 320″ is further configured to present the display portal remotely on a patient display device 334 allowing the patient to actuate the functions of the implanted medical device 100 through the display portal of the patient remote external device 320″ visualized on the patient display device 334. In FIG. 35fd, a further wireless connection 423 between the patient remote external device 320″ and the patient EID external device 320′″ is provided. This further wireless connection 423 could be a wireless connection according to any one of the wireless signaling methods and protocols described herein, and the communication can be encrypted.

FIG. 35fd further shows a scenario in which the external system comprises a first external device in the form of the HCP EID external device 320′ and a second external device in the form of patient EID external device 320″′. The HCP EID external device 320′ and the patient EID external device 320″′ have a wireless or wired connection 416′ to each other and external system is configured for providing remote instructions to the implantable medical device 100. The HCP EID external device 320′ or the patient EID external device 320′″ is configured to, derive a checksum from the instructions that will be sent to the implant and electronically sign the instructions and the checksum using at least one of a patient private key device 333′ or a HCP private key device 333′″. The HCP EID external device 320′ or the patient EID external device 320″′ is then configured to form a data packet from the instructions, the electronic signature and the checksum. In the embodiment shown in FIG. 35fd, the patient EID external device 320′″ comprises a wireless transmitter configured to wirelessly send the data packet to the implantable medical device 100. The HCP EID external device 320′ or the patient EID external device 320″′ may further be configured encrypt the data packet prior to transmission. If the HCP EID external device 320′ creates and signed the instructions, the patient EID external device 320′″ may be configured to transmit the data packet wirelessly to the implantable medical device without changing the data packet and/or without full decryption of the data packet. In the embodiment shown in FIG. 35fd, the patient private key and the HCP private key are placed on a patient private key device 333′ and a HCP private key device 333′″. However, the patient private key and the HCP private key may be placed directly on the HCP EID external device 320′ or the patient EID external device 320′″. Either way, the patient private key and the HCP private key may be placed on the EIDs or the key devices by the manufacturers and may be placed on the EIDs or the key devices in the form of software or hardware. The key may be a non-extractable key.

In the example when the HCP EID external device 320′ communicates directly with the patient EID external device 320′″, the external system is configured to function without connection to the Internet which greatly reduces the risk that the system is hacked. As the system is not connected to the Internet, the system cannot depend on a synchronized time e.g. for time-out of log-in functionality. As such, the external system is configured to communicate with the implantable medical device 100 independently of time. The authentication and verification may thus be based entirely on the possession of keys. In an alternative embodiment, the log-in of signing functionality offered by the key devices 333″, 333′″ may be complemented or replaced by an input button on one or both of the HCP EID external device 320′ or the patient EID external device 320″′, configured to be used for verifying user presence. I.e., a user presses the input button on request from the HCP EID external device 320′ or the patient EID external device 320″′ and thereby verifies presence.

The implantable medical device 100 is in this embodiment configured to receive remote instructions from the external system by a wireless receiver configured to receive wirelessly transmitted data packets from the external system, i.e. the HCP EID external device 320′ or the patient EID external device 320′″. The implantable medical device 100 is configured to: verify the electronic signature, and use a checksum provided in the data packet to verify the integrity of the instructions.

A verification query operation may further be built into the external system or between the external system and the implantable medical device 100. The verification query operation comprising: transmitting, from the HCP EID external device 320′, the patient EID external device 320″′, or the implantable medical device 100, a query comprising a computational challenge to at least one other of the HCP EID external device 320′, the patient EID external device 320″′, or the implantable medical device 100 and receiving, at the first or second external devices, a response based on the transmitted computational challenge, and verifying at the HCP EID external device 320′, the patient EID external device 320″′, or the implantable medical device 100, the received response. The verification query operation may be in the form of a proof of possession operation comprising: receiving a public key, the public key being associated with a private key, transmitting a computational challenge to the first or second key device, based on the public key received from the first or second key device, receiving a response from the first or second key device based on the possession of the private key in the first or second key device, and verifying that the response based on the possession of the private key matches the query based on a public key. The verification query operation may also be performed between one of the HCP EID external device 320′ or the patient EID external device 320″′ and one of the first and second key devices.

In an alternative authentication or verification method for providing remote instructions from the external system to the implantable medical device 100, the implantable medical device comprises a list of codes and the external system comprises a list of codes. The method comprising encrypting the instructions at the external system using a code from a position on the list of codes, wirelessly sending the encrypted instructions to the implantable medical device, and decrypting, at the implantable medical device, the instructions using a code from a position on the list of codes. The same authentication or verification method may be used for authentication or verification or s signature applied to a communication which may comprise at least one instruction.

The scenario described with reference to FIG. 35fd may in alternative embodiments be complemented with additional units or communication connections, or combined with any of the scenarios described with reference to FIG. 35fb, 35fc, or 35fe.

FIG. 35fe shows a portion of FIG. 35fa, in which some of the components have been omitted to outline a specific scenario. In the scenario outlined in FIG. 35fe, a system configured for providing information from an implantable medical device 100, when implanted in a patient, from a distant remote location in relation to the patient is described. The system comprises at least one patient EID external device 320″′ adapted to receive information from the implant 100, and adapted to send such information further on to a server or dedicated data infrastructure, DDI, 330. The patient EID external device 320″′ is further adapted to be activated and authenticated and allowed to receive said information from the implanted medical device 100 by the patient providing a private key. The patient private key device comprises the private key adapted to be provided to the patient EID external device 320″′ via at least one of: a reading slot or comparable for the patient private key device, an RFID communication or other close distance wireless activation communication or direct electrical connection, The patient EID external device 320′″ comprises at least one of; a reading slot or comparable for the patient private key device, an RFID communication and other close distance wireless activation communication or direct electrical contact, The patient EID external device 320″′ further comprises at least one wireless transceiver 328 configured for communication with the DDI 330, through a first network protocol.

The scenario described with reference to FIG. 35fe may in alternative embodiments be complemented with additional units or communication connections, or combined with any of the scenarios described with reference to FIGS. 35fb-35fd.

FIG. 35ff shows a portion of FIG. 35fa, in which some of the components have been omitted to outline a specific scenario. In the scenario outlined in FIG. 35ff a system configured for changing pre-programmed treatment settings in steps of an implantable medical device 100, when implanted in a patient, by a health care provider, HCP, either in the physical presence of the patient or remotely with the patient on distance is described. The system comprising at least one HCP EID external device 320′ adapted to receive a command directly or indirectly from the HCP to change said pre-programmed treatment settings in steps of the implantable medical device 100, when implanted, wherein the HCP EID external device 320′ is further adapted to be activated, authenticated, and allowed to perform said command by the HCP providing a HCP private key device comprising a HCP private key, comprising at least one of: a smart card, a keyring device, a watch, an arm or wrist band, a necklace, and any shaped device. The system further comprises a patient private key device comprising a patient private key comprising at least one of: a smart card, a keyring device, a watch, an arm or wrist band, a necklace, and any shaped device. Both the HCP and patient private key is required for performing said action by the HCP EID external device 320′ to change the pre-programmed settings in the implant 100 and to update software of the implantable medical device 100, when the implantable medical device 100 is implanted. The patient private key is adapted to activate, be authenticated, and allowed to perform said command provided by the HCP, either via the HCP EID external device or when the action is performed remotely via a patient EID external device 320′. In the embodiment shown in FIG. 35ff, the communication is routed over the DDI server 330.

The scenario described with reference to FIG. 35ff may in alternative embodiments be complemented with additional units or communication connections, or combined with any of the scenarios described with reference to FIGS. 35fb-35fe.

FIG. 35fg shows an overview of an embodiment of the system, similar to that described with reference to FIG. 35fa, the difference being that the HCP EID and the HCP DDD are combined into a single device.

FIG. 35fh shows an overview of an embodiment of the system, similar to that described with reference to FIG. 35fa, the difference being that the HCP EID 320′″ and the HCP DDD 332 are combined into a single device and the P EID 320″′ and the patient remote control external device 320″ are combined into a single device.

One probable scenario/design of the communication system is for the purpose of changing pre-programmed treatment settings of an implantable medical device, when implanted in a patient, from a distant remote location in relation to the patient. The system comprises at least one health care provider, HCP, external device 320′ adapted to receive a command from the HCP to change said pre-programmed treatment settings of an implanted medical device, further adapted to be activated and authenticated and allowed to perform said command by the HCP providing a HCP private key device 333″ adapted to be provided to an HCP EID external device via at least one of: a reading slot or comparable for the HCP private key device, a RFID communication or other close distance wireless activation communication. The HCP EID external device comprising at least one of: a reading slot or comparable for the HCP private key device, a RFID communication, and other close distance wireless activation communication or electrical direct contact. The HCP EID external device further comprises at least one wireless transceiver configured for communication with a patient EID external device, through a first network protocol, wherein the system comprises the patient EID external device, the patient EID external device being adapted to receive command from said HCP external device, and to relay the received command without modifying said command to the implanted medical device. The patient EID external device comprising one wireless transceiver configured for communication with said patient external device. The patient EID is adapted to send the command to the implanted medical device, to receive a command from the HCP to change said pre-programmed treatment settings of the implanted medical device, and further to be activated and authenticated and allowed to perform said command by the patient providing a patient private key device comprising a patient private key.

In another scenario, the implantable medical device may be configured to transmit information. Such information may, for example, relate to a function of the implantable medical device, a parameter of the body of the patient, measurements, among others. In that scenario, the implantable medical device may be configured to only transmit such data in response to a received authentication. The authentication may be received from the patient EID, or from another external device. The implantable medical device may verify that the authenticated device is authorized to request data, for example through a cryptographic verification, which in some examples is based on a key stored at the implantable medical device.

The patient EID (alternatively patient external device) may provide the authentication based on a patient private key provided to the patient EID. The implantable medical device may in that scenario verify that the authentication is based on a patient private key associated with a patient that is authorized to request information from the implant. Based on a valid authorization, the implantable medical device may send data to the patient external device. The data may in some examples be encrypted, for example in any of the ways of encrypting data from the medical implant are described herein. The authorization may be a one-time authorization, an authorization for a predetermined time interval or an authorization that is valid until withdrawn. For example, the authorization may be provided once a day, or at the time of requesting the data from the implantable medical device.

While FIGS. 35F and 35FA-FH and the description thereof discloses different scenarios, also other combinations of authentication or authorization are conceivable. For example, for changing settings of an implantable medical device, it may be required that a health care provider provides an authentication for the changes using their HCP private key. In some examples, an authorization is necessary also from the patient by the patient providing a patient private key device in order for the changes to be accepted by the implantable medical implant. In some examples, the patient may delegate its authorization to the health care provider by using the patient private key.

In some cases, when both an authorization from the health care provider and the patient are required, the health care provider and the patient could be in the same location. To provide an authorization showing that both the health care provider and the patient are at the same location, either the HCP external device or the patient external device may be adapted to receive both the patient private key and the HCP private key in order to authorize a command or a change for the implantable medical implant. Alternatively, the HCP external device or the patient external device may be configured to communicate via a short range communications technology to verify that the other device is present and authenticated before sending the changes to the implantable medical device. This added security may be beneficial, for example, when the medical implant is re-programmed, or software of the implantable medical device is otherwise changed.

In other examples, both an authorization from a patient and from a health care provider may be required, but without the requirement that they are at the same location. In those examples, the authorization may be given using their respective external device. This may be beneficial, for example, when making changes to treatment settings or updating a software is considered to be low risk. Different programs comprised in the implantable medical implant may be considered to have a different risk level associated with them. A risk determination may be programmed into the implantable medical implant as conditions for accepting an update. If the implantable medical implant determines that an update fulfils the conditions, it may install it, otherwise, if the implantable medical implant determines that the conditions are not fulfilled, it may reject the update.

In some examples, it may be sufficient to only require an authorization from at least one of a health care provider and a patient. For example, changes associated with a lower risk, such as changing pre-programmed settings or treatment settings within pre-determined ranges, may be performed using only one authorization. Although the different scenarios outlined in FIGS. 35fb-35fh are described with specific units and method of signaling, these scenarios may very well be combined with each other or complemented with additional units or communication connections.

As have been discussed before in this application, communication with a medical implant needs to be reliable and secure. For this purpose, it is desirable to have a standalone device as an external remote control (for example described as 320″ in FIGS. 35f-35fh) for the medical implant, such that no other programs or applications run on the same device which may disturb or corrupt the communication to the medical implant. However, the smartphone or tablet (for example described as 334 in FIGS. 35f-35fh) has become an integrated part of everyday life for most people. This means that we almost always have our smartphones at hand. For this reason, it would have been convenient for the patient to communicate with the medical implant directly using the smartphone, such that no additional standalone device would have to be carried. However, as a lot of other applications are running on the smartphone, it does not fulfill the requirement of being a secure and reliable communication tool without interference from other communication. It is therefore desirable to split the tasks of providing secure communication between the external device and the implant from the task of communicating with the Internet and providing a familiar and intuitive user interface. For this purpose, and external device providing secure communication and tamperproof soft- and hardware, where the display device allows for intuitive and easy use is provided. In the embodiments described with reference to FIGS. 35g-35k a device fulfilling these combinatory needs will be described in the form of a standalone remote control external device integrated in a housing unit 320″ connectable to a smartphone or another display device 334, such as a smart watch or a tablet.

FIG. 35g shows the housing unit 320″ in an elevated perspective view form the left, and FIG. 35h shows the housing unit 320″ in a plain view from the left. In the embodiment shown in FIG. 35g, the housing unit 320″ has a rectangular shape with rounded edges, having a height 1521 which is more than 1.5 times the width 1522. The housing unit 320″ comprises recess 1525 configured to receive a display device 334, in the form of a smartphone, configured to be fitted in the housing unit 320″ for mechanically, disconnectably connecting the display device 334 to the housing unit 320″. The boundaries of the recess 1525 in the housing unit 320″ forms an edge 1528 configured to encircle the display device 334, when the display device 334 is inserted into the recess 1525. In the embodiment shown in FIG. 35g, the recess 1525 has a depth 1526 configured to allow the display device 334 to be entirely inserted into the recess 1525. As such, the depth 1526 of the recess 1525 exceeds the depth 1531 of the display device 334. In the embodiment shown in FIGS. 35g and 35h, the edge is relatively thin, and has a width 1527 which is in the range ⅛- 1/100 of the width of the display device 334, as such, the housing unit 320″ has a width in the range 1.02-1.25 times the width 1522 of the housing unit 320″. In the same way, the housing unit 320″ has a height 1521 in the range 1.01-1.25 times the height 1521 of the display device 334. In the embodiment shown in FIGS. 35g-35h, the edges 1528 are configured to clasp the display device 334 and thereby mechanically fixate the display device 334 in the housing unit 320″. The minimum bounding box of the housing unit 320″ and the display device 334 when mechanically connected, is no more than, 10% wider, 10% longer or 100% higher, than the minimum bounding box of the display device 334.

For creating a clasping fixation, the edges of the housing unit 320″ is made from an elastic material crating a tension between the edge 1528 and the display device 334 holding the display device 334 in place. The elastic material could be an elastic polymer material, or a thin sheet of elastic metal. For the purpose of further fixating the display device 334 in the housing unit 320″, the inner surface of the edges 1528 may optionally comprise a recess or protrusion (not shown) corresponding to a recess or protrusion of the outer surface of the display device 334. The edges 1528 may in the alterative comprise concave portions for creating a snap-lock clasping mechanical fixation between the housing unit 320″ and the display device 334.

In the embodiment shown in FIGS. 35g and 35h, the housing unit 320″ functions as a remote control for communicating with an implanted medical device, including receiving information from, and providing instructions and updates to, the implanted medical device. Information could be information related to a state of the implanted medical device including any functional parameter of the implanted medical device or could be related to a state of the patient, including any physiological parameter pertaining to the body of the patient (further described on other sections of this disclosure). For the purpose of providing input to the implanted medical device and controlling and updating the functions of the housing unit 320″, the housing unit 320″ comprises a control interface comprising switches in the form of control buttons 335. The control buttons 335 are configured to be used when the external device is disconnected from the display device 334. The control interface further comprises a display 1505, which is a smaller and typically less sophisticated display 1505 than the display of the display device 334. In an alternative embodiment, the control buttons 335 and display 1505 are integrated into a single touch-responsive (touchscreen) display on which the control buttons may be displayed. In the embodiment shown in FIGS. 35g and 35h, one of the control buttons 335 is a control button for activating the implanted medical device and another of the control buttons 335 is a control button for deactivating the implanted medical device. When the display device 334 is attached to the housing unit 320″, the control buttons 335 and the display is covered by the display device 334 and are as such not in an operational state. In the embodiment shown in FIGS. 35g and 35h, the housing unit 320″ is configured to transmit information pertaining to the display of the user interface to the display device 334 and the display device 334 is configured to receive input pertaining to communication to or from the implantable medical device from the patient, and transmit signals based on the received input to the housing unit 320″. The input may be a command to change the operational state of the implantable medical device. The display device 334 comprises a touch screen configured to display the user interface and receive the input from the patient. The display of the display device 334 may comprise one or more OLEDs or IPS LCDs elements. When the display device 334 is connected to the housing unit 320″, the display device 334 is configured to display a control interface which is used to communicate with the housing unit 320″, i.e. providing input to and receiving information from the housing unit 320″. The input provided the housing unit 320″ is then relayed to the implanted medical device—and in the same way information communicated from the implanted medical device to the housing unit 320″ may be relayed or displayed on the display device 334. Having an external device comprising a combination of a housing unit 320″ comprising the communication means for communicating with the implanted medical device and a display device 334 basically only functioning as and Input/Output device connected to the housing unit 320″ makes it possible to have a secure communication between the housing unit 320″ and the display device 334, which is out of reach from the Internet connection of the display device 334, which makes it much harder for an external attacker to get access to any of the vital communication portions of the housing unit 320″. The communication between the housing unit and the display device 334 is very restricted and the only communication allowed from the display device 334 to the housing unit 320″ is input from the patient or a healthcare professional, and authentication parameters created by an authentication application running on the display device 334. The authentication application running on the display device 334 could be a number-generating authenticator or a biometric authenticator for authenticating the patient or health care professional, and the authentication parameters could for example be parameters derived from a facial image or a fingerprint. In the opposite direction, i.e. from the housing unit 320″ to the display device 334, the communication could be restricted to only communication needed for displaying information and/or a graphical user interface on the display device 334. The communication restrictions could for example be based on size of the communication packages or the frequency with which the communication takes place which reduces the risk that an unauthorized person makes multiple attempts to extract information from, or transit information to, the hand-held device.

In the embodiment shown with reference to FIGS. 35g and 35h, the housing unit 320″ comprises a first communication unit providing a wireless connection 413 to the display device 334. The wireless connection 413 is in the embodiment shown in FIGS. 35g and 35h based on NFC, but could in alternative embodiment be based on Bluetooth or any other communication pathway disclosed herein. The housing unit 320″ further comprises a second communication unit providing a wireless connection with the implanted medical device. The wireless communication between the housing unit 320″ and the implanted medical device is in the embodiment shown in FIGS. 35g and 35h based on Bluetooth, but could in alternative embodiments be based on NFC or UWB or any other communication pathway disclosed herein.

As mentioned, in the embodiment shown in FIGS. 35g and 35h, the wireless communication between the housing unit 320″ and the display device 334 is based on NFC, while the wireless communication between the housing unit 320″ and the is based on Bluetooth. As such, the first communication unit of the housing unit 320″ is configured to communicate wirelessly with the display device 334′ using a first communication frequency and the second communication unit of the housing unit 320″ is configured to communicate wirelessly with the implantable medical device using a second different communication frequency. For this purpose, the first communication unit of the housing unit 320″ comprises a first antenna configured for NFC-based wireless communication with the display device 334, and the second communication unit comprises a second antenna configured for Bluetooth-based wireless communication with the implantable medical device. The first and second antennae may be a wire-based antennae or a substrate-based antennae. As such, the first communication unit is configured to communicate wirelessly with the display device 334 on a first frequency and the second communication unit is configured to communicate wirelessly with the implantable medical device using a second different communication frequency. Also, first communication unit of the housing unit 320′ is configured to communicate wirelessly with the display device 334 using a first communication protocol (the NFC-communication protocol), and the second communication unit is configured to communicate wirelessly with the implantable medical device using a second communication protocol (the Bluetooth communication protocol). The first and second communication protocols are different which adds an additional layer of security as security structures could be built into the electronics and/or software enabling the transfer from a first to a second communication protocol.

In an alternative embodiment, the second communication unit may be configured to communicate wirelessly with the implantable medical device using electromagnetic waves at a frequency below 100 kHz, or preferably at a frequency below 40 kHz. The second communication unit may thus be configured to communicate with the implantable medical device using “Very Low Frequency” communication (VLF). VLF signals have the ability to penetrate a titanium housing of the implant, such that the electronics of the implantable medical device can be completely encapsulated in a titanium housing. In yet further embodiments, the first and second communication units may be configured to communicate by means of an RFID type protocol, a WLAN type protocol, a BLE type protocol, a 3G/4G/5G type protocol, or a GSM type protocol.

In yet other alternative embodiments, it is conceivable that the mechanical connection between the housing unit 320″ and the display device 334 comprises an electrical connection for creating a wire-based communication channel between the housing unit 320″ and the display device 334. The electrical connection could also be configured to transfer electric energy from the display device 334 to the housing unit, such that the housing unit 320″ may be powered or charged by the display device 334. A wired connection is even harder to access for a non-authorized entity than an NFC-based wireless connection, which further increases the security of the communication between the housing unit 320″ and the display device 334.

In the embodiment shown with reference to FIGS. 35g and 35h, the display device 334 comprises a first communication unit providing a wireless connection 413 to the housing unit 320″ based on NFC. The display device 334 further comprises a second communication unit providing a wireless connection with a further external device and/or with the Internet. The second external device may be far away, for example at a hospital or a place where a medical professional practice. The wireless communication between the display device 334 and a further external device is in the embodiment shown in FIGS. 35g and 35h based on WiFi, but could in alternative embodiments be based on for example Bluetooth.

As mentioned, in the embodiment shown in FIGS. 35g and 35h, the wireless communication between the display device 334 and the housing unit 320″ is based on NFC, while the wireless communication between the display device and a further external unit is based on WiFi. As such, the first communication unit of the display device 334 is configured to communicate wirelessly with the housing unit 320″ using a first communication frequency and the second communication unit of the display device 334 is configured to communicate wirelessly with a further external device using a second different communication frequency. For this purpose, the first communication unit of the display device 334 comprises a first antenna configured for NFC-based wireless communication with the housing unit 320″, and the second communication unit comprises a second antenna configured for WiFi-based wireless communication with a further external device. The first and second antennae may be wire-based antennae or substrate-based antennae. As such, the first communication unit is configured to communicate wirelessly with the housing unit 320″ on a first frequency and the second communication unit is configured to communicate wirelessly with the further external device using a second different communication frequency. Also, the first communication unit of the display device 334 is configured to communicate wirelessly with the housing unit 320″ using a first communication protocol (the NFC communication protocol), and the second communication unit is configured to communicate wirelessly with the further external device using a second communication protocol (the WiFi communication protocol). The first and second communication protocols are different which adds an additional layer of security as security structures could be built into the electronics and/or software enabling the transfer from a first to a second communication protocol.

In alternative embodiments, the second communication unit of the display device 334 may be configured to communicate with the further external device by means of, a WLAN type protocol, or a 3G/4G/5G type protocol, or a GSM type protocol.

In the embodiment shown in FIGS. 35g and 35h, the communication range of the first communication unit of the housing unit 320″ is less than a communication range of the second communication unit of the housing unit 320′, such that the communication distance between the housing unit 320″ and the medical implant may be longer than the communication distance between the housing unit 320″ and the display device 334. In the embodiment shown in FIGS. 35g and 35h, the communication range of the first communication unit may be constrained to a length that is less than five times the longest dimension of the minimal bounding box of the display device 334, or more precisely constrained to a length that is less than three times the longest dimension of the minimal bounding box of the display device 334.

In the embodiment shown in FIGS. 35g and 35h, communication between the housing unit 320″ and the display device 334 is only enabled when the housing unit 320″ is connected to the display device 334. I.e. at least one of the housing unit 320″ and the display device 334 is configured to allow communication between the housing unit 320″ and the display device 334 on the basis of the distance between the housing unit 320″ and the display device 334. In the alternative, the housing unit 320″ and/or the display device 334 may comprise a sensor configured to estimate whether the housing unit 320″ is attached to the display device 334 or not, such as a mechanically activated switch or a photo resistive sensor which providing sensor input when the housing unit 320″ and display device 334 are mechanically connected to each other. The signal from the at least one sensor then may be used to permit usage of the communication unit configured for communication with the display device 334.

In the embodiment shown in FIGS. 35g and 35h, communication between the housing unit 320″ and the implantable medical device is only enabled on the basis of a distance between the housing unit 320″ and the implantable medical device. In the embodiment shown in FIGS. 35g and 35h, the distance should be less than twenty times the longest dimension of the minimal bounding box of the display device, or more specifically less than ten times the longest dimension of the minimal bounding box of the display device. The distance between the housing unit 320″ and the medical implant may be measured using electromagnetic waves, or acoustic waves. The process of measuring the distance may comprise triangulation.

In the embodiment shown in FIGS. 35g and 35h, the second communication unit of the display device 334 need to be disabled to enable communication between the display device 334 and the housing unit 320″, and further the second communication unit of the display device 334 needs to be disabled to enable communication between the housing unit 320″ and the medical implant. Also, the second communication unit of the housing unit 320″ needs to be disabled to enable communication between the housing unit 320″ and the medical implant.

In the embodiment shown in FIGS. 35g and 35h, the housing unit 320″ further comprises an encryption unit configured to encrypt communication received from the display device 334 before transmitting the communication to the implanted medical device. The encryption unit may for example be based on one of the following algorithms: AES, Blowfish, DES, Kalyna, Serpent or Twofish. For the purpose for handling the communication, I/O and encryption, the housing unit 320″ comprises a processor which could be a general-purpose microprocessor and/or an instruction set processor and/or related chips sets and/or special purpose microprocessors such as ASICs (Application Specific Integrated Circuit). The processor also comprise memory for storing instruction and/or data.

The housing unit 320″ may comprise a storage unit, such as a battery, for storing energy. The storage unit may be adapted to be charged by the display device 334, or another external device. In some examples, the charging is performed using reverse wireless charging. To that end, the housing unit 320″ may comprise an energy receiver connected to the storage unit, the energy receiver being adapted to wirelessly receive energy from another device. The display device 334 may comprise a primary coil and the housing unit comprise a secondary coil connected to an energy storage of the housing unit, wherein the display device 334 is adapted to wirelessly charge the housing unit using the first coil, and wherein the housing unit is adapted to receive wirelessly transmitted energy through the second coil and store the energy in the storage unit. In one example, the wireless charging may be performed using the Qi standard for wireless charging.

FIGS. 35i and 35j shows an embodiment of the external unit similar to the embodiment described with reference to FIGS. 35g and 35h. The difference being that in the embodiment of FIGS. 35i and 35j, the housing unit 320″ does not clasp the display device 334. Instead, the housing unit comprises two magnets 1510 for magnetically fixating the display device 334 to the housing unit 320″. In alternative embodiments, it is equally conceivable that the external device comprises an intermediate portion, which is fixedly fixated to the housing unit for providing a detachable connection with the display device 334. In the alternative, the intermediate device could be fixedly fixated to the display device 334 and provide a detachable connection with the housing unit 320″.

FIG. 35k shows a system overview of the external device (which could be the external device of the embodiment described with reference to FIGS. 35g and 35h, or of the embodiment described with reference to FIGS. 35i and 35j). The housing unit 320″ is connected to the display device 334. A wireless connection 413 is provided between the housing unit 320″ and the display device 334, and a further wireless connection 413 is provided between the housing unit 320″ and the implanted medical device 100, such that the housing unit can send instructions and updates to the implanted medical device 100, and receive information, parameters (such as sensor values) and alarms from the implanted medical device 100. The communication between the external device and the medical implant 100 is further described in other portions of this disclosure.

In some examples shown in any of FIGS. 35A-35L, the patient remote control or the patient EID may comprise a wireless transceiver for communication with the implant, and a second wireless receiver for communication with a communications network over which the patient remote control or patient EID may communicate with a patient remote control, patient EID, HCP EID or DDI.

In some examples, the patient remote control or the patient EID may be wirelessly charged. Thus, the patient remote control or the patient EID may comprise a first coil for receiving wireless energy to be used or stored at the patient remote control or the patient EID.

### Dual Remote Controls

FIG. 35L shows one embodiment of a communication system for transmission of data to and/or from an implantable medical device 100. The communication system comprises an implantable medical implant, a first remote control 320″, and a second remote control 320″″. The first remote control 320″ (also referred to as patient external device 320″) is operable by a user and comprises a first wireless communication unit configured for wireless transmission of data to and/or from the implantable medical device 100. The second remote control 320″″ is inoperable by a user (i.e. may not comprise any input means such as buttons, switches, or user interface to receive any input directly from a user) and comprises a second wireless communication unit configured for wireless transmission of control commands and/or data to and/or from the implantable medical device 100. The second remote control 320″″ further comprises a third communication unit for communicating with a patient display device 334 (e.g. a smartphone, smartwatch, tablet, and/or the like).

By having two separate remote controls, the security of the implant may be improved, as there are two separate ways of controlling the implant. Thus, in case of a malfunction of either of the remote controls, the implant may still be controlled. Furthermore, this allows for the second remote control to be smaller or more compact since it is inoperable by the user other than through a patient display device or another external device. The second remote control may thus be smaller and potentially less expensive.

The first and second remote controls 320″, 320″″ each comprise a wireless transceiver 328 for communicating with the implantable medical device 100. The first and/or second remote control 320″, 320″″ is capable of controlling the operation of the implantable medical device 100 via the controller 300 (for controlling the implantable medical device and for communicating with devices external to the body of the patient and/or implantable sensors). The first and/or second remote control 320″, 320″″ may control the operation of the implantable medical device 100 by controlling pre-set functions of the implantable medical device 100, e.g. for operating an active portion of the implantable medical device 100 for performing the intended function of the implantable medical device 100.

The first and/or second remote control 320″, 320″″ is able to communicate with implantable medical device 100 using any standard or proprietary protocol designed for the purpose. At least one of the first remote control 320″, the second remote control 320″″, and the implantable medical device 100 may, e.g., comprise a Bluetooth (BT) transceiver. In particular, the wireless transceiver 328 may comprise a BT transceiver, and the and/or second remote control 320″, 320″″ may be configured to communicate with implantable medical device 100 using BT. In one embodiment, the first and/or second remote control 320″, 320″″ is configured to communicate with implantable medical device 100 using NFMI.

In an alternative configuration, the first and/or second remote control 320″, 320″″ may communicate with the implantable medical device 100 using a combination of Ultra-Wide Band (UWB) wireless communication, NFMI and/or BT. For example, at least one of first remote control 320″, the second remote control 320″″, and the implantable medical device 100 may comprise a UWB transceiver. The use of UWB technology enables positioning of the first and/or second remote control 320″, 320″″ which can be used by the implantable medical device 100 as a way to establish that the first and/or second remote control 320″, 320″ is at a position which the implantable medical device 100 and/or the patient can acknowledge as being correct, e.g. in the direct proximity to the medical device 100 and/or the patient, such as within reach of the patient and/or within 1 or 2 meters of the implantable medical device 100.

When a combination of BT and UWB and/or NFMI technology is used, the UWB or NFMI technology may be used for location-based authentication of the first and/or second remote control 320″, 320″″, whereas the communication and/or data transfer could take place using BT or any other way of communicating different from the UWB or NFMI. The UWB or NFMI signal could in some embodiments also be used as a wake-up signal for the controller 300, or for the BT transceiver, such that the BT transceiver in the implantable medical device 100 can be turned off when not in use, which eliminates the risk that the BT is intercepted, or that the controller 300 of the implantable medical device 100 is hacked by means of BT communication. In embodiments in which a BT (or alternatives)/UWB combination is used, the UWB connection may be used also for the transmission of data. In the alternative, the UWB connection could be used for the transmission of some portions of the data, such as sensitive portions of the data, or for the transmission of keys for the unlocking of encrypted communication sent over BT.

The first remote control 320″ may be configured to control functions of the implantable medical device 100 based on user input to the first remote control 320″. In particular, the first remote control 320″ may comprise an input device for receiving a first user input, wherein the first remote control 320″ is configured to transmit the first user input to the implantable medical device 100. The first remote control 320″ may comprise a computing unit 326 which runs a software application for communicating with the implantable medical device 100. The computing unit 326 may receive the first user input directly from control buttons 335 arranged on the first remote control 320″. The computing unit 326 may be configured to encrypt control commands before transmission to the implantable medical device 100. The computing unit 326 is further configured to transform the received first user input into control commands for wireless transmission to the implantable medical device 100.

The second remote control 320″″ may comprise a wireless transmitter 325 configured for transferring energy wirelessly. The energy may be in the form of a magnetic field or any other signal such as electromagnetic, radio, light, sound or any other type of signal to transfer energy wirelessly to a wireless receiver 395 of the implantable medical device 100. The wireless receiver 395 of the implantable medical device 100 is configured to receive the energy in the form of the magnetic field and transform the energy into electric energy for storage in an implantable energy storage unit 40 of the implantable medical device 100, and/or for consumption in an energy consuming part of the implantable medical device 100 (such as the operation device, controller 300 etc.). In other words, the implantable energy storage unit 40 may be adapted to be wirelessly charged. The first remote control may similarly comprise a wireless transmitter for transferring energy wirelessly to the implantable medical implant. The implantable energy storage unit 40 may particularly be connected to the wireless receiver 395 for receiving wireless energy from the first and/or second remote control 320″, 320″″.

In the embodiment shown in FIG. 35L, the second remote control 320″″ is configured to communicate with a patient display device 334. Here, the patient display device 334 comprises the consumer electronics device. In particular, the second remote control 320″″ is configured to receive a second user input from the patient display device 334 and to transmit the second user input to the implantable medical implant. The second remote control 320″″ may receive the second user input from a control interface 334i displayed on the patient display device 334 operated by the patient.

The patient display device 334 may for example be a mobile phone, a tablet or a smart watch. The display device 334 may, for example, communicate with the second remote control 320″″ by means of BT, but any wireless or wired communication means may be used. The control interface 334i, e.g. in the form of a web-view portal, may be transmitted from the second remote control 320″″ to the patient display device 334 over BT. Control commands in the form of inputs from the patient to the control interface 334i are transmitted from the patient display device 334 to the second remote control 320″″, providing input to the second remote control 320″″ equivalent to the input that may be provided using the control buttons 335 or other input means of the first remote control 320″. The control commands created in the patient display device 334 may be encrypted in the patient display device 334 and transmitted to the second remote control 320″ using BT or any other communication protocol.

The second remote control 320″ may be implemented and/or integrated in an accessory to the patient display device 334. The second remote control 320″″ may, e.g., form part of a mobile phone case (i.e. smartphone case) for a mobile phone. Alternatively, the second remote control 320″″ may be integrated in a case for a personal computer, or a body worn camera, or any other suitable type of external device as described herein. The case may for example be connected to the patient display device 334 (e.g. mobile phone) using a wire from the case and connected to the patient display device (e.g. a charging port).

The second remote control 320″″ may not be connected to the DDI or the Internet, thereby increasing security. The second remote control 320″″ may have a private key, in particular the second remote control 320″″ may be activated by a private key 333′ of the patient for a certain time period. This may activate the function of the patient display device 334 and the remote wed-view display portal supplied by the second remote control to the patient display device 334.

The patient's private key 333′ may be supplied in a patient private key device comprising a smartcard that may be inserted or provided close to the first remote control 320″ and/or close to the second remote control 320″″ to activate a permission to communicate with the implantable medical device 100 for a certain time period. The patient's private key 333′ is in the embodiment shown in FIG. 35L in the form of a key card having an interface for communicating with the first remote control 320″, the second remote control 320″″, and/or another device or control. The NFC-transmitter 339 and/or the printed QR-code 344 may be used as means for accessing the control interface 334i of the display device 334. In addition, the display device 334 may require a pin-code and/or a biometric input, such as face recognition or fingerprint recognition, for controlling the implantable medical implant.

The patient display device 334 may comprise auxiliary radio transmitters for providing auxiliary radio connection, such as Wi-Fi or mobile connectivity (e.g. according to the 3G, 4G or 5G standards). The auxiliary radio connection(s) may have to be disconnected to enable communication with the second remote control 320″″. Disconnecting the auxiliary radio connections reduces the risk that the integrity of the control interface 334i displayed on the patient display device 334 is compromised, or that the control interface 334i displayed on the patient's display device 334 is remote controlled by an unauthorized device.

The data transmitted in the communication system may comprises a control command for the medical implant. Hence, real-time, remote management of patient care is provided and settings of the medical implant may be adjusted, e.g., based on the patient's current health status. Thus, invasive procedures may be averted while efficiency of healthcare delivery and patient comfort may be improved. Furthermore, more responsive and/or personalized healthcare may be provided, as adjustments can be made promptly in response to changes in the patient's condition.

At least one of the first wireless communication unit of the first remote control 320″ and the second wireless communication unit of the second remote control 320″″ may be configured to send and/or receive data using near-field magnetic induction (NFMI). Thus, enhanced security and reliability of the communication system may be provided. NFMI creates a private, secure communication link that is difficult to intercept or disrupt due to the magnetic field being spatially confined and thus less susceptible to interference compared to traditional radio frequency communication. Furthermore, NFMI penetrate materials such as water and body tissue, making it particularly suitable for communication with medical implants.

Further, at least one of the first wireless communication unit and the second wireless communication unit may comprise a transmitter coil for modulating a magnetic field for transmitting the data. In turn, the implantable medical implant may comprise a receiving coil and an NFMI receiver connected to the receiving coil to receive the data. The transmitter coil(s), in conjunction with the receiving coil and NFMI receiver of the implantable medical implant, may provide efficient and reliable data transfer. The use of a magnetic field for data transmission, which is typically more energy-efficient than traditional radio frequency communication, may additionally reduce power consumption and thereby extend an operational period of the implantable medical implant.

The transmitter coil(s) may be configured to modulate a magnetic field, and the NFMI receiver may be adapted to measure the magnetic field in the receiving coil. A modulated magnetic field may enable the construction of specific signal patterns for the data transmission such that transmission of complex data sets is enabled.

At least one of the first wireless communication unit and the second wireless communication unit may further be configured to wirelessly charge the implantable medical implant using NFMI. In particular, at least one of the first wireless communication unit and the second wireless communication unit may be, and/or act as, the wireless transmitter 325 configured for transferring energy wirelessly

Similarly, the implantable medical implant may comprise a coil for receiving wireless energy for charging the implant via NFMI. The coil of the implantable medical implant may, e.g., form part of, or be, the wireless receiver 395.

The second and third communication units of the second control unit 320″″ may be configured to transmit and/or receive data using different network protocols. In other words, the second and third communication units may be designed to send and/or receive data using separate and/or alternate networking standards. Thus, the communication system can communicate across a variety of network environments and conditions. A multi-protocol support may enhance interoperability of the second remote control 320″″, allowing for communicate with a wide range of devices and systems (such as the patient display device 334 and the implantable medical device 100). Alternatively, or additionally, the second and third communication units may for the same reasons be configured to transmit and/or receive data using different frequency bands. The standard, communication, and/or network protocols discussed herein may be any one or more from the list of: Radio Frequency type protocol, RFID type protocol, WLAN type protocol, Bluetooth type protocol, BLE type protocol, NFC type protocol, 3G/4G/5G type protocol, and GSM type protocol.

In an example, the second communication unit has a longer effective range than the third communication unit. In other words, the second communication unit may be able to communicate with a device (e.g., the implantable medical device 100) from a further distance than the distance at which the third communication unit is able to communicate with another device (e.g., the patient display device 334). For example, the second communication unit may use a network protocol with a longer effective range than the network protocol of the third communication unit.

In the specific embodiment disclosed in FIG. 35L, the wireless connections between the different units are as follows. The wireless connection 413 between the patient display device 334 and the second remote control 320′″ is based on BT or any other communication protocol disclosed herein. The wireless connection 414 between the second remote control 320″″ and the implantable medical device 100 is based on BT and UWB or any other communication protocol disclosed herein. The wireless connection 416 between the first remote control 320″ and the implantable medical device 100 is based on BT, UWB, and the charging signal, or any other communication or energizing pathway disclosed herein.

The wireless connections specifically described in the embodiment shown in FIG. 35L may be replaced or assisted by wireless connections based on radio frequency identification (RFID), near field communication (NFC), Bluetooth, Bluetooth low energy (BLE), or wireless local area network (WLAN). The wireless connections may further be based on modulation techniques such as amplitude modulation (AM), frequency modulation (FM), phase modulation (PM), or quadrature amplitude modulation (QAM). The wireless connection may further feature technologies such as time-division multiple access (TDMA), frequency-division multiple access (FDMA), or code-division multiple access (CDMA). The wireless connection may also be based on infra-red (IR) communication. The wireless connection may feature radio frequencies in the high frequency band (HF), very-high frequency band (VHF), and the ultra-high frequency band (UHF) as well as essentially any other applicable band for electromagnetic wave communication. The wireless connection may also be based on ultrasound communication to name at least one example that does not rely on electromagnetic waves. An embodiment in which the second remote control 320″″ is comprised in a housing is shown in FIG. 35LL.

### Voice Control

FIG. 35M schematically shows a medical implant 10 when implanted in a patient 1. The medical implant comprises a processor 1300 connected to a medical device 100, which may be comprised in the implant 100. The medical device may be any medical device or implant discussed herein, and may be configured to control or monitor a function of the body of a patient. The medical implant 10 may further comprise or be connected to a microphone 1369. The microphone 1369 may receive audio and transfer that audio to the processor 1300. The processor 1300 may thus receive audio via microphone 369.

The processor 1300 may have two modes of operation, a learning mode for learning voice commands and an operational mode for recognizing and transmitting voice commands to the medical device 110 or the medical implant 1100. The processor 1300 may be configured to, when in the learning mode, receive a first audio training phrase and creating a transfer function, the transfer function being based on the first audio training phrase, wherein the transfer function is configured to adjust the amplitude of at least one frequency of audio received at the medical device 110 for enhancing audio received at the medical implant 100 to facilitate detection of voice commands. To this end, the processor 1300 may comprise a transfer function unit 1370. The processor 1300 may be further adapted to receive a second audio training phrase, the second audio training phrase comprising a voice command, wherein the voice command comprises an instruction for the control of the medical implant 1100 and/or the medical device 110. The processor 300 may be further configured to use the transfer function for generating an enhanced second audio training phrase in the medical implant, and associating the enhanced second audio training phrase with the instruction for the control of the medical implant. Thus, the medical implant 110 has learned that the voice command comprised in the enhanced audio training phrase corresponds to the instruction.

In some examples, the audio training phrases are inputted into a transfer function unit 1370 for creating the transfer function. The processor may further comprise a learning unit 1371 for associating the enhanced second audio training phrase with the instruction for the control of the medical implant. The learning unit 1371 may, for example, comprise an algorithm based on machine learning for learning to associate the enhanced audio training phrase with the correct instruction for the medical device 1100. The voice commands, the instructions and any association between the voice commands may be stored in a memory unit 1373 comprised in or connected to the processor 1300.

The processor 1300 may be further adapted to receive audio input, process the audio input in order to determine an instruction and to transfer that instruction to the medical device 1100. In order to determine the instruction, the processor may use the transfer function 1370 to enhance the audio input and then determine the instruction associated with the enhanced audio input (as associated by the method described herein). The instruction may also be called a control command or a command. The instruction may be determined by and/or be transferred to the medical device 1100 via a command unit 1372 comprised in or connected to the processor 1300. The instruction may relate to a function of the medical device 1100 and may cause the medical device 1100 to perform an action, or it may relate to any other function of the medical implant 10, such as the processor 1300.

By learning voice commands, it may be meant that the processor associates an audio input with a control command for the medical device.

The processor 1300 may be further configured to, when in the operational mode, receive an audio command phrase for the medical device 1100 or implant 110. The processor 1300 may be further configured to apply a transfer function to create an enhanced audio command phrase. The transfer function may have been created as discussed above. The processor 1300 may determine a corresponding command for the medical based on the enhance audio command phrase, and send the command to the medical device 1100 or the medical implant 110. The medical device 1100 or implant 110 may then execute the command.

When the medical implant is implanted in the body, typically the medical implant stays in the same place in the body. Thus, it has been realized that any noise or distortion created by the body to audio commands may be substantially the same. By creating a transfer function based on a first audio training phase when the medical implant is implanted in the body, any noise created by the body or any distortions to the audio training phase caused by the body itself can be accounted for. The method thus accounts for that that the noise and distortions created by the body is substantially the same over time. Thus, the transfer function may account for those disturbances when enhancing any audio received by the medical implant. In this way, audio received by the medical implant may be enhanced, i.e., any known disturbances created by the body to the audio may be accounted for, before the medical implant does any further processing. Since the audio is enhanced before any training or processing, the process of recognizing which command for the medical implant the audio relates to may be simplified. That is, the processing power needed for recognizing voice commands may be reduced, which is advantageous in medical implants since the size of the implant may be decreased.

FIG. 35n shows a flow chart for a method for training a medical implant to recognize a voice command, according to some embodiments. The method 200 comprises receiving 210, by a medical implant, a first audio training phrase, when the medical implant is implanted in the body of the patient.

The method further comprises creating 220 a transfer function, the transfer function being based on the first audio training phrase, wherein the transfer function is configured to adjust the amplitude of at least one frequency of audio received at the medical device for enhancing audio received at the medical implant to facilitate detection of voice commands. Creating 220 a transfer function based on a first audio input phase when the implant has been implanted in a patient allows for specifically correcting the audio input phrase for noise and/or distortion caused by the patient's body specifically.

The creation of the transfer function may be based on training a machine learning model. A purpose of the transfer function may be to adjust the audio input for distortions or noise specific to the body the implant has been implanted into. After the audio input has been adjusted, or enhanced, the audio input may be in a better condition for use in later steps of the method, such as for recognizing a command for the medical implant comprised in the audio input. In that way, there may be a two-step method for training the medical implant to recognize commands. Since the audio input has been adjusted or enhanced, the voice recognition of the command in the audio input may be easier, which may allow for using less processing power.

In some embodiments, the creating 220 a transfer function may further comprise to compare 221 the first audio training phrase with a stored audio phrase to determine a difference between them. Based on the difference, the transfer function may be created 222. In other words, the method may comprise creating a transfer function based on a difference between a stored audio phrase and the first audio phrase.

As an illustrative example only, the stored audio phrase may comprise a specific command or test phrase. When in a training session, a user of the implant or another person that the implant should be trained for, may speak the same specific command or test phrase. The command or test phrase may then be captured by the microphone of the implant, and transferred to the learning unit of the processor. The learning unit may then compare the received command or test phrase with the stored command or test phrase, and then, based on the difference(s), create a transfer function which takes the differences into account. The differences between the received command or test phrase and the stored command or test phrase may be indicative of a noise or distortion created by the body in which the implant has been implanted.

The method 200 may further comprise inputting 230 a second audio training phrase to the medical implant, the second audio training phrase comprising the voice command, the voice command comprising an instruction for the control of the medical implant.

The second audio training phrase may be used as input to the transfer function in order to create an enhanced audio training phrase. In this way, any noise or distortion created by the body may be alleviated by the transfer function, thus resulting in an enhanced audio training phrase. Thus, the method may further comprise using 240 the transfer function for generating an enhanced second audio training phrase in the medical implant.

The enhanced audio training phrase may then be associated 250 with the instruction for the control of the medical implant. That is, the method may comprise training a command unit to associate the second audio training phrase to a command for the medical device. The training may comprise training a machine learning model to associate enhanced audio training phrases with commands for the medical implant.

By first creating a transfer function, any following audio input may be enhanced by using the transfer function, and thus the associating of a second audio training phrase with a command may be simpler, i.e. less computationally intense, as the quality of the enhanced audio may be better that the originally audio received by the microphone of the implant. The method also allows for avoiding training the medical implant on distorted audio or audio with a lot of noise, thus improving the quality of the training.

FIG. 35o shows a flow chart for a method 300 for using voice commands to control a medical implant, according to some embodiments. In some examples, the voice commands have been learnt with the method described with reference to FIGS. 35m and 35n.

The method 300 comprises receiving 310 an audio command phrase for the medical device. The method further comprises applying 320 a transfer function to create an enhanced audio command phrase.

The method may further comprise to determine 320 a corresponding command for the medical based on the enhance audio command phrase, and send 340 the command to the medical device. The medical device may then execute 350 the command. By running the audio command phrase through the transfer function, the audio quality of the audio command phrase may be improved, thus allowing for an easier recognition of the corresponding command. This may make the recognition or determination of the command for the medical device less computationally intensive.

### Controlling Energy Transfer at the Implant

Any of the implantable medical implants described herein are configured to wirelessly receive energy for powering or charging the implantable medical implant. When transferring energy to an implantable medical implant it is important to adequately control the energy transfer. If the energy transferred or received at the medical implant is excessive, it may harm the patient. For example, if the position of external device relative to the receiving unit changes during energy transfer, the energy transferred may also increase or decrease drastically. This situation could cause severe problems since the implant cannot “consume” the suddenly very high amount of supplied energy. Unused excessive energy must be absorbed in some way, resulting in the generation of heat, which is highly undesirable as it may harm the patient. Hence, if excessive energy is transferred from external device the receiving unit, the temperature of the implant will increase, which may damage the surrounding tissue or otherwise have a negative effect on body functions. It is therefore highly desirable to always supply the right amount of energy to an implanted medical device during operation. Similarly, if too much energy is received by the implant, there may be temperature increases which may harm the patient. It has thus been realized that controlling the energy transfer at the medical implant may be advantageous.

An embodiment of a system for transferring energy to an implantable medical device will now be described with reference to FIGS. 35A-C. Alternatively, “transferring energy” may be referred to as “charging”. A corresponding a method for wireless energy transfer from an external energy source located outside the patient to an internal energy receiver 305 located inside the patient is also provided.

The system comprises an external energy source, or a charger, and an internal energy receiver 305. The external energy source may be comprised in any of the external devices, i.e. devices arranged outside of the body of a patient, described herein. The internal energy receiver 305 is connected to an implantable medical device 300 for supplying received energy thereto. Internal energy receiver 305 may be configured to determine an accumulated amount of received energy; determine a current change in the received energy, determine a control signal reflecting the accumulated received energy and the change in the received energy, and controlling the energy transfer based on the control signal. As an alternative, the determination of the control signal may be omitted, and the controlling may be performed based on the accumulated amount of energy and the current change.

By “controlling the energy transfer” it may be meant or include adjusting the energy transfer efficiency, controlling switches affecting the energy transfer, controlling a part of the internal energy receiver, controlling a part of the external energy source, turning the energy transfer off completely, or any other way of affecting the energy transfer.

In one embodiment the external energy source or the internal energy receiver 305 may comprise an energy transfer controller for controlling the energy transfer. The energy transfer controller may be configured to determine the rate of change of the received energy and/or the accumulated amount of received energy, and adjust the energy transfer based on the determined parameters.

Advantageously, the energy transfer may be controlled or adjusted by the internal energy receiver 305, as the internal energy receiver 305 is capable of directly determining how much energy is received in the internal energy receiver and faster determine if there is a risk to the patient or the medical implant. Thus, the internal energy receiver 305 may be configured to determine an accumulated amount of transferred energy is determined by the internal energy receiver 305. The internal energy receiver 305 may alternatively or in combination, be configured to determine a current change in the energy transfer. Further, the internal energy receiver 305 may be configured to determine a control signal for controlling the energy transfer. The control signal may be used in the internal energy receiver 305 for adjusting the receiving of energy, or it may be transmitted to the external energy source, and the external energy source may be configured to adjust the transmitted energy based on the control signal. That is, the controlling of the energy transfer may be performed by the internal energy receiver 305.

In some examples, the controlling of the energy transfer may be performed by the external energy source.

In some examples, the internal energy receiver 305 is configured to measure, via a measuring unit, an accumulated energy received a period of time and/or to measure a current change in energy received, and to control the energy received based on the accumulated energy and/or the current change. In some examples, this may be performed using a PID regulator, which will be described in the following.

In some examples, the controller comprised in the internal energy receiver comprises a PID regulator. Such a PID regulator may be used to control the difference between a received voltage and a desired voltage level. The PID regulator may control a switch to signal to selectively de-tune the receiving coil of the internal energy receiver. Alternatively, or in combination, the PID may regulate the switch to modulate the power signal. The PID regulator may respond quickly to changes in the power levels and provides increased control over the pulse width modulation of the power signal.

A PID regulator may be used for controlling any energy transfer as discussed herein.

### Pulse Width Modulation (PWM)

In some embodiments, the energy is supplied from the primary coil to the secondary coil using energy pulses. The pulses are achieved using modulation techniques. For example, modulation (PWMT—Pulse width modulation technique) of the pulses may be created with a system that controls the power using a continuous square wave pulse signal with a constant frequency where the duty cycle of the pulses is varied or a system that controls power using a continuous square wave pulse train signal with both constant frequency and constant pulse with and thereby adjusting the duty cycle width of the train of pulses. The PWMT may be used to digitally vary the amount of power from the power amplifier that drives the transmitting coil. Thus, the amount of energy transferred from the primary coil to the secondary coil may be controlled.

In some examples, the energy is supplied using a pulse pattern. In those examples, the receiving unit 305 may be configured to receive transcutaneously transferred energy in pulses according to a pulse pattern, and the measurement unit may be configured to measure a parameter related to the pulse pattern. In some examples, the controller is configured to control the energy received (for example by a variable impedance or via switches as described below) in response to the pulse pattern deviating from a predefined pulse pattern.

In some examples, the energy transmitted may be varied by varying the width of the energy pulses and having constant frequency and constant amplitude. The pulse width is achieved with a modulation technique. (hereafter PWMT) (in the preferred embodiment many times per second), to control the amount of energy transferred from the external energy transmitting coil in the system to the implanted receiver. The PWMT is used to digitally vary the amount of power from a power amplifier that drives the transmitting coil. There are several different ways to achieve the PWMT to control the amount of output energy from the power amplifier to the transmitting coil. Generally, modulation of the pulse width may be created with a system that controls the power using a continuous square wave pulse signal with a constant frequency where the duty cycle of the pulses are varied or a system that controls power using a continuous square wave pulse train signal with both constant frequency and a constant pulse width and thereby adjusting the duty cycle width of the train of pulses. These two basic techniques as well as most modifications of them can be used to control the output power of the transmitting coil.

The transmission of wireless energy from the external energy transmitting device may be controlled by applying to the external energy transmitting device electrical pulses from a first electric circuit to transmit the wireless energy, the electrical pulses having leading and trailing edges, varying the lengths of first time intervals between successive leading and trailing edges of the electrical pulses and/or the lengths of second time intervals between successive trailing and leading edges of the electrical pulses, and transmitting wireless energy, the transmitted energy generated from the electrical pulses having a varied power, the varying of the power depending on the lengths of the first and/or second time intervals.

Advantageously, the PWM embodiments described herein may be combined with any embodiment relating to controlling energy transfer to an implantable medica device, variable impedance, resonant circuit, NFMI, large coil, or any other implantable medical device being in any way configured to receive energy wirelessly, as described herein.

### Variable Impedance

According to one embodiment described with reference to FIG. 35A-35C, the controller 300 of the implantable system 10 comprises a receiving unit 305 or energy receiver 305 comprising a coil 192 (specifically shown in FIG. 35B′) configured for receiving transcutaneously transferred energy. The receiving unit 305 further comprises a measurement unit 194 configured to measure a parameter related to the energy received by the coil 192 and a variable impedance 193 electrically connected to the coil 192. The receiving unit 305 further comprises a switch 195a placed between the variable impedance 193 and the coil 192 for switching off the electrical connection between the variable impedance 193 and the coil 192. The controller 300 is configured to control the variable impedance 193 for varying the impedance and thereby tune the coil 192 based on the measured parameter. The controller 300 is further configured to control the switch 195a for switching off the electrical connection between the variable impedance 193 and the coil 192 in response to the measured parameter exceeding a threshold value. As such, the coil can be tuned or switched off to reduce the amount of received energy if the amount of received energy becomes excessive.

The controller 300 may further be configured to vary the variable impedance in response to the measured parameter exceeding a threshold value. By varying the variable impedance, the tuning of the coil may be varied, thus affecting the resonant frequency of the receiving coil. In this way, the efficiency of the reception of energy may be varied.

The measurement unit 194 is configured to measure a parameter related to the energy received by the coil 192 over a time period and/or measure a parameter related to a change in energy received by the coil 192 by for example measure the derivative of the received energy over time. The variable impedance 193 is in the embodiment shown in FIG. 35B′ placed in series with the coil 192. In alternative embodiments it is however conceivable that the variable impedance is placed parallel to the coil 192.

The first switch 195a is placed at a first end portion 192a of the coil 192, and the receiving unit 305 further comprises a second switch 195b placed at a second end portion of the coil 192, such that the coil 192 can be completely disconnected from other portions of the implantable system 10. The receiving unit 305 is configured to receive transcutaneously transferred energy in pulses according to a pulse pattern. The measurement unit 194 is in the embodiment shown in FIG. 35B′ configured to measure a parameter related to the pulse pattern. The controller 300 is configured to control the variable impedance in response to the pulse pattern deviating from a predefined pulse pattern. The controller 300 is configured to control the switch 195a for switching off the electrical connection between the variable impedance 193 and the coil 192 in response to the pulse pattern deviating from a predefined pulse pattern. The measurement unit is configured to measure a temperature in the implantable system 10 or in the body of the patient, and the controller 300 is configured to control the first and second switch 195a, 195b in response to the measured temperature.

The variable impedance 193 may comprise a resistor and a capacitor and/or a resistor and an inductor and/or an inductor and a capacitor. The variable impedance 193 may comprise a digitally tuned capacitor or a digital potentiometer. The variable impedance 193 may comprise a variable inductor. The first and second switch comprises a semiconductor, such as a MOSFET. The variation of the impedance is configured to lower the active power that is received by the receiving unit. As can be seen in FIG. 35B′, the variable impedance 193, the first and second switch 195a, 195b and the measurement unit 194 are connected to the communication unit/controller 300 and the receiving unit 305 is connected to an energy storage unit 40 such that the energy storage unit 40 can store energy received by the receiving unit 305.

### Resonant Circuit

FIG. 35P schematically illustrates a system 500 comprising an external unit 510 and an implantable medical device 505. The external unit 510 is adapted to be arranged outside of a body. The implantable medical device 505 is adapted to be implanted into a body of a patient. The external unit 510 comprises a transmitting circuit 512, a transmitting coil 514 and electrical connections 502. The electrical connections 502 electrically connect the transmitting circuit 512 with the transmitting coil 514. The electrical connections 502 may, for example, be wires or any electrically conducting material, or they may be wireless electrical connections. The electrical connections 502 may have intrinsic electrical resistance.

The implantable medical device 505 comprises an energy consuming part 528. The implantable medical device 505 further comprises receiving units 530 for receiving transcutaneously transferred energy, wherein the receiving unit 530 is configured to transfer the received energy to the energy consuming part 528. In FIG. 35P, three receiving units 530 are illustrated. However, the medical device 505 may comprise any number of receiving units 530. The medical device 505 preferably comprises two or more receiving units 530. Each receiving unit 530 comprises a receiving circuit 522, an impedance unit 526 and a receiving coil, or coil, 524, and electrical connections 502. The electrical connections 502 in the receiving unit 530 electrically connect the receiving circuit 522 to the impedance unit 526 and to the coil 524. Electrical connections 502 electrically connect the receiving unit 530 to the energy consuming part 528. The receiving unit 530 may be directly connected to the energy consuming part 528. The receiving unit 530 may be connected to intermediate circuits, wherein the intermediate circuits are connected to the energy consuming part 528. The intermediate circuits may process the output from the receiving unit 530 and prepare it for the energy consuming part 528. The intermediate circuit may comprise amplifiers, switches, filtering, modulators, other signal transformers, or a combination thereof. In FIG. 35P, the impedance units 526 are connected in parallel to the coil 524. The impedance units 526 and the respective coil 524 may instead be connected in series, partially in series and partially in parallel, or in any other way. I FIG. 35P, the impedance units 526 are capacitors. The impedance unit 526 may consist of inductors, capacitors, capacitors and resistances, inductors and resistances, or a mixture thereof. The impedance unit 526 may have a variable impedance.

The implantable medical device may further comprise a measurement unit 521 and a controller 520. The measurement unit 521 may be configured to measure a parameter related to energy transfer from the external unit 510 to the implantable medical device 505. The controller 520 may be configured to control the subcutaneously received energy to the energy consuming part 528. The controller 520 may be configured to control the subcutaneously received energy based on the parameter measured by the measurement unit 521. The controller 520 may control the impedance units 526. The controller 520 may control a variable impedance of the impedance unit 526.

The implantable medical device 505 and the external unit 510 are electrically coupled. The transmitting circuit 512 generates an alternating current in the transmitting coil 514. The alternating current of the transmitting coil 514 induces a current in the coil(s) 524. The receiving unit 530 is configured to receive transcutaneously transferred energy from the external unit 505 via the coil 524. One external unit 510 may transfer energy to many receiving units 530 having a respective coil 524.

The inductance of the coil 524 and the impedance of the corresponding impedance unit 526 contributes to a resonance frequency of the receiving unit 530. The inductance of the coils 524 and/or the impedance of the corresponding impedance unit 526 may differ in size between the respective receiving units 530. This may cause receiving units 530 to have different resonance frequencies in relation to each other. A variable impedance of the impedance unit 526 may allow the resonance frequencies of the receiving unit 530 to be tuned. The controller 520 may be able to tune the resonance frequency of each of the receiving units 530 individually by controlling the respective impedance unit 526. The receiving unit 530 may transfer different amounts of energy to the energy consuming device 528 depending on the frequency of an alternating magnetic field generated by the external device 510 and the resonance frequency of the receiving unit 530. By having different resonance frequencies for receiving units 530, a better energy transfer efficiency of the implantable medical device 505 may be obtained. Each receiving unit 530 may be designed to, or be fined tuned to, have the resonance frequency adapted to different frequencies of the external unit 510. By having different resonance frequencies of the receiving units 530, different external units 510 may be used, which is illustrated in FIG. 35Q.

FIG. 35Q schematically illustrates a system 540 comprising a second external unit 511 and an implantable medical device 545. The second external unit 511 comprises several transmitting units 509, wherein each transmitting unit 509 is similar to the external unit 510 illustrated in FIG. 35P, described above. The implantable medical device 545 illustrated in FIG. 35Q comprises an energy consuming part 528 and receiving units 530. The energy consuming part 528 of FIG. 35Q is similar to the energy consuming part 528 of FIG. 35P, described above. The receiving units 530 of FIG. 35Q are similar to the receiving units 530 of FIG. 35P, described above. The implantable medical device 545 of FIG. 35Q may further comprise a measurement unit and/or a controller similar to the measurement unit 521 and the controller 520 of FIG. 35P, described above.

FIG. 35Q illustrates an implantable medical device 545 comprising two receiving units 530. As mentioned above, there may be any number of receiving units 530 in the implantable medical device 545. As mentioned above, the receiving units 530 are connected to the energy consuming part 528, directly or via an intermediate circuit. The receiving units 530 are adapted to receive transcutaneously transferred energy from the external device 511. The external device 511 is adapted to transmit energy to the receiving unit 530 via the transmitting units 509 in the external device 511.

The external unit 511 of FIG. 35Q illustrates several transmitting units 509. The transmitting units 509 may all be comprised in one device, in separate devices, or a combination thereof. Each transmitting unit 509 may be adapted to send energy on a separate frequency. The transmitting units 509 illustrated in FIG. 35Q may be configured to transmit different frequencies. A transmitting unit 509 may be comprised in any external device or remote control described herein, a charging device, such as a smartphone, a qi charger, wireless charging pad, any device comprising a coil configured to send out energy, or any device configured to produce an oscillating a magnetic field. Each receiving unit 530 may be adapted to receive energy from a different transmitting unit 509 by having its resonance frequency configured to match the frequency of the transmitting unit 509. The transmitting units 509 could transmit sequentially, simultaneously, or have one or more transmissions partially overlap.

An advantage of multiple transmitting units 509 is that a better energy transfer efficiency of the implantable medical device 545 may be obtained. Each receiving unit 530 may be tuned to receive energy of a specific frequency of a corresponding transmitting unit 509, so that the receiving units 530 could be charged by their respective transmitting unit 509. Each receiving unit 530 may receive a respective transmitted energy sequentially, simultaneously, and/or independently of the other receiving units 530. An advantageous transmitted energy for a receiving unit 530 may be energy with the frequency of the resonance frequency of the respective receiving unit 530, energy with a frequency within a symmetric or nonsymmetric range around the resonance frequency of the respective receiving unit 530, or energy with a frequency that is at an offset from the resonance frequency of the respective receiving unit 530.

Each receiving unit 530 comprises a coil 524 and a resonance frequency. The resonance frequency is a function of the coil 524. Instead of a coil 524 and one resonance frequency, a part of a coil 524 may contribute to a resonance frequency, meaning that a coil 524 may have several resonance frequencies. This is illustrated in FIG. 35R.

FIG. 35R schematically illustrates an implantable medical device 565. The implantable medical device 565 is adapted to be implanted into a body of a patient. The implantable medical device 565 comprises an energy consuming part 528, similar to the energy consuming parts of FIGS. 35R and 35Q, described above. The implantable medical device 565 of FIG. 35R may further comprise a measurement unit and/or a controller similar to the measurement unit 521 and the controller 520 of FIG. 35P, described above. The medical device 565 may be configured to receive energy from an external unit, such as the external unit 510 of FIG. 35P and/or the external unit 511 of FIG. 35Q.

The implantable medical device 565 further comprises a receiving unit 535 for receiving transcutaneously transferred energy, wherein the receiving unit 535 is configured to transfer the received energy to the energy consuming part 528. The receiving unit 535 comprises a receiving circuit 523. The receiving unit 535 comprises a receiver coil, wherein the receiver coil comprises a coil with one or more center taps, a multitude of coils in parallel, or a combination thereof. Center taps do not have to be positioned in the center of a coil. Parts of a receiver coil are coil portions 525. The receiving unit 535 of FIG. 35R further comprises impedance units 526, similar to the impedance units 526 of FIG. 35P, described above. The receiving unit 535 of FIG. 35R further comprises electrical connections 502, similar to the electrical connections 502 of FIG. 35P, described above. The electrical connections 502 connect the receiving unit 535 to the energy consuming part 528. As mentioned for the receiving unit 530 of FIG. 35P, the receiving unit 535 of FIG. 35R may be connected directly to the energy consuming part 528 or connected first to intermediate circuits, wherein the intermediate circuits are connected to the energy consuming part 528. Intermediate circuits may be the same as described for FIG. 35P above.

The electrical connections 502 in the receiving unit 535 connect the receiving circuit 523 to the impedance units 526 and the coil portions 525 so that each impedance unit 526 is connected to a respective coil portion 525. The impedance unit 526 and the respective coil portions 525 form a receiving portion. The receiving portions may be seen as akin to the receiving units 530 of FIG. 35P, described above. FIG. 35R illustrates each impedance unit 526 being connected in parallel to the respective coil portion 525. The impedance unit 526 may be connected in series, or partially in series and partially in parallel, with the respective coil portion 525. The receiving circuit 523 is connected in parallel to the impedance unit 526. The receiving circuit 523 may be connected in parallel to a portion of the impedance unit 526. The receiving circuit 523 may be connected in series with the entire, or a portion of the, impedance unit 526.

The inductance of the coil portion 525 and the impedance of the corresponding impedance unit 526 contribute to a resonance frequency of the corresponding receiving portion. The inductance of the coil portions 525 and/or the impedance of the corresponding impedance unit 526 may differ in size between the respective receiving portions. This may cause receiving portions to have different resonance frequencies in relation to each other. A variable impedance of the impedance unit 526 may be individually controlled by a controller to change the resonance frequencies of the respective receiving portions. Each receiving portion may transfer different amounts of energy to the energy consuming device 528 depending on the resonance frequency of the receiving portion and the frequency of the transcutaneous transferred energy. By having different resonance frequencies of the receiving portions, a better energy transfer efficiency of the implantable medical device 565 may be obtained. Consecutive, sequential, or independent charging may be performed, where each receiving portion receives energy of different frequencies. Each receiving portion may have a resonance frequency adapted to different transcutaneously transferred energy frequencies, from one or more external units.

Advantages of having coil portions 525 include that it may reduce the required amount of coils and the amount of material needed. FIG. 35R illustrates sequential coil portions 525, where the coil portions 525 are adjacent but not overlapping. Coil portions 525 may overlap, be separate, or be partially overlapping segments of the receiving coil. This is illustrated in FIG. 35S.

FIG. 35S illustrates an implantable medical device 575. The implantable medical device 575 comprises an energy consuming part 528, a receiving circuit 523, and impedance units 526, similar to the energy consuming part 528, receiving circuit 523, and impedance units 526 of FIG. 35R, respectively. The implantable medical device of FIG. 35S may further comprise a measurement unit and/or a controller similar to the measurement unit 521 and the controller 520 of FIG. 35P, described above. Similar to the receiving unit 530 of FIG. 35P, the receiving unit 575 of FIG. 35S may be connected directly to the energy consuming part 528 or connected first to intermediate circuits, wherein the intermediate circuits are connected to the energy consuming part 528. Intermediate circuits may be the same as described for FIG. 35P above. The medical device 575 of FIG. 35S may be configured to receive energy from an external unit, such as the external unit 510 of FIG. 35P and/or the external unit 511 of FIG. 35Q.

The implantable medical device 575 further comprises coil portions 525, similar to the coil portions 525 of FIG. 35R, described above. FIG. 35S illustrates coil portions 525 that are overlapping with each other. Each coil portion 525 is connected to the respective impedance unit 526, as mentioned for the coil portions 525 and the impedance units 526 of FIG. 35R. An advantage of having overlapping coil portions 525 is that shorter or fewer coils may be used. Overlapping coil portions allow for larger inductances of the coil portions for a set receiver coil. Overlapping coil portions allows for a better energy transfer efficiency.

FIG. 35T illustrates an implantable medical device 585. The implantable medical device 585 comprises an energy consuming part 528, similar to the energy consuming part 528 of any of FIGS. 35P-35S. The implantable medical device 585 of FIG. 35T further comprise a first receiving unit 530, similar to the receiving unit 530 of FIGS. 35P and 35Q. The implantable medical device 585 of FIG. 35T further comprise a second receiving unit 535, similar to the receiving unit 535 of any of FIGS. 35R and 35S. The first receiving unit 530 and the second receiving unit 535 are electrically connected to the energy consuming part 528 by electrical connections 502. As mentioned for the receiving unit 530 of FIG. 35P, the receiving unit 585 of FIG. 35T may be connected directly to the energy consuming part 528 or connected first to intermediate circuits, wherein the intermediate circuits are connected to the energy consuming part 528. Intermediate circuits may be the same as described for FIG. 35P above.

FIG. 35T illustrates a implantable medical device 585 comprising one first receiving unit 530 and one second receiving unit 535. The implantable medical device 585 may comprise more than one first receiving unit 530. The implantable medical device 585 may comprise more than one second receiving unit 535. A mix of first receiving units 530 and second receiving units 535 may allow the implantable medical device 585 to be compact and customizable. The implantable medical device 585 may comprise more than one second receiving unit 535 and no first receiving units 530.

The implantable medical device 585 of FIG. 35T may further comprise a measurement unit and/or a controller similar to the measurement unit 521 and the controller 520 of FIG. 35P. The medical device 585 may be configured to receive energy from an external unit, such as the external unit 510 of FIG. 35P and/or the external unit 511 of FIG. 35Q.

In some examples, a coil comprised in the receiving unit 530 may comprise a plurality of windings. The plurality of windings may be connected to a respective variable impedance (as described above). An internal controller may control each of the variable impedances individually, thus providing for adjusting the resonant frequency of each of the windings separately. For examples, the secondary coil may comprise a first and a second winding, each connected to a respective variable impedance.

### Large Coil

A system for wirelessly charging an implantable medical implant, when implanted in a body of a patient is provided. The system comprises an internal energy receiver comprising a secondary coil, the internal energy receiver being connected to the implantable medical implant and an external energy transmitter comprising a primary coil for wirelessly transmitting energy to the internal energy receiver via the secondary coil. The diameter of the primary coil is larger than a diameter of the secondary coil.

According to embodiments described with reference to FIG. 35A-35C, the controller 300 of the implantable system 10 comprises a receiving unit 305 or internal energy receiver 305 comprising a secondary coil 192 (specifically shown in FIG. 35B′) configured for receiving transcutaneously transferred energy. The implantable system may receive the energy from an external device (also called an external energy transmitter), the external device being arranged outside of the body of the patient. The external device may comprise a primary coil for inducing a current in the coil 192 of the energy receiver 304 for wirelessly transfer energy to the receiving unit 305.

According to some embodiments, the primary coil is larger than the coil 192. By having the primary coil being larger than the secondary coil 192, the energy transmission may be improved. By having a diameter of the primary coil being larger than a diameter of the secondary coil, the wireless charging may be improved. For example, in previous wireless charging solution, there is a need for a great precision of arrangement of the secondary coil in relation to the primary coil. By having a larger diameter of the secondary coil, the need for precision may be reduced. Furthermore, having a larger primary coil wirelessly transmitting energy to a small secondary coil may provide for an improved energy transfer efficiency.

The implantable medical device may further comprise an internal controller connected to the internal energy receiver, for controlling the amount of energy received by the internal energy receiver. In some examples, the internal energy receiver further comprises a measurement unit for measuring a parameter related to the implantable medical implant or the body of the patient. The controller may be configured to measure the accumulated energy received by the internal energy receiver over a period of time and to measure a current change in energy received, and to control the energy received based on the accumulated energy and the current change. In some examples, the controlled comprises a Proportional-Integral-Derivative, PID, regulator for controlling the received energy.

The implantable medical device may comprise a variable impedance and/or a switch as described above.

With regards to the primary coil, the diameter of the primary coil may be more than 0.5 cm, more than 10 cm, more than 15 cm, more than 20 cm, more than 30 cm, or more than 50 cm. Alternatively, or in combination, the area of the primary coil is more than 0.5 cm², more than 2 cm², more than 10 cm², more than 100 cm², more than 300 cm², more than 500 cm², or more than 800 cm².

Advantageously, any of the embodiments relating to wireless charging, for example, controlling energy transfer, PID regulation, variable impedance, large coil, and emergency backup function, among others, may be combined with any embodiment related to energy transfer described herein, for example Aspects 432, 433, 434, for an increased energy transfer safety mechanism.

### Emergency Backup Function

Another risk associated with an energized implantable medical device is that the implantable medical device's battery or energy storage is depleted and thus unable to energize the implantable medical device. Further, there is a risk that the internal energy receiver malfunctions, also resulting in a malfunction of the powering of the implantable medical device.

Thus, there is provided a safety mechanism that may be advantageously combined with any embodiment or aspect relating to an energized implantable medical implant described herein.

FIG. 35V shows a schematic illustration of an implantable medical device 100. The implantable medical device 100 may have an active portion 112 and an internal energy receiver 395. The active portion 112 may, for example, be configured to treat, monitor or perform a function of a body of a patient. The implantable medical implant 100 may comprise or be connected to a backup system 113, the backup system 113 being adapted to perform a backup function related to the active portion 112 of the implantable medical device 100.

In some examples, the backup function relates to switching a function of the active portion 112 off. The backup function may be any function relating to the function of the active portion, such as, but not limited to: opening an artificial sphincter, stopping a stretching a stomach portion, or stopping a stimulation of tissue. In some examples, the backup system is configured to reverse a function of the medical devices. For example, if the implanted medical device is used to constrict the urethra of a patient having urinary incontinence, the user must naturally be capable of opening said constriction, in order to perform urination, even if the implantable medical device 100 is malfunctioning.

The backup system 113 may, for example, comprise a backup energy receiver 114 to receive energy from an external device (such as any of the external devices or remote controls described herein), or to perform a function of the active portion. The backup energy receiver 114 may be adapted to receive wirelessly transferred energy from an external device (which may also be referred to as an external energy transmitter). To this end, the backup energy receiver may comprise a second secondary coil for receiving such energy. For example, in a case where the implantable medical device 100 malfunctions, an external device may wirelessly transfer energy to the backup energy receiver. The backup energy receiver 114 may receive the wirelessly transferred energy and the received energy may be used by the backup system 113 to perform the backup function.

In some examples, the function of the backup system 113 is to transfer the energy received via the backup energy receiver 114 for powering the medical device 100, or it may be used to charge a battery or accumulator of the medical device 100.

In some examples, the backup system 113 may use a battery or energy storage used by the active portion 112.

The backup function may be triggered by an external device 320′″. The external device may be any external device or remote control as described herein. The external device 320′″ may be adapted to wirelessly transfer energy to the backup system, and/or be configured to trigger the backup function of the backup system 113. The backup function may thus comprise a backup internal communications unit 115 for receiving a command from the external device, and be configured to execute the received command.

In some examples, the backup function may be triggered by an error detected by a measuring unit or a controller comprised in the medical implant. Such an error may, for example, be detected by a pressure being too high or too low, a temperature being too high or low, a battery charge status being too low, a measurement value deviating from a predetermined interval, or something else.

In other examples, a malfunction of the implantable medical device 100 may relate to the programming of the implantable medical device. In that case, the backup function of the backup system may be to re-program the malfunctioning program of the implantable medical device 100. The re-programming may be performed using any of the methods described herein.

In some examples, the backup energy receiver 114 comprises a passive or active RFID circuit adapted to be powered by the external device. In some examples, the backup energy receiver 114 comprises an NFMI energy receiver adapted to receive energy from the external device. The backup energy receiver 114 and the backup internal communication unit 115 may in some examples be comprised in the same unit, for example, in the cases where energy transmission and wireless communication may be performed using the same hardware.

### NFMI Communication and Wireless Energy Transfer

Any one of the medical devices described herein which utilize wireless communication in any way may be comprised in a system for communicating information from or to an implantable medical device, wherein the implantable medical device is implanted in a body of a patient. The system may comprise an internal communications unit comprised in or connected to the implantable medical device, and an external communications unit, wherein the internal communications unit and the external communications units are configured to send or receive data using near-field magnetic induction.

NFMI is a short-range wireless technology that communicates using a tightly coupled magnetic field. By the term NFMI it may be meant a short range wireless physical layer using low-power and non-propagating magnetic field. NFMI systems are designed to contain transmission energy within the localized magnetic field, and the magnetic field energy resonates around the communication system, but does not radiate into free space. The power density of near-field transmissions is restrictive and attenuates or rolls off at a rate proportional to the inverse of the range to the sixth power (1/r6) or −60 dB per decade. Thus, NFMI in the typical use only has a reach of around 1.5 to 2 meters.

NFMI signal can penetrate through human body tissue with low absorption rate. For example, the specific absorption rate (SAR) may be 100 times lower than Bluetooth. It has been realized that NFMI has a communication range through body tissue of for example 50 cm, which thus makes it advantageous to use for medical implants, as compared to RF communication which is disturbed by passing though body tissue. Thus, NFMI allows for communication with implants implanted also implanted deeper in the body.

Since NFMI has such a short rage, the possibility of an adversary to eavesdrop on communication with an implant, or to hack an implant form a distance is greatly reduced, as any adversary must be very close to the implant.

FIG. 35U shows an example of a system 600 using NFMI communication between an external communications unit 601 and an implantable medical device 603. The communications unit 602 is configured to communicate with the implantable medical device 603 through the skin 624 of the patient using NFMI communication. The implantable medical device comprises an internal communications unit 610 (which may alternatively be referred to as a receiving unit 305 in other embodiments) and an active portion 612. The active portion 612 may be configured to monitor, treat or perform a function of a body of a patient, and may be any medical device or medical implant described herein. The communications unit 601 may be comprised in any external device described herein.

The external communications unit 601 comprises an external coil 604 connected to an external NFMI transceiver 606. The external NFMI transceiver 606 which may comprise an NFMI transmitter chip. The external coil 604 and the external NFMI transceiver are configured to modulate a magnetic field for sending data and/or energy to the implantable medical device 603. The external NFMI may further comprise a capacitor for tuning.

In turn, the internal communications unit 610 may comprise an internal coil 614 and an internal NFMI transceiver 616. To receive data, the magnetic field modulated by the external coil 604 induces a voltage on the internal coil 614, which may be measured by the internal NFMI transceiver 616 and be decoded at the internal NFMI transceiver or at another part of the implantable medical device 603. The NFMI transceiver 616 may comprise an NFMI receiver chip. The NFMI receiver chip may comprise a tunable resistor and capacitor tank. Both of the tunable capacitance and resistance may vary within a certain range to automatically compensate the detuning of NFMI antennas.

It will be appreciated that a similar method may be used for sending data from the implantable medical device via the internal communications unit 616 to the communications unit 601 via the external communications unit 606. In that examples, the internal communications unit may comprise an NFMI transmitter chip similar to the NFMI transmitter chip comprised in the external device, and the external NFMI transceiver may comprise an NFMI receiver chip similar to the NFMI receiver chip comprised in the internal NFMI transceiver, connected to a respective coil for transmitting and/or receiving data.

Modulation schemes such as amplitude modulation, phase modulation and frequency modulation typically used in RF communications may be used in NFMI communication.

In some embodiments, the active portion is not a pacemaker, hearing aid or a neurostimulation implant.

The internal communications unit is adapted to be implanted at a tissue depth of at least 8 or 10 cm. For example, the internal communications unit may be adapted to be implanted in an abdomen of a patient.

Thus, any internal wireless communication unit comprised in an implant described herein may use NFMI to communicate with an external device. For example, for transmitting data, receiving data, receiving new programming or changes to the software of the implant and/or receiving control commands. The short rage of NFMI and the tissue depth at which NFMI may be used, makes it advantageous to use for any communication between an external device, such as a patient EID 320″, a patient remote device 320′″, a HCP EID, a HCP remote device, and an implantable medica device.

While the communications security between an implant and an external device is improved by the use of NFMI (as compared to RF communication), the information security may advantageously be combined with any encryption, data integrity checks or the like described herein.

For example, the internal communications unit may be configured to encrypt any data to be transmitted to the external communications unit, and the external communications unit may be configured to receive the data transmitted from the internal communications unit. In some examples, the external communications unit may be further configured to transmit that data to a server.

In a more specific example, NFMI may be used for wireless communication between an implant and a patient external interrogation device, patient EID, as described herein.

In some examples the external communications unit is configured to transmit a control command to the internal communications unit, and the internal communications unit is configured to transmit the control command to the implantable medical device. The control command may cause the implantable medical device to perform an action. The internal communications unit may, for example, be configured to transmit data, the data relating to a function of the implantable medical device or a measurement obtained by the implant.

The magnetic field may in addition to or as an alternative be used for charging or powering the medical implant. The use of NFMI for changing is an alternative or addition to any wireless charging of a medical implant described herein. In those cases, the internal communications unit is configured to store the received energy in a battery or similar, or to directly forward the received energy to the active portion 612 or another energy consuming part of the implantable medical device 603.

Using NFMI for charging a medical implant also has the advantage, compared to previous methods of charging an implant, that it is not heavily affected by passing through body tissue. For example, with the use of NFMI for charging, an implant at a tissue depth of 8 and up to 13 cm or more may be charged. This allows for practically charging an implant in almost any part of a body. Advantageously, the NFMI communication system disclosed herein may be combined with any of aspects 250, 252, 255 and 284, and any of the embodiments described herein relating to wireless energy transfer using a coil.

According to one example, the system further comprises a second internal communications unit and a second external communications unit, wherein the second internal communications unit is adapted to receive and transmit data using a short range communications technology, and the second external communications unit is adapted to receive and transmit data using a short range communications technology, the short range communications technology having a shorter maximum range than NFMI. In one example, the short range communications technology is NFC, and the second internal communication unit comprises an NFC transceiver and the second external communication unit comprises an interrogation device for transmitting data to and from the RFID transceiver. By having these second internal and external communications unit, the implant may require a second authentication based on that the external communication unit is close to the implant, for example close enough to interrogate the NCF transceiver. Thus, it may be verified that the second external communication device is indeed close to the patient's body.

In the following, numbered aspect groups xx-xx of the present inventive concept are provided. The different aspects are numbered individually within the groups and the references to other aspects relate primarily to aspects within the same group. The scope of protection is however defined by the appended claims.

### Aspect Group 460PC: Stimulation_Electrical_General_Parasympathetic/Sympathetic

1. A system for affecting an effector response in a patient, comprising:
   a stimulation device configured to deliver, directly or indirectly a first stimulation signal to a sympathetic nerve innervating a first effector tissue of the patient and a second stimulation signal to a parasympathetic nerve innervating a second effector tissue; and
   a control unit configured to control an operation of the stimulation device such that:
   the first stimulation signal stimulates an activity of the sympathetic nerve and the second stimulation signal inhibits an activity of the parasympathetic nerve or
   the first stimulation signal inhibits an activity of the sympathetic nerve and the second stimulation signal stimulates an activity of the parasympathetic nerve.
2. The system according to aspect 1, wherein the control unit is configured to control the operation of the stimulation device such that at least one of the first stimulation signal and second stimulation signal is a periodic signal including at least one of: a variable frequency component, a variable duty cycle component, a variable amplitude component, and a variable pause component.
3. The system according to aspect 1 or 2, wherein:
   the first signal is a low-frequency signal configured to stimulate the activity of the sympathetic nerve and the second signal is a high-frequency signal configured to inhibit the activity of the parasympathetic nerve; or
   the first signal is a high-frequency signal configured to inhibit the activity of the sympathetic nerve and the second signal is a low-frequency signal configured to stimulate the activity of the parasympathetic nerve.
4. The system according to aspect 4, wherein an amplitude of the low-frequency signal varies with a frequency in a range of 0.1-100 Hz and wherein an amplitude of the high-frequency signal varies with a frequency in a range of 1-10 KHz.
5. The system according to any one of aspects 1 to 3, wherein at least one of the first and second stimulation signals comprises a series of pulses having a negative voltage relative to ground.
6. The system according to aspect 5, wherein the control unit is configured to operate the stimulation device to generate a positive voltage pulse following one or more negative voltage pulses.
7. The system according to any one of aspects 1 to 6, wherein at least one of the first stimulation signal and the second stimulation signal is an electric signal or a vibrational signal.
8. The system according to any one of aspects 1 to 7, wherein the control unit is configured to operate the stimulation device so as to alternatingly apply the first stimulation signal to the sympathetic nerve and the second stimulation signal to the parasympathetic nerve.
9. The system according to any one of aspects 1 to 7, wherein the control unit is configured to operate the stimulation device so as to simultaneously apply the first stimulation signal to the sympathetic nerve and the second stimulation signal to the parasympathetic nerve.
10. The system according to any one of aspects 1 to 9, wherein the control unit is configured to control the operation of the stimulation device to generate an effector response as at least one of a muscular response and a glandular response.
11. The system according to aspect 10, wherein:
   each of the first and second effector tissue is a muscular tissue; and
   the control unit is configured to control the operation of the stimulation device such that the first stimulation signal stimulates the activity of the sympathetic nerve and the second stimulation signal inhibits the activity of the parasympathetic nerve, thereby inducing contraction in the muscular tissue.
12. The system according to aspect 10, wherein:
   each of the first and second effector tissue is a muscular tissue; and
   the control unit is configured to control the operation of the stimulation device such that the first stimulation signal inhibits the activity of the sympathetic nerve and the second stimulation signal stimulates the activity of the parasympathetic nerve, thereby inducing relaxation in the muscular tissue.
13. The system according to aspect 11 or 12, wherein the first and second effector tissue is smooth muscle tissue.
14. The system according to aspect 13, wherein the first and second effector tissue form part of a blood vessel, an intestine, or a urine bladder of the patient.
15. The system according to any of the preceding aspects, further comprising:
   a sensor device configured to generate a sensor signal indicating an effector response in the effector tissue and
   a control unit configured to receive the sensor signal and control an operation of the stimulation device based at least in part on the sensor signal.
16. The system according to aspect 15, wherein the sensor device comprises a sensor electrode configured to measure an electric activity in the effector tissue.
17. The system according to aspect 15, wherein the sensor device comprises a sensor electrode configured to measure a change in electrical impedance in the effector tissue.
18. The system according to aspect 15, wherein the sensor device comprises an electromyographic sensor configured to measure an electric activity in the effector tissue and an electric impedance sensor configured to measure a change in electrical impedance in the effector tissue.
19. The system according to aspect 16 or 17, wherein:
   the sensor electrode is configured to be arranged at the effector tissue,
   the sensor device further comprises a reference electrode and
   the sensor device is configured to generate the sensor signal based on an electrical interaction between the sensor electrode and the reference electrode.
20. The system according to aspect 19, wherein the reference electrode is formed by a housing of the stimulation device or the sensor device.
21. The system according to any one of aspects 15 to 20, wherein the sensor device is configured to measure mechanical movement in the effector tissue.
22. The system according to aspect 21, wherein the sensor device comprises a strain gauge configured to measure a contraction or relaxation of the effector tissue.
23. The system according to any one of aspects 15 to 22, wherein the control unit is configured to determine a response measure based on the sensor signal, the response measure being indicative of the effector response.
24. The system according to aspect 23, wherein the control unit is configured to compare the response measure with a predetermined reference measure and control the stimulation device to:
   in response to the response measure being below the reference measure, one or both: (a) increase an intensity of the first stimulation signal to stimulate the activity in the sympathetic nerve and (b) increase an intensity of the second stimulation signal to inhibit the activity of the parasympathetic nerve, and
   in response to the response measure exceeding the reference measure, one or both: (a) reduce the intensity of the first stimulation signal to inhibit the activity of the sympathetic nerve and (b) stimulate the activity of the parasympathetic nerve.
25. The system according to aspect 24, wherein the predetermined reference measure is based on a previous measurement of the effector response in the patient.
26. The system according to aspect 24 or 25, wherein the predetermined reference measure is based on previous measurement of effector responses in other patients.
27. The system according to any one of aspects 23 to 26, wherein the control unit is configured to monitor the response measure of effector response over time and control the stimulation device based on a change rate in the effector response over time.
28. The system according to any one of aspects 23 to 26, wherein the control unit is configured to determine a calibration parameter of the stimulation device based on the response measure.
29. The system according to any one of aspects 1 to 28, wherein the stimulation device comprises a first stimulation arrangement configured to be coupled to the sympathetic nerve to deliver the first stimulation signal and a second stimulation arrangement configured to be coupled to the parasympathetic nerve to deliver the second stimulation signal.
30. The system according to aspect 29, wherein the first stimulation arrangement comprises a first stimulation electrode and a second stimulation electrode, wherein the first stimulation electrode and the second stimulation electrode are configured to be spaced apart along the sympathetic nerve.
31. The system according to aspect 29 or 30, wherein the stimulation device is configured to generate the first stimulation signal such that the first stimulation electrode serves as a cathode and
   the second stimulation electrode serves as an anode.
32. The system according to any one of aspects 29 to 31, further comprising a cuff configured to be at least partially arranged around the nerve and hold the first stimulation arrangement in place against the sympathetic nerve.
33. The system according to any one of aspects 29 to 32, wherein the second stimulation arrangement comprises a third electrode and a fourth electrode, the third electrode and the fourth electrode being configured to be arranged spaced apart along the parasympathetic nerve.
34. The system according to aspect 33, wherein the second stimulation arrangement further comprises a fifth electrode configured to be arranged spaced apart from the fourth electrode such that the fourth electrode is arranged between the third and fifth electrodes.
35. The system according to aspect 34, wherein the stimulation device is configured to generate the second stimulation signal such that the fourth electrode serves as a cathode and the third and fifth electrodes serve as anodes.
36. The system according to any one of aspects 29 to 35, further comprising a cuff configured to be at least partially arranged around the parasympathetic nerve and hold the second stimulation arrangement in place against the parasympathetic nerve.
37. The system according to any of the preceding aspects, wherein:
   the first and second effector tissue is smooth muscle tissue of a renal artery of the patient; and
   the control unit is configured to control the operation of the stimulation device to induce at least one of vasodilation and vasoconstriction in the renal artery, thereby affecting a blood pressure of the patient.
38. The system according to any of aspects 1-36, wherein:
   the first effector tissue forms part of an artery supplying erectile genital tissue with blood;
   the second effector tissue forms part of a vein draining the blood from the genital erectile tissue; and
   the control unit is configured to control the operation of the stimulation device to induce vasodilation in the artery and vasoconstriction in the vein, thereby inducing erection in the erectile genitalia.
39. The system according to aspect 38, wherein the artery is a penile artery, and the vein is a penile vein.
40. The system according to aspect 39, wherein each of the sympathetic nerve and the parasympathetic nerve form part of a cavernous nerve of the patient.
41. The system according to aspect 40, wherein:
   the first stimulation device is configured to deliver the first stimulation signal to the parasympathetic nerve at a position between the sacral spinal cord segments S2-S4 and the pelvic plexus; and
   the second stimulation device is configured to deliver the second stimulation signal to the sympathetic nerve at a position between the levels of the T11-L2 vertebrae and the pelvic plexus.
42. The system according to any of aspects 1-36, wherein:
   the effector tissue is smooth muscle tissue of a gastrointestinal tract of the patient; and
   the control unit is configured to control the operation of the stimulation device to affect a level of motility of the gastrointestinal tract, thereby affecting at least one of nutrition uptake and fecal texture.
43. The system according to aspect 42, wherein the control unit is configured to control the operation of the stimulation device to affect at least one of a peristalsis pattern and a segmentation pattern of an intestine of the patient.
44. The system according to any of aspects 1-36, wherein:
   the at least one of the first effector tissue and the second effector tissue is a glandular tissue; and
   the control unit is configured to control the operation of the stimulation device to affect a level of glandular secretion of the glandular tissue.
45. The system according to aspect 44, wherein the glandular tissue forms part of at least one of:
   a pancreas secreting insulin,
   a gallbladder secreting bile, and
   an adrenal gland secreting adrenaline, aldosterone, or cortisol.

### Aspect Group 461SE: Stimulation_Electrical_General_Frequency for Tissue

1. A system for affecting an effector response in a patient, comprising:
   a stimulation device configured to be coupled to at least one of an effector tissue and a nerve innervating the effector tissue of the patient,
   a control unit configured to operate the stimulation device to apply at least one of a first stimulation signal and a second stimulation signal to the effector tissue,
   wherein the first stimulation signal is a time-varying signal with an amplitude varying with a frequency lying in a first frequency interval,
   wherein the second stimulation signal is a time-varying signal with an amplitude varying with a frequency lying in a second frequency interval,
   wherein the first frequency interval is selected to induce the effector response in the effector tissue, and
   wherein the second frequency interval is selected to inhibit the effector response in the effector tissue.
2. The system according to aspect 1, wherein the first frequency interval is in a range of 0.1-100 Hz and the second frequency interval is in a range of 1-10 KHz.
3. The system according to aspect 1 or 2, wherein at least one of the first and second stimulation signals is an electric signal comprising a series of pulses having a negative voltage relative to ground.
4. The system according to aspect 3, wherein the control unit is configured to operate the stimulation device to generate a positive voltage pulse following one or more negative voltage pulses.
5. The system according to aspect 3 or 4, wherein the control unit is configured to operate the stimulation device to generate a first stimulation signal having a frequency in a range of 0.5-3 Hz, and wherein the effector tissue is cardiac muscle tissue.
6. The system according to aspect 3 or 4, wherein the control unit is configured to operate the stimulation device to generate a first stimulation signal having a frequency in a range of 1-10 Hz, and wherein the effector tissue is skeletal muscle tissue.
7. The system according to aspect 3 or 4, wherein the control unit is configured to operate the stimulation device to generate a first stimulation signal having a frequency in a range of 0.1-100 Hz, and wherein the effector tissue is smooth muscle tissue.
8. The system according to any one of aspects 1 to 7, wherein at least one of the first stimulation signal and the second stimulation signal is an electric signal or a vibrational signal.
9. The system according to any one of aspects 1 to 8, wherein the control unit is configured to operate the stimulation device to alternatingly apply the first stimulation signal and the second stimulation signal to the effector tissue.
10. The system according to any one of aspects 1 to 9, further comprising a sensor device configured to generate a sensor signal indicating an effector response in the effector tissue, wherein the control unit is further configured to:
   receive the sensor signal and
   control an operation of the stimulation device based at least in part on the sensor signal.
11. The system according to aspect 10, wherein the sensor device comprises a sensor electrode configured to measure an electric activity in the effector tissue.
12. The system according to aspect 10, wherein the sensor device comprises a sensor electrode configured to measure a change in electrical impedance in the effector tissue.
13. The system according to aspect 10, wherein sensor device comprises an electromyographic sensor electrode configured to measure an electric activity in the effector tissue and an electric impedance sensor electrode configured to measure a change in electrical impedance in the effector tissue.
14. The system according to aspect 11 or 12, wherein:
   the sensor electrode is configured to be arranged at the effector tissue,
   the sensor device further comprises a reference electrode, and
   the sensor device is configured to generate the sensor signal based on an electrical interaction between the sensor electrode and the reference electrode.
15. The system according to aspect 14, wherein the reference electrode is formed by a housing of the stimulation device or the sensor device.
16. The system according to any one of aspects 10 to 15, wherein the sensor device is configured to measure mechanical movement in the effector tissue.
17. The system according to aspect 16, wherein the sensor device comprises a strain gauge configured to measure a contraction or relaxation of the effector tissue.
18. The system according to any one of aspects 10 to 17, wherein the control unit is configured to determine a response measure based on the sensor signal, the response measure being indicative of the effector response.
19. The system according to aspect 18, wherein the control unit is configured to compare the response measure with a predetermined reference measure and control the stimulation device to:
   in response to the response measure being below the reference measure, increase an intensity of the first stimulation signal to stimulate the activity in the in the effector tissue, and
   in response to the response measure exceeding the reference measure, increase the intensity of the second stimulation signal to inhibit the activity of the effector tissue.
20. The system according to aspect 19, wherein the predetermined reference measure is based on a previous measurement of the effector response in the patient.
21. The system according to aspect 19 or 20, wherein the predetermined reference measure is based on a previous measurement of effector responses in other patients.
22. The system according to any one of aspects 18 to 21, wherein the control unit is configured to monitor the response measure of effector response over time and control the stimulation device based on a change rate in the effector response over time.
23. The system according to any one of aspects 18 to 21, wherein the control unit is configured to determine a calibration parameter of the stimulation device based on the response measure.
24. The system according to any one of aspects 1 to 23, wherein the stimulation device comprises a first stimulation arrangement configured to deliver the first stimulation signal and a second stimulation arrangement configured to deliver the second stimulation signal.
25. The system according to aspect 24, wherein the first stimulation arrangement comprises a first stimulation electrode and a second stimulation electrode, wherein the first stimulation electrode and the second stimulation electrode are configured to be spaced apart along the nerve innervating the effector tissue.
26. The system according to aspect 24 or 25, wherein the stimulation device is configured to generate the first stimulation signal such that the first stimulation electrode serves as a cathode and the second stimulation electrode serves as an anode.
27. The system according to any one of aspects 24 to 26, further comprising a cuff configured to be at least partially arranged around the nerve and hold the first stimulation arrangement in place against the nerve.
28. The system according to any one of aspects 24 to 27, wherein the second stimulation arrangement comprises a third electrode and a fourth electrode, wherein the third electrode and the fourth electrode is configured to be arranged spaced apart along the nerve.
29. The system according to aspect 28, wherein the stimulation device is configured to generate the second stimulation signal such that the third electrode serves as a cathode and the fourth electrode serves as an anode.
30. The system according to any one of aspects 24 to 29, further comprising a cuff configured to be at least partially arranged around the nerve and hold the second stimulation arrangement in place against the nerve.
31. The system according to any one of aspects 1 to 30, further comprising a suppression arrangement configured to be coupled to the nerve in order to apply a suppression signal which is configured to suppress action potentials propagating in the nerve in a direction towards the central nervous system.
32. The system according to aspect 31, wherein the control unit is configured to regulate the suppression signal so as to suppress the action potentials induced in response to the stimulation device applying the first stimulation signal.
33. The system according to aspect 31 or 32, wherein stimulation device is configured to be coupled to the nerve at a position between the effector tissue and the suppression arrangement so as to induce action potentials travelling in the nerve in a direction towards the effector tissue.
34. The system according to any one of aspects 31 to 33, wherein the control unit is configured to regulate the suppression of the action potentials so as to inhibit an undesired response of the nervous system of the patient, wherein the undesired response is generated responsive to the stimulation device applying the first stimulation signal.
35. The system according to any one of aspects 31 to 34, wherein the control unit is configured to drive the stimulation device and the suppression arrangement such that each of the stimulation device and the suppression arrangement is actuated in sequence, with a delay of the suppression signal timed to generally match a conduction velocity of the first stimulation signal.
36. The system according to any one of aspects 31 to 34, wherein the control unit is configured to drive the stimulation device and the suppression arrangement so as to apply the first stimulation signal and the suppression signal substantially at the same time.
37. The system according to any one of aspects 31 to 36, wherein the control unit is configured to drive the stimulation device and the suppression arrangement such that each of the first stimulation signal and the suppression signal is a time-varying signal, wherein the first stimulation signals is a low-frequency signal and the suppression signal is a high-frequency signal.
38. The system according to any one of aspects 31 to 37, wherein an amplitude of the first stimulation signal varies with a frequency in a range of 0.1-100 Hz and wherein an amplitude of the suppression signal varies with a frequency in a range of 1-10 KHz.

### Aspect Group 462SE: Stimulation_Electrical_General_Autonomic Tone

1. A method for affecting a balance between a sympathetic tone and parasympathetic tone of the autonomic nervous system of a patient, comprising:
   delivering, by a first stimulation arrangement, a first stimulation signal to a sympathetic nerve of the patient to increase a level of activity of the sympathetic nerve and move the balance towards a sympathetic dominance, or
   delivering, by a second stimulation arrangement, a second stimulation signal to the parasympathetic nerve to increase a level of activity of the parasympathetic nerve and move the balance towards a parasympathetic dominance.
2. The method according to aspect 1, wherein each of the sympathetic nerve and the parasympathetic nerve forms part of at least one of: a cardiovascular system, a respiratory system, a gastrointestinal tract, a urinary system, an immune system, a sexual function, and a stress response system.
3. The method according to aspect 1 or 2, wherein the first stimulation signal is delivered to the vagus nerve.
4. The method according to aspect 3, wherein the first stimulation signal is delivered to the celiac branch of the vagus nerve.
5. The method according to aspect 1 or 2, wherein the first stimulation signal is delivered to the sacral plexus.
6. The method according to aspect 1 or 2, wherein at least one of the first stimulation signal and the second stimulation signal is delivered to the cardiac plexus.
7. The method according to aspect 1 or 2, wherein at least one of the first stimulation signal and the second stimulation signal is delivered to the pulmonary plexus.
8. The method according to aspect 1 or 2, wherein the second stimulation signal is delivered to the celiac plexus.
9. The method according to aspect 1 or 2, wherein the second stimulation signal is delivered to the hypogastric plexus.
10. The method according to aspect 1 or 2, wherein the first stimulation signal is delivered to the pelvic plexus.
11. The method according to aspect 1 or 2, wherein at least one of the first stimulation signal and the second stimulation signal is delivered to the hypothalamus.
12. The method according to aspect 1 or 2, wherein at least one of the first stimulation signal and the second stimulation signal is delivered to the brainstem.
13. The method according to aspect 12, wherein at least one of the first stimulation signal and the second stimulation signal is delivered to the midbrain, the pons, or the medulla oblongata.
14. The method according to any of the preceding aspects, wherein each of the first and second stimulation signals is a periodic signal including at least one of: a variable frequency component, a variable duty cycle component, a variable amplitude component, and a variable pause component.
15. The method according to any of the preceding aspects, wherein each of the first stimulation signal and the second stimulation signal comprises an amplitude varying with a frequency in the range of 0.1-100 Hz.
16. The method according to any of the preceding aspects, wherein at least one of the first and second stimulation signals comprises series of pulses having a negative voltage relative to ground.
17. The method according to aspect 16, comprising generating a positive voltage pulse following one or more negative voltage pulses.
18. The method according to any of the preceding aspects, wherein at least one of the first stimulation signal and the second stimulation signal is an electric signal or a vibrational signal.
19. The method according to any of the preceding aspects, further comprising:
   receiving a sensor signal indicating an activity in the sympathetic nerve in response to the first stimulation signal, or an activity in the parasympathetic nerve in response to the second stimulation signal; and
   adjusting an intensity of at least one of the first stimulation signal and the second stimulation signal based at least in part on the sensor signal.
20. The method according to aspect 19, wherein the sensor signal indicates an electric activity in the sympathetic nerve and/or the parasympathetic nerve.
21. The method according to aspect 19, wherein the sensor signal indicates a change in electrical impedance in the sympathetic nerve and/or the parasympathetic nerve.
22. The method according to any of the preceding aspects, comprising delivering the first stimulation signal by a first stimulation electrode and a second stimulation electrode arranged spaced apart along the sympathetic nerve.
23. The method according to aspect 22, comprising delivering the first stimulation signal such that the first stimulation electrode serves as a cathode and the second stimulation electrode serves as an anode.
24. The method according to aspects 22-23, comprising delivering the second stimulation signal by a third stimulation electrode and a fourth stimulation electrode arranged spaced apart along the parasympathetic nerve.
25. The method according to aspect 24, comprising delivering the second stimulation signal such that the third electrode serves as a cathode and the fourth electrodes serves as an anode.
26. The method according to any of the preceding aspects, further comprising:
   applying a first suppression signal to the sympathetic nerve to suppress action potentials generated by the first stimulation signal and propagating in a direction towards the central nervous system.
27. The method according to aspect 26, comprising:
   regulating the suppression of the action potentials to inhibit an undesired response of the nervous system of the patient, the undesired response being generated responsive to the first stimulation signal.
28. The method according to aspects 26 or 27, comprising:
   applying the first stimulation signal and the first suppression signal in sequence, with a delay of the first suppression signal timed to generally match a conduction velocity of the first stimulation signal in the sympathetic nerve.
29. The method according to any of aspects 26-28, wherein an amplitude of the first suppression signal varies with frequency in the range of 1-10 KHz.
30. The method according to any of the preceding aspects, further comprising:
   applying a second suppression signal to the parasympathetic nerve to suppress action potentials generated by the second stimulation signal and propagating in a the direction towards the central nervous system.
31. The method according to aspect 30, comprising:
   regulating the suppression of the action potentials to inhibit an undesired response of the nervous system of the patient, the undesired response being generated responsive to the second stimulation signal.
32. The method according to aspect 30 or 31, comprising:
   applying the second stimulation signal and the second suppression signal in sequence, with a delay of the second suppression signal timed to generally match a conduction velocity of the second stimulation signal in the parasympathetic nerve.
33. The method according to any of aspects 30-32, wherein an amplitude of the second suppression signal varies with frequency in the range of 1-10 KHz.

### Aspect Group 463SE: Stimulation_Electrical_General_Unidirectional

1. A system for affecting an effector response in a patient, comprising:
   a stimulation device comprising a first stimulation arrangement and a second stimulation arrangement, each configured to be coupled to at least one of an effector tissue and a nerve innervating the effector tissue of the patient,
   a control unit configured to drive the stimulation device to apply, by means of the first stimulation arrangement, a stimulation signal inducing the effector response in the effector tissue,
   and, by means of the second stimulation arrangement, a suppression signal suppressing action potentials propagating in the nerve towards the central nervous system (CNS),
   wherein the control unit is configured to regulate the suppression signal so as to suppress the action potentials induced in response to the stimulation device applying the stimulation signal.
2. The system according to aspect 1, wherein the first stimulation arrangement is configured to be coupled to the nerve at a position between the effector tissue and the second electrode so as to induce action potentials travelling in the nerve in a direction towards the effector tissue.
3. The system according to aspect 2, wherein the control unit is configured to regulate the suppression of the action potentials so as to inhibit an undesired response of the nervous system of the patient, wherein the undesired response is generated responsive to the first electrode applying the stimulation signal.
4. The system according to aspect 2 or 3, wherein the control unit is configured to drive the stimulation device such that each of the first and second stimulation arrangements are actuated in sequence, with a delay of the suppression signal timed to match a conduction velocity of the stimulation signal.
5. The system according to any of aspects 1 to 3, wherein the control unit is configured to drive the stimulation device to apply the stimulation signal and the suppression signal substantially at the same time.
6. The system according to any one of aspects 1 to 5, wherein the control unit is configured to drive the stimulation device such that each of the stimulation signal and the suppression signal is a time-varying signal, wherein the stimulation signal is a low-frequency signal and the suppression signal is a high-frequency signal.
7. The system according to aspect 6, wherein an amplitude of the stimulation signal varies with a frequency in a range of 0.1-100 Hz and wherein an amplitude of the suppression signal varies with a frequency in a range of 1-10 KHz.
8. The system according to any one of aspects 1 to 7, wherein the first and second stimulation arrangements are configured to be spaced apart along the nerve.
9. The system according to any one of aspects 1 to 8, wherein the first stimulation arrangement comprises a first stimulation electrode and a second stimulation electrode configured to apply the stimulation signal to the effector tissue or the nerve.
10. The system according to aspect 9, wherein the first stimulation electrode and the second stimulation electrode are configured to be spaced apart along the nerve.
11. The system according to aspect 9 or 10, wherein the stimulation device is configured to generate the stimulation signal such that the first stimulation electrode serves as a cathode and the second stimulation electrode serves as an anode.
12. The system according to any one of aspects 1 to 11, further comprising a cuff configured to be at least partially arranged around the nerve and hold the first stimulation arrangement in place against the nerve.
13. The system according to any one of aspects 1 to 12, wherein the second stimulation arrangement comprises a first suppression electrode and a second suppression electrode configured to apply the suppression signal to the nerve.
14. The system according to aspect 13, wherein the first suppression electrode and the second suppression electrode are configured to be spaced apart along the nerve.
15. The system according to aspect 13 or 14, wherein the second stimulation arrangement further comprises a third suppression electrode configured to be arranged spaced apart from the second suppression electrode such that the second suppression electrode is arranged between the first and third suppression electrodes.
16. The system according to aspect 15, wherein the stimulation device is configured to generate the suppression signal such that the second suppression electrode serves as a cathode and the first and third suppression electrodes serve as anodes.
17. The system according to any one of aspects 13 to 16, further comprising a cuff configured to be at least partially arranged around the nerve and hold the second stimulation arrangement in place against the nerve.
18. The system according to any one of aspects 1 to 17, further comprising a sensor device configured to generate a sensor signal indicating an effector response in the effector tissue, wherein the control unit is further configured to:
   receive the sensor signal and
   control an operation of the stimulation device based at least in part on the sensor signal.
19. The system according to aspect 18, wherein the sensor device comprises a sensor electrode configured to measure an electric activity in the effector tissue.
20. The system according to aspect 18, wherein the sensor device comprises a sensor electrode configured to measure a change in electrical impedance in the effector tissue.
21. The system according to aspect 18, wherein sensor device comprises an electromyographic sensor electrode configured to measure an electric activity in the effector tissue and an electric impedance sensor electrode configured to measure a change in electrical impedance in the effector tissue.
22. The system according to aspect 19 or 20, wherein:
   the sensor electrode is configured to be arranged at the effector tissue;
   the sensor device further comprises a reference electrode, and
   the sensor device is configured to generate the sensor signal based on an electrical interaction between the sensor electrode and the reference electrode.
23. The system according to aspect 22, wherein the reference electrode is formed by a housing of the stimulation device or the sensor device.
24. The system according to any one of aspects 18 to 23, wherein the sensor device is configured to measure mechanical movement in the effector tissue.
25. The system according to aspect 24, wherein the sensor device comprises a strain gauge configured to measure a contraction or relaxation of the effector tissue.
26. The system according to any one of aspects 18 to 23, wherein the control unit is configured to determine a response measure based on the sensor signal, wherein the response measure is indicative of the effector response.
27. The system according to aspect 26, wherein the control unit is configured to compare the response measure with a predetermined reference measure and control the stimulation device to:
   in response to the response measure being below the reference measure, increase an intensity of the stimulation signal to stimulate the activity in the in the effector tissue, and
   in response to the response measure exceeding the reference measure, reduce the intensity of the stimulation signal to inhibit the activity of the effector tissue.
28. The system according to aspect 27, wherein the predetermined reference measure is based on a previous measurement of the effector response in the patient.
29. The system according to aspect 26 or 27, wherein the predetermined reference measure is based on a previous measurement of effector responses in other patients.
30. The system according to any one of aspects 26 to 29, wherein the control unit is configured to monitor the response measure of effector response over time, and to control the stimulation device based on a change rate in the effector response over time.
31. The system according to any one of aspects 26 to 29, wherein the control unit is configured to determine a calibration parameter of the stimulation device based on the response measure.
32. The system according to any of the preceding aspects, wherein:
   the effector tissue is smooth muscle tissue of a renal artery of the patient; and
   the control unit is configured to control the operation of the stimulation device to induce at least one of vasodilation and vasoconstriction in the renal artery, thereby affecting a blood pressure of the patient.
33. The system according to any of aspects 1-31, wherein:
   the effector tissue forms part of an artery supplying erectile genital tissue with blood; and
   the control unit is configured to control the operation of the stimulation device to induce vasodilation in the artery.
34. The system according to aspect 33, wherein the artery is a penile artery.
35. The system according to aspect 33 or 34, wherein the stimulation device is configured to deliver the stimulation signal to a parasympathetic nerve of a cavernous nerve of the patient, at a position between the sacral spinal cord segments S2-S4 and the pelvic plexus.
36. The system according to any of aspects 1-31, wherein:
   the effector tissue forms part of a vein draining the blood from the genital tissue; and
   the control unit is configured to control the operation of the stimulation device to induce vasoconstriction in the vein.
37. The system according to aspect 36, wherein the vein is a penile vein.
38. The system according to aspect 36 or 37, wherein the stimulation device is configured to deliver the stimulation signal to a sympathetic nerve of a cavernous nerve of the patient, at a position between the levels of the T11-L2 vertebrae and the pelvic plexus.
39. The system according to any of aspects 1-31, wherein:
   the effector tissue is smooth muscle tissue of a gastrointestinal tract of the patient; and
   the control unit is configured to control the operation of the stimulation device to affect a level of motility of the gastrointestinal tract, thereby affecting at least one of nutrition uptake and fecal texture.
40. The system according to aspect 39, wherein the control unit is configured to control the operation of the stimulation device to affect at least one of a peristalsis pattern and a segmentation pattern of an intestine of the patient.
41. The system according to any of aspects 1-31, wherein:
   the effector tissue is a glandular tissue; and
   the control unit is configured to control the operation of the stimulation device to affect a level of glandular secretion of the glandular tissue.
42. The system according to aspect 41, wherein the glandular tissue forms part of at least one of:
   a pancreas secreting insulin,
   a gallbladder secreting bile, and
   an adrenal gland secreting adrenaline, aldosterone, or cortisol.

### Aspect Group 464SE: Stimulation_Electrical_Feedback_Measured Effect

1. A system for affecting an effector response in a patient, comprising:
   a stimulation device configured to deliver, directly or indirectly, a stimulation signal to at least one of an effector tissue and a nerve innervating the effector tissue of the patient,
   a sensor device configured to generate a sensor signal indicating an effector response in the effector tissue, and
   a control unit configured to receive the sensor signal and control an operation of the stimulation device based at least in part on the sensor signal.
2. The system according to aspect 1, wherein the sensor device comprises a sensor electrode configured to measure an electric activity in the effector tissue in response to the stimulation signal.
3. The system according to aspect 1, wherein the sensor device comprises a sensor electrode configured to measure a change in electrical impedance in the effector tissue in response to the stimulation signal.
4. The system according to aspect 1, wherein sensor device comprises an electromyographic sensor configured to measure an electric activity in the effector tissue and an electric impedance sensor configured to measure a change in electrical impedance in the effector tissue.
5. The system according to aspect 2 or 3, wherein:
   the sensor electrode is configured to be arranged at the effector tissue,
   the sensor device further comprises a reference electrode, and
   the sensor device is configured to generate the sensor signal based on an electrical interaction between the sensor electrode and the reference electrode.
6. The system according to aspect 5, wherein the reference electrode is formed by a housing of the stimulation device or the sensor device.
7. The system according to any one of aspects 1 to 6, wherein the sensor device is configured to measure mechanical movement in the effector tissue in response to the stimulation signal.
8. The system according to aspect 7, wherein the sensor device comprises a strain gauge configured to measure a contraction or relaxation of the effector tissue in response to the stimulation signal.
9. The system according to aspect 1, wherein the sensor device is configured to measure a heart rate of the patient.
10. The system according to aspect 1, wherein the sensor device is configured to measure a blood pressure of the patient.
11. The system according to aspect 1, wherein the sensor device is configured to measure a rate of respiration of the patient.
12. The system according to any one of aspects 1 to 11, wherein the control unit is configured to determine a response measure based on the sensor signal, wherein the response measure is indicative of the effector response.
13. The system according to aspect 12, wherein the control unit is configured to compare the response measure with a predetermined reference measure and control the stimulation device to:
   increase an intensity of the stimulation signal in response to the response measure being below the reference measure and
   reduce the intensity of the stimulation signal in response to the response measure exceeding the reference measure.
14. The system according to aspect 13, wherein the control unit is configured to:
   increase the intensity of the stimulation signal by increasing at least one of a frequency, current amplitude, and voltage amplitude of the stimulation signal; and
   reduce the intensity of the stimulation signal by reducing at least one of the frequency, current amplitude, and voltage amplitude of the stimulation signal.
15. The system according to aspect 13 or 14, wherein the predetermined reference measure is based on a previous measurement of the effector response in the patient.
16. The system according to aspect 13 or 14, wherein the predetermined reference measure is based on previous measurements of effector responses in other patients.
17. The system according to aspect 12, wherein the control unit is configured to monitor the level of effector response over time and control the stimulation device based on a change rate in the effector response over time.
18. The system according to aspect 12, wherein the control unit is configured to determine a calibration parameter of the stimulation device based on the response measure.
19. The system according to any one of aspects 1 to 18, wherein the control unit is configured to control the operation of the stimulation device to generate an effector response being at least one of a muscular response and a glandular response.
20. The system according to aspect 19, wherein the effector tissue is smooth muscle tissue.
21. The system according to aspect 20, wherein the effector tissue forms part of a blood vessel, an intestine, or a urine bladder of the patient.
22. The system according to any one of aspects 1 to 21, wherein the control unit is configured to control the operation of the stimulation device such that the stimulation signal is a periodic signal including at least one of: a variable frequency component, a variable duty cycle component, a variable amplitude component, and a variable pause component.
23. The system according to aspect 22, wherein the stimulation signal is one of a low-frequency signal with an amplitude varying in a range of 0.1-100 Hz and a high-frequency signal with an amplitude varying in a range of 1-10 KHz.
24. The system according to any one of aspects 1 to 23, wherein the stimulation signal comprises series of pulses having a negative voltage relative to ground.
25. The system according to aspect 24, wherein the control unit is configured to operate the stimulation device to generate a positive voltage pulse following one or more negative voltage pulses.
26. The system according to any one of aspects 1 to 25, wherein the stimulation signal is an electric signal or a vibrational signal.
27. The system according to any one of aspects 1 to 26, wherein the stimulation device comprises a first stimulation electrode and a second stimulation electrode, wherein the first stimulation electrode and the second stimulation electrode are configured to be spaced apart along the nerve innervating the effector tissue.
28. The system according to aspect 27, wherein the stimulation device is configured to generate the stimulation signal such that the first stimulation electrode serves as a cathode and the second stimulation electrode serves as an anode.
29. The system according to aspect 27 or 28, further comprising a cuff configured to be at least partially arranged around the nerve and hold the first and second stimulation electrodes in place against the nerve.
30. The system according to any of the preceding aspects, further comprising a suppression arrangement configured to be coupled to the nerve so as to apply a suppression signal which is configured to suppress action potentials propagating in the nerve in a direction towards the central nervous system.
31. The system according to aspect 30, wherein the control unit is configured to regulate the suppression signal so as to suppress the action potentials induced in response to the stimulation device applying the stimulation signal.
32. The system according to aspect 30 or 31, wherein stimulation device is configured to be coupled to the nerve at a position between the effector tissue and the suppression arrangement so as to induce action potentials travelling in the nerve in a direction towards the effector tissue.
33. The system according to any one of aspects 30 to 32, wherein the control unit is configured to regulate the suppression of the action potentials so as to inhibit an undesired response of the nervous system of the patient, wherein the undesired response is generated responsive to the stimulation device applying the stimulation signal.
34. The system according to any one of aspects 30 to 33, wherein the control unit is configured to drive the stimulation device and the suppression arrangement such that each of the stimulation device and the suppression arrangement is actuated in sequence, with a delay of the suppression signal timed to generally match a conduction velocity of the stimulation signal in the nerve.
35. The system according to any one of aspects 30 to 33, wherein the control unit is configured to drive the stimulation device and the suppression arrangement so as to apply the first stimulation signal and the suppression signal substantially at the same time.
36. The system according to any one of aspects 30 to 35, wherein the control unit is configured to drive the stimulation device and the suppression arrangement such that each of the stimulation signal and the suppression signal is a time-varying signal, wherein the stimulation signals is a low-frequency signal and the suppression signal is a high-frequency signal.
37. The system according to any one of aspects 30 to 36, wherein an amplitude of the stimulation signal varies with a frequency in a range of 0.1-100 Hz and wherein an amplitude of the suppression signal varies with a frequency in a range of 1-10 KHz.

### Aspect Group 465SE: Stimulation_Electrical_Pain_Phantom Pain

1. A system for reducing pain in a patient, comprising:
   a stimulation device configured to deliver, directly or indirectly, a suppression signal to a nerve of the patient;
   a control unit configured to control an operation of the stimulation device to suppress or block a propagation of action potentials in an afferent direction of the nerve, thereby reducing the patient's sensation of pain originating from the action potentials.
2. The system according to aspect 1, wherein the pain is a phantom pain.
3. The system according to aspect 1 or 2, wherein the control unit is configured to control the operation of the stimulation device such that the suppression signal is a time-varying signal with a frequency in the range of 1-10 KHz.
4. The system according to any of the preceding aspects, further comprising a sensor device configured to generate a sensor signal indicating the action potentials propagating in the nerve.
5. The system according to aspect 4, wherein the control unit is configured to receive the sensor signal and to control an operation of the stimulation device based at least in part on the sensor signal.
6. The system according to aspect 4 or 5, wherein the stimulation device comprises a suppression electrode configured to be coupled to the nerve to deliver the suppression signal to the nerve, and wherein the sensor device comprises a sensor electrode configured to be arranged cranial to the suppression electrode.
7. The system according to aspect 6, wherein the control unit is configured to increase an intensity of the suppression signal in response to action potentials passing by the suppression electrode in the cranial direction.
8. The system according to any of the preceding aspects, wherein the control unit is configured to control the operation of the stimulation device such that the suppression signal is a periodic signal including at least one of: a variable frequency component, a variable duty cycle component, a variable amplitude component, and a variable pause component.
9. The system according to any of the preceding aspects, wherein the suppression signal comprises a series of pulses having a negative voltage relative to ground.
10. The system according to aspect 9, wherein the control unit is configured to operate the stimulation device to generate a positive voltage pulse following one or more negative voltage pulses.
11. The system according to any of the preceding aspects, wherein the stimulation device comprises a first suppression electrode and a second suppression electrode, the first suppression electrode and the second suppression electrode being configured to be spaced apart along the nerve.
12. The system according to aspect 11, wherein the stimulation device is configured to generate the suppression signal such that the first suppression electrode serves as a cathode and the second suppression electrode serves as an anode.
13. The system according to aspect 11 or 12, further comprising a cuff configured to be at least partially arranged around the nerve and to hold the first suppression electrode and/or the second suppression electrode in place against the nerve.

### Aspect Group 466SE: Stimulation_Electrical_Inhibition/Denervation_Method

1. A method for at least partly denervating an effector tissue of a patient, comprising:
   temporarily inhibiting a nerve innervating the effector tissue,
   determining a response measure indicative of an effector response in the effector tissue, wherein the effector response is at least partly induced by the inhibiting of the nerve,
   comparing the response measure with a predetermined reference measure, and
   at least partly denervating the effector tissue based at least in part on the comparison.
2. The method according to aspect 1, wherein temporarily inhibiting the nerve comprises cooling the nerve.
3. The method according to aspect 1, wherein temporarily inhibiting the nerve comprises delivering a neurotoxin to the nerve.
4. The method according to aspect 3, wherein the neurotoxin comprises botulinum toxin.
5. The method according to aspect 1, wherein temporarily inhibiting the nerve comprises delivering a vibrational signal to the nerve.
6. The method according to aspect 1, wherein temporarily inhibiting the nerve comprises delivering an electric inhibition signal to the nerve.
7. The method according to aspect 6, wherein the inhibition signal is a periodic signal including at least one of: a variable frequency component, a variable duty cycle component, a variable amplitude component, and a variable pause component.
8. The method according to any one of aspects 5 to 7, wherein the inhibition signal comprises an amplitude varying in a range of 1-10 KHz.
9. The method according to any one of aspects 6 to 8, wherein the inhibition signal is an electric signal comprising a series of pulses having a negative voltage relative to ground.
10. The method according to aspect 9, comprising generating a positive voltage pulse following one or more negative voltage pulses.
11. The method according to one of aspects 6 to 10, comprising generating the inhibition signal such that a first inhibition electrode serves as a cathode and a second inhibition electrode serves as an anode, wherein the first and second inhibition electrodes are arranged spaced apart along the nerve.
12. The method according to any one of aspects 6 to 10, further comprising delivering a suppression signal to the nerve to suppress action potentials propagating in the nerve in a direction towards the central nervous system.
13. The method according to aspect 12, comprising regulating the suppression signal to suppress the action potentials induced in the nerve in response to the inhibition signal.
14. The method according to aspect 12 or 13, comprising delivering the inhibition signal at a position between the effector tissue and a position in which the suppression signal is applied to the nerve.
15. The method according to any one of aspects 12 to 14, comprising regulating the suppression of the action potentials to inhibit an undesired response of the nervous system of the patient, wherein the undesired response is generated responsive to the electric inhibition signal.
16. The method according to any one of aspects 12 to 15, comprising applying the electric inhibition signal and the suppression signal in sequence, with a delay of the suppression signal being timed to generally match a conduction velocity of the electric inhibition signal in the nerve.
17. The method according to any one of aspects 1 to 16, comprising receiving a sensor signal indicative of the effector response and determining the response measure based on the received sensor signal.
18. The method according to aspect 17, wherein the sensor signal is indicative of an electric activity in the effector tissue.
19. The method according to aspect 17 or 18, wherein the sensor signal is indicative of a change in electrical impedance in the effector tissue.
20. The method according to any one of aspects 1 to 19, wherein the effector tissue forms part of a renal artery of the patient, and wherein the sensor signal is indicative of a vasodilation or vasoconstriction of the renal artery.
21. The method according to any one of aspects 1 to 19, wherein the effector tissue forms part of a gastrointestinal tract of the patient, and wherein the sensor signal is indicative of a level of motility of the gastrointestinal tract.
22. The method according to any one of aspects 1 to 19, wherein the effector tissue is glandular tissue, and wherein the sensor signal is indicative of a level of glandular secretion of the glandular tissue.
23. The method according to aspect 22, wherein the glandular tissue forms part of at least one of: a pancreas secreting insulin, a gallbladder secreting bile, and an adrenal gland secreting adrenaline, aldosterone, or cortisol.
24. The method according to any one of aspects 1 to 19, wherein the effector tissue is muscle tissue, and wherein the sensor signal is indicative of mechanical movement of the muscle tissue.
25. The method according to aspect 24, wherein the sensor signal is generated by a strain gauge measuring a contraction or relaxation of the muscle tissue.
26. The method according to any one of aspects 1 to 19, wherein the sensor signal is indicative of a heart rate of the patient.
27. The system according to any one of aspects 1 to 19, wherein the sensor device is indicative of a blood pressure of the patient.
28. The system according to any one of aspects 1 to 19, wherein the sensor signal is indicative of a rate of respiration of the patient.
29. The method according to any one of aspects 1 to 28, wherein the at least partly denervating of the effector tissue comprises ablating the effector tissue.
30. The method according to aspect 29, wherein the ablating comprises at least one of: surgical ablation, radiofrequency ablation, cryoablation, heat ablation, laser ablation, electrocautery, and chemical ablation.

### Aspect Group 467SE: Stimulation_Electrical_Inhibition/Denervation_System

1. A system for at least partly denervating an effector tissue of a patient, comprising:
   an inhibition device configured to temporarily inhibit a nerve innervating the effector tissue,
   a sensor configured to generate a sensor signal indicative of an effector response in the effector tissue, wherein the effector response is at least partly induced by the inhibiting of the nerve, and
   a processing unit configured to:
      determine a response measure based on the sensor signal, wherein the response measure is indicative of the effector response;
      compare the response measure with a predetermined reference measure, and
      determine, based on the comparison, whether a desired effector response has been achieved,
   wherein the system further comprises a denervation device configured to at least partly denervate the effector tissue.
2. The system according to aspect 1, wherein the inhibition device comprises a cooling device configured to cool the nerve to cause a temporary inhibition of the nerve.
3. The system according to aspect 1, wherein the inhibition device is a toxin administration device configured to deliver a neurotoxin to the nerve to cause a temporary inhibition of the nerve.
4. The system according to aspect 1, wherein the inhibition device is a vibrational device configured to deliver an inhibition signal to the nerve to cause a temporary inhibition of the nerve.
5. The system according to aspect 1, wherein the inhibition device is an electric stimulation device configured to deliver an inhibition signal to the nerve to cause a temporary inhibition of the nerve.
6. The system according to aspect 4 or 5, wherein the inhibition signal is a periodic signal including at least one of: a variable frequency component, a variable duty cycle component, a variable amplitude component, and a variable pause component.
7. The system according to any one of aspects 4 to 6, wherein the inhibition signal comprises an amplitude varying in a range of 1-10 KHz.
8. The system according to any one of aspects 5 to 7, wherein the inhibition signal is an electric signal comprising a series of pulses having a negative voltage relative to ground.
9. The system according to aspect 8, wherein the electric stimulation device is configured to generate a positive voltage pulse following one or more negative voltage pulses.
10. The system according to any one of aspects 5 to 9, wherein the inhibition device comprises a first inhibition electrode and a second inhibition electrode, wherein the first inhibition electrode and the second inhibition electrode are configured to be arranged spaced apart along the nerve innervating the effector tissue.
11. The system according to aspect 10, wherein the inhibition device is configured to generate the inhibition signal such that the first inhibition electrode serves as a cathode and the second inhibition electrode serves as an anode.
12. The system according to any one of aspects 5 to 11, wherein the inhibition device further comprises a suppression arrangement configured to be coupled to the nerve to apply a suppression signal which is configured to suppress action potentials propagating in the nerve in a direction towards the central nervous system.
13. The system according to aspect 12, wherein the inhibition device is configured to regulate the suppression signal so as to suppress the action potentials induced in response to the electric inhibition signal.
14. The system according to aspect 12 or 13, wherein the inhibition device is configured to apply the electric inhibition signal to the nerve at a position between the effector tissue and a position in which the suppression signal is applied to the nerve.
15. The system according to any one of aspects 12 to 14, wherein the inhibition device is configured to regulate the suppression of the action potentials so as to inhibit an undesired response of the nervous system of the patient, the undesired response being generated responsive to the electric inhibition signal.
16. The system according to any one of aspects 12 to 15, wherein the inhibition device is configured to apply the electric inhibition signal and the suppression signal in sequence, with a delay of the suppression signal timed to generally match a conduction velocity of the electric inhibition signal in the nerve.
17. The system according to any one of aspects 1 to 16, wherein the sensor device comprises a sensor electrode configured to measure an electric activity in the effector tissue.
18. The system according to any one of aspects 1 to 17, wherein the sensor device comprises a sensor electrode configured to measure a change in electrical impedance in the effector tissue.
19. The system according to aspect 17 or 18, wherein:
   the sensor electrode is configured to be arranged at the effector tissue;
   the sensor device further comprises a reference electrode, and
   the sensor device is configured to generate the sensor signal based on an electrical interaction between the sensor electrode and the reference electrode.
20. The system according to any one of aspects 1 to 19, wherein the inhibition device is configured to temporarily inhibit a nerve innervating effector tissue forming part of a renal artery of the patient, and wherein the sensor is configured to generate a sensor signal indicative of a vasodilation or vasoconstriction of the renal artery.
21. The system according to any one of aspects 1 to 19, wherein the inhibition device is configured to temporarily inhibit a nerve innervating effector tissue forming part of a gastrointestinal tract of the patient, and wherein the sensor is configured to generate a sensor signal indicative of a level of motility of the gastrointestinal tract.
22. The system according to any one of aspects 1 to 19, wherein the inhibition device is configured to temporarily inhibit a nerve innervating effector tissue being glandular tissue, and wherein the sensor is configured to generate a sensor signal indicative of a level of glandular secretion of the glandular tissue.
23. The system according to aspect 22, wherein the glandular tissue forms part of at least one of: a pancreas secreting insulin, a gallbladder secreting bile, and an adrenal gland secreting adrenaline, aldosterone, or cortisol.
24. The system according to any one of aspects 1 to 19, wherein the inhibition device is configured to temporarily inhibit a nerve innervating effector tissue being muscle tissue, and wherein the sensor is configured to generate a sensor signal indicative of mechanical movement of the muscle tissue.
25. The system according to aspect 24, wherein the sensor device comprises a strain gauge configured to measure a contraction or relaxation of the muscle tissue.
26. The system according to any one of aspects 1 to 19, wherein the sensor device is configured to measure a heart rate of the patient.
27. The system according to any one of aspects 1 to 19, wherein the sensor device is configured to measure a blood pressure of the patient.
28. The system according to any one of aspects 1 to 19, wherein the sensor device is configured to measure a rate of respiration of the patient.
29. The system according to any one of aspects 1 to 28, wherein the denervation device is an ablation device.
30. The system according to aspect 29, wherein the ablation device is configured to at least partly denervate the effector tissue by means of at least one of: surgical ablation, radiofrequency ablation, cryoablation, laser ablation, heat ablation, electrocautery, and chemical ablation.

### Aspect Group 468SE: Stimulation_Electrical_Impotence_Method

1. A method of creating engorgement of an erectile body of a male person, using a pre-implanted stimulation device comprising configured to deliver a first stimulation signal to a sympathetic nerve innervating a penile vein of the male person and to deliver a second stimulation signal to a parasympathetic nerve innervating a penile artery of the male person, the method comprising:
   stimulating, by means of the first stimulation signal, the sympathetic nerve to cause vasoconstriction of the penile vein;
   stimulating, by means of the second stimulation signal, the parasympathetic nerve to cause vasodilation of the penile artery;
   wherein the vasodilation of the penile artery increases a blood flow entering the erectile body of the penis and the vasoconstriction of the penile vein reduces a blood flow exiting the erectile body, thereby inducing engorgement of the erectile body.
2. The method according to aspect 1, comprising:
   delivering the first stimulation signal to the sympathetic nerve at a position between a level of the T11-L2 vertebrae and the pelvic plexus; and
   delivering the second stimulation signal to the parasympathetic nerve at a position between a level of sacral spinal cord segments S2-S4 and the pelvic plexus.
3. The method according to aspect 1 or 2, wherein:
   each of the first and second stimulation signals is a periodic signal including at least one of: a variable frequency component, a variably duty cycle component, a variable amplitude component, and a variable pause component.
4. The method according to any of the preceding aspects, comprising:
   delivering a first inhibition signal to the sympathetic nerve to cause vasodilation of the penile vein; and
   delivering a second inhibition signal to the parasympathetic nerve to cause vasoconstriction of the penile artery.
5. The method according to aspect 4, wherein:
   the first stimulation signal is a low-frequency signal configured to stimulate the activity of the sympathetic nerve;
   the second stimulation signal is a low-frequency signal configured to stimulate the activity of the parasympathetic nerve;
   the first inhibition signal is a high-frequency signal configured to inhibit the activity of the sympathetic nerve; and
   the second inhibition signal is a high-frequency signal configured to inhibit the activity of the parasympathetic nerve.
6. The method according to aspect 5, comprising:
   varying an amplitude of the low-frequency signal with a frequency in the range of 0.1-100 Hz; and
   varying an amplitude of the high-frequency signal with a frequency in the range of 1-10 kHz.
7. The method according to any of the preceding aspects, wherein at least one of the first and second stimulation signals comprises series of pulses having a negative voltage relative to ground.
8. The method according to aspect 7, wherein a positive voltage pulse follows one or more negative voltage pulses.
9. The method according to any of the preceding aspects, wherein at least one of the first stimulation signal and the second stimulation signal is an electric signal or a vibrational signal.
10. The method according to any of the preceding aspects, further comprising:
   receiving a sensor signal indicating at least one of vasoconstriction of the penile vein, vasodilation of the penile artery, and engorgement of the erectile body; and
   adjusting an intensity of at least one of the first and second stimulation signal based at least in part on the sensor signal.
11. The method according to aspect 10, wherein the sensor signal indicates an electric activity in smooth muscle tissue of the penile vein and/or the penile artery.
12. The method according to aspect 10, wherein the sensor signal indicates a change in electrical impedance in smooth muscle tissue of the penile vein and/or the penile artery.
13. The method according to any of aspects 10-12, wherein the sensor signal is generated by a strain gauge and wherein the sensor signal indicates an engorgement of the erectile body.
14. The method according to any of aspects 10-13, comprising determining a response measure based on the sensor signal, the response measure being indicative of said at least one of vasoconstriction of the penile vein, vasodilation of the penile artery, and engorgement of the erectile body.
15. The method according to aspect 14, comprising:
   comparing the response measure with a predetermined reference measure; and
   in response to the response measure being below the reference measure, increasing an intensity of the first stimulation signal to stimulate the activity in the in the sympathetic nerve and/or increasing an intensity of the second stimulation signal to increase the activity of the parasympathetic nerve, and
   in response to the response measure exceeding the reference measure, reducing the intensity of the first stimulation signal to inhibit the activity of the sympathetic nerve and/or reducing the intensity of the second stimulation signal to inhibit the activity of the parasympathetic nerve.
16. The method according to aspect 15, wherein the predetermined reference measure is based on a previous measurement on the male person.
17. The method according to aspect 15 or 16, wherein the predetermined reference measure is based on previous measurement on other male persons.
18. The method according to any of the preceding aspects, further comprising:
   applying a first suppression signal to the sympathetic nerve to suppress action potentials generated by the first stimulation signal and propagating in a direction towards the central nervous system.
19. The method according to aspect 18, comprising:
   applying the first stimulation signal at a position between the penile vein and a position in which the first suppression signal is applied.
20. The method according to aspect 18 or 19, comprising:
   regulating the suppression of the action potentials to inhibit an undesired response of the nervous system of the male person, the undesired response being generated responsive to the first stimulation signal.
21. The method according to any of aspects 18-20, comprising:
   applying the first stimulation signal and the first suppression signal in sequence, with a delay of the first suppression signal timed to generally match a conduction velocity of the first stimulation signal in the sympathetic nerve.
22. The method according to any of aspects 18-21, wherein an amplitude of the first suppression signal varies with frequency in the range of 1-10 KHz.
23. The method according to any of the preceding aspects, further comprising:
   applying a second suppression signal to the parasympathetic nerve to suppress action potentials generated by the second stimulation signal and propagating in the direction towards the central nervous system.
24. The method according to aspect 23, comprising:
   applying the second stimulation signal at a position between the penile artery and a position in which the second suppression signal is applied.
25. The method according to aspect 23 or 24, comprising:
   regulating the suppression of the action potentials to inhibit an undesired response of the nervous system of the male person, the undesired response being generated responsive to the second stimulation signal.
26. The method according to any of aspects 23-25, comprising:
   applying the second stimulation signal and the second suppression signal in sequence, with a delay of the second suppression signal timed to generally match a conduction velocity of the second stimulation signal in the parasympathetic nerve.
27. The method according to any of aspects 23-26, wherein an amplitude of the second suppression signal varies with frequency in the range of 1-10 KHz.
28. The method according to any of the preceding aspects, comprising delivering the first stimulation signal by means of a first electrode and a second electrode arranged spaced apart along the sympathetic nerve.
29. The method according to aspect 27, comprising stimulating the sympathetic nerve by operating the first electrode as a cathode and the second electrode as an anode.
30. The method according to any of the preceding aspects, comprising delivering the second stimulation signal by means of a third electrode and a fourth electrode arranged space apart along the parasympathetic nerve.
31. The method according to aspect 30, comprising stimulating the parasympathetic nerve by operating the third electrode as a cathode and the fourth electrode as an anode.

### Aspect Group 469SE: Stimulation_Electrical_Impotence_System

1. A system for treating erectile dysfunction in a male person, comprising:
   a stimulation device configured to deliver, directly or indirectly:
   a first stimulation signal to a sympathetic nerve innervating a penile vein of the person;
   a second stimulation signal to a parasympathetic nerve innervating a penile artery of the person; and
   a control unit configured to control an operation of the stimulation device such that:
   the first stimulation signal stimulates an activity of the sympathetic nerve, thereby causing vasoconstriction of the penile vein; and
   the second stimulation signal stimulates an activity of the parasympathetic nerve, thereby causing vasodilation of the penile artery;
   wherein the vasodilation of the penile artery increases a blood flow entering an erectile body of the penis and the vasoconstriction of the penile vein reduces a blood flow exiting the erectile body, thereby inducing engorgement of the erectile body.
2. The system according to aspect 1, comprising:
   a first stimulation arrangement configured to deliver the first stimulation signal, the first stimulation arrangement being configured to be coupled to the sympathetic nerve at a position between a level of the T11-L2 vertebrae and the pelvic plexus; and
   a second stimulation arrangement configured to deliver the second stimulation signal, the second stimulation arrangement being configured to be coupled to the second stimulation signal to the parasympathetic nerve at a position between a level of sacral spinal cord segments S2-S4 and the pelvic plexus.
3. The system according to aspect 1 or 2, wherein the control unit is configured to control the operation of the stimulation device such that at least one of the first stimulation signal and the second stimulation signal is a periodic signal including at least one of: a variable frequency component, a variable duty cycle component, a variable amplitude component, and a variable pause component.
4. The system according to any of the preceding aspects, wherein:
   each of the first stimulation signal and the second stimulation signal comprises an amplitude varying with a frequency in the range of 0.1-100 Hz.
5. The system according to any of the preceding aspects, wherein at least one of the first and second stimulation signals comprises series of pulses having a negative voltage relative to ground.
6. The system according to aspect 5, wherein the control unit is configured to operate the stimulation device to generate a positive voltage pulse following one or more negative voltage pulses.
7. The system according to any of the preceding aspects, wherein at least one of the first stimulation signal and the second stimulation signal is an electric signal or a vibrational signal.
10. The system according to any of the preceding aspects, further comprising:
   a sensor device configured to generate a sensor signal indicating at least one of vasoconstriction of the penile vein, vasodilation of the penile artery, and engorgement of the erectile body;
   wherein the control unit is configured to receive the sensor signal and to control an operation of the stimulation device based at least in part on the sensor signal.
11. The system according to aspect 10, wherein the sensor device comprises a sensor electrode configured to measure an electric activity in smooth muscle tissue of the penile vein and/or the penile artery.
12. The system according to aspect 10, wherein the device comprises a sensor electrode configured to measure a change in electrical impedance in smooth muscle tissue of the penile vein and/or the penile artery.
13. The system according to aspect 11 or 12, wherein:
   the sensor electrode is configured to be arranged at the penile vein and/or the penile artery;
   the sensor further comprises a reference electrode, and
   the sensor device is configured to generate the sensor signal based on an electrical interaction between the sensor electrode and the reference electrode.
14. The system according to aspect 13, wherein the reference electrode is formed by a housing of the stimulation device or the sensor device.
15. The system according to aspect 10, wherein the sensor signal is generated by a strain gauge and wherein the sensor signal indicates an engorgement of the erectile body.
16. The system according to any of aspects 10-15, wherein the control unit is configured to determine a response measure based on the sensor signal, the response measure being indicative of said at least one of vasoconstriction of the penile vein, vasodilation of the penile artery, and engorgement of the erectile body.
17. The system according to aspect 16, wherein the control unit is configured to:
   compare the response measure with a predetermined reference measure; and
   in response to the response measure being below the reference measure, increase an intensity of the first stimulation signal to stimulate the activity in the in the sympathetic nerve and/or increase an intensity of the second stimulation signal to increase the activity of the parasympathetic nerve, and
   in response to the response measure exceeding the reference measure, reduce the intensity of the first stimulation signal to inhibit the activity of the sympathetic nerve and/or reduce the intensity of the second stimulation signal to inhibit the activity of the parasympathetic nerve.
18. The method according to aspect 17, wherein the predetermined reference measure is based on a previous measurement on the person.
19. The method according to aspect 17 or 18, wherein the predetermined reference measure is based on previous measurement on other persons.
20. The system according to any of the preceding aspects, wherein the stimulation device comprises a first stimulation arrangement configured to deliver the first stimulation signal and a second stimulation arrangement configured to deliver the second stimulation signal.
21. The system according to aspect 20, wherein the first stimulation arrangement comprises a first stimulation electrode and a second stimulation electrode, the first stimulation electrode and the second stimulation electrode are configured to be spaced apart along the sympathetic nerve innervating the penile vein of the person.
22. The system according to aspect 20 or 21, wherein the stimulation device is configured to generate the first stimulation signal such that the first stimulation electrode serves as a cathode and the second stimulation electrode serves as an anode.
23. The system according to any of aspects 20-22, further comprising a cuff configured to be at least partially arranged around the sympathetic nerve and to hold the first stimulation arrangement in place against the sympathetic nerve.
24. The system according to any of aspects 20-23, wherein the second stimulation arrangement comprises a third electrode and a fourth electrode, the third electrode and the fourth electrode being configured to be arranged spaced apart along the parasympathetic nerve.
25. The system according to aspect 24, wherein the stimulation device is configured to generate the second stimulation signal such that the third electrode serves as a cathode and the fourth electrodes serves as an anode.
26. The system according to any of aspects 20-25, further comprising a cuff configured to be at least partially arranged around the parasympathetic nerve and to hold the second stimulation arrangement in place against the parasympathetic nerve.
27. The system according to any of the preceding aspects, further comprising:
   a suppression device comprising a first suppression arrangement configured to apply a first suppression signal to the sympathetic nerve to suppress action potentials generated by the first stimulation signal and propagating in a direction towards the central nervous system.
28. The system according to aspect 27, wherein the stimulation device is configured to deliver the first stimulation signal to the sympathetic nerve at a position between the penile vein and the first suppression arrangement.
29. The system according to aspect 27 or 28, wherein the control unit is configured to
   regulate the suppression of the action potentials to inhibit an undesired response of the nervous system of the person, the undesired response being generated responsive to the first stimulation signal.
30. The system according to any of aspects 27-29, wherein the control unit is configured to apply the first stimulation signal and the first suppression signal in sequence, with a delay of the first suppression signal timed to generally match a conduction velocity of the first stimulation signal in the sympathetic nerve.
31. The system according to any of aspects 27-30, wherein the suppression device is configured to vary an amplitude of the first suppression signal with frequency in the range of 1-10 kHz.
32. The system according to any of aspects 27-31, wherein the first suppression arrangement comprises a first suppression electrode and a second suppression electrode configured to be spaced apart along the sympathetic nerve.
33. The system according to aspect 32, wherein the first suppression arrangement further comprises a third suppression electrode configured to be arranged spaced apart from the first and second suppression electrodes along the sympathetic nerve such that the second suppression electrode is arranged between the first and third suppression electrodes.
34. The system according to aspect 32, wherein the suppression device is configured to generate the first suppression signal such that the second suppression electrode serves as a cathode and the first and third suppression electrodes serve as anodes.
35. The system according to any of aspect 1-26, further comprising:
   a suppression device comprising a second suppression arrangement configured to apply a second suppression signal to the parasympathetic nerve to suppress action potentials generated by the second stimulation signal and propagating in a the direction towards the central nervous system.
36. The system according to aspect 35, wherein the stimulation device is configured to deliver the second stimulation signal to the parasympathetic nerve at a position between the penile artery and the second suppression arrangement.
37. The system according to aspect 35 or 36, wherein the control unit is configured to regulate the suppression of the action potentials to inhibit an undesired response of the nervous system of the person, the undesired response being generated responsive to the second stimulation signal.
38. The system according to any of aspects 35-37, wherein the suppression device is configured to apply the second stimulation signal and the second suppression signal in sequence, with a delay of the second suppression signal timed to generally match of conduction velocity of the second stimulation signal in the parasympathetic nerve.
39. The system according to any of aspects 35-38, wherein the suppression device is configured to vary an amplitude of the second suppression signal with frequency in the range of 1-10 kHz.
40. The system according to any of aspects 35-39, wherein the second suppression arrangement comprises a first suppression electrode and a second suppression electrode configured to be spaced apart along the parasympathetic nerve.
41. The system according to aspect 40, wherein the first suppression arrangement further comprises a third suppression electrode configured to be arranged spaced apart from the first and second suppression electrodes along the parasympathetic nerve such that the second suppression electrode is arranged between the first and third suppression electrodes.
42. The system according to aspect 41, wherein the suppression device is configured to generate the second suppression signal such that the second suppression electrode serves as a cathode and the first and third suppression electrodes serve as anodes.

### Aspect Group 470SE: Stimulation_Electrical_Appetite_Vagus Stimulation

1. A system for controlling appetite in a patient, comprising:
   a stimulation device configured to deliver, directly or indirectly a stimulation signal to the vagus nerve; and
   a control unit configured to control an operation of the stimulation device to affect a propagation of action potentials in the vagus nerve such that the patient's feeling of satiety or hunger is affected.
2. The system according to aspect 1, wherein the control unit is configured to control the operation of the stimulation device such that the stimulation signal suppresses the propagation of action potentials in a cranial direction, thereby hindering a hunger signal conveyed by the vagus nerve from reaching the patient's brain.
3. The system according to aspect 2, wherein the control unit is configured to control the operation of the stimulation device such that the stimulation signal is a time-varying signal with an amplitude varying with a frequency in the range of 1-10 KHz.
4. The system according to aspect 1, wherein the control unit is configured to control the operation of the stimulation device such that the stimulation signal generates action potentials propagating in a cranial direction, thereby causing a satiety signal to be conveyed by the vagus nerve to the patient's brain.
5. The system according to aspect 4, wherein the control unit is configured to control the operation of the stimulation device such that the stimulation signal is a time-varying signal with an amplitude varying with a frequency in the range of 0.1-100 Hz.
6. The system according to aspect 1, wherein the control unit is configured to control the operation of the stimulation device such that the stimulation signal affects the propagation of action potentials in the vagus nerve to suppress a stomach peristalsis.
7. The system according to aspect 6, wherein the control unit is configured to control the operation of the stimulation device such that the stimulation signal stimulates an activity of a parasympathetic nerve fiber of the vagus nerve innervating muscle tissue of the stomach wall.
8. The system according to aspect 7, wherein the control unit is configured to control the operation of the stimulation device such that the stimulation signal is a time-varying signal with an amplitude varying with a frequency in the range of 0.1-100 Hz.
9. The system according to aspect 6, wherein the control unit is configured to control the operation of the stimulation device such that the stimulation signal suppresses the propagation of action potentials in a sympathetic fiber of the vagus nerve innervating muscle tissue of the stomach wall.
10. The system according to aspect 9, wherein the control unit is configured to control the operation of the stimulation device such that the stimulation signal is a time-varying signal with an amplitude varying with a frequency in the range of 1-10 KHz.
11. The system according to any of the preceding aspects, wherein the stimulation device comprises a stimulation arrangement configured to deliver the stimulation signal to the vagus nerve, wherein the stimulation arrangement is configured to be coupled to the vagus nerve at a position caudal to the esophageal hiatus.
12. The system according to any of the preceding aspects 11, wherein the stimulation arrangement is configured to be coupled to an esophageal plexus of the vagus nerve.
13. The system according to aspect 11, wherein the stimulation arrangement is configured to be coupled to an anterior trunk of the vagus nerve.
14. The system according to aspect 11, wherein the stimulation arrangement is configured to be coupled to a gastric branch of the vagus nerve.
15. The system according to any of the preceding aspects, wherein the stimulation device comprises a suppression arrangement configured to be coupled to the vagus nerve, wherein the control unit is configured to drive the stimulation device to apply, by means of the suppression arrangement, a suppression signal suppressing action potentials propagating in a direction towards the central nervous system in response to the stimulation device applying the stimulation signal.
16. The system according to aspect 15, wherein the stimulation device comprises a stimulation arrangement configured to deliver the stimulation signal to the vagus nerve, wherein the suppression arrangement is configured to be coupled to the vagus nerve caudal to the stimulation arrangement.
17. The system according to aspect 16, wherein the control unit is configured to regulate the suppression of the action potentials so as to inhibit an undesired response of the nervous system of the patient, the undesired response being generated responsive to the stimulation arrangement applying the stimulation signal.
18. The system according to aspect 16 or 17, wherein the control unit is configured to drive the stimulation device such that the stimulation arrangement and the suppression arrangement are actuated in sequence, with a delay of the suppression signal timed to generally match a conduction velocity of the stimulation signal in the vagus nerve.
19. The system according to aspect 16 or 17, wherein the control unit is configured to drive the stimulation device to apply the stimulation signal and the suppression signal substantially at the same time.
20. The system according to any of aspects 16-19, wherein the control unit is configured to drive the stimulation device such that each f other stimulation signal and the suppression signal is a time-varying signal, wherein the stimulation signal is a low-frequency signal and the suppression signal is a high-frequency signal.
21. The system according to any of aspects 16-20, wherein an amplitude of the stimulation signal varies with a frequency in the range of 0.1-100 Hz and wherein an amplitude of the suppression signal varies with a frequency in the range of 1-10 KHz.
22. The system according to any of the preceding aspects, further comprising a sensor device configured to generate a sensor signal indicating an effector response in tissue innervated by the vagus nerve and/or the action potentials propagating in the vagus nerve.
23. The system according to aspect 22, wherein the control unit is configured to receive the sensor signal and to control an operation of the stimulation device based at least in part on the sensor signal.
24. The system according to aspect 23, wherein the control unit is configured to determine a response measure based on the sensor signal, the response measure being indicative of the effector response.
25. The system according to aspect 24, wherein the control unit is configured to:
   compare the response measure with a predetermined reference measure; and
   control the stimulation device to:
   increase an intensity of the stimulation signal in response to the response measure being below the reference measure, and
   reduce the intensity of the stimulation signal in response to the response measure exceeding the reference measure.
26. The system according to aspect 25, wherein the control unit is configured to:
   increase the intensity of the stimulation signal by increasing at least one of a frequency, current amplitude, and voltage amplitude of the stimulation signal; and
   reduce the intensity of the stimulation signal by reducing at least one of the frequency, current amplitude, and voltage amplitude of the stimulation signal.
27. The system according to aspect 25 or 26, wherein the predetermined reference measure is based on a previous measurement of the effector response in the patient.
28. The system according to aspect 25 or 26, wherein the predetermined reference measure is based on previous measurements of effector responses in other patients.
29. The system according to aspect 24, wherein the control unit is configured to monitor the level of effector response over time, and to control the stimulation device based on a change rate in the effector response over time.

### Aspect Group 471SE: Stimulation_Electrical_General_DC-Blocking Capacitor

1. A system for stimulating an effector tissue of a patient, comprising:
   a stimulation device configured to deliver a stimulation signal to at least one of the effector tissue and a nerve innervating the effector tissue of the patient,
   a source of energy configured to energize the stimulation device,
   a control unit operably connected to the stimulation device and configured to control an operation of the stimulation device such that the stimulation signal causes at least one of: stimulating an effector response in the effector tissue and inhibiting an effector response in the effector tissue, and
   a capacitor configured to reduce a current leakage of the system to 1 μA or less.
2. The system according to aspect 1, wherein the capacitor is configured to be connected in series with the body of the patient and at least one of the stimulation device, the source of energy, and the control unit.
3. The system according to aspect 1, wherein the stimulation device comprises a stimulation arrangement configured to be coupled to the effector tissue or nerve, and wherein the capacitor is configured to be connected in series with the body of the patient and the stimulation arrangement.
4. The system according to aspect 1, wherein the stimulation arrangement comprises a first stimulation electrode and a second stimulation electrode for applying the stimulation signal, and wherein the capacitor is configured to be connected in series with the first stimulation electrode and second stimulation electrode.
5. The system according any one of aspects 1 to 4, wherein the capacitor is integrated in a circuitry for controlling the operation of the stimulation device.
6. The system according to any one of aspects 1 to 5, further comprising a printed circuit board (PCB) supporting the capacitor and at least one of the stimulation device, the source of energy, and the control unit.
7. The system according to aspect 6, wherein the PCB is at least one of a multi-layer PCB, a flexible PCB, and a stretchable PCB.
8. The system according to any of the preceding aspects, wherein:
   the stimulation device is configured to deliver a first stimulation signal to a sympathetic nerve innervating a first effector tissue of the patient and a second stimulation signal to a parasympathetic nerve innervating a second effector tissue and
   the control unit is configured to control the operation of the stimulation device such that:
   the first stimulation signal stimulates an activity of the sympathetic nerve and the second stimulation signal inhibits an activity of the parasympathetic nerve, or
   the first stimulation signal inhibits an activity of the sympathetic nerve and the second stimulation signal stimulates an activity of the parasympathetic nerve.
9. The system according to aspect 8, wherein:
   the first signal is a low-frequency signal configured to stimulate the activity of the sympathetic nerve and the second signal is a high-frequency signal configured to inhibit the activity of the parasympathetic nerve, or
   the first signal is a high-frequency signal configured to inhibit the activity of the sympathetic nerve and the second signal is a low-frequency signal configured to stimulate the activity of the parasympathetic nerve.
9. The system according to aspect 9, wherein an amplitude of the low-frequency signal varies with a frequency in a range of 0.1-100 Hz and an amplitude of the high-frequency signal varies with a frequency in a range of 1-10 KHz.
10. The system according to aspect 1, wherein:
   the stimulation device comprises a first stimulation arrangement and a second stimulation arrangement, each configured to be coupled to at least one of an effector tissue and a nerve innervating the effector tissue of the patient,
   the control unit is configured to drive the stimulation device to apply, by means of the first stimulation arrangement, a stimulation signal inducing the effector response in the effector tissue, and, by means of the second stimulation arrangement, a suppression signal suppressing action potentials propagating in the nerve towards the central nervous system (CNS), and
   the control unit is further configured to regulate the suppression signal so as to suppress the action potentials induced in response to the stimulation device applying the stimulation signal.
11. The system according to aspect 10, wherein the first stimulation arrangement is configured to be coupled to the nerve at a position between the effector tissue and the second electrode so as to induce action potentials travelling in the nerve in a direction towards the effector tissue.
12. The system according to aspect 11, wherein the control unit is configured to regulate the suppression of the action potentials so as to inhibit an undesired response of a nervous system of the patient, the undesired response being generated responsive to the first electrode applying the stimulation signal.
13. The system according to aspect 11 or 12, wherein the control unit is configured to drive the stimulation device such that each of the first and second stimulation arrangements are actuated in sequence, with a delay of the suppression signal timed to generally match a conduction velocity of the stimulation signal.
14. The system according to any one of aspects 10 to 12, wherein the control unit is configured to drive the stimulation device to apply the stimulation signal and the suppression signal substantially at the same time.
15. The system according to any one of aspects 10 to 14, wherein the control unit is configured to drive the stimulation device such that each of the stimulation signal and the suppression signal is a time-varying signal, wherein the stimulation signal is a low-frequency signal and the suppression signal is a high-frequency signal.
16. The system according to aspect 15, wherein an amplitude of the stimulation signal varies with a frequency in a range of 0.1-100 Hz, and wherein an amplitude of the suppression signal varies with a frequency in a range of 1-10 KHz.
17. The system according to any one of aspects 1 to 16, further comprising a sensor device configured to generate a sensor signal indicating an effector response in the effector tissue, wherein the control unit is configured to:
   receive the sensor signal and
   control an operation of the stimulation device based at least in part on the sensor signal.
18. The system according to aspect 17, wherein the sensor device comprises a sensor electrode configured to measure an electric activity in the effector tissue in response to the stimulation signal.
19. The system according to aspect 17, wherein the sensor device comprises a sensor electrode configured to measure a change in electrical impedance in the effector tissue in response to the stimulation signal.
20. The system according to aspect 17, wherein sensor device comprises an electromyographic sensor configured to measure an electric activity in the effector tissue and an electric impedance sensor configured to measure a change in electrical impedance in the effector tissue.

### Aspect Group 472SE: Stimulation_Electrical_General_Multi-Layer PCB

1. A system for stimulating an effector tissue of a patient, comprising:
   a stimulation device configured to deliver a stimulation signal to at least one of the effector tissue and a nerve innervating the effector tissue of the patient,
   a source of energy configured to energize the stimulation device,
   a control unit operably connected to the stimulation device and configured to control an operation of the stimulation device such that the stimulation signal causes at least one of stimulating an effector response in the effector tissue and inhibiting an effector response in the effector tissue, and
   a printed circuit board (PCB) supporting at least one of the stimulation device, the source of energy, and the control unit,
   wherein the PCB is at least one of a multi-layer PCB, a flexible PCB, a stretchable PCB.
2. The system according to aspect 1, wherein the PCB comprises first multi-layer portion and a second multi-layer portion interconnected by a stretchable portion.
3. The system according to aspect 1, wherein the PCB comprises a first multi-layer portion and a second multi-layer portion interconnected by a flexible portion.
4. The system according to any one of aspects 1 to 3, further comprising a capacitor configured to reduce a current leakage of the system to 1 μA or less.
5. The system according to aspect 4, wherein the capacitor is connected in series with a part of the system and the body of the patient.
6. The system according to aspect 4, wherein the capacitor is connected in series with a first and a second stimulation electrode of the stimulation device.
7. The system according to aspect 4 or 5, wherein the capacitor is arranged on the PCB.
8. The system according to any one of aspects 1 to 7, wherein:
   the stimulation device is configured to deliver a first stimulation signal to a sympathetic nerve innervating a first effector tissue of the patient and a second stimulation signal to a parasympathetic nerve innervating a second effector tissue and
   the control unit is configured to control the operation of the stimulation device such that:
   the first stimulation signal stimulates an activity of the sympathetic nerve and the second stimulation signal inhibits an activity of the parasympathetic nerve or
   the first stimulation signal inhibits an activity of the sympathetic nerve and the second stimulation signal stimulates an activity of the parasympathetic nerve.
9. The system according to aspect 8, wherein:
   the first signal is a low-frequency signal configured to stimulate the activity of the sympathetic nerve and the second signal is a high-frequency signal configured to inhibit the activity of the parasympathetic nerve, or
   the first signal is a high-frequency signal configured to inhibit the activity of the sympathetic nerve and the second signal is a low-frequency signal configured to stimulate the activity of the parasympathetic nerve.
10. The system according to aspect 9, wherein an amplitude of the low-frequency signal varies with a frequency in a range of 0.1-100 Hz and wherein an amplitude of the high-frequency signal varies with a frequency in a range of 1-10 KHz.
11. The system according to any one of aspects 1 to 10, wherein:
   the stimulation device comprises a first stimulation arrangement and a second stimulation arrangement, each configured to be coupled to at least one of an effector tissue and a nerve innervating the effector tissue of the patient,
   the control unit is configured to drive the stimulation device to apply, by means of the first stimulation arrangement, a stimulation signal inducing the effector response in the effector tissue, and, by means of the second stimulation arrangement, a suppression signal suppressing action potentials propagating in the nerve towards the central nervous system (CNS), and
   the control unit is further configured to regulate the suppression signal so as to suppress the action potentials induced in response to the stimulation device applying the stimulation signal.
12. The system according to aspect 11, wherein the first stimulation arrangement is configured to be coupled to the nerve at a position between the effector tissue and the second electrode so as to induce action potentials travelling in the nerve in a direction towards the effector tissue.
13. The system according to aspect 12, wherein the control unit is configured to regulate the suppression of the action potentials so as to inhibit an undesired response of the nervous system of the patient, the undesired response being generated responsive to the first electrode applying the stimulation signal.
14. The system according to aspect 12 or 13, wherein the control unit is configured to drive the stimulation device such that each of the first and second stimulation arrangements are actuated in sequence, with a delay of the suppression signal timed to generally match a conduction velocity of the stimulation signal.
15. The system according to any one of aspects 11 to 13, wherein the control unit is configured to drive the stimulation device to apply the stimulation signal and the suppression signal substantially at the same time.
16. The system according to any one of aspects 11 to 15, wherein the control unit is configured to drive the stimulation device such that each of the stimulation signal and the suppression signal is a time-varying signal, wherein the stimulation signal is a low-frequency signal and the suppression signal is a high-frequency signal.
17. The system according to aspect 16, wherein an amplitude of the stimulation signal varies with a frequency in a range of 0.1-100 Hz, and wherein an amplitude of the suppression signal varies with a frequency in a range of 1-10 KHz.
18. The system according to any one of aspects 1 to 17, further comprising a sensor device configured to generate a sensor signal indicating an effector response in the effector tissue, wherein the control unit is configured to:
   receive the sensor signal and
   control an operation of the stimulation device based at least in part on the sensor signal.
19. The system according to aspect 18, wherein the sensor device comprises a sensor electrode configured to measure an electric activity in the effector tissue in response to the stimulation signal.
20. The system according to aspect 18, wherein the sensor device comprises a sensor electrode configured to measure a change in electrical impedance in the effector tissue in response to the stimulation signal.
21. The system according to aspect 18, wherein sensor device comprises an electromyographic sensor configured to measure an electric activity in the effector tissue and an electric impedance sensor configured to measure a change in electrical impedance in the effector tissue.

### Aspect Group X: Controlling Peristalsis/Segmentation

1. A system for affecting a flow of intestinal content in a gastrointestinal tract of a patient, comprising:
   a stimulation device configured to deliver a stimulation signal to a plurality of stimulation sites along the gastrointestinal tract to induce an effector response in smooth muscles of a wall of the gastrointestinal tract;
   a source of energy configured to energize the stimulation device; and
   a control unit operably connected to the stimulation device and configured to control an operation of the stimulation device such that the stimulation signal causes at least one of promoting and suppressing peristalsis or segmentation of the gastrointestinal tract;
   wherein the plurality of stimulation sites are spaced apart by a predetermined spacing.
2. The system according to aspect 1, wherein the plurality of stimulation sites are spaced apart by a spacing corresponding to a wavelength of a wave pattern of a movement of the smooth muscles, or by a multiple of such a wavelength.
3. The system according to aspect 1 or 2, wherein neighboring stimulation sites are spaced apart, along the gastrointestinal tract, by 2-18 cm, such as 6-14 cm, such as 8-12 cm, such as about 10 cm.
4. The system according to aspect 3, wherein the plurality of stimulation sites are located on the small intestine.
5. The system according to aspect 1 or 2, neighboring stimulation sites are spaced apart, along the gastrointestinal tract, by 10-30 cm, such as 14-26 cm, such as 18-22 cm, such as about 20 cm.
6. The system according to aspect 5, wherein the plurality of stimulation sites are located on the large intestine.
7. The system according to any of the preceding aspects, wherein the control unit is configured to control the application of the stimulation signals based on a wave pattern of a movement of the smooth muscles.
8. The system according to aspect 7, wherein the control unit is configured to apply the stimulation signals in pulses, or pulse trains, wherein a frequency of such pulses is determined based on a frequency of the wave pattern of the movement of the smooth muscles.
9. The system according to aspect 8, wherein the control unit is configured to apply the stimulation signal to induce contraction of a smooth muscle when the muscle is about to contract according to the wave pattern.
10. The system according to aspect 8 or 9, wherein the control unit is configured apply the stimulation signal to induce relaxation of a smooth muscle when the muscle is about to relax according to the wave pattern.
11. The system according to any of the preceding aspects, wherein the control unit is configured to apply the stimulation signal with a pulse separation or pulse train separation of 10-30 s, such as 14-26 s, such as 18-22 s, such as 20 s.
12. The system according to aspect 11, wherein the plurality of stimulation sites are located on the small intestine.
13. The system according to any of aspects 1-10, wherein the control unit is configured to apply the stimulation signal with a pulse separation or pulse train separation of 40-160 s, such as 60-140 s, such as 80-120 s, such as about 100 s.
14. The system according to aspect 13, wherein the plurality of stimulation sites are located on the large intestine.

### Aspect Group XI: Vibration Device_General

1. An implantable vibration device comprising
   a vibration generating unit (VGU) configured to cause the implantable vibration device to vibrate,
   wherein the vibration generating unit comprises at least one piezoelectric material, and
   a wireless energy receiver (R) configured to receive wireless energy to be used, directly or indirectly, by the vibration generating unit, and
   a casing enclosing at least the vibration generating unit.
2. The implantable vibration device according to aspect 1, wherein the casing further encloses the wireless energy receiver (R).
3. The implantable vibration device according to aspect 1, wherein the wireless energy receiver is provided outside the casing and coupled to the vibration generating unit through a lead.
4. The implantable vibration device according to any one of the preceding aspects, further comprising a rechargeable energy storage unit provided within the casing, for storing at least part of the received wireless energy.
5. The implantable vibration device according to any one of the preceding aspects, wherein the implantable vibration device (910) comprises an internal controller (CI).
6. The implantable vibration device of aspect 5, wherein the internal controller (CI) is configured to wirelessly receive vibration control data for controlling the vibration of the implantable vibration device.
7. The implantable vibration device according to aspect 6, wherein the internal controller (CI) is configured to receive the vibration control data wirelessly via the wireless energy receiver (R).
8. The implantable vibration device according to any one of aspects 5-7, wherein the casing further encloses the internal controller (Cr).
9. The implantable vibration device according to any one the preceding aspects, wherein the piezoelectric material is a ceramic piezoelectric material.
10. The implantable vibration device according to aspect 9, wherein the piezoelectric material is lead zirconate titanate, PZT.
11. The implantable vibration device according to aspect 9, wherein the piezoelectric material is barium titanate.
12. The implantable vibration device according to aspect 9, wherein the piezoelectric material is lead titanate.
13. The implantable vibration device according to any one aspect 1-8, wherein the piezoelectric material is a polymeric piezoelectric material.
14. The implantable vibration device according to aspect 13 wherein the polymeric piezoelectric material is polyvinylidene fluoride, PVDF.
15. The implantable vibration device according to any one of aspects 1-14, wherein the piezoelectric material is comprised in a piezoelectric motor.
16. The implantable vibration device according to aspect 14, wherein the piezoelectric motor is a piezoelectric inchworm motor.
17. The implantable vibration device according to aspect 14, wherein the piezoelectric motor is a piezoelectric inertial motor.
18. The medical device according to aspect 14, wherein the piezoelectric motor is a piezoelectric walk-drive motor.
19. The medical device according to any one of aspects 16-18, wherein the piezoelectric motor is a linear piezoelectric motor.
19. The medical device according to aspect 18, wherein the vibration generating unit is attached to the casing, so that vibrations generated by the vibration generating unit can travel to the casing.
20. The medical device according to any one of aspects 16-18, wherein the piezoelectric motor is a rotational piezoelectric motor.
21. The medical device according to aspect 20, wherein the vibration generating unit further comprises an weight configured to be eccentrically rotated by the rotational piezoelectric motor.
22. The implantable vibration device according to any one of the preceding aspects, wherein vibration generating unit is configured to cause the implantable vibration device to vibrate at a frequency in the range of 1-150 Hz, such as in the range of 35-150 Hz.
23. The implantable vibration device according to aspect any one of the preceding aspects, wherein vibration generating unit is configured to cause the implantable vibration device to vibrate at an amplitude of at least 1 mm.
24. The implantable vibration device of any one of the preceding aspects, wherein the implantable vibration device comprises an outer surface and a coating arranged on the outer surface.
25. The implantable vibration device according to aspect 24, wherein the coating comprises at least one layer of a biomaterial.
26. The implantable vibration device according to aspect 25, wherein the biomaterial comprises at least one drug or substance with one or more of the following characteristics: an antithrombotic, an antibacterial and an antiplatelet characteristic.
27. The implantable vibration device according to aspect 25 or 26, wherein the biomaterial is fibrin-based.
28. The implantable vibration device according to any one of aspects 25-27, further comprising a second coating arranged on the first coating.
29. The implantable vibration device according to aspect 28, wherein the second coating is of a different biomaterial than said first coating.
30. The implantable vibration device according to aspect 29, wherein the first coating comprises a layer of perfluorocarbon chemically attached to the surface, and wherein the second coating comprises a liquid perfluorocarbon layer.
31. The implantable vibration device according to any one of aspects 24 to 30, wherein the coating comprises a drug encapsulated in a porous material.
32. The implantable vibration device according to any one of aspects 24 to 31, wherein the surface comprises a metal.
33. The implantable vibration device according to aspect 32, wherein the metal comprises at least one of the following, titanium, cobalt, nickel, copper, zinc, zirconium, molybdenum, tin or lead.
34. The implantable vibration device according to any one of aspects 24 to 33, wherein the surface comprises a micro pattern.
35. The implantable vibration device according to aspect 34, further comprising a layer of a biomaterial coated on the micro pattern.
36. The medical device according to any one of aspects 1-35, wherein the vibration generating unit is substantially non-magnetic.
37. The medical device according to any one of aspects 1-36, wherein the vibration generating unit is substantially non-metallic.
38. The medical device according to any one of aspects 1-37, wherein the piezoelectric motor is a reversable piezoelectric motor.
   Aspect Group XII: eHealth_General
1. A medical system comprising an external device configured for communication with an implantable medical device when implanted in a patient, the medical device comprising a stimulation device according to any of aspect groups 460SE-472SE.

1. A system for treating a patient with hypertension, comprising:
a stimulation device comprising an electrode arrangement configured to be able to deliver an electric stimulation signal to at least one of: a wall portion of a renal artery and a parasympathetic nerve innervating the renal artery of the patient, to affect a vasomotor tone of a smooth muscle tissue of the renal artery;
an implantable source of energy configured to energize the electrode arrangement; and
a control unit operably connected to the stimulation device;
wherein the control unit is configured to control an operation of the stimulation device such that the electric stimulation signal causes a controlled vasodilation of the renal artery
a signal damping device configured to be arranged at the parasympathetic nerve, at a position between the stimulation device and the spinal cord, wherein the signal damping device comprises an electrode arrangement configured to deliver an electric damping signal to the parasympathetic nerve, and wherein the electric damping signal is configured to at least partly counteract the electrical stimulation signal generated by the stimulation device.

2. The system according to claim 1, wherein the electrode arrangement comprises a plurality of electrode elements, each of which being configured to engage and electrically stimulate the wall portion of the renal artery or the nerve innervating the renal artery.

3. The system according to claim 1, wherein the electrode arrangement is arranged on a surface portion of a support structure, and wherein the surface portion is configured to be placed on the wall portion of the renal artery or on the nerve innervating the renal artery.

4. The system according to claim 3, wherein the support structure comprises a cuff portion configured to be arranged at least partly around the wall portion of the renal artery or the nerve innervating the renal artery.

5. The system according to claim 4, wherein the electrode arrangement is arranged on an inner surface of the cuff.

6. The system according to claim 1, wherein the electrode arrangement is configured to electrically stimulate a sacral nerve.

7. The system according to claim 1, wherein the control unit is configured to generate a pulsed electrical stimulation signal for affecting the vasomotor tone of the smooth muscle tissue of the renal artery.

8. The system according to claim 7, wherein the electrical stimulation signal comprises a frequency of 30 Hz or less, such as 5-25 Hz, such as 10-20 Hz.

9. The system according to claim 7, wherein the electrical stimulation signal comprises a pulse width of 0.01-1 ms.

10. The system according to claim 7, wherein the electrical stimulation signal comprises a pulse amplitude of 1-15 mA.

11. The system according to claim 1, wherein the signal damping device further comprises a signal processing means configured to measure the electrical stimulation signal received at the signal damping device and generate the electric damping signal based on the received electrical stimulation signal.

12. The system according to claim 1, wherein the control unit is configured to be communicatively connected to a wireless remote control.

13. The system according to claim 1, wherein the control unit comprises an internal signal transmitter configured to receive and transmit communication signals from/to an external signal transmitter.

14. The system according to claim 1, further comprising a blood pressure sensor configured to generate a signal indicating a blood pressure of the patient.

15. The system according to claim 14, wherein the blood pressure sensor is configured to determine a local blood pressure in the renal artery.

16. The system according to claim 14, wherein the blood pressure sensor is configured to determine a systemic blood pressure.

17. The system according to claim 14, wherein the control unit is configured to receive the signal generated by the blood pressure sensor.

18. The system according to claim 17, wherein the control unit is configured to control the operation of the stimulation device based on the received signal.

19. A system for treating a patient with hypertension according to claim 1, wherein the stimulation device is adapted to stimulate the parasympathetic system, thereby causing vasodilation and lowering a blood pressure of the patient, wherein the stimulation device is further adapted to stimulate a parasympathetic nerve at least in a branch of a spinal cord dispatching number 10 and along the Coccygeal nerves originating at vertebrae S2-S4, preferably S4.

20. A system according to claim 19, wherein the vasomotor tone of the wall portion defines the flow in the renal artery and thereby indirect the blood pressure.
